SEQUENCE LISTING (16 sequences)

SEQ ID NO: 1 - DNA, 6978 bp, Homo sapiens
agggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgatgcccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttgtattaatatgtagcaaaggcttttccaatgggtgaataaaaacacattccattaagtcaagctgggagcagtggcatatacctatagtcccagctgcacaggaggctgagacaggaggattgcttgaagccaggaattggagatcagcctgggcaacacagcaagatcctatctcttaaaaaaagaaaaaaaaacctattaataataaaacagtataaacaaaagctaaataggtaaaatattttttctgaaataaaattattttttgagtctgatggaaatgtttaagtgcagtaggccagtgccagtgagaaaataaataacatcatacatgtttgtatgtgtttgcatcttgcttctactgaaagtttcagtgcaccccacttacttagaactcggtgacatgatgtactcctttatctgggacacagcacaaaagaggtatgcagtggggctgctctgacatgaaagtggaagttaaggaatctgggctcttatggggtccttgtgggccagcccttcaggcctattttactttcattttacatatagctctaattggtttgattatctcgttcccaaggcagtgggagatccccatttaaggaaagaaaaggggcctggcacagtggctcatgcctgtaatcccagcactttgggaggctgaggcaagtgtatcacctgaggtcaggagttcaagaccagcctggccaacatggcaaaatcccgtctctactaaaaatattaaaaaattggctgggcgtggtggttcgtgcctataatttcagctactcaggaggctgaggcaggagaatcgctgtaacctggggggtggaggttgcagtgagacgagatcatgccacttcactccagcctggccaacagagccatactccgtctcaaataaataaataaataaataaagggacttcaaacacatgaacagcagccaggggaagaatcaaaatcatattctgtcaagcaaactggaaaagtaccactgtgtgtaccaatagcctccccaccacagaccctgggagcatcgcctcatttatggtgtggtccagtcatccatgtgaaggatgagtttccaggaaaaggttattaaatattcactgtaacatactggaggaggtgaggaattgcataatacaatcttagaaaacttttttttcccctttctattttttgagacaggatctcactttggcactcaggctggaggacagtggtacaatcaaagctcatggcagcctcgacctccctgggcttgggcaatcctcccacaggtgtgcacctccatagctggctaatttgtgtattttttgtagagatggggtttcaccatgttgcccaggctggtctctaacacttaggctcaagtgatccacctgcctcgtcctcccaagatgctgggattacaggtgtgtgccacaggtgttcatcagaaagctttttctattatttttaccttcttgagtgggtagaacctcagccacatagaaaataaaatgttctggcatgacttatttagctctctggaattacaaagaaggaatgaggtgtgtaaaagagaacctgggtttttgaatcacaaatttagaatttaatcgaaactctgcctcttacttgtttgtagacactgacagtggcctcatgttttttttttttttaatctataaaatggagatatctaacatgttgagcctgggcccacaggcaaagcacaatcctgatgtgagaagtactcagttcatgacaactgttgttctcacatgcatagcataatttcatattcacattggaggacttctcccaaaatatggatgacgttccctactcaaccttgaacttaatcaaaatactcagtttacttaacttcgtattagattctgattccctggaaccatttatcgtgtgccttaccatgcttatattttacttgatcttttgcataccttctaaaactattttagccaatttaaaatttgacagtttgcattaaattataggtttacaatatgctttatccagctatacctgccccaaattctgacagatgcttttgccacctctaaaggaagacccatgttcatagtgatggagtttgtgtggactaaccatgcaaggttgccaaggaaaaatcgctttacgcttccaaggtacacactaagatgaaagtaattttagtccgtgtccagttggattcttggcacatagttatcttctgctagaacaaactaaaacagctacatgccagcaagggagaaaggggaaggaggggcaaagttttgaaatttcatgtaaatttatgctgttcaaaacgacgagttcatgactttgtgtatagagtaagaaatgccttttcttttttgagacagagtcttgctctgtcacccaggctggagtgcagtggcacgatctgggctcactacaacctccgcctcctgggttcaagcaattctctgcctcagcctcccgagtagctgggattacaggtgcctgccaccacacccggctaatttttgtatttttagtagagacggggtttcaccatcatggccaggctggtcttgaactcctgacctagtaatccacctgcctccgcctcccaaagtgctgggattacaggcgtgagccactgcacccagccagaaatgccttctaatctttggtttatcttaattagccaggacacttggagtgcatcccgaagtacctgatcagtggcccctttggaatgtgtaaaactcagctcacttatatccctgcatccgctacagagacagaatccaagctcatatgttccatcttctctggctgtatagtttaaggaatggaaggcaccagaacagatttattgaaatgtttattagctgaagatttatttagacagttgaggaaaacatcagcacccagcagtaaaattggctctcaaagattttcttctcctgtggaaagtcagacctctgaggccccatccaggtagaagtactagtgcaagaagggcctctgctgtccacttgtgtttctgtgatctgtgggaacattgttaacgccacatcttgacctcaaattgtttagctcctggccagacacggtggctcacacctgtaatcccagcactttgagaggctgaggcaggtggatcacctgaggttaggagttcgaggccagcctggtcaacatggtaaaaccccgcctctactaaaaatacaaaaattagctggccgtagtggcgcacgcctgttatcccagctactcgggaggctgaggcaggagaattgcttgaacctgggtggtggaggttgcagtgagccgagattacaccactgcactccagcctgggtgacaagagggaaactccattaaaaaaatgtaattcccgtgtctgccatcttaagtgtaaaggtggctaaattatatagaaaaataagacaatatcatttcccaattacattcctttcctaccgcactctatgatgctagctgagatttttccaaaagaaaatggcttaaataaaaccctaagagaaagaaaaactttaaatccctccaaagctcaaaagtaatagaaacagatgagtttggagtcaggatttctctgtaagattgcctaggctgtgtactgcacatctccaggtgccactgttgacagagattataactacaatgtgaagtgaatggtgccactgacagttatgcaaaccgtccagagcatagccacctgatcctgctgggattcctcttgccagtccatcagcagttccccttgaaagtttcaccaaacatcccttaaatctgccctctcctgcccgtccccagtggaggtcctcatcatttttcacctgcatttttgcaggagctttcttatatccaccttcctccttttctctcagcccatcatctagctacacagtctccagggtaagctttcagaaaggcaatctcttgtctgtaaaacctaagcaggaccaaggccaagtttcttagcctgaaaaatgtgcttttctgactgaactgttcaggcactgactctacatataattatgcttttctaccccctcacactcaacactttgactccagcaatcccaaatccccagatccctaagtgtgctgtgctattttcacgtggctctcagacttggccagtgctgtttccattttggtctttattccccacatctctgcctggggggtagattctaccctgaaaaatgttcttggcacagccttgcaaactcctcctccactcagcctctgcctggatgcccttgattgttccatgtcctcagcataccatgtttgtctttcccagcactgacctaccatgtgtcacccctgcttggctgtaccttccatgaggctaggactatgtgtctcctttgttgactgctgttgccctagcatcttgcacagttccttgcacacaattagagctctataaatgtcaaataaatgtgttataattatatgtttaagatagttgttcaaataaactctaaataaccccaa

SEQ ID NO: 2 - DNA, 7176 bp, Homo sapiens
agtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccaggtatgtagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgatgcccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttgtattaatatgtagcaaaggcttttccaatgggtgaataaaaacacattccattaagtcaagctgggagcagtggcatatacctatagtcccagctgcacaggaggctgagacaggaggattgcttgaagccaggaattggagatcagcctgggcaacacagcaagatcctatctcttaaaaaaagaaaaaaaaacctattaataataaaacagtataaacaaaagctaaataggtaaaatattttttctgaaataaaattattttttgagtctgatggaaatgtttaagtgcagtaggccagtgccagtgagaaaataaataacatcatacatgtttgtatgtgtttgcatcttgcttctactgaaagtttcagtgcaccccacttacttagaactcggtgacatgatgtactcctttatctgggacacagcacaaaagaggtatgcagtggggctgctctgacatgaaagtggaagttaaggaatctgggctcttatggggtccttgtgggccagcccttcaggcctattttactttcattttacatatagctctaattggtttgattatctcgttcccaaggcagtgggagatccccatttaaggaaagaaaaggggcctggcacagtggctcatgcctgtaatcccagcactttgggaggctgaggcaagtgtatcacctgaggtcaggagttcaagaccagcctggccaacatggcaaaatcccgtctctactaaaaatattaaaaaattggctgggcgtggtggttcgtgcctataatttcagctactcaggaggctgaggcaggagaatcgctgtaacctggggggtggaggttgcagtgagacgagatcatgccacttcactccagcctggccaacagagccatactccgtctcaaataaataaataaataaataaagggacttcaaacacatgaacagcagccaggggaagaatcaaaatcatattctgtcaagcaaactggaaaagtaccactgtgtgtaccaatagcctccccaccacagaccctgggagcatcgcctcatttatggtgtggtccagtcatccatgtgaaggatgagtttccaggaaaaggttattaaatattcactgtaacatactggaggaggtgaggaattgcataatacaatcttagaaaacttttttttcccctttctattttttgagacaggatctcactttggcactcaggctggaggacagtggtacaatcaaagctcatggcagcctcgacctccctgggcttgggcaatcctcccacaggtgtgcacctccatagctggctaatttgtgtattttttgtagagatggggtttcaccatgttgcccaggctggtctctaacacttaggctcaagtgatccacctgcctcgtcctcccaagatgctgggattacaggtgtgtgccacaggtgttcatcagaaagctttttctattatttttaccttcttgagtgggtagaacctcagccacatagaaaataaaatgttctggcatgacttatttagctctctggaattacaaagaaggaatgaggtgtgtaaaagagaacctgggtttttgaatcacaaatttagaatttaatcgaaactctgcctcttacttgtttgtagacactgacagtggcctcatgttttttttttttttaatctataaaatggagatatctaacatgttgagcctgggcccacaggcaaagcacaatcctgatgtgagaagtactcagttcatgacaactgttgttctcacatgcatagcataatttcatattcacattggaggacttctcccaaaatatggatgacgttccctactcaaccttgaacttaatcaaaatactcagtttacttaacttcgtattagattctgattccctggaaccatttatcgtgtgccttaccatgcttatattttacttgatcttttgcataccttctaaaactattttagccaatttaaaatttgacagtttgcattaaattataggtttacaatatgctttatccagctatacctgccccaaattctgacagatgcttttgccacctctaaaggaagacccatgttcatagtgatggagtttgtgtggactaaccatgcaaggttgccaaggaaaaatcgctttacgcttccaaggtacacactaagatgaaagtaattttagtccgtgtccagttggattcttggcacatagttatcttctgctagaacaaactaaaacagctacatgccagcaagggagaaaggggaaggaggggcaaagttttgaaatttcatgtaaatttatgctgttcaaaacgacgagttcatgactttgtgtatagagtaagaaatgccttttcttttttgagacagagtcttgctctgtcacccaggctggagtgcagtggcacgatctgggctcactacaacctccgcctcctgggttcaagcaattctctgcctcagcctcccgagtagctgggattacaggtgcctgccaccacacccggctaatttttgtatttttagtagagacggggtttcaccatcatggccaggctggtcttgaactcctgacctagtaatccacctgcctccgcctcccaaagtgctgggattacaggcgtgagccactgcacccagccagaaatgccttctaatctttggtttatcttaattagccaggacacttggagtgcatcccgaagtacctgatcagtggcccctttggaatgtgtaaaactcagctcacttatatccctgcatccgctacagagacagaatccaagctcatatgttccatcttctctggctgtatagtttaaggaatggaaggcaccagaacagatttattgaaatgtttattagctgaagatttatttagacagttgaggaaaacatcagcacccagcagtaaaattggctctcaaagattttcttctcctgtggaaagtcagacctctgaggccccatccaggtagaagtactagtgcaagaagggcctctgctgtccacttgtgtttctgtgatctgtgggaacattgttaacgccacatcttgacctcaaattgtttagctcctggccagacacggtggctcacacctgtaatcccagcactttgagaggctgaggcaggtggatcacctgaggttaggagttcgaggccagcctggtcaacatggtaaaaccccgcctctactaaaaatacaaaaattagctggccgtagtggcgcacgcctgttatcccagctactcgggaggctgaggcaggagaattgcttgaacctgggtggtggaggttgcagtgagccgagattacaccactgcactccagcctgggtgacaagagggaaactccattaaaaaaatgtaattcccgtgtctgccatcttaagtgtaaaggtggctaaattatatagaaaaataagacaatatcatttcccaattacattcctttcctaccgcactctatgatgctagctgagatttttccaaaagaaaatggcttaaataaaaccctaagagaaagaaaaactttaaatccctccaaagctcaaaagtaatagaaacagatgagtttggagtcaggatttctctgtaagattgcctaggctgtgtactgcacatctccaggtgccactgttgacagagattataactacaatgtgaagtgaatggtgccactgacagttatgcaaaccgtccagagcatagccacctgatcctgctgggattcctcttgccagtccatcagcagttccccttgaaagtttcaccaaacatcccttaaatctgccctctcctgcccgtccccagtggaggtcctcatcatttttcacctgcatttttgcaggagctttcttatatccaccttcctccttttctctcagcccatcatctagctacacagtctccagggtaagctttcagaaaggcaatctcttgtctgtaaaacctaagcaggaccaaggccaagtttcttagcctgaaaaatgtgcttttctgactgaactgttcaggcactgactctacatataattatgcttttctaccccctcacactcaacactttgactccagcaatcccaaatccccagatccctaagtgtgctgtgctattttcacgtggctctcagacttggccagtgctgtttccattttggtctttattccccacatctctgcctggggggtagattctaccctgaaaaatgttcttggcacagccttgcaaactcctcctccactcagcctctgcctggatgcccttgattgttccatgtcctcagcataccatgtttgtctttcccagcactgacctaccatgtgtcacccctgcttggctgtaccttccatgaggctaggactatgtgtctcctttgttgactgctgttgccctagcatcttgcacagttccttgcacacaattagagctctataaatgtcaaataaatgtgttataattatatgtttaagatagttgttcaaataaactctaaataaccccaac

SEQ ID NO: 3 - DNA, 980 bp, Homo sapiens
agtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccaggttaacgtggctcctgtggctgttccatccctgaggaaaagtgaggaccatgctctccaaacaggccatgtgctggactacctctgtttctgtctcctgggattccaatcagcaagtgagcaacgaagcaacccagacagtgtggttcataggatggctgggtaagtggctgtttgttttttccttactgtggatatgtatcagtgaaggaatctgtagaacattcttgatgggaacatttagtcatatcaagtcaataaattaatgtttaggctggg

SEQ ID NO: 4 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 5 - protein, 196 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSRYVVDLSVMTGLGKTGCTPTTACPSMSCWPQSSLKP

SEQ ID NO: 6 - protein, 171 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSRLTWLLWLFHP

SEQ ID NO: 7 - DNA, 109 bp, Artificial Sequence
  source: /note=“Description of Artificial Sequence Synthetic polynucleotide”
ttttggcaccaaaatcaacgggactttccaaaatgtcgtaacaactccgccccattgacgcaaatgggcggtaggcgtgtacggtgggaggtctatataagcagagctc

SEQ ID NO: 8 - DNA, 100 bp, Artificial Sequence
  source: /note=“Description of Artificial Sequence Synthetic polynucleotide”
ggtgcggcggcagccaatcagagcggcgcgctccgaaagtttccttttatggcgaggcggcggcggcggcggccctataaaaagcgaagcgcgcggcggg

SEQ ID NO: 9 - DNA, 332 bp, Artificial Sequence
  source: /note=“Description of Artificial Sequence Synthetic polynucleotide”
tacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 10 - DNA, 260 bp, Artificial Sequence
  source: /note=“Description of Artificial Sequence Synthetic polynucleotide”
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 11 - DNA, 4600 bp, Artificial Sequence
  source: /note=“Description of Artificial Sequence Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtttttggcaccaaaatcaacgggactttccaaaatgtcgtaacaactccgccccattgacgcaaatgggcggtaggcgtgtacggtgggaggtctatataagcagagctcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgactcgagccattgactagtacaaacaccattgtcacactccacacaaacaccattgtcacactccacacaaacaccattgtcacactccactgcagtcaggtctatcctgaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcctaggtttcttgagacctctacaagagttggagttgacacttggggtactttcttggtgtaacgaactaatagcctgaaaaaaagaagtcatgtgttttcagcaaggcaagaaactgtctaacatagtagataaaacagagaacacttggccggaatcaactaagatgttgctatgttccattcatcatattatctccatctgcagagtagtgggttagtggagggtagaaaacattctcctgaacaactagttaaacttggctttgagttccacctgtaccacttgcataatcttgggaaagtgagttgcctaattcagtgacattaataaatttattaatttcttctttcaataaaacctggagagagcttcatatgtatcagcatatgctaaacttgaaagatacaagtagaaaatggaaggaaatatatctgactcaatagggatagttcaagggttaaattaaaagtagtaaagtattataattaatctgacatggtacctaatatataataatcatgtattaagaatgccagtcaccattaaaagtcaatgtatgactttaatctactcgaggaaagaaactatgtcttgttcactgttattatctctaaaatccataatcagaagagcaccatgtgtatgagccacacaataaatatctactgtataatatgtctcttcttgtttttaaccttcatagataagactctattgaattgggacattagtccagcaagccattctgtctctgtctcttctatggagggaaaggtttaaccatcaaagactaggtgcatctcccaaacaacctgaatttaatattcaaatatgtatctaaattcatttgttacatttttgtgttcagcttacatattactttttgagcgacatctattcaaggcctactacttgctgctctacaaaatattgccatgctctatttgcccattaactatttcttaaccttcaagggacatgctcagttctgatataccaagatttggtatttaccctcccagcctacatacttccaatcttaagagaacaatttttagactacattcaaatatagacctctccaccccatcaactattttatctctcctctcctatctttcttgacaaagagtgattagaaatatgcaatgtattttcattcttaaaagttctatgctaggtggctcatatattaagtcttaaataattacaaattgataaaaaaaatcagtcaataaaaattacttacgcattctggaatttgtactctccaagctgctcaaaaagctcacaattttgtttgattaaattctgaggctcttccacaagaggtttaaattcatcgaactgaaacataaaagaattgtgttaatagtattatgcctcaggatcagatgcaggttgtttttcacccctctggtcagaatgatatcctttcttcttcctactcaaagccacatgaatagatgaatatcaatgccgctctgtcacaatacattcgaactatttatttggtgagcttgcatcatgtaagtggttaagaatattcaggctctggagttcaatgaccagtatttgagccaagtcatgtaactcattcttcagttgtgcaattttacttaccatctccacatttcagaatccttatccctaaaataggggcaaagatggcactcatctcatagagctgctctgtaaaccaggtaagctaatgaaattaaacagtgccttgcatataataaaagtttaataaatgatggctatgattgtacatattattattattagttcacctttccagttagattccaggctctccaaaatgagagattttacgttggtcattgtttgtgcatggtgcctggcatacagtagcttcttgaaagcaggaaaaatgagttttctctgattctgaatagttcagaaaccattacatatgctagtgggaaccctaaaaatcctccagaacagatttcctctcctaacttaatttcttgtaaaactattcctaaacatcctcaaaaagatttgaaaatattctaaagggataggttctataatttgccataagatactaattctagtacttgattaatcctggaatcaggttaactcactttacatctaagttaaatatcttctaattaacatttaaatttaatttttttttgttctcaggattgtgaaaaagagaaaaaaagatcaaaattttttagagattgctctattcagatctttctattctaactagtctaaattttcctaggtatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 4600 bp, Artificial Sequence
  source: /note=“Description of Artificial Sequence Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtggtgcggcggcagccaatcagagcggcgcgctccgaaagtttccttttatggcgaggcggcggcggcggcggccctataaaaagcgaagcgcgcggcgggcgggagcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgactcgagccattgactagtacaaacaccattgtcacactccacacaaacaccattgtcacactccacacaaacaccattgtcacactccactgcagtcaggtctatcctgaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcctaggtttcttgagacctctacaagagttggagttgacacttggggtactttcttggtgtaacgaactaatagcctgaaaaaaagaagtcatgtgttttcagcaaggcaagaaactgtctaacatagtagataaaacagagaacacttggccggaatcaactaagatgttgctatgttccattcatcatattatctccatctgcagagtagtgggttagtggagggtagaaaacattctcctgaacaactagttaaacttggctttgagttccacctgtaccacttgcataatcttgggaaagtgagttgcctaattcagtgacattaataaatttattaatttcttctttcaataaaacctggagagagcttcatatgtatcagcatatgctaaacttgaaagatacaagtagaaaatggaaggaaatatatctgactcaatagggatagttcaagggttaaattaaaagtagtaaagtattataattaatctgacatggtacctaatatataataatcatgtattaagaatgccagtcaccattaaaagtcaatgtatgactttaatctactcgaggaaagaaactatgtcttgttcactgttattatctctaaaatccataatcagaagagcaccatgtgtatgagccacacaataaatatctactgtataatatgtctcttcttgtttttaaccttcatagataagactctattgaattgggacattagtccagcaagccattctgtctctgtctcttctatggagggaaaggtttaaccatcaaagactaggtgcatctcccaaacaacctgaatttaatattcaaatatgtatctaaattcatttgttacatttttgtgttcagcttacatattactttttgagcgacatctattcaaggcctactacttgctgctctacaaaatattgccatgctctatttgcccattaactatttcttaaccttcaagggacatgctcagttctgatataccaagatttggtatttaccctcccagcctacatacttccaatcttaagagaacaatttttagactacattcaaatatagacctctccaccccatcaactattttatctctcctctcctatctttcttgacaaagagtgattagaaatatgcaatgtattttcattcttaaaagttctatgctaggtggctcatatattaagtcttaaataattacaaattgataaaaaaaatcagtcaataaaaattacttacgcattctggaatttgtactctccaagctgctcaaaaagctcacaattttgtttgattaaattctgaggctcttccacaagaggtttaaattcatcgaactgaaacataaaagaattgtgttaatagtattatgcctcaggatcagatgcaggttgtttttcacccctctggtcagaatgatatcctttcttcttcctactcaaagccacatgaatagatgaatatcaatgccgctctgtcacaatacattcgaactatttatttggtgagcttgcatcatgtaagtggttaagaatattcaggctctggagttcaatgaccagtatttgagccaagtcatgtaactcattcttcagttgtgcaattttacttaccatctccacatttcagaatccttatccctaaaataggggcaaagatggcactcatctcatagagctgctctgtaaaccaggtaagctaatgaaattaaacagtgccttgcatataataaaagtttaataaatgatggctatgattgtacatattattattattagttcacctttccagttagattccaggctctccaaaatgagagattttacgttggtcattgtttgtgcatggtgcctggcatacagtagcttcttgaaagcaggaaaaatgagttttctctgattctgaatagttcagaaaccattacatatgctagtgggaaccctaaaaatcctccagaacagatttcctctcctaacttaatttcttgtaaaactattcctaaacatcctcaaaaagatttgaaaatattctaaagggataggttctataatttgccataagatactaattctagtacttgattaatcctggaatcaggttaactcactttacatctaagttaaatatcttctaattaacatttaaatttaatttttttttgttctcaggattgtgaaaaagagaaaaaaagatcaaaattttttagagattgctctattcagatctttctattctaactagtctaaattttgtcctaggtatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - DNA, 4600 bp, Artificial Sequence
  source: /note=“Description of Artificial Sequence Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgttacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgactcgagccattgactagtacaaacaccattgtcacactccacacaaacaccattgtcacactccacacaaacaccattgtcacactccactgcagtcaggtctatcctgaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcctagtctcctgaacaactagttaaacttggctttgagttccacctgtaccacttgcataatcttgggaaagtgagttgcctaattcagtgacattaataaatttattaatttcttctttcaataaaacctggagagagcttcatatgtatcagcatatgctaaacttgaaagatacaagtagaaaatggaaggaaatatatctgactcaatagggatagttcaagggttaaattaaaagtagtaaagtattataattaatctgacatggtacctaatatataataatcatgtattaagaatgccagtcaccattaaaagtcaatgtatgactttaatctactcgaggaaagaaactatgtcttgttcactgttattatctctaaaatccataatcagaagagcaccatgtgtatgagccacacaataaatatctactgtataatatgtctcttcttgtttttaaccttcatagataagactctattgaattgggacattagtccagcaagccattctgtctctgtctcttctatggagggaaaggtttaaccatcaaagactaggtgcatctcccaaacaacctgaatttaatattcaaatatgtatctaaattcatttgttacatttttgtgttcagcttacatattactttttgagcgacatctattcaaggcctactacttgctgctctacaaaatattgccatgctctatttgcccattaactatttcttaaccttcaagggacatgctcagttctgatataccaagatttggtatttaccctcccagcctacatacttccaatcttaagagaacaatttttagactacattcaaatatagacctctccaccccatcaactattttatctctcctctcctatctttcttgacaaagagtgattagaaatatgcaatgtattttcattcttaaaagttctatgctaggtggctcatatattaagtcttaaataattacaaattgataaaaaaaatcagtcaataaaaattacttacgcattctggaatttgtactctccaagctgctcaaaaagctcacaattttgtttgattaaattctgaggctcttccacaagaggtttaaattcatcgaactgaaacataaaagaattgtgttaatagtattatgcctcaggatcagatgcaggttgtttttcacccctctggtcagaatgatatcctttcttcttcctactcaaagccacatgaatagatgaatatcaatgccgctctgtcacaatacattcgaactatttatttggtgagcttgcatcatgtaagtggttaagaatattcaggctctggagttcaatgaccagtatttgagccaagtcatgtaactcattcttcagttgtgcaattttacttaccatctccacatttcagaatccttatccctaaaataggggcaaagatggcactcatctcatagagctgctctgtaaaccaggtaagctaatgaaattaaacagtgccttgcatataataaaagtttaataaatgatggctatgattgtacatattattattattagttcacctttccagttagattccaggctctccaaaatgagagattttacgttggtcattgtttgtgcatggtgcctggcatacagtagcttcttgaaagcaggaaaaatgagttttctctgattctgaatagttcagaaaccattacatatgctagtgggaaccctaaaaatcctccagaacagatttcctctcctaacttaatttcttgtaaaactattcctaaacatcctcaaaaagatttgaaaatattctaaagggataggttctataatttgccataagatactaattctagtacttgattaatcctggaatcaggttaactcactttacatctaagttaaatatcttctaattaacatttaaatttaatttttttttgttctcaggattgtgaaaaagagaaaaaaagatcaaaattttttagagattgctctattcagatctttctattctaactagtctaaattttgtcctaggtatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 14 - DNA, 4600 bp, Artificial Sequence
  source: /note=“Description of Artificial Sequence Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgactcgagccattgactagtacaaacaccattgtcacactccacacaaacaccattgtcacactccacacaaacaccattgtcacactccactgcagtcaggtctatcctgaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcctaggatgttgctatgttccattcatcatattatctccatctgcagagtagtgggttagtggagggtagaaaacattctcctgaacaactagttaaacttggctttgagttccacctgtaccacttgcataatcttgggaaagtgagttgcctaattcagtgacattaataaatttattaatttcttctttcaataaaacctggagagagcttcatatgtatcagcatatgctaaacttgaaagatacaagtagaaaatggaaggaaatatatctgactcaatagggatagttcaagggttaaattaaaagtagtaaagtattataattaatctgacatggtacctaatatataataatcatgtattaagaatgccagtcaccattaaaagtcaatgtatgactttaatctactcgaggaaagaaactatgtcttgttcactgttattatctctaaaatccataatcagaagagcaccatgtgtatgagccacacaataaatatctactgtataatatgtctcttcttgtttttaaccttcatagataagactctattgaattgggacattagtccagcaagccattctgtctctgtctcttctatggagggaaaggtttaaccatcaaagactaggtgcatctcccaaacaacctgaatttaatattcaaatatgtatctaaattcatttgttacatttttgtgttcagcttacatattactttttgagcgacatctattcaaggcctactacttgctgctctacaaaatattgccatgctctatttgcccattaactatttcttaaccttcaagggacatgctcagttctgatataccaagatttggtatttaccctcccagcctacatacttccaatcttaagagaacaatttttagactacattcaaatatagacctctccaccccatcaactattttatctctcctctcctatctttcttgacaaagagtgattagaaatatgcaatgtattttcattcttaaaagttctatgctaggtggctcatatattaagtcttaaataattacaaattgataaaaaaaatcagtcaataaaaattacttacgcattctggaatttgtactctccaagctgctcaaaaagctcacaattttgtttgattaaattctgaggctcttccacaagaggtttaaattcatcgaactgaaacataaaagaattgtgttaatagtattatgcctcaggatcagatgcaggttgtttttcacccctctggtcagaatgatatcctttcttcttcctactcaaagccacatgaatagatgaatatcaatgccgctctgtcacaatacattcgaactatttatttggtgagcttgcatcatgtaagtggttaagaatattcaggctctggagttcaatgaccagtatttgagccaagtcatgtaactcattcttcagttgtgcaattttacttaccatctccacatttcagaatccttatccctaaaataggggcaaagatggcactcatctcatagagctgctctgtaaaccaggtaagctaatgaaattaaacagtgccttgcatataataaaagtttaataaatgatggctatgattgtacatattattattattagttcacctttccagttagattccaggctctccaaaatgagagattttacgttggtcattgtttgtgcatggtgcctggcatacagtagcttcttgaaagcaggaaaaatgagttttctctgattctgaatagttcagaaaccattacatatgctagtgggaaccctaaaaatcctccagaacagatttcctctcctaacttaatttcttgtaaaactattcctaaacatcctcaaaaagatttgaaaatattctaaagggataggttctataatttgccataagatactaattctagtacttgattaatcctggaatcaggttaactcactttacatctaagttaaatatcttctaattaacatttaaatttaatttttttttgttctcaggattgtgaaaaagagaaaaaaagatcaaaattttttagagattgctctattcagatctttctattctaactagtctaaattttgtcctaggtatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - protein, 743 aa, Artificial Sequence
  source: /note=“Description of Artificial Sequence Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 2232 bp, Artificial Sequence
  source: /note=“Description of Artificial Sequence Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

(19) United States
(12) Patent Application Publication
(10) Pub. No.: US 2022/0308072 A1
(43) Pub. Date: Sep. 29, 2022

(54) HIGH-SENSITIVITY IMMUNOASSAY FOR THE DETECTION OF FRATAXIN IN BIOFLUIDS

(71) Applicant: VOYAGER THERAPEUTICS, INC., Cambridge, MA (US)

(72) Inventors: Steven M. HERSCH, Cambridge, MA (US); Shawn CIOTTI, Reading, MA (US)

(21) Appl. No.: 17/634,385

(22) PCT Filed: Aug. 11, 2020
(86) PCT No.: PCT/US2020/045687

§ 371 Date: Feb. 10, 2022

Related U.S. Application Data
(60) Provisional application No. 62/885,413, filed on Aug. 12, 2019.
(60) Provisional application No. 62/935,442, filed on Nov. 14, 2019.
(60) Provisional application No. 63/035,390, filed on Jun. 5, 2020.

(51) Int. Cl. G01N 33/68; G01N 33/543

(52) U.S. Cl. CPC G01N 33/6893; G01N 33/54326; G01N 2800/2835; A61K 48/00

(57) ABSTRACT

Disclosed herein are methods for assaying the concentration of frataxin in biofluids, and kits, reagents, and uses thereof.

### CROSS REFERENCE TO RELATED APPLICATIONS

The present application claims the benefit of priority to U.S. Provisional Patent Application No. 62/885,413, filed Aug. 12, 2019; U.S. Provisional Patent Application No. 62/935,442, filed Nov. 14, 2019; and U.S. Provisional Patent Application No. 63/035,390, filed Jun. 5, 2020; the contents of which are incorporated by reference herein in their entirety.

### REFERENCE TO SEQUENCE LISTING

The instant application contains a Sequence Listing which has been filed electronically in ASCII format and is hereby incorporated by reference in its entirety. Said ASCII copy, created on Aug. 11, 2020, is named 1081PCTSEQLST.txt and is 61,779 bytes in size.

### FIELD OF THE DISCLOSURE

The present disclosure presents improved immunoassays and related methods to detect and quantify low concentrations of frataxin found in biofluids, including circulating biofluids, as well as corresponding methods of using the immunoassays in detecting, monitoring, and treating disorders.

### BACKGROUND OF THE DISCLOSURE

### Friedreich's Ataxia

Friedreich's Ataxia (FA), as first described by German physician Nikolas Friedreich in the 1860s, is an autosomal recessive inherited disease that causes progressive damage to the nervous system. See Parkinson et al., Journal of Neurochemistry, 2013, 126 (Suppl. 1), 103-117, the contents of which are herein incorporated by reference in their entirety. Onset usually occurs at puberty, and always by age 25. See Campuzano, et al., Science, 271.5254 (Mar. 8, 1996): 1423, the contents of which are herein incorporated by reference in their entirety. FA results from the degeneration of nervous tissue in the spinal cord due to reduced expression of the mitochondrial protein frataxin, (FXN) in sensory neurons that are essential (through connections with the cerebellum) for directing muscle movement of the arms and legs. See Koeppen, Arnulf; J Neurol Sci., 2011, Apr. 15; 303(1-2): 1-12, the contents of which are herein incorporated by reference in their entirety. The spinal cord becomes thinner and peripheral nerve cells lose some of their myelin sheath, which is the insulating covering on some nerve cells that helps conduct nerve impulses. Initial symptoms include poor coordination such as gait disturbance, poor balance, leg weakness, decreased walking, impaired coordination, dysarthria, nystagmus, impaired sensation, kyphoscoliosis, and foot deformities. See Parkinson et al., Journal of Neurochemistry, 2013, 126 (Suppl. 1), 103-117, the contents of which are herein incorporated by reference in their entirety. FA is also associated with scoliosis, heart disease, and diabetes. Symptoms further include progressive ataxia, weakness, fatigue, and impaired speech, vision, and hearing, causing profound functional impairment and increasing assistance needs. The disease generally progresses until a wheelchair is required for mobility. Incidence of FA among Caucasian populations is between about 1 in 20,000 and about 1 in 50,000, with a deduced carrier frequency of about 1 in 120 in European populations. See Nageshwaran and Festenstein, Frontiers in Neurology, Vol. 6, Art. 262 (2015); Campuzano, et al., Science, 271.5254 (Mar. 8, 1996): 1423, the contents of each of which are herein incorporated by reference in their entirety.

The expansion of an intronic GAA triplet repeat in the FXN gene is the genetic cause of reduced expression of frataxin resulting in FA. See Parkinson et al., Journal of Neurochemistry, 2013, 126 (Suppl. 1), 103-117, the contents of which are herein incorporated by reference in their entirety. Over time the deficiency causes the aforementioned symptoms, as well as frequent fatigue due to effects on cellular metabolism.

Sclerosis and degeneration are most frequent in dorsal root ganglia, spinocerebellar tracts, lateral corticospinal tracts, and posterior columns. See Sandi et al., Frontiers in Genetics, Vol. 5, Art. 165 (June 2014), the contents of which are herein incorporated by reference in their entirety.

Progressive destruction of dorsal root ganglia causes thinning of dorsal roots, degeneration of dorsal columns, trans-synaptic atrophy of nerve cells in Clarke's column and dorsal spinocerebellar fibers, atrophy of gracile and cuneate nuclei, and neuropathy of sensory nerves. See Koeppen, Arnulf; J Neurol Sci., 2011, Apr. 15; 303(1-2): 1-12, the contents of which are herein incorporated by reference in their entirety. The lesion of the dentate nucleus consists of progressive and selective atrophy of large glutamatergic neurons and grumose degeneration of corticonuclear synaptic terminals that contain gamma-aminobutyric acid (GABA). Small GABA-ergic neurons and their projection fibers in the dentato-olivary tract survive. Atrophy of Betz cells and corticospinal tracts constitute a second lesion. Currently, no effective treatments exist for FA and patients are most often simply monitored for symptom management. Patients are dependent on mobility aids by their teens to early 20s and cardiomyopathy often causes early death. Restoration of frataxin in affected tissues is a key therapeutic goal.

### Difficulty of Measuring Frataxin

Typically, frataxin measurement uses samples including muscle cells, buccal cells, and purified blood cell populations. See Guo, et al., Anal. Chem. 2018, 90, 2216-2223, the contents of which are herein incorporated by reference in their entirety. Measurements in cellular tissues could enable the assessment of target engagement for frataxin modulating treatments. However, cellular-level measurements may have limited utility in examining the amount of frataxin in tissues that cannot be readily biopsied such as the heart, central and peripheral nervous systems, and other relevant tissues. Current assays use either ELISA or laminar flow dipstick methods, which have lower limits of detection in the picogram range. Yet, none of the currently available assays have sufficient sensitivity to detect frataxin in circulating biofluids including plasma, serum, and cerebrospinal fluid (CSF). High detection sensitivity to accurately detect small differences in frataxin levels may be beneficial for potential use as a biomarker assay because 50% of normal frataxin expression levels are clinically relevant based on the lack of phenotype in heterozygous carriers of FXN mutations.

Measurement of frataxin in CSF could help assess whether brain frataxin levels can be increased by a treatment and may serve as a useful prognostic and/or response biomarker. Measurement in plasma or serum could provide an integrated whole-body measure for frataxin (including for tissues, like heart tissue, that are not readily biopsied) and a potential prognostic or response biomarker.

While there are many frataxin assays capable of detection in tissues, no options are readily available for detecting frataxin in circulating biofluids such as plasma, serum, and CSF. A means to quantify frataxin levels in a circulating biofluid, e.g., fractionated blood or CSF, would allow circulating frataxin to be measured directly and provide information about CNS or systemic levels of frataxin, as compared to current tissue-based standards using peripheral blood mononuclear cells (PBMC), purified erythrocytes, buccal cells, or muscle biopsy, which measure local levels of frataxin. With successful detection, frataxin levels in these biofluids could become potent biomarkers, and may be used as part of treatment regimens to determine and/or monitor appropriate therapies, for example gene therapies that restore normal frataxin levels.

### SUMMARY OF THE DISCLOSURE

In certain embodiments, the disclosure herein provides a method of determining the amount of frataxin protein in a biofluid. In some embodiments, the biofluid may be cerebrospinal fluid, blood, or a blood component. In certain embodiments, the blood component is plasma or serum.

In some embodiments, the method comprises performing an assay on a collected sample of a biofluid to determine the concentration of frataxin protein present in the collected sample; wherein the limit of detection of frataxin of the assay is about 2.0 pg/mL or less. In some embodiments, the limit of detection of frataxin is about 1.5 pg/mL or less. In some embodiments, the limit of detection of frataxin is about 1.0 pg/mL or less. In some embodiments, the limit of detection of frataxin is about 0.5 pg/mL or less.

In certain embodiments, the assay comprises a capture agent that specifically binds to frataxin or a fragment of frataxin, and a detection agent that specifically binds to frataxin or a fragment of frataxin. In some embodiments, the capture agent and/or the detection agent comprises an antibody or fragment thereof. In some embodiments, the capture agent and/or the detection agent comprises an anti-frataxin antibody or a frataxin-binding fragment thereof. In some embodiments, the capture agent and/or the detection agent comprises an antibody selected from the group consisting of Ab-1, Ab-2, Ab-3, Ab-4, Ab-5, and Ab-6. In some embodiments, the capture agent is Ab-1 and the detector agent is Ab-3.

In certain embodiments, the assay comprises the steps of i) incubating a substrate with the capture agent, ii) incubating the substrate with the collected sample and the detection agent and iii) incubating the substrate with a substance capable of engaging with the detection agent and creating a signal, wherein the signal is visible, detectable and/or quantifiable. In some embodiments, the signal is a fluorescence. In some embodiments, the incubating step of ii) occurs for about 10-150 minutes and the incubating step of iii) occurs for about 1-30 minutes. In some embodiments, the incubating step of ii) occurs for about 35 minutes and the incubating step of iii) occurs for about 5 minutes. In some embodiments, the incubating step of ii) occurs for about 75 minutes and the incubating step of iii) occurs for about 5 minutes. In some embodiments, the incubating step of ii) occurs for about 120 minutes and the incubating step of iii) occurs for about 30 minutes. In some embodiments, the incubating step of ii) occurs for about 120 minutes and the incubating step of iii) occurs for about 5 minutes.

In certain embodiments, the assay comprises assay beads and helper beads. In some embodiments, the ratio of helper beads to assay beads is about 1:5 to about 5:1. In some embodiments, the assay comprises a greater number of helper beads than assay beads. In some embodiments, the ratio of helper beads to assay beads is about 1:1, about 1.5:1, or about 2:1.

In certain embodiments, the disclosure herein provides a method of determining the efficacy of a treatment for increasing frataxin levels comprising the steps of a) assessing a baseline of frataxin in a first sample of a biofluid of a subject, b) treating the subject with a treatment that results in increased frataxin levels in the subject, c) assessing a subsequent concentration of frataxin in a second sample of a biofluid of the subject, d) comparing the baseline of frataxin to the subsequent concentration of frataxin; and e) determining an efficacy of the treatment for increasing frataxin levels. In certain embodiments, the treatment that results in increased frataxin levels in the subject is a frataxin gene therapy.

In some embodiments, the method further comprises determining the amount of neurofilament light chain in a sample of a biofluid. In some embodiments, the amount of frataxin protein and the amount of neurofilament light chain are determined from a single sample of a biofluid.

In some embodiments, the biofluid may be cerebrospinal fluid (CSF), blood, or a blood component. In certain embodiments, the biofluid is CSF. In certain embodiments, the blood component is plasma. In certain embodiments, the blood component is serum.

In certain embodiments, the disclosure herein provides a method of diagnosing Friedreich's Ataxia in a subject comprising the steps of a) performing an assay on a sample of a biofluid from the subject to assess the concentration of frataxin protein present in the sample, and b) diagnosing the subject with Friedreich's Ataxia if the concentration of frataxin protein present in the sample is lower than the limit of detection, e.g., if frataxin is not detectable in the biofluid sample. In certain embodiments, the disclosure herein provides a method of diagnosing Friedreich's Ataxia in a subject comprising the steps of a) performing an assay on a sample of a biofluid from the subject to determine the concentration of frataxin protein present in the sample, and b) diagnosing the subject with Friedreich's Ataxia if the concentration of frataxin protein present in the sample is at or just above the limit of detection, e.g., only minimally detected in the sample. In some embodiments, the limit of detection is a threshold described herein, e.g., about 0.5-2.0 pg/mL, e.g., only minimally detected in the sample.

In certain embodiments, the disclosure herein provides a method of diagnosing Friedreich's Ataxia in a subject, comprising the steps of performing an assay on a sample, e.g., a serum sample, from the subject to assess the concentration of frataxin protein present in the sample, wherein a limit of detection of frataxin of the assay is about 2.0 pg/mL or less, and wherein the assay comprises a capture agent that specifically binds to frataxin or a fragment of frataxin, and a detection agent that specifically binds to frataxin or a fragment of frataxin, and diagnosing the subject with Friedreich's Ataxia if the concentration of frataxin protein present in the sample from the subject is below 10 pg/mL, below 5 pg/mL, below 2 pg/mL, or less. In some embodiments, the subject is diagnosed with Friedreich's Ataxia if the concentration of frataxin protein present in the sample from the subject is below 1 pg/mL. In some embodiments, the subject is diagnosed with Friedreich's Ataxia if frataxin protein cannot be detected in the sample from the subject (e.g., the concentration of frataxin protein is below the level of detection).

In certain embodiments, the disclosure herein provides a method of diagnosing Friedreich's Ataxia in a subject, comprising the steps of performing an assay on a CSF sample from the subject to assess the concentration of frataxin protein present in the sample, wherein a limit of detection of frataxin of the assay is about 2.0 pg/mL or less, and wherein the assay comprises a capture agent that specifically binds to frataxin or a fragment of frataxin, and a detection agent that specifically binds to frataxin or a fragment of frataxin, and diagnosing the subject with Friedreich's Ataxia if the concentration of frataxin protein present in the CSF sample from the subject is below 5 pg/mL, below 2 pg/mL, or less. In some embodiments, the subject is diagnosed with Friedreich's Ataxia if the concentration of frataxin protein present in the CSF sample from the subject is below 1 pg/mL. In some embodiments, the subject is diagnosed with Friedreich's Ataxia if frataxin protein cannot be detected in the sample from the subject (e.g., the concentration of frataxin protein is below the level of detection).

In certain embodiments, the disclosure herein provides a method of diagnosing Friedreich's Ataxia in a subject suspected of having Friedreich's Ataxia comprising the steps of a) performing an assay on a sample of a biofluid from the subject suspected of having Friedreich's Ataxia to assess the concentration of frataxin protein present in the sample, b) performing an assay on a sample of a biofluid from a healthy subject to determine the concentration of frataxin protein present in the sample, and c) diagnosing the subject with Friedreich's Ataxia if the concentration of frataxin protein present in the sample from the subject suspected of having Friedreich's Ataxia is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in the sample from the healthy subject.

In certain embodiments, the disclosure herein provides a method of diagnosing Friedreich's Ataxia in a subject, comprising the steps of a) performing an assay on a first sample of a biofluid from the subject to assess the concentration of frataxin protein present in the sample, b) performing an assay on a second sample of a biofluid from the subject to assess the concentration of neurofilament light chain present in the sample, and c) diagnosing the subject with Friedreich's Ataxia if the concentration of frataxin protein present in the first sample is lower than the limit of detection and if neurofilament light chain is detected in the second sample at a concentration of at least 20 pg/mL in serum or at least 1200 pg/mL in CSF.

In certain embodiments, the disclosure herein provides a method of diagnosing Friedreich's Ataxia in a subject suspected of having Friedreich's Ataxia, comprising the steps of a) performing an assay on a first sample of a biofluid from the subject suspected of having Friedreich's Ataxia to assess the concentration of frataxin protein present in the sample, b) performing an assay on a second sample of a biofluid from the subject suspected of having Friedreich's Ataxia to assess the concentration of neurofilament light chain present in the sample, c) performing an assay on a first sample of a biofluid from an age-matched healthy subject to assess the concentration of frataxin protein present in the sample, d) performing an assay on a second sample of a biofluid from the age-matched healthy subject to assess the concentration of neurofilament light chain present in the sample, and e) diagnosing the subject with Friedreich's Ataxia if the concentration of frataxin protein present in the first sample from the subject suspected of having Friedreich's Ataxia is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in the first sample from the age-matched healthy subject and the concentration of neurofilament light chain present in the second sample from the subject suspected of having Friedreich's Ataxia is greater than or equal to the concentration of neurofilament light chain present in the second sample from the age-matched healthy subject.

In certain embodiments, the disclosure herein provides a method of diagnosing Friedreich's Ataxia in a subject suspected of having Friedreich's Ataxia, comprising the steps of: performing an assay on a first sample of serum from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and performing an assay on a second sample of serum from the subject suspected of having Friedreich's Ataxia to assess the concentration of neurofilament light chain present in the sample; and diagnosing the subject suspected of having Friedreich's Ataxia with Friedreich's Ataxia if the concentration of frataxin protein present in the first sample of serum is below 10 pg/mL and the concentration of neurofilament light chain present in the second sample of serum is at least 20 pg/mL. In some embodiments, the concentration of frataxin protein present in the first sample of serum is below 5 pg/mL, below 2 pg/mL, or less. In some embodiments, the concentration of frataxin protein present in the first sample of serum is below 1 pg/mL. In some embodiments, frataxin protein cannot be detected in the first sample of serum from the subject diagnosed with Friedreich's Ataxia (e.g., concentration of frataxin protein is below the level of detection).

In certain embodiments, the disclosure herein provides a method of diagnosing Friedreich's Ataxia in a subject suspected of having Friedreich's Ataxia, comprising the steps of: performing an assay on a first sample of CSF from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and performing an assay on a second sample of CSF from the subject suspected of having Friedreich's Ataxia to assess the concentration of neurofilament light chain present in the sample; and diagnosing the subject suspected of having Friedreich's Ataxia with Friedreich's Ataxia if the concentration of frataxin protein present in the first sample of CSF is below 5 pg/mL and the concentration of neurofilament light chain present in the second sample of CSF is at least 1200 pg/mL. In some embodiments, the concentration of frataxin protein present in the first sample of CSF is below 2 pg/mL or less. In some embodiments, the concentration of frataxin protein present in the first sample of CSF is below 1 pg/mL. In some embodiments, frataxin protein cannot be detected in the first sample of CSF from the subject diagnosed with Friedreich's Ataxia (e.g., concentration of frataxin protein is below the level of detection).

In some embodiments, the first sample and the second sample of a biofluid from the subject suspected of having Friedreich's Ataxia are the same sample or each is a portion of an initial sample and/or the first sample and the second sample of a biofluid from the healthy subject are the same sample or each is a portion of an initial sample.

In some embodiments, the limit of detection of frataxin is about 2.0 pg/mL or less. In some embodiments, the limit of detection is about 1.5 pg/mL or less. In some embodiments, the limit of detection is about 1.0 pg/mL or less. In some embodiments, the limit of detection is 0.5 pg/mL or less. In certain embodiments, the assay comprises a capture agent that specifically binds to frataxin or a fragment of frataxin, and a detection agent that specifically binds to frataxin or a fragment of frataxin.

In certain embodiments, the disclosure herein provides a method of treating Friedreich's Ataxia in a subject, comprising the steps of a) determining whether a subject has frataxin in a biofluid sample using an assay having a limit of detection of frataxin of about 2.0 pg/mL or less and b) administering a suitable treatment to a subject lacking detectable frataxin in the biofluid sample. In certain embodiments, the disclosure herein provides a method of treating Friedreich's Ataxia in a subject, comprising the steps of a) determining whether a subject has frataxin in a biofluid sample using an assay having a limit of detection of frataxin of about 2.0 pg/mL or less and b) administering a suitable treatment to a subject with deficient frataxin (e.g., less than 20% compared to the frataxin levels of a healthy individual) in the biofluid sample.

In certain embodiments, the disclosure herein provides a method of treating Friedreich's Ataxia in a subject, comprising the steps of a) determining the concentration of frataxin in a serum sample from the subject using an assay having a limit of detection of frataxin of about 2.0 pg/mL or less, wherein the assay comprises a capture agent that specifically binds to frataxin or a fragment of frataxin, and a detection agent that specifically binds to frataxin or a fragment of frataxin, and b) administering a suitable treatment to the subject if the concentration of frataxin protein present in the serum sample is below 10 pg/mL. In some embodiments, the subject is administered a suitable treatment if the concentration of frataxin protein present in the serum sample is below 5 pg/mL, below 2 pg/mL, or less. In some embodiments, the subject is administered a suitable treatment if the concentration of frataxin protein present in the serum sample is below 1 pg/mL. In some embodiments, the subject is administered a suitable treatment if frataxin protein cannot be detected in the serum sample (e.g., concentration of frataxin protein is below the level of detection). In some embodiments, the method further comprises the step of determining the concentration of neurofilament light chain in a serum sample from the subject. In some embodiments, the sample from which neurofilament light chain concentration is determined is the same serum sample or part of the same initial serum sample as the sample from which the concentration of frataxin is determined. In some embodiments, the concentration of neurofilament light chain prior to treatment is at least 20 pg/mL. In some embodiments, the subject is administered a suitable treatment if the concentration of frataxin protein present in the serum sample is below 10 pg/mL, below 5 pg/mL, below 2 pg/mL, below 1 pg/mL, or less (e.g., below level of detection), and if the concentration of neurofilament light chain in the serum sample is at least 20 pg/mL.

In certain embodiments, the disclosure herein provides a method of treating Friedreich's Ataxia in a subject, comprising the steps of a) determining the concentration of frataxin in a CSF sample from the subject using an assay having a limit of detection of frataxin of about 2.0 pg/mL, or less, wherein the assay comprises a capture agent that specifically binds to frataxin or a fragment of frataxin, and a detection agent that specifically binds to frataxin or a fragment of frataxin, and b) administering a suitable treatment to the subject if the concentration of frataxin protein present in the CSF sample is below 5 pg/mL. In some embodiments, the subject is administered a suitable treatment if the concentration of frataxin protein present in the CSF sample below 2 pg/mL or less. In some embodiments, the subject is administered a suitable treatment if the concentration of frataxin protein present in the CSF sample is below 1 pg/mL. In some embodiments, the subject is administered a suitable treatment if frataxin protein cannot be detected in the CSF sample (e.g., concentration of frataxin protein is below the level of detection). In some embodiments, the method further comprises the step of determining the concentration of neurofilament light chain in a CSF sample from the subject. In some embodiments, the sample from which neurofilament light chain concentration is determined is the same CSF sample or a part of the same initial CSF sample as the sample from which frataxin concentration is determined. In some embodiments, the concentration of neurofilament light chain prior to treatment is at least 1200 pg/mL. In some embodiments, the subject is administered a suitable treatment if the concentration of frataxin protein present in the CSF sample is below 5 pg/mL, below 2 pg/mL, below 1 pg/mL, or less (e.g., below level of detection), and if the concentration of neurofilament light chain in the CSF sample is at least 1200 pg/mL.

In certain embodiments, the assessing steps of the method comprise i) incubating a substrate with a capture agent, ii) incubating the substrate with the first or second sample and a detection agent, and iii) incubating the substrate with a substance capable of engaging with the detection agent and creating a signal, wherein the signal is detectable and quantifiable. In some embodiments, the capture agent and/or the detection agent comprises an antibody selected from the group consisting of Ab-1, Ab-2, Ab-3, Ab-4, Ab-5, and Ab-6. For example, the capture agent may be Ab-1 and the detector agent may be Ab-3. In certain embodiments, the substrate comprises assay beads and helper beads. In some embodiments, the incubating step of ii) occurs for about 10-150 minutes and the incubating step of iii) occurs for about 1-30 minutes. In some embodiments, the incubating step of ii) occurs for about 35 minutes and the incubating step of iii) occurs for about 5 minutes. In some embodiments, the incubating step of ii) occurs for about 75 minutes and the incubating step of iii) occurs for about 5 minutes. In some embodiments, the incubating step of ii) occurs for about 120 minutes and the incubating step of iii) occurs for about 30 minutes. In some embodiments, the incubating step of ii) occurs for about 120 minutes and the incubating step of iii) occurs for about 5 minutes. In some embodiments, the ratio of helper beads to assay beads is about 1:5 to about 5:1. In some embodiments, the assay comprises a greater number of helper beads when compared to the number of assay beads. In some embodiments, the ratio of helper beads to assay beads is about 1:1, about 1.5:1, or about 2:1.

In certain embodiments, frataxin levels (baseline or subsequent concentration) are quantifiable at a level of about 2.0 pg/mL or less, about 1.0 pg/mL or less, or about 0.5 pg/mL or less.

In certain embodiments, the disclosure herein provides a method of determining the amount of neurofilament light chain and frataxin protein from a sample of a biofluid. In some embodiments, the amount of frataxin protein and the amount of neurofilament light chain are determined from a single sample of a biofluid.

In some embodiments, the disclosure herein provides a treatment for Friedreich's Ataxia comprising frataxin gene therapy and a second therapy. The second therapy may comprise omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia, acetyl-L-carnitine (ALCAR), rosuvastatin, an incretin analog, indole-3-propionic acid, and/or erythropoietin or a modified (e.g., carbamylated) form thereof.

In certain embodiments, the disclosure herein provides a method of determining the efficacy of a treatment to increase frataxin levels in a subject having Friedreich's Ataxia, comprising the steps of a) assessing a baseline concentration of frataxin in a first sample of a biofluid of a subject, wherein the biofluid is optionally cerebrospinal fluid (CSF) or serum, b) treating the subject with a treatment which results in increased frataxin levels in the subject, c) assessing a subsequent concentration of frataxin in a second sample of the biofluid of the subject, d) comparing the baseline concentration of frataxin to the subsequent concentration of frataxin, and e) determining an efficacy of the treatment for increasing frataxin levels, wherein the treatment is effective if the subsequent concentration of frataxin is greater than the baseline concentration. In some embodiments, the treatment is effective if the subsequent concentration of frataxin is at least 0.5×-3× greater (e.g., 0.5-1×, 1-1.5×, 1-1.5×, 1.5-2×, 2-2.5×, 2.5-3× greater) than the baseline concentration. In some embodiments, the treatment is effective if the subsequent concentration of frataxin is at least 2× greater than the baseline concentration. In some embodiments, the treatment is effective if the subsequent concentration of frataxin is at least 3× greater than the baseline concentration. In some embodiments, the method further comprises a frataxin gene therapy.

In certain embodiments, the disclosure herein provides a method of determining the efficacy of a treatment to increase frataxin levels in a subject having Friedreich's Ataxia, comprising the steps of a) assessing a baseline concentration of frataxin in a first sample of a biofluid of a subject, wherein the biofluid is optionally cerebrospinal fluid (CSF) or serum, b) assessing a baseline concentration of neurofilament light chain in a second sample of a biofluid of the subject, wherein the biofluid is optionally CSF or serum, c) treating the subject with a treatment which results in increased frataxin levels in the subject, assessing a subsequent concentration of frataxin in a third sample of a biofluid of the subject, wherein the biofluid of d) is CSF if the biofluid of a) is CSF, and wherein the biofluid of d) is serum if the biofluid of a) is serum, e) assessing a subsequent concentration of neurofilament light chain in a fourth sample of a biofluid of the subject, wherein the biofluid of e) is CSF if the biofluid of b) is CSF, and wherein the biofluid of e) is serum if the biofluid of b) is serum, f) comparing the baseline concentration of frataxin to the subsequent concentration of frataxin and comparing the baseline concentration of neurofilament light chain to the subsequent concentration of neurofilament light chain, and g) determining an efficacy of the treatment for increasing frataxin levels, wherein the treatment is effective if the subsequent concentration of frataxin is greater than the baseline concentration of frataxin and the subsequent concentration of neurofilament light chain is lower than or equal to the baseline concentration of neurofilament light chain. In some embodiments, the treatment is effective if the subsequent concentration of frataxin is greater than the baseline concentration of frataxin and the subsequent concentration of neurofilament light chain is not significantly higher (e.g., no more than 1, 2, or 3 pg/mL higher; or no more than 3%, 5%, 7% higher) than the baseline concentration of neurofilament light chain. In some embodiments, the treatment is effective if the subsequent concentration of frataxin is at least 0.5×-3× greater than the baseline concentration or frataxin and the subsequent concentration of neurofilament light chain is not significantly higher than the baseline concentration of neurofilament light chain when controlled for aging (e.g., is not increased by more than 1, 2 or 3 pg/mL, or is not increased by more than 3%, 5%, or 7% of the baseline concentration). In some embodiments, the method further comprises a frataxin gene therapy.

In certain embodiments, the disclosure herein provides a method of determining the efficacy of a treatment to increase frataxin levels in a subject having Friedreich's Ataxia using an assay having a limit of detection of about 2.0 pg/mL or less, wherein the assay comprises a capture agent that specifically binds to frataxin or a fragment of frataxin, and a detection agent that specifically binds to frataxin or a fragment of frataxin, comprising the steps of: a) determining a baseline concentration of frataxin in a first serum sample from the subject; b) treating the subject with a treatment which results in increased frataxin levels in the subject; c) determining a subsequent concentration of frataxin in a second serum sample from the subject; d) comparing the baseline concentration of frataxin in the first serum sample to the subsequent concentration of frataxin in the second serum sample; and e) determining treatment efficacy, wherein the treatment is effective if the subsequent concentration of frataxin in the second serum sample is greater than the baseline concentration in the first serum sample. In some embodiments, the baseline concentration of frataxin in the first serum sample is below 10 pg/mL, below 5 pg/mL, below 2 pg/mL, or less. In some embodiments, the baseline concentration of frataxin in the first serum sample is below 1 pg/mL. In some embodiments, the baseline concentration of frataxin in the first serum sample is about 0.5-1.5 pg/mL.

In certain embodiments, the disclosure herein provides a method of determining the efficacy of a treatment to increase frataxin levels in a subject having Friedreich's Ataxia using an assay having a limit of detection of about 2.0 pg/mL or less, wherein the assay comprises a capture agent that specifically binds to frataxin or a fragment of frataxin, and a detection agent that specifically binds to frataxin or a fragment of frataxin, comprising the steps of: a) determining a baseline concentration of frataxin in a first CSF sample from the subject; b) treating the subject with a treatment which results in increased frataxin levels in the subject; c) determining a subsequent concentration of frataxin in a second CSF sample from the subject; d) comparing the baseline concentration of frataxin in the first CSF sample to the subsequent concentration of frataxin in the second CSF sample; and e) determining treatment efficacy, wherein the treatment is effective if the subsequent concentration of frataxin in the second CSF sample is greater than the baseline concentration in the first CSF sample. In some embodiments, the baseline concentration of frataxin in the first CSF sample is below 5 pg/mL, below 2 pg/mL, or less. In some embodiments, the baseline concentration of frataxin in the first CSF sample is below 1 pg/mL. In some embodiments, the baseline concentration of frataxin in the first CSF sample is about 0.5-1.5 pg/mL.

In certain embodiments, the disclosure herein provides a method of determining the efficacy of a treatment to increase frataxin levels in a subject having Friedreich's Ataxia using an assay having a limit of detection of about 2.0 pg/mL or less, wherein the assay comprises a capture agent that specifically binds to frataxin or a fragment of frataxin, and a detection agent that specifically binds to frataxin or a fragment of frataxin, comprising the steps of: a) determining a baseline concentration of frataxin in a first serum sample of the subject; b) determining a baseline concentration of neurofilament light chain in a second serum sample of the subject; c) treating the subject with a treatment which results in increased frataxin levels in the subject; d) determining a subsequent concentration of frataxin in a third serum sample of the subject; e) determining a subsequent concentration of neurofilament light chain in a fourth serum sample of the subject; f) comparing the baseline concentration of frataxin to the subsequent concentration of frataxin, comparing the baseline concentration of neurofilament light chain to the subsequent concentration of neurofilament light chain; and g) determining treatment efficacy, wherein the treatment is effective if the subsequent concentration of frataxin is greater than the baseline concentration of frataxin and the subsequent concentration of neurofilament light chain is lower than or equal to the baseline concentration of neurofilament light chain. In some embodiments, the baseline concentration of frataxin is below 10 pg/mL, below 5 pg/mL, below 2 pg/mL, or less. In some embodiments, the baseline concentration of frataxin is below 1 pg/mL. In some embodiments, the baseline concentration of frataxin is about 0.5-1.5 pg/mL. In some embodiments, the baseline concentration of neurofilament light chain is at least 20 pg/mL. In some embodiments, the second serum sample may be the same sample or part of the same initial sample as the first serum sample. In some embodiments, the fourth serum sample may be the same sample or part of the same initial sample as the third serum sample.

In certain embodiments, the disclosure herein provides a method of determining the efficacy of a treatment to increase frataxin levels in a subject having Friedreich's Ataxia using an assay having a limit of detection of about 2.0 pg/mL or less, wherein the assay comprises a capture agent that specifically binds to frataxin or a fragment of frataxin, and a detection agent that specifically binds to frataxin or a fragment of frataxin, comprising the steps of: a) determining a baseline concentration of frataxin in a first CSF sample of the subject; b) determining a baseline concentration of neurofilament light chain in a second CSF sample of the subject; c) treating the subject with a treatment which results in increased frataxin levels in the subject; d) determining a subsequent concentration of frataxin in a third CSF sample of the subject; e) determining a subsequent concentration of neurofilament light chain in a fourth CSF sample of the subject; f) comparing the baseline concentration of frataxin to the subsequent concentration of frataxin, comparing the baseline concentration of neurofilament light chain to the subsequent concentration of neurofilament light chain; and g) determining treatment efficacy, wherein the treatment is effective if the subsequent concentration of frataxin is greater than the baseline concentration of frataxin and the subsequent concentration of neurofilament light chain is lower than or equal to the baseline concentration of neurofilament light chain. In some embodiments, the baseline concentration of frataxin is below 5 pg/mL, below 2 pg/mL, or less. In some embodiments, the baseline concentration of frataxin is below 1 pg/mL. In some embodiments, the baseline concentration of frataxin is about 0.5-1.5 pg/mL. In some embodiments, the baseline concentration of neurofilament light chain is at least 1200 pg/mL. In some embodiments, the second CSF sample may be the same sample or part of the same initial sample as the first CSF sample. In some embodiments, the fourth CSF sample may be the same sample or part of the same initial sample as the third CSF sample.

In some embodiments, the treatment is effective if the subsequent concentration of frataxin is at least 0.5×-3× greater than the baseline concentration. In some embodiments, the treatment is effective if the subsequent concentration of frataxin is at least 3× greater than the baseline concentration.

In certain embodiments, the disclosure herein provides a method of treating Friedreich's Ataxia in a subject, comprising the steps of a) assessing a baseline concentration of frataxin in a first sample of a biofluid of a subject, wherein the biofluid is optionally cerebrospinal fluid (CSF) or serum, b) treating the subject with a frataxin gene therapy, c) assessing a subsequent concentration of frataxin in a second sample of the biofluid of the subject, d) comparing the baseline concentration of frataxin to the subsequent concentration of frataxin, and e) determining an efficacy of the treatment for increasing frataxin levels, wherein the frataxin gene therapy is effective if the subsequent concentration of frataxin is greater than the baseline concentration.

In some embodiments, the biofluid is serum. In some embodiments, the baseline concentration of frataxin in a first sample of serum is below 10 pg/mL, below 5 pg/mL, below 2 pg/mL, or less. In some embodiments, the baseline concentration of frataxin in a first sample of serum is below 1 pg/mL. In some embodiments, the baseline concentration of frataxin in a first sample of serum is about 0.5-1.5 pg/mL.

In some embodiments, the biofluid is CSF. In some embodiments, the baseline concentration of frataxin in a first sample of CSF is below 5 pg/mL, below 2 pg/mL, or less. In some embodiments, the baseline concentration of frataxin in a first sample of CSF is below 1 pg/mL. In some embodiments, the baseline concentration of frataxin in a first sample of CSF is about 0.5-1.5 pg/mL.

In some embodiments, if the frataxin gene therapy is not effective, the subject may be administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is not effective, the subject may be administered a treatment comprising a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a treatment comprising managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a treatment comprising a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In certain embodiments, the disclosure herein provides a method of treating Friedreich's Ataxia in a subject, comprising the steps of a) assessing a baseline concentration of frataxin in a first sample of a biofluid of a subject and assessing a baseline concentration of neurofilament light chain in a second sample of a biofluid of the subject, wherein the biofluid is optionally cerebrospinal fluid (CSF) or serum and wherein the first sample and the second sample are optionally the same sample or each is a portion of an initial sample, b) treating the subject with a frataxin gene therapy, c) assessing a subsequent concentration of frataxin in a third sample of a biofluid of the subject and assessing a subsequent concentration of neurofilament light chain in a fourth sample of a biofluid in the subject, wherein the biofluid of c) is CSF if the biofluid of a) is CSF, and wherein the biofluid of c) is serum if the biofluid of a) is serum, and wherein the third sample and the fourth sample are optionally the same sample or each is a portion of an initial sample, d) comparing the baseline concentration of frataxin to the subsequent concentration of frataxin and comparing the baseline concentration of neurofilament light chain to the subsequent concentration of neurofilament light chain, and e) determining an efficacy of the treatment for increasing frataxin levels, wherein the frataxin gene therapy is effective if the subsequent concentration of frataxin is greater than the baseline concentration and the subsequent concentration of neurofilament light chain is lower than or equal to, or not significantly higher than (e.g., no more than 1, 2, or 3 pg/mL higher than; no more than 3%, 5%, or 7% than), the baseline concentration of neurofilament light chain.

In some embodiments, the biofluid is serum. In some embodiments, the baseline concentration of frataxin in a first sample of serum is below 10 pg/mL, below 5 pg/mL, below 2 pg/mL, or less. In some embodiments, the baseline concentration of frataxin in a first sample of serum is below 1 pg/mL. In some embodiments, the baseline concentration of frataxin in a first sample of serum is about 0.5-1.5 pg/mL. In some embodiments, the baseline concentration of neurofilament light chain in a second sample of serum is at least 20 pg/mL.

In some embodiments, the biofluid is CSF. In some embodiments, the baseline concentration of frataxin in a first sample of CSF is below 5 pg/mL, below 2 pg/mL, or less. In some embodiments, the baseline concentration of frataxin in a first sample of CSF is below 1 pg/mL. In some embodiments, the baseline concentration of frataxin in a first sample of CSF is about 0.5-1.5 pg/mL. In some embodiments, the baseline concentration of neurofilament light chain in a second sample of CSF is at least 1200 pg/mL.

In some embodiments, if the frataxin gene therapy is not effective, the subject may be administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is not effective, the subject may be administered a treatment comprising a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a treatment comprising managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a treatment comprising a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, the second therapy comprises omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia), acetyl-L-carnitine (ALCAR), rosuvastatin, an incretin analog, indole-3-propionic acid, and/or erythropoietin or a modified (e.g., carbamylated) form thereof.

In some embodiments, omaveloxolone is administered as a second therapy at a low dose of 5 mg/day. In some embodiments, omaveloxolone is administered as a second therapy at a high dose of 300 mg/day.

In certain embodiments, assessing the baseline or subsequent concentrations of frataxin comprises i) incubating a substrate with a capture agent, ii) incubating the substrate with a sample and a detection agent, iii) incubating the substrate with a substance capable of engaging with the detection agent and creating a signal, wherein the signal is detectable and quantifiable. In some embodiments, the capture agent comprises an antibody selected from the group consisting of Ab-1, Ab-2, Ab-3, Ab-4, Ab-5, and Ab-6; and the detection agent comprises an antibody selected from the group consisting of Ab-1, Ab-2, Ab-3, Ab-4, Ab-5, and Ab-6. For example, the capture agent may be Ab-1 and the detector agent may be Ab-3. In some embodiments, the assay comprises a greater number of helper beads than assay beads, wherein a ratio of helper beads to assay beads is about 1:5 to about 5:1, optionally about 1:1 to about 2:1, e.g., about 1:1, about 1.5:1, or about 2:1. In some embodiments, wherein the incubating step of ii) occurs for about 10-150 minutes and the incubating step of iii) occurs for about 1-30 minutes. In some embodiments, the incubating step of ii) occurs for about 35 minutes and the incubating step of iii) occurs for about 5 minutes, wherein the incubating step of ii) occurs for about 75 minutes and the incubating step of iii) occurs for about 5 minutes, wherein the incubating step of ii) occurs for about 120 minutes and the incubating step of iii) occurs for about 30 minutes, or wherein the incubating step of ii) occurs for about 120 minutes and the incubating step of iii) occurs for about 5 minutes, In some embodiments, the incubating step of ii) occurs for about 120 minutes and the incubating step of iii) occurs for about 5 minutes.

In some embodiments, the frataxin biofluid assay described herein (alone or in combination with a neurofilament light chain assay) further used in combination with an additional measure, such as, but not limited to, a clinical or neuroimaging assessment.

For example, any of the methods of the above embodiments may further comprise a neuroimaging assessment of one or more regions of a subject's central nervous system. In some embodiments, the neuroimaging assessment comprises one or more of magnetic resonance imaging (MRI) or spectroscopy (MRS), position emission tomography (PET), computed tomography (CT), ultrasound, and/or diffusion tensor imaging (DTI). In some embodiments, the neuroimaging assessment comprises brain morphometry of the cerebellum and brainstem, spinal cord morphometry, brain and spinal cord diffusion, dentate iron content (brain QSM), and/or spinal cord spectroscopy. In some embodiments, the one or more regions of the subject's central nervous system is selected from the group consisting of lobule V, lobule VI, lobule VIII, crus of cerebellum, posterior lobe of vermis, flocculi bilaterally, (cerebellar) left tonsil, cervical spinal cord, thoracic spinal cord, cerebellar peduncles, dentate nucleus, and a combination thereof.

In certain aspects, the disclosure provides a method of diagnosing Friedreich's Ataxia in a subject, comprising the steps of: performing an assay on a sample of a biofluid from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing a neuroimaging assessment on the subject; and diagnosing the subject with Friedreich's Ataxia if the concentration of frataxin protein present in the sample is just above, below, or equal to the limit of detection and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei.

In some embodiments, the method comprises performing an assay on a sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing a neuroimaging assessment on the subject suspected of having Friedreich's Ataxia; performing an assay on a sample of a biofluid from a healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing a neuroimaging assessment on the healthy subject; and diagnosing the subject with Friedreich's Ataxia if the concentration of frataxin protein present in the sample from the subject suspected of having Friedreich's Ataxia is at least 2-fold less to at least 2000-fold less than the concentration of frataxin protein present in the sample from the healthy subject and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei in the subject suspected of having Friedreich's Ataxia compared to the healthy subject. In some embodiments, the biofluid is serum. In some embodiments, the concentration of frataxin in a sample of serum from the subject suspected of having Friedreich's Ataxia is below 10 pg/mL, below 5 pg/mL, below 2 pg/mL, or less. In some embodiments, the concentration of frataxin in a sample of serum from the subject suspected of having Friedreich's Ataxia is below 1 pg/mL. In some embodiments, the concentration of frataxin in a sample of serum from the subject suspected of having Friedreich's Ataxia is below the level of detection. In some embodiments, the biofluid is CSF. In some embodiments, the concentration of frataxin in a sample of CSF from the subject suspected of having Friedreich's Ataxia is below 5 pg/mL, below 2 pg/mL, or less. In some embodiments, the concentration of frataxin in a sample of CSF from the subject suspected of having Friedreich's Ataxia is below 1 pg/mL. In some embodiments, the concentration of frataxin in a sample of CSF from the subject suspected of having Friedreich's Ataxia is below the level of detection.

For example, the subject suspected of having Friedreich's Ataxia may have gray matter volume loss of at least 5% relative to a healthy subject, e.g., an age-matched healthy subject. In some embodiments, the subject suspected of having Friedreich's Ataxia has lobular atrophy of at least 5% relative to a healthy subject, e.g., an age-matched healthy subject. In some embodiments, the subject suspected of having Friedreich's Ataxia has a decreased mean volume in the dentate nucleus of at least 20% relative to a healthy subject, e.g., an age-matched healthy subject. In some embodiments, the subject suspected of having Friedreich's Ataxia has increased mean diffusivity in cervical spinal cord and/or superior, middle, and inferior cerebellar peduncles of at least 15% relative to a healthy subject, e.g., an age-matched healthy subject. In some embodiments, the subject suspected of having Friedreich's Ataxia has increased radial diffusivity in cervical spinal cord and/or superior, middle, and inferior cerebellar peduncles of at least 30% relative to a healthy subject, e.g., an age-matched healthy subject. In some embodiments, the subject suspected of having Friedreich's Ataxia has increased axial diffusivity in cervical spinal cord and/or superior, middle, and inferior cerebellar peduncles of at least 5%, relative to a healthy subject, e.g., an age-matched healthy subject. In some embodiments, the subject suspected of having Friedreich's Ataxia has reduced fractional anisotropy of white matter of at least relative to a healthy subject, e.g., an age-matched healthy subject. In some embodiments, the cervical and/or thoracic spinal cord cross-sectional area of the subject suspected of having Friedreich's Ataxia is reduced at least 5% relative to that of a healthy subject, e.g., an age-matched healthy subject. In some embodiments, the cervical and/or thoracic spinal cord volume of the subject suspected of having Friedreich's Ataxia is reduced at least 5% relative to that of a healthy subject, e.g., an age-matched healthy subject.

In some embodiments, the method of diagnosing Friedreich's Ataxia in a subject suspected of having Friedreich's Ataxia comprises performing an assay on a sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing a neuroimaging assessment on the subject suspected of having Friedreich's Ataxia; performing an assay on a sample of a biofluid from a healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing a neuroimaging assessment on the healthy subject; and diagnosing the subject with Friedreich's Ataxia if the concentration of frataxin protein present in the sample from the subject suspected of having Friedreich's Ataxia is at least 2-fold less to at least 2000-fold less than the concentration of frataxin protein present in the sample from the healthy subject and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei in the subject suspected of having Friedreich's Ataxia compared to the healthy subject. In some embodiments, the biofluid is serum. In some embodiments, the concentration of frataxin in a sample of serum from the subject suspected of having Friedreich's Ataxia is below 10 pg/mL, below 5 pg/mL, below 2 pg/mL, or less. In some embodiments, the concentration of frataxin in a sample of serum from the subject suspected of having Friedreich's Ataxia is below 1 pg/mL. In some embodiments, the concentration of frataxin in a sample of serum from the subject suspected of having Friedreich's Ataxia is below the level of detection. In some embodiments, the biofluid is CSF. In some embodiments, the concentration of frataxin in a sample of CSF from the subject suspected of having Friedreich's Ataxia is below 5 pg/mL, below 2 pg/mL, or less. In some embodiments, the concentration of frataxin in a sample of CSF from the subject suspected of having Friedreich's Ataxia is below 1 pg/mL. In some embodiments, the concentration of frataxin in a sample of CSF from the subject suspected of having Friedreich's Ataxia is below the level of detection.

In some aspects, the disclosure provides a method of diagnosing Friedreich's Ataxia in a subject, comprising the steps of: performing an assay on a first sample of a biofluid from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing an assay on a second sample of a biofluid from the subject to assess the concentration of neurofilament light chain present in the sample; performing a neuroimaging assessment on the subject; and diagnosing the subject with Friedreich's Ataxia if the concentration of frataxin protein present in the first sample is just above, below, or equal to the limit of detection, if neurofilament light chain is detected in the second sample at a concentration of at least 20 pg/mL in serum or at least 1200 pg/mL in CSF, and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei. In some embodiments, the biofluid is serum. In some embodiments, the concentration of frataxin in a first sample of serum from the subject is below 10 pg/mL, below 5 pg/mL, below 2 pg/mL, or less. In some embodiments, the concentration of frataxin in a first sample of serum from the subject is below 1 pg/mL. In some embodiments, the concentration of frataxin in a first sample of serum from the subject is below the level of detection. In some embodiments, the concentration of neurofilament light chain in a second sample of serum from the subject is at least 20 pg/mL. In some embodiments, the biofluid is CSF. In some embodiments, the concentration of frataxin in a first sample of CSF from the subject is below 5 pg/mL, below 2 pg/mL, or less. In some embodiments, the concentration of frataxin in a first sample of CSF from the subject is below 1 pg/mL. In some embodiments, the concentration of frataxin in a first sample of CSF from the subject is below the level of detection. In some embodiments, the concentration of neurofilament light chain in a second sample of CSF from the subject is at least 1200 pg/mL.

In some aspects, the disclosure provides a method of diagnosing Friedreich's Ataxia in a subject suspected of having Friedreich's Ataxia, comprising the steps of performing an assay on a first sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing an assay on a second sample of a biofluid from the subject suspected of having Friedreich's Ataxia to assess the concentration of neurofilament light chain present in the sample; performing a neuroimaging assessment on the subject suspected of having Friedreich's Ataxia; performing an assay on a first sample of a biofluid from an age-matched healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; performing an assay on a second sample of a biofluid from the age-matched healthy subject to assess the concentration of neurofilament light chain present in the sample; performing a neuroimaging assessment on the age-matched healthy subject; and diagnosing the subject with Friedreich's Ataxia if the concentration of frataxin protein present in the first sample from the subject suspected of having Friedreich's Ataxia is at least 2-fold less to at least 2000-fold less than the concentration of frataxin protein present in the first sample from the age-matched healthy subject; the concentration of neurofilament light chain present in the second sample from the subject suspected of having Friedreich's Ataxia is greater than or equal to the concentration of neurofilament light chain present in the second sample from the age-matched healthy subject; and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei in the subject suspected of having Friedreich's Ataxia compared to the age-matched healthy subject. In some embodiments, the biofluid is serum. In some embodiments, the concentration of frataxin in a first sample of serum from the subject suspected of having Friedreich's Ataxia is below 10 pg/mL, below 5 pg/mL, below 2 pg/mL, or less. In some embodiments, the concentration of frataxin in a first sample of serum from the subject suspected of having Friedreich's Ataxia is below 1 pg/mL. In some embodiments, the concentration of frataxin in a first sample of serum from the subject suspected of having Friedreich's Ataxia is below the level of detection. In some embodiments, the concentration of neurofilament light chain in a second sample of serum from the subject suspected of having Friedreich's Ataxia is at least 20 pg/mL. In some embodiments, the biofluid is CSF. In some embodiments, the concentration of frataxin in a first sample of CSF from the subject suspected of having Friedreich's Ataxia is below 5 pg/mL, below 2 pg/mL, or less. In some embodiments, the concentration of frataxin in a first sample of CSF from the subject suspected of having Friedreich's Ataxia is below 1 pg/mL. In some embodiments, the concentration of frataxin in a first sample of CSF from the subject suspected of having Friedreich's Ataxia is below the level of detection. In some embodiments, the concentration of neurofilament light chain in a second sample of CSF from the subject suspected of having Friedreich's Ataxia is at least 1200 pg/mL.

In some embodiments, the first sample and the second sample of a biofluid from the subject suspected of having Friedreich's Ataxia are the same sample or each is a portion of an initial sample. In some embodiments, the first sample and the second sample of a biofluid from the healthy subject are the same sample or each is a portion of an initial sample. In certain embodiments, the assessing steps of the method comprise i) incubating a substrate with a capture agent, ii) incubating the substrate with the first or second sample and a detection agent, and iii) incubating the substrate with a substance capable of engaging with the detection agent and creating a signal, wherein the signal is detectable and quantifiable. In some embodiments, the capture agent and/or the detection agent comprises an antibody selected from the group consisting of Ab-1, Ab-2, Ab-3, Ab-4, Ab-5, and Ab-6. For example, the capture agent may be Ab-1 and the detector agent may be Ab-3. In certain embodiments, the substrate comprises assay beads and helper beads. In some embodiments, the incubating step of ii) occurs for about 10-150 minutes and the incubating step of iii) occurs for about 1-30 minutes. In some embodiments, the incubating step of ii) occurs for about 35 minutes and the incubating step of iii) occurs for about 5 minutes. In some embodiments, the incubating step of ii) occurs for about 75 minutes and the incubating step of iii) occurs for about 5 minutes. In some embodiments, the incubating step of ii) occurs for about 120 minutes and the incubating step of iii) occurs for about 30 minutes. In some embodiments, the incubating step of ii) occurs for about 120 minutes and the incubating step of iii) occurs for about 5 minutes. In some embodiments, the ratio of helper beads to assay beads is about 1:5 to about 5:1. In some embodiments, the assay comprises a greater number of helper beads when compared to the number of assay beads. In some embodiments, the ratio of helper beads to assay beads is about 1:1, about 1.5:1, or about 2:1. In certain embodiments, frataxin levels (baseline or subsequent concentration) are quantifiable at a level of about 2.0 pg/mL, or less, about 1.0 pg/mL or less, or about 0.5 pg/mL or less.

In some embodiments, the subject suspected of having Friedreich's Ataxia has gray matter volume loss of 5%, 10%, 15%, 20%, 25%, 30%, 40%, 50%, 60%, or more than 60% relative to the healthy subject, e.g., the age-matched healthy subject. In some embodiments, the subject suspected of having Friedreich's Ataxia has lobular atrophy of 5%, 10%, 15%, 20%, 25%, 30%, 40%, 50%, 60%, or more than 60% relative to the healthy subject, e.g., the age-matched healthy subject. In some embodiments, the subject suspected of having Friedreich's Ataxia has a decreased mean volume in the dentate nucleus of 20%, 25%, 30%, 35%, 40%, 45%, or more than 45% relative to the healthy subject, e.g., the age-matched healthy subject. In some embodiments, the subject suspected of having Friedreich's Ataxia has increased mean diffusivity in cervical spinal cord and/or superior, middle, and inferior cerebellar peduncles of 15%, 20%, 25%, 30%, 35%, 40%, 45%, 50%, 60%, 70%, or more than 70% relative to the healthy subject, e.g., the age-matched healthy subject. In some embodiments, the subject suspected of having Friedreich's Ataxia has increased radial diffusivity in cervical spinal cord and/or superior, middle, and inferior cerebellar peduncles of 30%, 35%, 40%, 45%, 50%, 55%, 60%, 65%, 70%, 75%, or more than 75% relative to the healthy subject, e.g., the age-matched healthy subject. In some embodiments, the subject suspected of having Friedreich's Ataxia has increased axial diffusivity in cervical spinal cord and/or superior, middle, and inferior cerebellar peduncles of 5%, 10%, 15%, 20%, 25%, or more than 25% relative to the healthy subject, e.g., the age-matched healthy subject. In some embodiments, the subject suspected of having Friedreich's Ataxia has reduced fractional anisotropy of white matter of 15%, 20%, 25%, 30%, 35%, 40%, 45%, 50%, 60%, 70%, or more than 70% relative to the healthy subject, e.g., the age-matched healthy subject. In some embodiments, the cervical and/or thoracic spinal cord cross-sectional area of the subject suspected of having Friedreich's Ataxia is reduced 5%, 10%, 15%, 20%, 25%, 30%, 35%, 40%, 50%, 60%, or more than 60% relative to that of the healthy subject, e.g., the age-matched healthy subject. In some embodiments, the cervical and/or thoracic spinal cord volume of the subject suspected of having Friedreich's Ataxia is reduced 5%, 10%, 15%, 20%, 25%, 30%, 35%, 40%, 50%, 60%, or more than 60% relative to that of the healthy subject, e.g., the age-matched healthy subject.

In some aspects, the disclosure provides a method of determining the efficacy of a treatment to increase frataxin levels in a subject having Friedreich's Ataxia, comprising: determining a baseline concentration of frataxin in a first sample of a biofluid of a subject, wherein the biofluid is optionally cerebrospinal fluid (CSF) or serum; performing a baseline neuroimaging assessment on the subject; treating the subject with a treatment which results in increased frataxin levels in the subject; determining a subsequent concentration of frataxin in a second sample of the biofluid of the subject; performing a subsequent neuroimaging assessment on the subject; comparing the baseline concentration of frataxin to the subsequent concentration of frataxin; comparing the baseline neuroimaging assessment to the subsequent neuroimaging assessment; and determining treatment efficacy, wherein the treatment is effective if the subsequent concentration of frataxin is greater than the baseline concentration and the subsequent neuroimaging assessment shows no significant worsening of atrophy and/or diffusivity compared to the baseline neuroimaging assessment. In some embodiments, the treatment is effective if the subsequent concentration of frataxin is at least 0.5×, e.g., at least 0.5×-3× greater than the baseline concentration and the subsequent neuroimaging assessment shows no significant worsening of atrophy and/or diffusivity compared to the baseline neuroimaging assessment. In some embodiments, the treatment is effective if the subsequent concentration of frataxin is at least 3× greater than the baseline concentration and the subsequent neuroimaging assessment shows no significant worsening of atrophy and/or diffusivity compared to the baseline neuroimaging assessment. In some embodiments, the method further comprises determining and comparing a baseline and subsequent neurofilament light chain concentration in a sample of a biofluid of the subject, such that the treatment is effective if the subsequent concentration of neurofilament light chain is lower than, equal to, or not significantly higher than the baseline concentration of neurofilament light chain. In some embodiments, the biofluid is serum. In some embodiments, the baseline concentration of frataxin in a first sample of serum from the subject is below 10 pg/mL, below 5 pg/mL, below 2 pg/mL, or less. In some embodiments, the baseline concentration of frataxin in a first sample of serum from the subject is below 1 pg/mL. In some embodiments, the baseline concentration of frataxin in a first sample of serum from the subject is about 0.5-1.5 pg/mL. In some embodiments, the baseline concentration of neurofilament light chain in a sample of serum from the subject is at least 20 pg/mL. In some embodiments, the biofluid is CSF. In some embodiments, the baseline concentration of frataxin in a first sample of CSF from the subject is below 5 pg/mL, below 2 pg/mL, or less. In some embodiments, the baseline concentration of frataxin in a first sample of CSF from the subject is below 1 pg/mL. In some embodiments, the baseline concentration of frataxin in a first sample of CSF from the subject is about 0.5-1.5 pg/mL. In some embodiments, the baseline concentration of neurofilament light chain in a sample of CSF from the subject is at least 1200 pg/mL.

In some embodiments, no significant worsening of atrophy and/or diffusivity is indicated by no more than 5% worsening in atrophy and/or diffusivity. For example, in some embodiments, no worsening of atrophy means that atrophy has not increased more than 5% compared to atrophy prior to treatment. In some embodiments, no worsening of diffusivity means that diffusivity has not increased more than 5% compared to diffusivity prior to treatment.

In any of these embodiments, the treatment may comprise a frataxin gene therapy.

In some embodiments, the frataxin gene therapy comprises a nucleic acid construct as disclosed in International Publication No. WO2020/069461, the contents of which are herein incorporated by reference in their entirety.

In some embodiments, the frataxin gene therapy comprises a nucleic acid construct comprising a 5′ ITR sequence region, a promoter region, an intron/exon region, a payload region, an optional tag, up to three miR binding sites, a polyA sequence region, an optional filler sequence, and a 3′ ITR sequence region. In some embodiments, the 5′ ITR of the 5′ ITR sequence region and/or the 3′ ITR of the 3′ ITR sequence region is an AAV2 ITR. In some embodiments the human frataxin expressed by the frataxin gene therapy is encoded by any of SEQ ID NOs: 1-3, e.g., SEQ ID NO: 1, or a fragment thereof. In some embodiments, the human frataxin expressed by the frataxin gene therapy comprises or consists of any of SEQ ID NOs: 4-6, e.g., SEQ ID NO: 4. In some embodiments, the frataxin gene therapy comprises a nucleic acid construct (e.g., a viral genome) comprised in an adeno-associated virus (AAV), such that the frataxin gene therapy comprises an AAV comprising the nucleic acid construct (e.g., the viral genome) and an AAV capsid. Representative human frataxin sequences are listed in Table 1.

In some embodiments, the frataxin gene therapy construct (e.g., the nucleic acid construct) comprises a truncated CMV promoter driving expression of human frataxin, e.g., wherein the truncated CMV promoter comprises or consists of a CMV-D7 promoter (SEQ ID NO: 7). In some embodiments, the frataxin gene therapy construct comprises a truncated CBA promoter driving expression of human frataxin, e.g., wherein the truncated CBA promoter comprises or consists of a CBA-D8 promoter (SEQ ID NO: 8), a CBA-D4 promoter (SEQ ID NO: 9), or a CBA-D6 promoter (SEQ ID NO: 10). Representative promoters are listed in Table 49.

In some embodiments, the frataxin gene therapy construct comprises SEQ ID NO: 11, which comprises a 5′ inverted terminal repeat (ITR) sequence region and a 3′ ITR sequence region, a CMV-D7 promoter region, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence.

In some embodiments, the frataxin gene therapy construct comprises SEQ ID NO: 12, which comprises a 5′ inverted terminal repeat (ITR) sequence region and a 3′ ITR sequence region, a CBA-D8 promoter region, a junction sequence, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence.

In some embodiments, the frataxin gene therapy construct comprises SEQ ID NO: 13, which comprises a 5′ inverted terminal repeat (ITR) sequence region and a 3′ ITR sequence region, a CBA-D4 promoter region, a junction sequence, a human beta globin intron/exon region comprising an ie1 exon 1 region, an ie1 intron 1 region, a human beta-globin intron region, a human beta-globin exon region, a human frataxin payload sequence, a mat binding site series comprising three repeats of single miR122 binding site sequences, a human growth hormone polyadenylation sequence, and an albumin filler sequence.

In some embodiments, the frataxin gene therapy construct comprises SEQ ID NO: 14, which comprises a 5′ inverted terminal repeat (ITR) sequence region and a 3′ ITR sequence region, a CBA-D6 promoter region, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence. Representative frataxin gene therapy constructs are listed in Table 50.

In some embodiments, the frataxin gene therapy comprises an AAV comprising a VOY101 capsid, e.g., of amino acid sequence SEQ ID NO: 15 and/or encoded by nucleic acid sequence SEQ ID NO: 16. In some embodiments, the frataxin gene therapy comprises an AAV comprising a sequence that is at least 95%, 96%, 97%, 98%, or 99% identical to VOY101, e.g., comprising an amino acid sequence that is at least 95%, 96%, 97%, 98%, or 99% identical to SEQ ID NO: 15 and/or encoded by a nucleic acid sequence that is at least 95%, 96%, 97%, 98%, or 99% identical to SEQ ID NO: 16. In some embodiments, the frataxin gene therapy comprises an AAV comprising a sequence that is at leak 99% identical to VOY101, e.g., comprising an amino acid sequence that is at least 99% identical to SEQ ID NO: 15 and/or encoded by a nucleic acid sequence that is at least 99% identical to SEQ ID NO: 16.

In some embodiments, the frataxin gene therapy is formulated to comprise sodium chloride, sodium phosphate, potassium chloride, potassium phosphate and poloxamer 188. In some embodiments, the frataxin gene therapy is formulated to comprise 192 mM sodium chloride, 10 mM sodium phosphate, 2.7 mM potassium chloride, 2 mM potassium phosphate and 0.001% poloxamer 188 (v/v), wherein the pH of the formulation is 7.4.

In some aspects, the disclosure provides a method of treating Friedreich's Ataxia in a subject, comprising determining a baseline concentration of frataxin in a first sample of a biofluid of a subject, wherein the biofluid is optionally cerebrospinal fluid (CSF) or serum; performing a baseline neuroimaging assessment on the subject; treating the subject with a treatment which results in increased frataxin levels, e.g., a frataxin gene therapy; determining a subsequent concentration of frataxin in a second sample of the biofluid of the subject; performing a subsequent neuroimaging assessment on the subject; comparing the baseline concentration of frataxin to the subsequent concentration of frataxin and comparing the baseline neuroimaging assessment to the subsequent neuroimaging assessment; wherein the frataxin gene therapy is effective if the subsequent concentration of frataxin is greater than the baseline concentration of frataxin and the subsequent neuroimaging assessment shows no significant worsening of atrophy and/or diffusivity compared to the baseline neuroimaging assessment; and pursuing a suitable therapy according to the effectiveness assessment. In some embodiments, the biofluid is serum. In some embodiments, the baseline concentration of frataxin in a first sample of serum from the subject is below 10 pg/mL, below 5 pg/mL, below 2 pg/mL, or less. In some embodiments, the baseline concentration of frataxin in a first sample of serum from the subject is below 1 pg/mL. In some embodiments, the baseline concentration of frataxin in a first sample of serum from the subject is about 0.5-1.5 pg/mL. In some embodiments, the biofluid is CSF. In some embodiments, the baseline concentration of frataxin in a first sample of CSF from the subject is below 5 pg/mL, below 2 pg/mL, or less. In some embodiments, the baseline concentration of frataxin in a first sample of CSF from the subject is below 1 pg/mL. In some embodiments, the baseline concentration of frataxin in a first sample of CSF from the subject is about 0.5-1.5 pg/mL.

For example, if the frataxin gene therapy is not effective, the method may further comprise administering a second therapy, e.g., a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy. In some embodiments, the second therapy is administered at a high dose, as disclosed herein.

In another example, if the frataxin gene therapy is effective, the method may further comprise managing one or more symptoms of Friedreich's Ataxia using occupational therapy, speech therapy, orthopedic care, bracing, surgery, treatment for heart disease, an antioxidant, treatment to lower blood sugar, and/or physical therapy. As a further example, if the frataxin gene therapy is effective, the method may further comprise administering a second therapy comprising administering a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy. In some embodiments, the method further comprises determining and comparing a baseline and subsequent neurofilament light chain concentration in a sample of a biofluid of the subject, such that treatment efficacy is also determined in part if the subsequent concentration of neurofilament light chain is not significantly different from the baseline concentration of neurofilament light chain.

In any of the above embodiments, the frataxin gene therapy may be as described herein or in International Application Publication No. WO2020/069461. For example, in some embodiments, the frataxin gene therapy may comprise a nucleic acid construct having any of SEQ IDs NO: 11-14 and/or an AAV capsid having SEQ ID NO: 15 or encoded by SEQ ID NO: 16. In some embodiments, the AAV capsid comprises an amino acid sequence at least 95%, 96%, 97%, 98%, or 99% identical to SEQ ID NO: 15 or is encoded by a nucleic acid sequence at least 95%, 96%, 97%, 98%, or 99% identical to SEQ ID NO: 16. In some embodiments, the AAV capsid comprises an amino acid sequence at least 99% identical to SEQ ID NO: 15 or is encoded by a nucleic acid sequence at least 99% identical to SEQ ID NO: 16.

In any of the above embodiments, the second therapy may comprise omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia), acetyl-L-carnitine (ALCAR), rosuvastatin, an incretin analog, indole-3-propionic acid, and/or erythropoietin or a modified (e.g., carbamylated) form thereof. For example, the second therapy may comprise omaveloxolone, e.g., at a low dose of about 5 mg/day, or at a high dose of about 300 mg/day.

In some embodiments, a frataxin biofluid assay described herein may be used for assessing or monitoring the impact of a therapy in cardiac tissue. In some embodiments, the therapy is a frataxin gene therapy. In some embodiments, the frataxin gene therapy comprises a nucleic acid construct as disclosed in International Publication No. WO2020/069461. In some embodiments, the frataxin biofluid assay may be used in combination with at least one additional assessment, such as a clinical or cardiac imaging assessment. In some embodiments, the at least one additional assessment may include structural imaging (e.g. quantification of Meissner Corpuscle density), quantitative sensory testing, corticokinematic coherence (CKC), and/or other assessments of sensory polyneuropathy. In some embodiments, the at least one additional assessment may include detection of disease diagnostic markers (e.g. GAA repeat expansions). In some embodiments, the at least one additional assessment may include detection of disease predictive markers (e.g. methylation in the frataxin gene). In some embodiments, the at least one additional assessment may include measurement of disease prognostic biomarkers (e.g. cardiac serum biomarkers). In some embodiments, the at least one additional assessment may include evaluation of clinical endpoints, such as, but are not limited to, the FARS upright stability scoring, accelerometry measurement, balance evaluation using the Biodex Balance System, speech measures, assessments using the Scale for Assessment and Rating of Ataxia (SARA) or Friedreich's Ataxia Rating Scale (FARS), the 9 hole peg test, and/or the 25 foot walk test.

In some embodiments, the disclosure provides a frataxin gene therapy for use in the treatment of Friedreich's Ataxia in a subject, wherein the treatment comprises administering the frataxin gene therapy to a subject lacking detectable frataxin in a biofluid sample at a limit of detection of about 2.0 pg/mL or less, wherein the amount of frataxin is determined using an assay as disclosed herein.

In some embodiments, the disclosure provides a frataxin gene therapy for use in the treatment of Friedreich's Ataxia in a subject, wherein the treatment comprises the steps of: a) determining whether a subject has frataxin in a biofluid sample using an assay having a limit of detection of frataxin of about 2.0 pg/mL or less; and b) administering the frataxin gene therapy to a subject determined as having frataxin in the biofluid sample at a concentration at just above, below, or equal to the limit of detection of the assay.

In some embodiments, the disclosure provides a frataxin gene therapy for use in the treatment of Friedreich's Ataxia in a subject, wherein the treatment comprises the steps of: a) determining a baseline concentration of frataxin in a first sample of a biofluid of a subject using an assay as disclosed herein (e.g., an assay having a limit of detection of frataxin of about 2.0 pg/mL or less), wherein the biofluid is optionally cerebrospinal fluid (CSF) or serum; b) treating the subject with the frataxin gene therapy; c) determining a subsequent concentration of frataxin in a second sample of the biofluid of the subject using an assay as disclosed herein (e.g., an assay having a limit of detection of frataxin of about 2.0 pg/mL or less); and d) comparing the baseline concentration of frataxin to the subsequent concentration of frataxin; wherein the frataxin gene therapy is effective if the subsequent concentration of frataxin is greater than the baseline concentration of frataxin.

In some embodiments, if the frataxin gene therapy is not effective, the subject may be administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is not effective, the subject may be administered a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a treatment comprising managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered treatment comprising a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, the disclosure provides a frataxin gene therapy for use in the treatment of Friedreich's Ataxia in a subject, wherein the treatment comprises the steps of: a) determining a baseline concentration of frataxin in a first sample of a biofluid of a subject using an assay as disclosed herein (e.g., an assay having a limit of detection of frataxin of about 2.0 pg/mL or less) and determining a baseline concentration of neurofilament light chain in a second sample of a biofluid of the subject using an assay as disclosed herein, wherein the biofluid is optionally cerebrospinal fluid (CSF) or serum and wherein the first sample and the second sample are optionally the same sample; b) treating the subject with the frataxin gene therapy; c) determining a subsequent concentration of frataxin in a third sample of a biofluid of the subject using an assay as disclosed herein (e.g., an assay having a limit of detection of frataxin of about 2.0 pg/mL or less) and determining a subsequent concentration of neurofilament light chain in a fourth sample of a biofluid in the subject using an assay as disclosed herein, wherein the biofluid of c) is CSF if the biofluid of a) is CSF, and wherein the biofluid of c) is serum if the biofluid of a) is serum, and wherein the third sample and the fourth sample are optionally the same sample; and d) comparing the baseline concentration of frataxin to the subsequent concentration of frataxin and comparing the baseline concentration of neurofilament light chain to the subsequent concentration of neurofilament light chain; wherein the frataxin gene therapy is effective if the subsequent concentration of frataxin is greater than the baseline concentration and the subsequent concentration of neurofilament light chain is not significantly higher than, is lower than, or is equal to the baseline concentration of neurofilament light chain.

In some embodiments, the disclosure provides frataxin gene therapy for use in the treatment of Friedreich's Ataxia in a subject, wherein the treatment comprises the steps of: a) determining a baseline concentration of frataxin in a first sample of a biofluid of a subject using an assay as disclosed herein (e.g., an assay having a limit of detection of frataxin of about 2.0 pg/mL or less) and determining a baseline concentration of neurofilament light chain in a second sample of a biofluid of the subject using an assay as disclosed herein, wherein the biofluid is optionally cerebrospinal fluid (CSF) or serum and wherein the first sample and the second sample are optionally the same sample; b) performing a baseline neuroimaging assessment on the subject; c) treating the subject with the frataxin gene therapy; d) determining a subsequent concentration of frataxin in a third sample of a biofluid of the subject using an assay as disclosed herein (e.g., an assay having a limit of detection of frataxin of about 2.0 pg/mL or less) and determining a subsequent concentration of neurofilament light chain in a fourth sample of a biofluid in the subject using an assay as disclosed herein, wherein the biofluid of d) is CSF if the biofluid of a) is CSF, and wherein the biofluid of d) is serum if the biofluid of a) is serum, and wherein the third sample and the fourth sample are optionally the same sample; e) performing a subsequent neuroimaging assessment on the subject; and f) comparing the baseline concentration of frataxin to the subsequent concentration of frataxin, comparing the baseline concentration of neurofilament light chain to the subsequent concentration of neurofilament light chain, and comparing the baseline neuroimaging assessment to the subsequent neuroimaging assessment; wherein the frataxin gene therapy is effective if the subsequent concentration of frataxin is greater than the baseline concentration, the subsequent concentration of neurofilament light chain is not significantly higher than, is lower than, or is equal to the baseline concentration of neurofilament light chain, and the subsequent neuroimaging assessment shows no significant worsening (e.g., no more than 5% worsening) of atrophy and/or diffusivity compared to the baseline neuroimaging assessment.

In some embodiments, if the frataxin gene therapy is not effective, the subject may be administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is not effective, the subject may be administered a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a treatment comprising managing one or more symptoms of Friedreich's Ataxia using occupational therapy, speech therapy, orthopedic care, bracing, surgery, treatment for heart disease, an antioxidant, treatment to lower blood sugar, and/or physical therapy.

In some embodiments, if the frataxin gene therapy is effective, the subject may be administered a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy. In some embodiments, the disclosure provides a frataxin gene therapy for use in the treatment of Friedreich's Ataxia in a subject, wherein the treatment comprises the steps of: a) determining the concentration of frataxin in a serum sample from the subject using an assay having a limit of detection of frataxin of about 2.0 pg/mL or less; wherein the assay comprises a capture agent that specifically binds to frataxin or a fragment of frataxin, and a detection agent that specifically binds to frataxin or a fragment of frataxin; and b) administering the frataxin gene therapy to the subject if the concentration of frataxin protein present in the serum sample is below 10 pg/mL.

In some embodiments, the disclosure provides a frataxin gene therapy for use in the treatment of Friedreich's Ataxia in a subject, wherein the treatment comprises the steps of: a) determining the concentration of frataxin in a CSF sample from the subject using an assay having a limit of detection of frataxin of about 2.0 pg/mL or less; wherein the assay comprises a capture agent that specifically binds to frataxin or a fragment of frataxin, and a detection agent that specifically binds to frataxin or a fragment of frataxin; and b) administering the frataxin gene therapy to the subject if the concentration of frataxin protein present in the CSF sample is below 5 pg/mL.

### BRIEF DESCRIPTION OF THE DRAWINGS

FIG. 1A shows a representative calibration curve and results from five quality control (QC) samples run at four concentrations. FIG. 1B shows an exemplary setup for an assay to measure frataxin concentration in a biofluid, as described herein. Ab=antibody; RGP=resorufin β-galactopyranoside.

FIG. 2 shows detection of frataxin protein in CSF and serum.

FIG. 3 shows detection of neurofilament light chain proteins in CSF and serum.

FIG. 4A-4B show detection of frataxin (FIG. 4A) and neurofilament light chain (FIG. 4B) proteins in the CSF and serum of pre-clinical animal species.

FIG. 5A-5B show quantification of frataxin (pg/mL) in patient serum (FIG. 5A) and CSF (FIG. 5B) samples.

FIG. 6A-6B show quantification of neurofilament light chain (pg/mL) in patient serum (FIG. 6A) and CSF (FIG. 6B) samples.

### DETAILED DESCRIPTION OF THE DISCLOSURE

### I. INTRODUCTION

### Low Levels of Frataxin

In various embodiments, the disclosure provided herein allows for detection and/or quantification of frataxin levels in biofluids. Without being bound by theory, this may allow for improved detection, monitoring, and/or better assessment of the impact of therapies, e.g., on brain, cardiac tissue, etc. and on whole-body data.

In certain embodiments, a method of the present disclosure is used for detecting and/or quantifying frataxin protein levels in a sample. The sample may be a tissue sample, a sample of cells (e.g., IPSCs), or a biofluid. As used herein, the term “biofluid” refers to a liquid from the body of a subject (e.g., human or other animal). Non-limiting examples of biofluids include cerebrospinal fluid (CSF), plasma, serum, blood, saliva, excreta, sweat, tears, vaginal fluid, semen, breast milk, urine, bile, peritoneal fluid, pericardial fluid, pleural fluid, amniotic fluid, synovial fluid, aqueous humor, vitreous humor, gastric fluid, mucus, sputum, nasal discharge, fluid of the throat or lungs, intracellular fluid, extracellular fluid, interstitial fluid, transcellular fluid, and/or lymphatic fluid (e.g., endolymph, perilymph). The biofluid may be a combination of two or more of the aforementioned bodily fluids. The biofluid may be from a human, or may be from another animal (e.g., non-human primate, dog, cat, mouse, rat, rabbit, guinea pig, etc.). A human biofluid may be from a healthy individual or control subject or from a patient, wherein the patient may be suffering from, suspected of having, susceptible to, or diagnosed with one or more diseases. While not wishing to be bound by theory, a biofluid may also include a supernatant collected from cells prepared or maintained in vitro or ex-vivo.

In certain embodiments, the frataxin protein detected by the method of the present disclosure is a human frataxin protein, or a fragment thereof. In other embodiments, the frataxin protein detected by the method of the present disclosure is a non-human frataxin protein, or a fragment thereof (e.g., non-human primate, dog, cat, pig, ferret, mouse, rat, etc.).

Representative sequences of human frataxin are shown in Table 1. SEQ ID NO: 4 is commonly considered the canonical, or major isoform, human frataxin protein sequence, encoded by SEQ ID NO: 1. The 210 amino acid sequence given by SEQ ID NO: 4 may be cleaved into one or more shorter variants, known as frataxin intermediate form (42-210), frataxin (56-210), frataxin (78-210) and frataxin mature form (81-210), as given by the respective amino acids of SEQ ID NO: 4.

TABLE 1
Representative Frataxin Sequences

| | | SEQ |
|---|---|---|
| Description | Reference | ID NO: |
| Homo sapiens frataxin (FXN) | NM_000144.5 | 1 |
| transcript variant 1, mRNA | | |
| Homo sapiens frataxin (FXN), | NM_181425.2 | 2 |
| transcript variant 2, mRNA | | |
| Homo sapiens frataxin (FXN), | NM_001161706.1 | 3 |
| transcript variant 3, mRNA | | |
| Homo sapiens frataxin, | NP_000135.2 | 4 |
| mitochondrial isoform 1 preproprotein | | |
| Homo sapiens frataxin, | NP_852090.1 | 5 |
| mitochondrial isoform 2 preproprotein | | |
| Homo sapiens frataxin, | NP_001155178.1 | 6 |
| mitochondrial isoform 3 preproprotein | | |

In certain embodiments, the method of the present disclosure may be used to detect frataxin in a sample from a healthy individual or an individual considered a control in a clinical setting. in certain embodiments, the method of the present disclosure may be used to detect frataxin protein in a sample from a subject diagnosed with disease. In certain embodiments, the disease is Friedreich's Ataxia.

In certain embodiments, a method of the present disclosure may be used for determining a treatment protocol for and/or a prognostic indication of a subject's recovery from Friedreich's Ataxia by performing an assay on a plurality of samples to assess a measure of the concentration of frataxin protein in each sample and determining a prognostic indication of the subject's recovery from Friedreich's Ataxia and/or a method of treatment based at least in part on the measure of the concentration of frataxin present in the samples. For example, the concentration of frataxin protein in the subject's biofluid sample may be used to determine a gene therapy treatment regimen suitable to increase frataxin levels. The gene therapy treatment may comprise administration of an AAV comprising a capsid and a viral genome, wherein the viral genome encodes frataxin or a fragment of frataxin.

In some embodiments, the concentration of frataxin protein in the subject's biofluid sample may be used to determine the selection and/or dose of a therapy, e.g., a small molecule therapy, suitable to reduce mitochondrial dysfunction, restore mitochondrial function, reduce inflammation, or resolve inflammation. For example, the therapy may restore mitochondrial transmembrane potential in fibroblasts. In some embodiments, the small molecule therapy activates the Keap1/Nrf2 pathway. In some embodiments, the therapy increases Nrf2 signaling. In some embodiments, the therapy reduces oxidative stress, e.g., by reducing production of reactive oxidation species. In some embodiments, the concentration of frataxin protein in the subject's biofluid sample may be used to determine the selection and/or dose of a therapy, e.g., a small molecule therapy, suitable to at least partially restore motor function, manual dexterity, and/or activities of daily living.

In some embodiments, the concentration of frataxin protein in the subject's biofluid sample may be used to determine the selection and/or dose of a therapy suitable to treat at least one symptom of Friedreich's Ataxia. In some embodiments, the therapy suitable to treat at least one symptom of Friedreich's Ataxia is gene therapy, stem cell therapy, small molecule therapy, frataxin replacement, frataxin enhancement, frataxin stabilization, an oligonucleotide-based therapy, therapy to increase frataxin gene expression, occupational therapy, speech therapy, orthopedic care, bracing, surgery, heart disease medication (e.g., a beta blocker, an angiotensin-converting-enzyme [ACE] inhibitor), an antioxidant (e.g., vitamin E, coenzyme Q₁₀, idebenone, an extract from Ginkgo biloba leaves), treatment to lower blood sugar (e.g., insulin, an oral anti-diabetic drug, modified diet), physical therapy, or a combination thereof.

In some embodiments, the therapy is omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy (e.g., to increase frataxin expression and/or treat cardiac disease associated with Friedreich's Ataxia), acetyl-L-carnitine (ALCAR), rosuvastatin, an incretin analog, indole-3-propionic acid, or erythropoietin or a modified (e.g., carbamylated) form thereof.

The sample, in some embodiments, may comprise a sample from a circulating biofluid. In some embodiments the biofluid is cerebrospinal fluid (CSF). In some embodiments, the biofluid is a blood sample from the subject. In some embodiments the biofluid is plasma and/or serum derived from a blood sample. In some embodiments, the biofluid is another bodily fluid. In some cases, the concentration of frataxin in the sample (e.g., the biofluid) is less than about or about 1000 pg/mL, less than about or about 900 pg/mL, less than about or about 800 pg/mL, less than about or about 700 pg/mL, less than about or about 600 pg/mL, less than about or about 500 pg/mL, less than about or about 400 pg/mL, less than about or about 300 pg/mL, less than about or about 200 pg/mL, less than about or about 100 pg/mL, less than about or about 50 pg/mL, less than about or about 30 pg/mL, less than about or about 20 pg/mL, less than about or about 10 pg/mL, less than about or about 5 pg/mL, less than about or about 1 pg/mL, or less.

In some cases, the assay has a limit of quantification of less than about or about 100 pg/mL, less than about or about 50 pg/mL, less than about or about 40 pg/mL, less than about or about 30 pg/mL, less than about or about 20 pg/mL, less than about or about 10 pg/mL, less than about or about 5 pg/mL, less than about or about 4 pg/mL, less than about or about 3 pg/mL, less than about or about 2 pg/mL, less than about or about 1 pg/mL, less than about or about 0.8 pg/mL, less than about or about 0.7 pg/mL, less than about or about 0.6 pg/mL, less than about or about 0.5 pg/mL, less than about or about 0.4 pg/mL, less than about or about 0.3 pg/mL, less than about or about 0.2 pg/mL, less than about or about 0.1 pg/mL, less than about or about 0.05 pg/mL, less than about or about 0.04 pg/mL, less than about or about 0.02 pg/mL, less than about or about 0.01 pg/mL, or less. In some cases, the assay has a limit of detection of less than about or about 100 pg/mL, less than about or about 50 pg/mL, less than about or about 40 pg/mL, less than about or about 30 pg/mL, less than about or about 20 pg/mL, less than about or about 10 pg/mL, less than about or about 5 pg/mL, less than about or about 4 pg/mL, less than about or about 3 pg/mL, less than about or about 2 pg/mL, less than about or about 1 pg/mL, less than about or about 0.8 pg/mL, less than about or about 0.7 pg/mL, less than about or about 0.6 pg/mL, less than about or about 0.5 pg/mL, less than about or about 0.4 pg/mL, less than about or about 0.3 pg/mL, less than about or about 0.2 pg/mL, less than about or about 0.1 pg/mL, less than about or about 0.05 pg/mL, less than about or about 0.04 pg/mL, less than about or about 0.02 pg/mL, less than about or about 0.01 pg/mL, or less.

In certain embodiments, the methods of the present disclosure may be used to detect or quantify the level of frataxin protein in a sample such as a tissue sample, sample of cells, or biofluid. In certain embodiments, the methods of the present disclosure may be used to detect or quantify the level of frataxin protein in cultured cells and/or the supernatant thereof.

In certain embodiments, the methods of the present disclosure may be used as a high-throughput testing assay.

### II. ASSAY METHOD AND SYSTEM

Methods of the disclosure include methods of detecting the presence, measurement of levels, and or changes in levels of various factors described herein. Such factors may include, but are not limited to, target proteins, post-translational modifications, and standard agents. “Levels” may refer to an actual number of factors detected, a concentration of a factor, or a relative level (e.g., through comparison of detection signals between a detected factor and a surrogate factor or standard agent). Such methods may include the use of a capture agent to capture frataxin. As used herein, the term “capture agent” refers to any compound capable of binding an assay component (e.g., assay target protein or compound). Capture agents may include capture antibodies. Capture antibodies may include any of the antibodies described herein. In some cases, capture agents are lectins capable of binding glycosylated assay components.

Additional methods may include the use of a detection agent. As used herein, the term “detection agent” refers to any compound capable of binding an analyte or otherwise indicating the presence and/or level of an analyte in an assay. In some embodiments, detection agents are antibodies (detection antibodies). Detection agents may include antibodies used to detect frataxin. Detection antibodies may include any antibodies described herein. Further, detection agents may include lectins capable of detecting glycosylated assay components. Detection agents may comprise a detectable label. Such detectable labels may include, but are not limited to biotin, streptavidin, avidin, fluorescent labels, enzymatic labels, luminescent labels, and radioactive labels.

Methods may include standard immunological assay formats for capturing proteins and detecting one or more specific epitopes present on such proteins. Such epitopes may include post-translationally modified epitopes. Post-translationally modified epitopes may include glycated and/or glycosylated epitopes.

Methods may include immunological assays. As used herein, an “immunological assay” refers to any assay that utilizes at least one antibody for detection of an analyte. Immunological assays may include, but are not limited to, enzyme linked immunosorbent assays (ELISAs), immunoprecipitation assays, immunofluorescence assays, enzyme immunoassays (EIA), radioimmunoassays (RIA), and Western blot analysis. Methods of the present disclosure may include the use of surface-associated formats or solution-based formats. As used herein, the term “surface-associated format” refers to a method that includes immobilization of one or more assay components on a surface. Such surfaces may include, but are not limited to assay plates, membranes, sensors, or other substrates that include a surface. In some embodiments, the surface-associated format may include a magnetic interaction surface, bead, or coated magnetic beads. In some embodiments, they may include the use of ferromagnetic labels.

Those of ordinary skill in the art will be aware of a variety of assay methods and systems that may be used in connection with the methods of the present disclosure. One type of assay utilized for detection and quantification of a substance is enzyme-linked immunosorbent assay (ELISA). ELISAs are often designed to detect and quantify substances like peptides, proteins, antibodies, and hormones. ELISAs require a capture agent, such as an antigen, to be immobilized on a solid surface and then complexed with an antibody that selectively binds to the antigen. The bound antibody is then linked to a detection agent, such as an antibody, which is further linked to a detectable label, which, when incubated with a substrate, produces a quantifiable and measurable product. The product is often a visual or radioactive signal. ELISAs are often performed in polystyrene plates often with 8 wells, 10 wells, 96 wells, or 384 wells. The wells passively bind reactants, such as antibodies and proteins, which allows for relatively easy separation and removal of bound and non-bound antigens from the assay.

Detection agents can be linked directly to the capture agent, such as a primary antibody, already bound to the substrate, or introduced to the reaction via another detection agent, such as a secondary antibody, which recognizes the primary antibody. The secondary antibody may also be bound to a reporter such as streptavidin, if the secondary antibody is biotin labeled. Streptavidin can bind up to 4 biotin molecules, which may be linked to a reporter. The streptavidin-biotin complex allows for an increased number of reporters associated with each antigen, and thus can increase the signal strength. The most commonly used enzyme labels are horseradish peroxidase (HRP), β-galactosidase, and alkaline phosphatase.

As used herein, the term “solution-based format” refers to a method that includes immobilization of one or more assay components on a substrate that is able to move freely in a liquid medium. Substrates suitable for solution-based formats may include beads or other particles that are mobile in liquid media. Methods utilizing solution-based formats may be analyzed by forcing liquid media through tubes, channels, or other passageways where detection of bound analytes may be carried out. Assay components may be immobilized on surface or solution-based formats in varying levels and/or coating densities. Immobilization may be facilitated through the formation of any number of interactions that include, but are not limited to, covalent bonds, non-covalent bonds, hydrogen bonds, and hydrophobic bonds.

In some embodiments, methods of the disclosure include the use of a capture antibody to immobilize frataxin to a surface or substrate. According to some methods of the present disclosure, frataxin antibodies may be used as a capture antibody to immobilize frataxin to a surface or substrate.

### Antibody Selection

For the purposes of the present disclosure, any functional antibody to a target protein (e.g., frataxin) may be used as either a capture or a detection antibody, or both. Antibodies may be generated by any method known to one with skill in the art, such as, but not limited to, immunization (e.g., hybridoma) and display technologies (e.g., phage display, yeast display, and ribosomal display). Antibodies may be developed, for example, using any naturally occurring or synthetic antigen (e.g., frataxin). As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

Antibodies of the present disclosure may be generated de novo or may be commercially available. In certain embodiments, commercially available antibodies to frataxin are used. Non-limiting examples of antibodies used in the method described herein for the detection of frataxin protein levels in a biological sample (e.g., biofluid or tissue) are described in Table 2.

TABLE 2
Antibody selection

| Purpose | Antibody Name | Details | Vendor | Cat# |
|---|---|---|---|---|
| Capture or | Ab-1 | Unconjugated mouse | LSBio | LS- |
| detection | | monoclonal; to human Frataxin | | C760752 |
| Capture or | Ab-2 | Unconjugated mouse | LSBio | LS- |
| detection | | monoclonal to Human Frataxin | | C755462 |
| | | FXN | | |
| Capture or | Ab-3 | Mouse monoclonal [18A5DB1] | Abcam | ab110328 |
| detection | | to Frataxin | | |
| Capture or | Ab-4 | Mouse monoclonal [17A11] to | Abcam | ab113691 |
| detection | | Frataxin | | |
| Capture or | Ab-5 | Rabbit polyclonal, Recombinant | Abcam | ab175402 |
| detection | | full-length corresponding to | | |
| | | Human Frataxin | | |
| Capture or | Ab-6 | Mouse monoclonal to human | NeuroMab | clone |
| detection | | frataxin protein (81-210) | | N191/7 |
| Standard | N/A | Recombinant, his tagged full | Abcam | ab95502 |
| | | length human frataxin protein | | |

In certain embodiments, the capture antibody is Ab-1 (LS-C760752; PAT10E11AT, ANT-671), an IgG1κ unconjugated mouse monoclonal to human frataxin (42-210), generated by hybridization of mouse F0 myeloma cells with spleen cells from BALBc mice immunized with human frataxin 42-210 purified from E. coli.

In certain embodiments, the detection antibody is Ab-1 (LS-C760752; PAT10E11AT, ANT-671), an IgG1κ unconjugated mouse monoclonal to human frataxin (42-210), generated by hybridization of mouse F0 myeloma cells with spleen cells from BALBc mice immunized with human frataxin 42-210 purified from E. coli. In certain embodiments, the detection agent Ab-1 is capable of engaging with a substance to create a signal, wherein the signal is detectable and quantifiable. In certain embodiments, the detection agent is Ab-1 further comprising a detectable label.

In certain embodiments, the capture antibody is Ab-2 (LS-C755462; AT10E11; IBATGA0440), an IgG1κ unconjugated mouse monoclonal to human frataxin (42-210), generated by hybridization of mouse F0 myeloma cells with spleen cells from BALBc mice immunized with recombinant human frataxin 42-210 purified from E. coli, described in Campuzano et al 1996 Science. 1996; 271(5254):1423-1427 and Shan Y., et al. 2007 Hum Mol Genet. 16(8): 929-41, the contents of each of which are herein incorporated by reference in their entirety.

In certain embodiments, the detection antibody is Ab-2 (LS-C755462; AT10E11; IBATGA0440), an IgG1κ unconjugated mouse monoclonal to human frataxin (42-210), generated by hybridization of mouse F0 myeloma cells with spleen cells from BALBc mice immunized with recombinant human frataxin 42-210 purified from E. coli, described in Campuzano et al 1996 Science. 1996; 271(5254).1423-4427 and Shan Y., et al. 2007 Hum Mol Genet. 16(8): 929-41, the contents of each of which are herein incorporated by reference in their entirety. In certain embodiments, the detection agent Ab-2 is capable of engaging with a substance to create a signal, wherein the signal is detectable and quantifiable. In certain embodiments, the detection agent is Ab-2 further comprising a detectable label.

In certain embodiments, the capture antibody is Ab-3 (ab110328; 18A5DB1), an IgG1κ unconjugated mouse monoclonal to human frataxin (56-210), produced in vitro using hybridomas grown in serum-free medium, then purified using biochemical fractionation.

In certain embodiments, the detection antibody is Ab-3 (ab110328; 18A5DB1), an IgG1κ unconjugated mouse monoclonal to human frataxin (56-210), produced in vitro using hybridomas grown in serum-free medium, then purified using biochemical fractionation. In certain embodiments, the detection agent Ab-3 is capable of engaging with a substance to create a signal, wherein the signal is detectable and quantifiable. In certain embodiments, the detection agent is Ab-3 further comprising a detectable label.

In certain embodiments, the capture antibody is Ab-4 (ab113691; 17A11), an IgG1κ unconjugated mouse monoclonal to human full length frataxin, produced in vitro using hybridomas grown in serum-free medium, then purified using affinity purification.

In certain embodiments, the detection antibody is Ab-4 (ab113691; 17A11), an IgG1κ unconjugated mouse monoclonal to human full length frataxin, produced in vitro using hybridomas grown in serum-free medium, then purified using affinity purification. In certain embodiments, the detection agent Ab-4 is capable of engaging with a substance to create a signal, wherein the signal is detectable and quantifiable. In certain embodiments, the detection agent is Ab-4 further comprising a detectable label.

In certain embodiments, the capture antibody is Ab-5 (ab175402), an IgG unconjugated rabbit polyclonal to human full length frataxin.

In certain embodiments, the detection antibody is Ab-5 (ab175402), an IgG unconjugated rabbit polyclonal to human full length frataxin. In certain embodiments, the detection agent Ab-5 is capable of engaging with a substance to create a signal, wherein the signal is detectable and quantifiable. In certain embodiments, the detection agent is Ab-5 further comprising a detectable label.

In certain embodiments, the capture antibody is Ab-6 (NeuroMab clone N191/7), an IgG2b unconjugated mouse monoclonal antibody to human frataxin (81-210), prepared using BALBc mice and SP2/0 myeloma cells.

In certain embodiments, the detection antibody is Ab-6 (NeuroMab clone N191/7), an IgG2b unconjugated mouse monoclonal antibody to human frataxin (81-210), prepared using BALBc mice and SP2/0 myeloma cells. In certain embodiments, the detection agent Ab-6 is capable of engaging with a substance to create a signal, wherein the signal is detectable and quantifiable. It certain embodiments, the detection agent is Ab-6 further comprising a detectable label.

In certain embodiments, the capture antibody is Ab-1 and the detection antibody is Ab-1. In certain embodiments, the capture antibody is Ab-1 and the detection antibody is Ab-2. In certain embodiments, the capture antibody is Ab-1 and the detection antibody is Ab-3. In certain embodiments, the capture antibody is Ab-1 and the detection antibody is Ab-4. In certain embodiments, the capture antibody is Ab-1 and the detection antibody is Ab-5. In certain embodiments, the capture antibody is Ab-1 and the detection antibody is Ab-6. In certain embodiments, the capture antibody is Ab-2 and the detection antibody is Ab-1. In certain embodiments, the capture antibody is Ab-2 and the detection antibody is Ab-2. In certain embodiments, the capture antibody is Ab-2 and the detection antibody is Ab-3. In certain embodiments, the capture antibody is Ab-2 and the detection antibody is Ab-4. In certain embodiments, the capture antibody is Ab-2 and the detection antibody is Ab-5. In certain embodiments, the capture antibody is Ab-2 and the detection antibody is Ab-6. In certain embodiments, the capture antibody is Ab-3 and the detection antibody is Ab-1. In certain embodiments, the capture antibody is Ab-3 and the detection antibody is Ab-2. In certain embodiments, the capture antibody is Ab-3 and the detection antibody is Ab-3. In certain embodiments, the capture antibody is Ab-3 and the detection antibody is Ab-4. In certain embodiments, the capture antibody is Ab-3 and the detection antibody is Ab-5. In certain embodiments, the capture antibody is Ab-3 and the detection antibody is Ab-6. In certain embodiments, the capture antibody is Ab-4 and the detection antibody is Ab-1. In certain embodiments, the capture antibody is Ab-4 and the detection antibody is Ab-2. In certain embodiments, the capture antibody is Ab-4 and the detection antibody is Ab-3. In certain embodiments, the capture antibody is Ab-4 and the detection antibody is Ab-4. in certain embodiments, the capture antibody is Ab-4 and the detection antibody is Ab-5. In certain embodiments, the capture antibody is Ab-4 and the detection antibody is Ab-6. In certain embodiments, the capture antibody is Ab-5 and the detection antibody is Ab-1. In certain embodiments, the capture antibody is Ab-5 and the detection antibody is Ab-2. in certain embodiments, the capture antibody is Ab-5 and the detection antibody is Ab-3. In certain embodiments, the capture antibody is Ab-5 and the detection antibody is Ab-4. In certain embodiments, the capture antibody is Ab-5 and the detection antibody is Ab-5. In certain embodiments, the capture antibody is Ab-5 and the detection antibody is Ab-6. In certain embodiments, the capture antibody is Ab-6 and the detection antibody is Ab-1. In certain embodiments, the capture antibody is Ab-6 and the detection antibody is Ab-1. in certain embodiments, the capture antibody is Ab-6 and the detection antibody is Ab-2. In certain embodiments, the capture antibody is Ab-6 and the detection antibody is Ab-3. In certain embodiments, the capture antibody is Ab-6 and the detection antibody is Ab-4. In certain embodiments, the capture antibody is Ab-6 and the detection antibody is Ab-5. In certain embodiments, the capture antibody is Ab-6 and the detection antibody is Ab-6.

In developing an assay for the detection of human frataxin protein in biofluids such as CSF, a matrix of 25 combinations of the above antibodies were tested (excluding Ab-6).

In certain embodiments, variants of any of the aforementioned antibodies may be used in any of the methods described herein.

In some embodiments, the antibodies disclosed herein are compatible with a platform for detecting FXN in a biofluid, e.g., the Simoa® platform, and are used as capture and/or detection agents. In some embodiments, the capture and/or detection agent is NovusBio H00002395-M01 or NovusBio H00002395-M03. In some embodiments, the capture and/or detection agent does not comprise GST-tagged antibodies, biotinylated antibodies, fluorescently tagged antibodies, antibodies formulated in a buffer with BSA or other carrier protein, and/or antibodies prepared as ascites. Table 3, below, provides examples of less preferred antibodies for capture and/or detection agents in embodiments of the disclosure.

TABLE 3
Exemplary Antibodies Less Suitable as Capture/Detection
Agents in Simoa ® Assay

| Antibody | Vendor | Catalog Number |
|---|---|---|
| Human Frataxin Matched | Abcam | ab212062 |
| Antibody Pair Kit | | |
| Anti-Frataxin Antibody, | Millipore | MAB1594 |
| Ascites | | |
| Anti-Frataxin antibody | Abcam | ab124680 |
| [EPR6107] | | |
| Frataxin (human) | PerkinElmer ® | AL322 |
| AlphaLISA ® Detection Kit | | |

A reference human frataxin protein standard may be used for any of the methods described herein. A non-limiting example of a reference standard comprises Abcam ab95502, a his-tagged full length human frataxin (42-210). Another non-limiting example of a reference standard comprises the recombinant human frataxin from Abcam: ab110353 (56-210).

### Antibody Fragments and Variants

Antibodies of the present disclosure may include antibody fragments (e.g., antigen binding regions) from intact antibodies. Examples of antibody fragments may include, but are not limited to Fab, Fab′, F(ab′)₂, and Fv fragments; diabodies; linear antibodies; and single-chain antibody molecules. In some embodiments, antibodies of the present disclosure include multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Antibodies of the present disclosure may comprise one or more of these fragments. As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., the ability to bind an antigen).

In one embodiment, the Fc region may be a modified Fc region, as described in US Patent Publication US20150065690, the contents of which are herein incorporated by reference in their entirety, wherein the Fc region may have a single amino acid substitution as compared to the corresponding sequence for the wild-type Fc region, wherein the single amino acid substitution yields an Fc region with preferred properties to those of the wild-type Fc region. Non-limiting examples of Fc properties that may be altered by the single amino acid substitution include binding properties and response to pH conditions (e.g., altered stability and/or target affinity).

As used herein, the term “native antibody” refers to a usually heterotetrameric glycoprotein of about 150,000 Daltons, composed of two identical light (L) chains and two identical heavy (H) chains. Genes encoding native antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain typically have regularly spaced intrachain disulfide bridges. Each heavy chain has at one end a heavy chain variable domain (V_{H}) followed by a number of constant domains. Each light chain has a light chain variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that may differ extensively in sequence among antibodies and are used in the binding and specificity of each particular antibody for its particular antigen. Variable domains typically include hypervariable regions. As used herein, the term “hypervariable region” refers to a region within a variable domain having amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody having a structure that is complementary to its target antigen or epitope. Other portions of the variable domain, not interacting with the antigen, are referred to as framework (FW) regions. The antigen-binding site (also known as the antigen combining site or paratope) typically includes amino acid residues necessary to interact with a particular antigen. The exact residues making up antigen-binding sites are typically elucidated by co-crystallography with bound antigen, however computational assessments can also be used based on comparisons with other antibodies (Strohl, W. R. Therapeutic Antibody Engineering. Woodhead Publishing, Philadelphia Pa. 2012. Ch. 3, p 47-54, the contents of which are herein incorporated by reference in their entirety). Determining residues making up CDRs may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and Al-Lazikani, B. et al., 1997, J. Mol. Biol. 273(4):927-48, the contents of each of which are herein incorporated by reference in their entirety], and Lefranc (Lefranc, M. P. et al., 2005, Immunome Res. 1:3) and Honegger (Honegger, A. and Pluckthun, A. 2001. J. Mol. Biol. 309(3):657-70, the contents of each of which are herein incorporated by reference in their entirety).

V_{H} and V_{L} domains typically have three CDRs each. V_{L} CDRs are referred to herein as CDR-L1, CDR-L2 and CDR-L3, in order of occurrence when moving from N- to C-terminus along the variable domain polypeptide. V_{H} CDRs are referred to herein as CDR-H1, CDR-H2 and CDR-H3, in order of occurrence when moving from N- to C-terminus along the variable domain polypeptide. Each of the CDRs typically have favored canonical structures with the exception of the CDR-H3, which may include amino acid sequences that may be highly variable in sequence and length between antibodies resulting in a variety of three-dimensional structures in antigen-binding domains (Nikoloudis, D. et al., 2014. Peer J. 2:e 456; the contents of which are herein incorporated by reference in their entirety). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p. 47-54, the contents of which are herein incorporated by reference in their entirety).

In some embodiments, antibodies of the present disclosure may be humanized. In some embodiments, antibodies of the present disclosure may be formatted as Fv fragments. As used herein, the term “Fv” refers to an antibody fragment comprising the minimum fragment of an antibody needed to form a complete antigen-binding site. These regions consist of a dimer of one heavy chain and one light chain variable domain in tight, non-covalent association. Fv fragments can be generated by proteolytic cleavage but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia Pa. 2012. Ch. 3, p 46-47, the contents of which are herein incorporated by reference in their entirety).

In some embodiments of the disclosure, the nucleic acid sequence or amino acid sequence may be optimized for expression.

As used herein, the term “light chain” refers to a component of an antibody from any vertebrate species assigned to one of two clearly distinct types, called kappa and lambda based on amino acid sequences of constant domains.

In some embodiments, antibodies of the present disclosure may be formatted as scFvs. As used herein, the term “single chain Fv” or “scFv” refers to a fusion protein of V_{H} and V_{L} antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with antibody display methods (e.g., phage display, yeast display or other display format) where they may be expressed in association with a surface member (e.g. phage coat protein or cell surface molecule) and used in the identification of high affinity peptides for a given antigen.

Antibodies of the present disclosure may be formatted as bispecific antibodies. As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

In some embodiments, antibodies of the present disclosure may be produced as diabodies. As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP404097; WO1993011161; and Hollinger et al. (Hollinger, P. et al., “Diabodies”: Small bivalent and bispecific antibody fragments. PNAS. 1993. 90:6444-8) the contents of each of which are incorporated herein by reference in their entirety.

Antibodies of the present disclosure may be monoclonal antibodies. As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

In some embodiments, antibodies of the present disclosure may be humanized antibodies. As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. Antibody humanization may be carried out according to any of the methods taught in US Publication No. US20150284455, the contents of which are herein incorporated by reference in their entirety.

Antibodies of the present disclosure may include antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but not limited to affibody molecules, affilins, affitins, anticalins, avimers, Centyrins, DARPINS™, Fynomers and Kunitz and domain peptides. In other embodiments, antibody mimetics may include one or more non-peptide regions.

In some embodiments, antibodies of the present disclosure may include antibody variants. As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

In some embodiments, antibodies of the present disclosure may include “unibodies,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. Other antibodies may be “miniaturized” antibodies, which are compacted 100 kDa antibodies (see, e.g., Nelson, A. L., MAbs., 2010. January-February; 2(1):77-83).

The preparation of antibodies, whether monoclonal or polyclonal, may be carried out using any methods known in the art. Techniques for the production of antibodies may be carried out as described, e.g. in Harlow and Lane “Antibodies, A Laboratory Manual,” Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012, the contents of each of which are herein incorporated by reference in their entirety.

### Multispecific Antibodies

In some embodiments, antibodies of the present disclosure may bind more than one epitope. As used herein, the terms “multibody” or “multi specific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

### ELISA

### Direct

In various embodiments, a direct ELISA assay may be used with the methods disclosed herein. The direct ELISA procedure proceeds accordingly, in some embodiments: an antigen is bound to the substrate or detection plate, then a binding wash is incubated with the antigen-bound plate. The binding wash includes primary antibodies to the antigen. After the incubation, the binding wash is removed, and any non-bound antibodies are washed away. The primary antibody may have an active detection modality bound to it. Excess detection wash is removed and washed. Finally, a reporter-specific substrate is washed over the plate which causes a measurable signal to be created by the bound complex. The signal may be read via different instrumentation, such as spectrophotometer, fluorometer, or a luminometer. The signal may be light, radiation, or any other qualifiable or quantifiable noticeable characteristic. The direct detection method uses a labeled primary antibody that reacts directly with the antigen. Direct detection can be performed with an antigen that is directly immobilized on the assay plate or with the capture assay format.

Other variations of the direct ELISA procedure may be performed depending on the antigen and detection needs. ELISAs can be performed with a number of modifications to the basic procedure. In some embodiments, immobilization of the antigen of interest, can be accomplished by direct adsorption to the assay plate or indirectly via a capture antibody that has been attached to the plate. The antigen is then detected either directly (labeled primary antibody) or indirectly (labeled secondary antibody).

### Indirect

An indirect ELISA procedure uses a labeled secondary antibody as the detection agent. The procedure begins similarly to a direct procedure, so that an antigen is bound to a substrate and a solution with the primary antibody is incubated on the substrate, then washed off. A second solution containing a secondary antibody is then incubated on the substrate so that the secondary antibody binds to the primary. The secondary antibody in a second detection wash is then incubated on the plate. The detection wash includes secondary antibodies, which are linked to a reporter complex, such as the streptavidin-biotin reporter. The secondary antibody is intended to bind to the primary antibody, which should be bound to the antigen.

The indirect detection method uses a labeled secondary antibody for detection and is the most popular format for ELISA. The secondary antibody has specificity for the primary antibody. In a sandwich ELISA, it is critical that the secondary antibody be specific for the detection primary antibody only (and not the capture antibody) or the assay will not be specific for the antigen. Generally, this is achieved by using capture and primary antibodies from different host species (e.g., mouse IgG and rabbit IgG, respectively). For sandwich assays, it is beneficial to use secondary antibodies that have been cross-adsorbed to remove any secondary antibodies that might have affinity for the capture antibody.

### Sandwich

In various embodiments, a sandwich ELISA assay may be used with the methods disclosed herein. This type of capture assay is called a “sandwich” assay because the analyte to be measured is typically bound between two primary antibodies—the capture antibody and the detection antibody. As a non-limiting example protocol for a sandwich ELISA, a surface may first be prepared for coating with a capture antibody. Non-specific binding sites may be blocked and then antigen containing sample added to the container (e.g., 96 well plate) for antigen binding to the adhered antibody. A wash may be used to remove any unbound antigen before applying a specific antibody for detection of the antigen, thereby “sandwiching” the antibody between the capture and detection antibodies. A secondary antibody with a detectable label may then be used for detecting or quantifying a signal identifying the presence of the targeted antigen.

Besides the direct, indirect, and sandwich formats described above, several other styles of ELISAs exist and may be used, in some embodiments, with the methods disclosed herein. For instance, competitive ELISA is a strategy that is commonly used when the antigen is small and has only one epitope, or antibody binding site. One variation of this method consists of labeling purified antigen instead of the antibody. Unlabeled antigen from samples and the labeled antigen compete for binding to the capture antibody. A decrease in signal from the purified antigen indicates the presence of the antigen in samples when compared to assay wells with labeled antigen alone.

ELISPOT (enzyme-linked immunospot assay) refers to ELISA-like capture and measurement of proteins secreted by cells that are plated in PVDF-membrane-backed microplate wells. It is a “sandwich” assay in which the proteins are captured locally as they are secreted by the plated cells, and detection is performed with a precipitating substrate. ELISPOT is like a Western blot in that the result is spots on a membrane surface.

In-cell ELISA is performed with cells that are plated and cultured overnight in standard microplates. After the cultured cells are fixed, permeabillized and blocked, target proteins are detected with antibodies. This is an indirect assay, not a sandwich assay. The secondary antibodies are either fluorescent (for direct measurement by a fluorescent plate reader or microscope) or enzyme-conjugated (for detection with a soluble substrate using a plate reader).

### Single Molecule Array

Though ELISAs described to date are considered to have low limits of detection and/or limits of quantification as compared to bulk analysis techniques, a need still exists for assay methods that have even lower limits of detection and/or limits of quantification, allowing for sub-picogram detection of a target protein, not previously determinable and/or apparent. For example, in the method described below, for the detection of the presence of frataxin in circulating biofluids, the limits of detection (LOD), and/or limits of quantification (LOQ) are substantially lower than the LOD and/or LOQ provided by previously described ELISA techniques.

In various embodiments, a sandwich assay according to the methods disclosed herein may be used to detect frataxin in circulating biofluids, which generally are at levels too low for detection via previously described ELISAs. In aspects of the disclosure, an unlabeled antibody is immobilized on a solid substrate (e.g., microtiter plate wells or another solid support) and the sample to be tested is brought into contact with the bound molecule for a period of time sufficient to allow formation of an antibody-antigen complex. In certain embodiments, the sample to be tested is brought into contact with the bound molecule for about 10-60 minutes, e.g., about 35 minutes. In certain embodiments, the sample to be tested is brought into contact with the bound molecule for about 60-150 minutes, e.g., about 75 minutes, or about 120 minutes. A second antibody, labeled with a reporter molecule (capable of inducing a detectable signal) or unlabeled, may be then added to the mixture contained on or in the solid substrate and contacted for a period of time sufficient to allow for the formation of a second antibody-antigen complex (to form an antibody-frataxin-antibody complex). In certain embodiments, the period of time sufficient to allow for formation of a second antibody-antigen complex is about 1-30 minutes, e.g., about 5 minutes, about 30 minutes. In certain embodiments, the period of time sufficient to allow for formation of a second antibody-antigen complex is about 10-60 minutes, e.g., about 30 minutes. Unreacted material can then be washed away from the antibody-frataxin-antibody complex and the presence of frataxin is determined by observation of a signal, which may be quantitated by comparison with a control sample containing known amounts of frataxin. For example, if a labeled antibody is used, the signal can be detected directly via the addition of a substance that allows for the generation of a signal. If an unlabeled antibody is used as the second antibody, then a third antibody (specific for the second antibody that is bound to frataxin) that is labeled is used to contact the antibody-frataxin-antibody complex to generate a signal. Simultaneous assays (where both sample and antibody are added simultaneously to the solid substrate bound antibody) or reverse sandwich assays (where the labeled antibody and sample to be tested are first combined, incubated and added to the unlabeled surface bound antibody) can be used for the detection of frataxin in biological samples.

As discussed above, a first antibody may be bound to a solid support. The solid support can be metal, glass, or a polymer, including, but not limited to cellulose, polyacrylamide, nylon, polystyrene, polyvinylchloride, or polypropylene. The metal may be magnetic. The solid supports may be in the form of tubes, beads, discs microplates, or any other surfaces suitable for conducting an immunoassay. After attachment of the first antibody to the solid support, the solid support may be washed and a biological sample containing frataxin may be then added to the antibody containing the solid support for a period of time sufficient to allow binding of any frataxin protein present to the antibody bound to the solid support. In certain embodiments, the period of time sufficient to allow binding of any frataxin protein present to the antibody bound to the solid support is about 35 minutes. In certain embodiments, the period of time sufficient to allow binding of any frataxin protein present to the antibody bound to the solid support is about 75 minutes. In certain embodiments, the period of time sufficient to allow binding of any frataxin protein present to the antibody bound to the solid support is about 120 minutes. A second antibody can then be added and incubated for an additional period of time sufficient to allow the second antibody to bind to the frataxin bound by the antibody attached to the solid support. In certain embodiments, the period of time sufficient to allow for the second antibody to bind to the frataxin bound by the antibody attached to the solid support is about 5 minutes. In certain embodiments, the period of time sufficient to allow for the second antibody to bind to the frataxin bound by the antibody attached to the solid support is about 30 minutes. The second antibody can be linked to a label that allows of the generation of a signal or the second antibody can be unlabeled. If the second antibody is unlabeled, a labeled third antibody that specifically binds to the second antibody can be used to detect the antibody-frataxin-antibody complex formed in an immunoassay.

Non-limiting examples of labels suitable for use in this aspect of the disclosure include radioisotopes, enzymes, or fluorophores. Commonly used enzymes include horseradish peroxidase, glucose oxidase, β-galactosidase, and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product. In cases where the second antibody is labeled, the labeled antibody may be added to the first antibody-frataxin protein complex and allowed to bind to the complex, and then the excess reagent is washed away. A solution containing the appropriate substrate is then added for the development of a signal.

The terms “limit of detection” (or LOD) and “limit of quantification” (or LOQ) are given their ordinary meaning in the art. The LOD refers to the lowest analyte concentration likely to be reliably distinguished from background noise and at which detection is feasible. The LOD as used herein is defined as 2.5× standard deviations (SD) above background signal, assuming a 10% coefficient of variation (CV). The LOQ may be subdivided to an “upper limit of quantification” (or ULOQ) and a “lower limit of quantification” or (LLOQ), typically defined as the highest and lowest standard curve points wherein the concentration of an analyte may still be accurately measured. As used herein, LOQ and LLOQ may be used interchangeably, unless context indicates otherwise, to refer to the lowest concentration at which the analyte can not only be reliably detected but at which some predefined goals for bias and imprecision are met (e.g., based on % CV). Generally, as is used herein, the LOQ refers to the average of a series of measurements of the lowest concentration above the LOD wherein the CV is less than about 20% and did not have a previous dilution with a CV greater than 20%.

In some embodiments, the Single Molecule Array for frataxin in circulating biofluids may follow any of the methods and protocols described in U.S. Pat. Nos. 9,932,626, 8,222,047, U.S. Patent Publication 2017/0160292, International Application Publication WO2010039180, and embodied by the Simoa® Bead technology (Quanterix, Billerica, Mass.) all of which are incorporated herein by reference in their entirety. In some cases, an assay method employed has a limit of detection and/or a limit of quantification of less than about or about 500 pg/mL, less than about or about 250 pg/mL, less than about or about 100 pg/mL, less than about or about 50 pg/mL, less than about or about 40 pg/mL, less than about or about 30 pg/mL, less than about or about 20 pg/mL, less than about or about 10 pg/mL, less than about or about 5 pg/mL, less than about or about 4 pg/mL, less than about or about 3 pg/mL, less than about or about 2 pg/mL, less than about or about 1 pg/mL, less than about or about 0.8 pg/mL, less than about or about 0.7 pg/mL, less than about or about 0.6 pg/mL, less than about or about 0.5 pg/mL, less than about or about 0.4 pg/mL, less than about or about 0.3 pg/mL, less than about or about 0.2 pg/mL, less than about or about 0.1 pg/mL, less than about or about 0.05 pg/mL, less than about or about 0.04 pg/mL, less than about or about 0.02 pg/mL, less than about or about 0.01 pg/mL, or less. In some cases, an assay method employed has a limit of quantification and/or a limit of detection between about 100 pg/mL and about 0.01 pg/mL, between about 50 pg/mL, and about 0.02 pg/mL, between about 25 pg/mL and about 0.02 pg/mL, between about 10 pg/mL and about 0.02 pg/mL. As will be understood by those of ordinary skill the art, the LOQ and/or LOD may differ for each assay method and/or each biomarker determined with the same assay. In some embodiments, the LOD of an assay employed for detecting frataxin is about equal to or less than 0.02 pg/mL. In some embodiments, the LOQ for an assay employed for detecting frataxin is equal to or less than 0.04 pg/mL.

In some embodiments, the concentration of frataxin in the circulating biofluid sample that may be substantially accurately determined is less than about or about 5000 fM, less than about or about 3000 fM, less than about or about 2000 fM, less than about or about 1000 fM, less than about or about 500 fM, less than about or about 300 fM, less than about or about 200 fM, less than about or about 100 fM, less than about or about 50 fM, less than about or about 25 fM, less than about or about 10 fM, less than about or about 5 fM, less than about or about 2 fM, less than about or about 1 fM, less than about or about 0.5 fM, less than about or about 0.1 fM, or less. In some embodiments, the concentration of frataxin in the fluid sample that may be substantially accurately determined is between about 5000 fM and about 0.1 fM, between about 3000 fM and about 0.1 fM, between about 1000 fM and about 0.1 fM, between about 1000 fM and about 1 fM, between about 100 fM and about 1 fM, between about 100 fM and about 0.1 fM, or the like. The concentration of analyte molecules or particles in a fluid sample may be considered to be substantially accurately determined if the measured concentration of the frataxin in the fluid sample is within about 10% of the actual (e.g., true) concentration of the frataxin in the fluid sample. In certain embodiments, the measured concentration of the frataxin in the fluid sample may be within about 5%, within about 4%, within about 3%, within about 2%, within about 1%, within about 0.5%, within about 0.4%, within about 0.3%, within about 0.2% or within about 0.1%, of the actual concentration of the frataxin in the fluid sample. In some cases, the measure of the concentration determined differs from the true (e.g., actual) concentration by no greater than about 20%, no greater than about 15%, no greater than 10%, no greater than 5%, no greater than 4%, no greater than 3%, no greater than 2%, no greater than 1%, or no greater than 0.5%. The accuracy of the assay method may be determined, in some embodiments, by determining the concentration of frataxin in a fluid sample of a known concentration using the selected assay method.

In certain embodiments, a method for detection and/or quantifying frataxin in a sample comprises immobilizing a plurality of frataxin with respect to a plurality of capture objects (e.g., beads) that each include a binding surface having affinity for at least one type of frataxin epitope. For example, the capture objects may comprise a plurality of beads comprising a plurality of capture components (e.g., an antibody having specific affinity for frataxin). At least some of the capture objects (e.g., having at least some association with at least one frataxin molecule) may be spatially separated/segregated into a plurality of locations, and at least some of the locations may be addressed/interrogated (e.g., using an imaging system). A measure of the concentration of frataxin in the fluid sample may be assessed based on the information received when addressing the locations (e.g., using the information received from the imaging system and/or processed using a computer implemented control system). In some embodiments, a measure of the concentration may be based at least in part on the number of locations determined to contain a capture object that is or was associated with at least one frataxin molecule. In other embodiments and/or under differing conditions, a measure of the concentration may be based at least in part on an intensity level of at least one signal indicative of the presence of a plurality of frataxin molecules and/or capture objects associated with a frataxin molecule at one or more of the addressed locations.

An exemplary assay method may proceed as follows. A sample biofluid (such as human serum, human plasma, or human CSF) containing or suspected of containing frataxin molecules is provided. An assay binding surface comprising a plurality of assay sites is exposed to the sample biofluid. In some cases, the frataxin molecules may be provided in a manner (e.g., at a concentration) such that a statistically significant fraction of the assay sites contains a single frataxin molecule and a statistically significant fraction of the assay sites do not contain any biomarker molecules. The assay sites may optionally be exposed to a variety of reagents (e.g., using a reagent loader) and or rinsed. The assay sites may then optionally be sealed and imaged (see for example, U.S. Pat. No. 9,952,237, which is incorporated by reference in its entirety). The images are then analyzed (e.g., using a computer implemented control system) such that a measure of the concentration of the frataxin molecules in the fluid sample may be obtained, based at least in part by determination of the number/fraction/percentage of assay sites that contain a frataxin molecule and/or the number/fraction/percentage of sites that do not contain any frataxin molecules. In some cases, the frataxin molecules are provided in a manner (e.g., at a concentration) such that at least some assay sites comprise more than one frataxin molecule. In some embodiments, a measure of the concentration of frataxin molecules in the fluid sample may be obtained at least in part on an intensity level of at least one signal indicative of the presence of a plurality of frataxin molecules at one or more of the assay sites.

In some embodiments, the methods optionally comprise exposing the fluid sample to a plurality of capture objects, for example, beads. At least some of the frataxin molecules are immobilized on a bead. The bead may be polystyrene or magnetic or be made up of any other substance known in the art. In some cases, the frataxin molecules are provided in a manner (e.g., at a concentration) such that a statistically significant fraction of the beads associates with a single frataxin molecule and a statistically significant fraction of the beads do not associate with any frataxin molecules. At least some of the plurality of beads (e.g., those associated with a single frataxin molecule or not associated with any frataxin molecules) may then be spatially separated/segregated into a plurality of assay sites (e.g., of an assay consumable). The assay sites may optionally be exposed to a variety of reagents and/or rinsed. At least some of the assay sites may then be addressed to determine the number of assay sites containing a frataxin molecule. In some cases, the number of assay sites containing a bead not associated with a frataxin molecule, the number of assay sites not containing a bead and/or the total number of assay sites addressed may also be determined. Such determination(s) may then be used to determine a measure of the concentration of frataxin molecules in the fluid sample. In some cases, more than one frataxin molecule may associate with a bead and/or more than one bead may be present in an assay site. In some cases, the plurality of frataxin molecules may be exposed to at least one additional reaction component prior to, concurrent with, and/or following spatially separating at least some of the frataxin molecules into a plurality of locations.

The frataxin molecules may be directly detected or indirectly detected. In the case of direct detection, a frataxin molecule may comprise a molecule or moiety that may be directly interrogated and/or detected (e.g., a fluorescent entity). In the case of indirect detection, an additional component is used for determining the presence of the frataxin molecule. For example, the frataxin molecules (e.g., optionally associated with a bead) may be exposed to at least one type of binding ligand. A “binding ligand” is any molecule, particle, or the like that specifically binds to or otherwise specifically associates with a frataxin molecule to aid in the detection of the biomarker molecule. In certain embodiments, a binding ligand may be adapted to be directly detected (e.g., the binding ligand comprises a detectable molecule or moiety) or may be adapted to be indirectly detected (e.g., including a component that can convert a precursor labeling agent into a labeling agent). A component of a binding ligand may be adapted to be directly detected in certain embodiments where the component comprises a measurable property (e.g., a fluorescence emission, a color, etc.). A component of a binding ligand may facilitate indirect detection, for example, by converting a precursor labeling agent into a labeling agent (e.g., an agent that is detected in an assay). A “precursor labeling agent” is any molecule, particle, or the like that can be converted to a labeling agent upon exposure to a suitable converting agent (e.g., an enzymatic component). A “labeling agent” is any molecule, particle, or the like, that facilitates detection, by acting as the detected entity, using a chosen detection technique. In some embodiments, the binding ligand may comprise an enzymatic component (e.g., horseradish peroxidase, beta-galactosidase, alkaline phosphatase, etc.). A first type of binding ligand may or may not be used in conjunction with additional binding ligands (e.g., second type, etc.).

The assay methods and systems may employ a variety of different components, steps, and/or other aspects that will be known and understood by those of ordinary skill in the art. For example, a method may further comprise determining at least one background signal determination (e.g., and further comprising subtracting the background signal from other determinations), wash steps, and the like. In some cases, the assays or systems may include the use of at least one binding ligand, as described herein. In some cases, the measure of the concentration of biomarker molecules in a fluid sample is based at least in part on comparison of a measured parameter to a calibration curve. In some embodiments, a calibration curve is formed at least in part by determination of at least one calibration factor, as described above.

In some embodiments, a binding ligand and/or a biomarker may comprise an enzymatic component. The enzymatic component may convert a precursor labeling agent (e.g., an enzymatic substrate) into a labeling agent (e.g., a detectable product). A measure of the concentration of biomarker molecules in the fluid sample can then be determined based at least in part by determining the number of locations containing a labeling agent (e.g., by relating the number of locations containing a labeling agent to the number of locations containing a biomarker molecule (or number of capture objects associated with at least one biomarker molecule to total number of capture objects)). Non-limiting examples of enzymes or enzymatic components include horseradish peroxidase, beta-galactosidase, and alkaline phosphatase.

### Exemplary Single Molecule Array Protocol

In some embodiments, a non-limiting example of a single molecule array is used to evaluate frataxin in a biofluid sample. Briefly, paramagnetic beads conjugated to a primary capture antibody are mixed with sample for target binding. Beads are subsequently exposed to a fluorescing detection antibody and the sample, now containing beads with full immunocomplexes, is run on the Quanterix microarray disk on which individual beads are captured in femtoliter microwells and optically analyzed for the presence or absence of signal in each well.

In some embodiments, the single molecule array protocol comprises associating capture agents, e.g., antibodies (to frataxin), with the surface of paramagnetic beads (e.g., 2.7 μm beads) in order to concentrate a dilute solution of molecules (i.e., a circulating biofluid containing frataxin). It is contemplated that biofluids include any fluids from a body, including nascent fluids as well as liquids made from a previously solid element, such as cell lysate. Circulating biofluids may include non-solid liquids that traverse the body, such as blood serum, blood plasma, and CSF. In some embodiments, the fluids and circulating biofluids are from human samples, although, fluids and circulating biofluids may also originate in some embodiments from other animals. In some embodiments, the beads (Simoa®, Quanterix, Billerica, Mass.) may contain approximately 250,000 attachment sites, which creates a field of capture molecules.

In some embodiments, antibodies may be selected or modified for use in the assay, e.g., using a screening matrix of 25 antibody combinations (such as Ab-1, Ab-2, Ab-3, Ab-4 and Ab-5) to test for sensitivity, with the final assay conditions leading to sub-picogram levels of detection per milliliter of sample, e.g., with a detection range of 0.6-500 pg/mL in homogenized human motor cortex tissue.

In some embodiments, the capture agent and/or detection agent is or comprises Ab-1. Other capture agents and/or detection agents include Ab-2, Ab-3, Ab-4, Ab-5, or Ab-6, as described in Table 2 above. Combinations of one or more capture agents and/or one or more detection agents, such as those selected from Table 2, may also be used.

In some embodiments, capture agents are present on assay beads. The prepared beads can be added to the dilute solution containing frataxin. The frataxin protein standard may include the Recombinant Human Frataxin from Abcam cat #ab95502. The beads may be prepared in a Homebrew Bead Diluent (supplied by Quanterix, Billerica, Mass.). According to certain embodiments of the method, approximately 500,000 beads can be added to a 100 μL sample. The addition of a high number of beads, at approximately a 10:1 bead-to-molecule ratio, results in a Poisson-like distribution for the percentage of beads containing a labeled immunocomplex (e.g., comprising at least one capture antibody bound to frataxin). In some embodiments, each paramagnetic bead of the plurality of paramagnetic beads comprises either one such immunocomplex or zero immunocomplexes. In some embodiments, a high number of beads is used in the solution, such that the distance between beads in solution is very small, so that every target molecule (i.e., frataxin) encounters a bead in less than a minute. Without being bound by theory, increased encounters between a frataxin molecule increases the likelihood that all the frataxin molecules, even in low concentration solutions, will interact and bind to a capture molecule on a bead, which may improve assay efficacy.

In various embodiments, after incubation of the beads with the solution, the beads are washed to remove non-specifically bound proteins. The washed beads may then be incubated with biotinylated detection antibodies (i.e., to frataxin) and further with β-galactosidase-labeled streptavidin. In some embodiments, the detection agent is or comprises Ab-3. In some embodiments, the detection agent is or comprises Ab-1, Ab-2, Ab-4, Ab-5, or Ab-6, as described in Table 2 above. As a non-limiting example, each detection antibody may be biotinylated at a molar excess ration of 40×. Overall, this process allows each bead that has captured a single frataxin molecule to be labeled with an enzyme. Beads that do not capture a frataxin molecule do not receive a label.

In various embodiments, after the streptavidin-incubation step, the beads are loaded into arrays (Simoa®, Quanterix, Billerica, Mass.) of approximately 216,000 femtoliter-sized wells that hold no more than one bead per well (4.25 μm width, 3.25 μm depth). In some embodiments, the number of paramagnetic beads may be 4×10⁶ to 8×10⁶ assay beads, and may further include 6×10⁶ to 12×10⁶ helper beads. Helper beads do not have affinity for the sample biomarker, such as frataxin (e.g., do not comprise a capture or detection agent with affinity for frataxin). In some embodiments, the number of paramagnetic beads may be 8×10⁶ assay beads, and may further include 12×10⁶ helper beads. In some embodiments, the number of paramagnetic beads may be 4×10⁶ assay beads, and may further include 6×10⁶ helper beads. The beads (assay beads, or assay beads and helper beads) are then sealed into the array with substrate, reactive with the label bound to the detection antibody. In some embodiments, the enzyme may be soluble β-galactosidase (SBG), from Quanterix. The arrays are then imaged with a Simoa® HD-1 Analyzer (Quanterix, Billerica, Mass.). The beads that have captured a frataxin molecule will cause the complexed enzymes to convert the substrate to release a fluorescent product. The Analyzer captures the fluorescent product, which signals the presence of frataxin. The ratio of wells containing an enzyme-complexed bead compared to the total number of wells containing beads corresponds to the analyte (frataxin) concentration in the original sample. The wells that contain beads that did not capture a frataxin molecule will remain dark. In this manner, this exemplary Single Molecule Array assay permits the detection of very low concentrations of analyte and associated enzyme labels, by confining the fluorophores to very small volumes thereby allowing the differentiation of an “on” well (bead+analyte/enzyme complex) from an “off” well (bead with no analyte/enzyme complex).

In certain embodiments, assay conditions following the Simoa® 2-step protocol include a 10-minute to 150-minute (e.g., 10-minute, 20-minute, 30-minute, 35-minute, 40-minute, 45-minute, 50-minute, 60-minute, 70-minute, 75-minute, 80-minute, 90-minute, 100-minute, 115-minute, 120-minute, 125-minute, 135-minute, 140-minute, or 150-minute) incubation of the beads with the sample and detection antibody, followed by a wash step, then a 1-minute to 30-minute (e.g., 1-minute, 2-minute, 3-minute, 4-minute, 5-minute, 6-minute, 7-minute, 8-minute, 9-minute, 10-minute, 15-minute, 20-minute, 30-minute) incubation of the beads with the soluble β-galactosidase (SBG) reagent. In certain embodiments, the ratio of helper beads to assay beads is about 1:5 to 5:1, e.g., is about 1:1, about 1.5:1, or about 2:1. For example, in some embodiments, the ratio of helper beads to assay beads is about 1.5:1. In some embodiments, the assay uses 4×10⁶ to 8×10⁶ assay beads per mL and 6×10⁶ to 12×10⁶ helper beads per mL. In certain embodiments, the assay uses 8×10⁶ assay beads per mL and 12×10⁶ helper beads per mL. In certain embodiments, the assay uses 4×10⁶ assay beads per mL and 6×10⁶ helper beads per mL.

In certain embodiments, assay conditions following the Simoa® 2-step protocol include a 35-minute incubation of the beads with the sample and detection antibody, followed by a wash step, then a 5-minute incubation of the beads with the soluble β-galactosidase (SBG) reagent. In certain embodiments, the ratio of helper beads to assay beads is about 1:5 to 5:1, e.g., is about 1:1, about 1.5:1, or about 2:1. For example, in some embodiments, the ratio of helper beads to assay beads is about 1.5:1. In some embodiments, the assay uses 4×10⁶ to 8×10⁶ assay beads per mL and 6×10⁶ to 12×10⁶ helper beads per mL. In certain embodiments, the assay uses 8×10⁶ assay beads per mL and 12×10⁶ helper beads per mL. In certain embodiments, the assay uses 4×10⁶ assay beads per mL and 6×10⁶ helper beads per mL.

In certain embodiments, assay conditions following the Simoa® 2-step protocol include an incubation of the beads with the sample and detection antibody, followed by a wash step, then a shorter incubation of the beads with a soluble β-galactosidase (SBG) reagent. For example, the assay conditions may comprise a 75-minute incubation of the beads with the sample and detection antibody, followed by a wash step, then a 5-minute incubation of the beads with the soluble β-galactosidase (SBG) reagent. In certain embodiments, the ratio of helper beads to assay beads is about 1:5 to 5:1, e.g., is about 1:1, about 1.5:1, or about 2:1. For example, in some embodiments, the ratio of helper beads to assay beads is about 1.5:1. In some embodiments, the assay uses 4×10⁶ to 8×10⁶ assay beads per mL and 6×10⁶ to 12×10⁶ helper beads per mL. In certain embodiments, the assay uses 8×10⁶ assay beads per mL and 12×10⁶ helper beads per mL. In certain embodiments, the assay uses 4×10⁶ assay beads per mL and 6×10⁶ helper beads per mL.

In certain embodiments, assay conditions following the Simoa® 2-step protocol include a 120-minute incubation of the beads with the sample (e.g., a diluted biofluid sample) and detection antibody, followed by a wash step, then a 30-minute incubation of the beads with the soluble β-galactosidase (SBG) reagent. In certain embodiments, the ratio of helper beads to assay beads is about 1:5 to 5:1, e.g., is about 1:1, about 1.5:1, or about 2:1. For example, in some embodiments, the ratio of helper beads to assay beads is about 1.5:1. In some embodiments, the assay uses 4×10⁶ to 8×10⁶ assay beads per mL and 6×10⁶ to 12×10⁶ helper beads per mL. In certain embodiments, the assay uses 8×10⁶ assay beads per mL and 12×10⁶ helper beads per mL. In certain embodiments, the assay uses 4×10⁶ assay beads per mL and 6×10⁶ helper beads per mL.

In certain embodiments, assay conditions following the Simoa® 2-step protocol include a 120-minute incubation of the beads with the sample (e.g., a diluted biofluid sample) and detection antibody, followed by a wash step, then a 5-minute incubation of the beads with the soluble β-galactosidase (SBG) reagent. In certain embodiments, the ratio of helper beads to assay beads is about 1:5 to 5:1, e.g., is about 1:1, about 1.5:1, or about 2:1. For example, in some embodiments, the ratio of helper beads to assay beads is about 1.5:1. In some embodiments, the assay uses 4×10⁶ to 8×10⁶ assay beads per mL and 6×10⁶ to 12×10⁶ helper beads per mL. In certain embodiments, the assay uses 8×10⁶ assay beads per mL and 12×10⁶ helper beads per mL. In certain embodiments, the assay uses 4×10⁶ assay beads per mL and 6×10⁶ helper beads per mL.

In some embodiments the Simoa® 3-step protocol is used. In certain embodiments, assay conditions following the Simoa® 3-step protocol include a 10-minute to 150-minute (e.g., 10-minute, 20-minute, 30-minute, 35-minute, 40-minute, 45-minute, 50-minute, 60-minute, 70-minute, 75-minute, 80-minute, 90-minute, 100-minute, 110-minute, 120-minute, 125-minute, 135-minute, 140-minute, or 150-minute) incubation of the beads with the sample (e.g., a diluted biofluid sample), followed by a wash step, then a 1-minute to 120-minute (e.g., 1-minute, 5-minute, 10-minute, 20-minute, 30-minute, 35-minute, 40-minute, 45-minute, 50-minute, 60-minute, 70-minute, 75-minute, 80-minute, 90-minute, 100-minute, 110-minute, 120-minute, 125-minute, 135-minute, 140-minute, or 150-minute) incubation of the beads with biotinylated detection antibody, followed by another wash step, and a final 1-minute to 30-minute (e.g., 1-minute, 2-minute, 3-minute, 4-minute, 5-minute, 6-minute, 7-minute, 8-minute, 9-minute, 10-minute, 15-minute, 20-minute, or 30-minute) incubation of the beads with the soluble β-galactosidase (SBG) reagent.

In certain embodiments, assay conditions following the Simoa® 3-step protocol include a 30-minute incubation of the beads with the sample, followed by a wash step, then a 5-minute incubation of the beads with biotinylated detection antibody, followed by another wash step, and a final 5-minute incubation of the beads with the soluble β-galactosidase (SBG) reagent.

In certain embodiments, assay conditions following the Simoa® 3-step protocol include the use of helper beads as well as assay beads. In certain embodiments, the ratio of helper beads to assay beads is about 1:5 to 5:1, e.g., is about 1:1, about 1.5:1, or about 2:1. For example, in some embodiments, the ratio of helper beads to assay beads is about 1.5:1. In some embodiments, the assay uses 4×10⁶ to 8×10⁶ assay beads per mL and 6×10⁶ to 12×10⁶ helper beads per mL. In certain embodiments, the assay uses 8×10⁶ assay beads per mL and 12×10⁶ helper beads per mL. In certain embodiments, the assay uses 4×10⁶ assay beads per mL and 6×10⁶ helper beads per mL.

In certain embodiments, assay conditions may further comprise any combination of the following conditions, such as, but not limited to, a bead diluent comprising a Homebrew Bead Diluent with a Bead Reaction Volume of 25 μL. The concentration of detector in the bottle may be 1.45 μg/mL to 2.0 μg/mL (e.g., 1.45 μg/mL or 2.0 μg/mL), the detector diluent may be Simoa® Diluent 1 or SuperBlock™ PBS, and the detector reaction volume may be 20 μL. An SBG reaction volume of 100 μL may be used, comprising a concentration of 50 pM SBG and SBG Dilution buffer as diluent. In some embodiments, 150 pM SBG may be used. In some embodiments, Calibrator Levels may be set to 0, 0.686, 2.06, 6.17, 18.5, 55.6, 167, 500 pg/mL. In some embodiments, Calibrator Levels may be set to 0, 0.14, 0.41, 1.23, 3.70, 11.1, 33.3, 100 pg/mL. In some embodiments, Calibrator Levels may be set to 0, 2.74, 8.23, 24.7, 74.1, 222, 667, 2000 pg/mL. In certain embodiments, the Calibrator Diluent is Simoa® Diluent 1. Sample Diluent may also be Simoa® Diluent 1 and the calibrator/sample reaction volume may be 100 μL to 170 μL (e.g., 100 μL or 170 μL) with a sample dilution factor (MRD) of 2× or 4×. In certain embodiments, the Calibrator Diluent is SuperBlock™ PBS. Sample Diluent may also be SuperBlock™ PBS and the calibrator/sample reaction volume may be 100 μL to 170 μL (e.g., 100 μL or 170 μL) with a sample dilution factor (MRD) of 2× or 4×. In some embodiments, the Calibrator and/or Sample Diluent may further comprise Newborn Calf Serum (NCS) (e.g., 5% NCS in Simoa® Diluent 1 or 10% NCS in Simoa® Diluent 1).

An assay was tested on a set of control samples obtained from biorepositories (BioIVT, Westbury, N.Y. and Dx Biosamples, San Diego, Calif.) including samples of serum, plasma, and CSF. Frataxin was detected in 19 of the 22 samples tested at ranges of 2.29-282.13 pg/mL in serum, 1.59-5.11 pg/mL in plasma, and 1.89-4.74 pg/mL in CSF. In future work, we contemplate procuring a panel of biofluid samples of both FA subjects and age-matched controls to establish the magnitude of difference in frataxin within these matrices and how these levels might relate to biological and clinical features of FA. Additionally, a contemplated example will expand sample size to better establish baseline frataxin levels in biofluids.

### Single Molecule Counting

In some embodiments of the disclosure, frataxin in a biofluid is detected and/or quantified by counting single molecules bound to frataxin. For instance, a single molecule counting (SMC) assay and protocol, as described by Fischer et al., AAPS J. January; 17(1): 93-101 or Fodale et al., J Huntingtin's Dis. 2017; 6(4):349-361 may be used, the contents of each of which are herein incorporated by reference in their entirety. In some embodiments, the SMC assay has a sensitivity level in the femtogram to picogram levels, similar to that of the Single Molecule Array. The sensitivity of the SMC may be at a level suitable for detection of frataxin in serum, plasma, or CSF. Such methods may include any of those described or claimed in U.S. Pat. No. 7,571,640, the contents of which are incorporated by reference in their entirety. Such assays include labeling the molecule of interest and detecting the presence or absence of the label with a single molecule detector. The single molecule detector may shine a laser at the detector, which is capable of emitting a quantifiable signal in response. A SMC assay may have a level of detection of the single molecule in the sample less than about 10, 1, 0.1, 0.01, or 0.001 femtomolar. The SMC array may use a capture antibody specific for binding frataxin; the capture antibody may also be bound to a signal moiety. Each moiety may further comprise one or more fluorescent entity. The moiety may also include fluorescent dyes, quantum dots, or other signal generating entities. The SMC utilizes a single molecule detector which may include a source of motive force for moving the labeled sample, an electromagnetic source, and an interrogation space to read the signal from the moiety.

### Multiplex Immunoassay Platform

In some embodiments, frataxin and/or neurofilament light chain is detected and/or quantified using an immunoassay on a microfluidic multi-analyte platform. In some embodiments, the immunoassay on a microfluidic multi-analyte platform is the ProteinSimple® Simple Plex Platform. An immunoassay protocol may follow or be adapted from, for example, the protocol as described by Gupta et al., Bioanalysis. 2016 December; 8(23):2415-2428, the contents of which are herein incorporated by reference in their entirety. In some embodiments, the assay has a low- to sub-picogram level sensitivity. The sensitivity of the assay may be at a level suitable for detection of frataxin and/or neurofilament light chain in a biofluid such as blood, serum, plasma, or CSF. In some embodiments, an assay using the ProteinSimple® Simple Plex platform comprises using a microfluidic cartridge preloaded with one or more capture antibody directed to the target analyte (e.g., frataxin, neurofilament light chain) and one or more labeled detection antibody. The detection antibody may also be directed to the target analyte (e.g., frataxin, neurofilament light chain). The detector may shine a laser at the labeled detection antibody, which is capable of emitting a quantifiable signal in response. In some embodiments, the assay uses fluorescent dyes or other signal generating entities.

In some embodiments, the capture and/or detection antibody used in the immunoassay is an anti-frataxin antibody. In some embodiments, the capture and/or detection antibody is Ab-1, Ab-2, Ab-3, Ab-4, Ab-5, or Ab-6. In some embodiments, the capture antibody is Ab-1 and the detection antibody is Ab-3. In some embodiments, the capture and/or detection antibody is an anti-neurofilament light chain antibody.

In some embodiments, the assay may be used for detecting and/or quantifying more than one target analyte (e.g., frataxin and neurofilament light chain) from the same biofluid sample. In some embodiments, the assay has a limit of detection and/or limit of quantification of frataxin and/or neurofilament light chain from the sample of less than about or about 10 pg/mL, less than about or about 1 pg/mL, less than about or about 0.5 pg/mL, less than about or about 0.3 pg/mL, less than about or about 0.1 pg/mL, less than about or about 0.01 pg/mL, less than about or about 0.001 pg/mL, or less. In some embodiments, the assay has a limit of quantification and/or a limit of detection between about 100 pg/mL and about 0.01 pg/mL, between about 50 pg/mL and about 0.02 pg/mL, between about 25 pg/mL and about 0.02 pg/mL, between about 10 pg/mL and about 0.02 pg/mL. In some embodiments, the LOQ and/or LOD may differ for different target analytes assessed with the same assay. In some embodiments, the LOD of an assay employed for detecting frataxin is about equal to or less than 2 pg/mL, about equal to or less than 1.5 pg/mL, or about equal to or less than 1 pg/mL. In some embodiments, the LOD of an assay employed for detecting frataxin is about equal to or less than 0.5 pg/mL. In some embodiments, the LOQ for an assay employed for detecting frataxin is about equal to or less than 2 pg/mL, about equal to or less than 1.5 pg/mL, or about equal to or less than 1 pg/mL. In some embodiments, the LOQ for an assay employed for detecting frataxin is about equal to or less than 0.5 pg/mL. In some embodiments, the LOQ for an assay employed for detecting frataxin is about equal to or less than 0.3 pg/mL.

### Dual Assessment

In some embodiments, any of the methods described herein useful for determining the amount of frataxin protein in a biofluid or sample (e.g., CSF) may be used in combination with one or more additional measures (e.g., dual assessment). As used herein, an “additional measure” refers to any method useful for evaluating or quantifying a metric of interest e.g., quantifying neurofilament light concentration in a biofluid or assessing brain region health or volumes by neuroimaging. Non-limiting examples of additional measures include assays for alternative biomarkers, neuroimaging techniques, and/or clinical assessments of cognitive, motor, sensory or quality of life type metrics.

In certain embodiments, the additional measure may comprise an assay for the quantification of one or more alternative biomarkers, such as but not limited to, neurofilament light chain.

In certain embodiments, the additional measure may be one or more neuroimaging techniques, such as, but not limited to magnetic resonance imaging (MRI) or spectroscopy (MRS), position emission tomography (PET), computed tomography (CT), ultrasound, or diffusion tensor imaging (DTI). In some embodiments, the additional measure (e.g., neuroimaging technique) comprises brain morphometry of the cerebellum and brainstem, spinal cord morphometry, brain and spinal cord diffusion, dentate iron content (brain QSM), and/or spinal cord spectroscopy. For example, in some embodiments, neuroimaging may be used to assess morphometry of the dentate nuclei. In some embodiments, neuroimaging may be used to assess whole cerebellum and brainstem morphometry. In some embodiments, neuroimaging may be used to assess cervical and thoracic spinal cord morphometry. In some embodiments, neuroimaging, e.g., DTI, may be used to assess diffusion in superior, middle, and inferior cerebellar peduncles. In some embodiments, neuroimaging, e.g., DTI, may be used to assess cervical spinal cord diffusion. In some embodiments, neuroimaging, e.g., MRS, may be used to assess cervical spinal cord atrophy.

In some embodiments, the additional measure assesses volume or volume loss. In some embodiments, the volume loss is gray matter volume loss. For example, gray matter loss may be assessed in one or more regions of the CNS. In some embodiments, gray matter loss may be assessed in the spinal cord. In some embodiments, gray matter loss may be assessed in one or more regions of the cerebellum: lobule V, lobule VI, lobule VII, lobule VIII (secondary sensorimotor cerebellum), crus of cerebellum, posterior lobe of vermis, flocculi bilaterally, and/or cerebellar left tonsil. See Vavla et al. Functional and Structural Brain Damage in Friedreich's Ataxia. Front. Neurol. 2018. 9:747, the contents of which are herein incorporated by reference in their entirety. The gray matter volume loss may be at least 5%, e.g., 5%, 10%, 15%, 20%, 25%, 30%, 40%, 50%, 60%, or more than 60% relative to a healthy, age-matched subject.

In some embodiments, the additional measure assesses mean, radial, and/or axial diffusivity (e.g., increased mean, radial and/or axial diffusivity) in white matter. Id. In some embodiments, mean diffusivity is increased by at least 15%, e.g., 15%, 20%, 25%, 30%, 35%, 40%, 45%, 50%, 60%, 70%, or more than 70% relative to a healthy, age-matched subject. In some embodiments, radial diffusivity is increased by at least 30%, e.g., 30%, 35%, 40%, 45%, 50%, 55%, 60%, 65%, 70%, 75%, or more than 75% relative to a healthy, age-matched subject. In some embodiments, axial diffusivity is increased by at least 5%, e.g., 5%, 10%, 15%, 20%, 25%, or more than 25% relative to a healthy, age-matched subject. In some embodiments, the additional measure assesses fractional anisotropy (e.g., reduced fractional anisotropy) in white matter. In some embodiments, fractional anisotropy is reduced by at least 15%, e.g., 15%, 20%, 25%, 30%, 35%, 40%, 45%, 50%, 60%, 70%, or more than 70% relative to a healthy, age-matched subject. In some embodiments, the additional measure assesses lobular atrophy. In some embodiments, the additional measure assesses T2 signal (e.g., T2 signal decrease), for example of dentate nuclei. See Lindig et al., Pattern of Cerebellar Atrophy in Friedreich's Ataxia-Using the SUIT Template. Cerebellum. 2019. 18(3):435-447, the contents of which are herein incorporated by reference in their entirety. For example, in some embodiments, mean volume of the dentate nucleus is decreased by at least 20%, e.g., 20%, 25%, 30%, 35%, 40%, 45%, or more than 45% relative to a healthy, age-matched subject. In some embodiments, the additional measure is accumulation of iron in dentate nuclei. In some embodiments, the additional measure is an increased admixture of iron, copper, and zinc in dentate nuclei. See Koeppen et al. Friedreich's Ataxia Causes Redistribution of Iron, Copper, and Zinc in the Dentate Nucleus. Cerebellum. 2012. 11(4):845-860, the contents of which are herein incorporated by reference in their entirety. In some embodiments, the additional measure assesses spinal cord atrophy, e.g., thoracic and cervical spinal cord flattening, compression, or shrinkage. See Dogan et al. Structural characteristics of the central nervous system in Friedreich's ataxia: an in vivo spinal cord and brain MRI study. Journal of Neurology, Neurosurgery & Psychiatry 2019; 90:615-617, the contents of which are herein incorporated by reference in their entirety. In some embodiments, the thoracic spinal cord cross-sectional area is reduced by at least 5%, e.g., 5%, 10%, 15%, 20%, 25%, 30%, 35%, 40%, 50%, 60%, or more than 60% relative to that of a healthy, age-matched subject. In some embodiments, the thoracic spinal cord volume is reduced by at least 5%, e.g., 5%, 10%, 15%, 20%, 25%, 30%, 35%, 40%, 50%, 60%, or more than 60% relative to that of a healthy, age-matched subject. In some embodiments, the cervical spinal cord cross-sectional area is reduced by at least 5%, e.g., 5%, 10%, 15%, 20%, 25%, 30%, 35%, 40%, 50%, 60%, or more than 60% relative to that of a healthy, age-matched subject. In some embodiments, the cervical spinal cord volume is reduced by at least 5%, e.g., 5%, 10%, 15%, 20%, 25%, 30%, 35%, 40%, 50%, 60%, or more than 60% relative to that of a healthy, age-matched subject.

In certain embodiments, the additional measure may be a clinical assessment or scale. In some embodiments, the clinical assessment is associated with a specific disease or disorder, such as, but not limited to Friedreich's Ataxia. Non-limiting examples of clinical assessments that may be used include the Scale for Assessment and Rating of Ataxia (SARA) or Friedreich's Ataxia Rating Scale (FARS).

In certain embodiments, the readout of the assay for quantifying frataxin concentration in a biofluid may be correlated to the readout of one or more additional measures. In some embodiments, this correlation may be utilized as a biomarker, prognostic, diagnostic, or measure of efficacy of treatment for a disease, disorder, or condition, such as, but not limited to Friedreich's Ataxia.

In some embodiments, one or more biofluids from a subject may be assessed for both frataxin concentration and for neurofilament light chain concentration. Without being bound by theory, neurofilament light chain may serve as a biomarker of neuroaxonal injury, and thus provide a proxy for nervous system integrity (or lack thereof), See Khalil M. “Neurofilaments as biomarkers in neurological disorders,” Nat. Rev. Neurol. 2018, 14:577-589. Measuring neurofilament light chain concentration may provide a confirmatory indication of a subject's disease severity and/or the efficacy of a treatment for Friedreich's Ataxia. Without being bound by theory, when neurons are damaged or dying, neurofilament light chain may leak from the brain into the CSF, then into the bloodstream, and may be detected in biofluids such as CSF, blood, or blood components (e.g., serum). Neurofilament light chain concentrations may be assessed according to methods known in the art, e.g., using electrochemiluminescence or a single molecule array such as the Simoa® platform (e.g., Quanterix Simoa® NF-Light®), according to the manufacturer's instructions.

In some aspects, dual assessment comprises measuring, in one or more collected samples of a biofluid from the subject, a concentration of frataxin protein and a concentration of neurofilament light chain. In some embodiments, the concentration of frataxin protein and the concentration of neurofilament light chain are measured from a single biofluid sample. In some embodiments, the biofluid sample is a CSF sample. In some embodiments, the biofluid sample is a serum sample. In some embodiments, the biofluid sample is a blood sample. In some embodiments, the concentration of frataxin protein and the concentration of neurofilament light chain are measured from a single biofluid sample type, which may be the same sample or may be different samples (e.g., collected at different times from the same subject). In some embodiments, the biofluid sample type is a CSF sample. In some embodiments, the biofluid sample type is a serum sample. In some embodiments, the concentration of frataxin protein and the concentration of neurofilament light chain are measured from different biofluid samples and/or sample types. In some embodiments, the concentration of frataxin protein is measured from a serum sample and the concentration of neurofilament light chain is measured from a CSF sample. In some embodiments, the concentration of frataxin protein is measured from a CSF sample and the concentration of neurofilament light chain is measured from a serum sample.

In some embodiments, dual assessment assesses whether frataxin and/or neurofilament light chain are detectable (e.g., measurable above the limit of detection) in a subject's biofluid sample. In some embodiments, frataxin is detectable.

In some embodiments, neurofilament light chain is detectable at an LOQ no greater than 1 pg/mL, no greater than 0.1 pg/mL, or no greater than 0.01 pg/mL. In certain embodiments, neurofilament light chain may be detectable at a concentration in the biofluid sample of about 0.001 pg/mL to about 0.1 pg/mL, about 0.1 pg/mL to about 10 pg/mL, about 1 pg/mL to about 100 pg/mL, or about 10 pg/mL to about 1000 pg/mL.

In some embodiments, dual assessment assesses whether frataxin is detectable in a subject's biofluid above a given threshold. For example, the threshold may be the baseline frataxin levels detected in the subject's biofluid sample before being administered a therapy suitable for increasing frataxin expression (e.g., gene therapy). In some embodiments, the threshold may be a 0.5× to 3× (e.g., 0.5×, 1×, 1.5×, 2×, 2.5×, or 3×) increase from baseline (e.g., an increase over a frataxin concentration prior to administration of a gene therapy). In some embodiments, the threshold may be at least 20%, at least 30%, at least 40%, at least 50%, at least 60%, at least 70%, at least 80%, at least 90%, at least 95%, at least 99%, or 100% of the frataxin concentration in a biofluid sample from a normal healthy individual (not having Friedreich's Ataxia), optionally an age-matched healthy individual.

In some embodiments, dual assessment assesses whether neurofilament light chain is detectable in a subject's biofluid above or below a given threshold. For example, the threshold may be the baseline neurofilament light chain levels detected in the subject's biofluid sample before being administered a therapy suitable for increasing frataxin expression (e.g., gene therapy). In some embodiments, the threshold may be a 0.5×-2× (e.g., 0.5×, 1×, 1.5×, 2×) or greater decrease in neurofilament light chain concentration compared to baseline (e.g., neurofilament light chain concentration prior to administration of gene therapy). In some embodiments, the threshold is about 30 pg/mL, about 20 pg/mL, about 10 pg/mL, or less neurofilament light chain detected in a subject's biofluid sample. In some embodiments, the threshold is about 40 pg/mL, about 50 pg/mL, or more neurofilament light chain detected in a subject's biofluid sample. In some embodiments, the threshold is stable (no change in) neurofilament light chain concentration in the subject's biofluid sample before and after gene therapy. In some embodiments, the threshold is the absence of a significant increase in neurofilament light chain concentration in the subject's biofluid sample after gene therapy compared to before gene therapy (e.g., a relative increase in neurofilament light chain concentration of no more than 1, 2, or 3 pg/mL, or a relative increase of no more than 3%, 5%, or 7%). In some embodiments, the neurofilament light chain concentration for a given subject is compared to that of a healthy individual (an individual not having a neurological or neurodegenerative condition and not having brain injury) of the same age, for example, to normalize for age-related (rather than disease-related) neuron death.

In certain embodiments, assessment of frataxin concentration in a biofluid or sample from a subject using a method or assay described herein may be combined with one or more neuroimaging techniques. Any neuroimaging modality known to one with skill in the art may be utilized, for example, see Blair I A, “The current state of biomarker research for Friedreich's ataxia: a report from the 2018 FARA biomarker meeting,” Future Science OA. 2019, 5(6):FSO398, the contents of which are herein incorporated by reference in their entirety. As a non-limiting example, imaging techniques useful for the assessment of the structural integrity or function of one or more components of the central nervous system (CNS) may be used, such as MRI, MRS and/or DTI.

Non-limiting examples of CNS regions that may be assessed include the spinal cord, corpus callosum, dentate nucleus, cerebellar lobules, cerebellar peduncles, cerebellum, and/or the corticospinal tracts. The neuroimaging technique may be used to quantify or assess brain morphometry (e.g., of the cerebellum and brainstem), spinal cord morphometry, brain and spinal cord diffusion, dentate iron content (brain QSM), spinal cord spectroscopy, volume or cross-sectional area of a region of interest, atrophy, eccentricity, relative quantification of biochemical measures such as N-acetylaspartate (tNAA), myo-inositol (mIns) or iron, myelination, fiber density, axonal diameter, anisotropic diffusion, fractional anisotropy, and/or diffusivity (e.g., radial diffusivity). These measurements may be collected cross-sectionally across a group of healthy individuals and/or subjects diagnosed with a disease, such as, but not limited to Friedreich's Ataxia, or longitudinally over time.

Subjects diagnosed with Friedreich's Ataxia undergoing such assessments may be of any age. In certain embodiments, a subject diagnosed with Friedreich's Ataxia undergoing such assessments may be between 5 and 10 years old. In certain embodiments, a subject diagnosed with Friedreich's Ataxia undergoing such assessments may be between 10 and 18 years old. In certain embodiments, a subject diagnosed with Friedreich's Ataxia undergoing such assessments may be 18 years of age or older. Subjects diagnosed with Friedreich's Ataxia undergoing such assessments may have early or late stage disease.

In certain embodiments, results of neuroimaging assessments may be correlated with frataxin protein and/or neurofilament light chain quantification in a biofluid or sample from the same or other subject.

The methods and/or assays described herein alone or in combination with one or more additional measures may be used in the treatment, prevention, management, diagnosis and/or research of disease, disorders and/or conditions. In certain embodiments, the disease is Friedreich's Ataxia. For example, in some embodiments, one or more neuroimaging assessments may be combined with the frataxin biofluid assay as disclosed herein in a method of diagnosing Friedreich's Ataxia or a frataxin deficiency in a subject. In some embodiments, one or more neuroimaging assessments may be combined with the frataxin biofluid assay as disclosed herein in a method of selecting an effective treatment for Friedreich's Ataxia or a frataxin deficiency in a subject. In some embodiments, one or more neuroimaging assessments may be combined with the frataxin biofluid assay as disclosed herein in a method of treating Friedreich's Ataxia or a frataxin deficiency in a subject. In some embodiments, one or more neuroimaging assessments may be combined with the frataxin biofluid assay as disclosed herein in a method of assessing the effectiveness of a treatment for Friedreich's Ataxia or a frataxin deficiency in a subject. In any of these embodiments, one or more neuroimaging assessments may be combined with the frataxin biofluid assay as disclosed herein as well as an assessment of another biomarker, such as neurofilament light chain.

### Methods of Diagnosing Friedreich's Ataxia

In certain aspects, the frataxin biofluid assay as disclosed herein may be used in a method of diagnosing Friedreich's Ataxia or a frataxin deficiency in a subject, and/or identifying a subject as a candidate for a therapy such as frataxin gene therapy. In some embodiments, the frataxin biofluid assay as disclosed herein may be used in conjunction with a genetic test for Friedreich's Ataxia and/or clinical assessment of a subject for symptoms consistent with Friedreich's Ataxia. In some embodiments, the genetic test comprises determining whether both alleles of the gene encoding frataxin comprises a repeat expansion.

In certain embodiments, the disclosure herein provides a method of diagnosing Friedreich's Ataxia or a frataxin deficiency in a subject comprising assessing the concentration of frataxin protein in a biofluid from the subject. In certain embodiments, the disclosure herein provides a method of diagnosing Friedreich's Ataxia or a frataxin deficiency in a subject comprising assessing the concentration of frataxin protein and neurofilament light chain in one or more biofluids or biofluid samples from the subject, and optionally in the same biofluid or biofluid sample.

In certain embodiments, the disclosure herein provides a method of diagnosing Friedreich's Ataxia or a frataxin deficiency in a subject suspected of having Friedreich's Ataxia comprising the steps of a) performing an assay on a sample of a biofluid from the subject suspected of having Friedreich's Ataxia or a frataxin deficiency to assess the concentration of frataxin protein present in the sample, b) performing an assay on a sample of a biofluid from a healthy subject to assess the concentration of frataxin protein present in the sample, and c) diagnosing the subject with Friedreich's Ataxia or a frataxin deficiency if the concentration of frataxin protein present in the sample from the subject suspected of having Friedreich's Ataxia or a frataxin deficiency is just above, at or, below the limit of detection (i.e., is not detected in a biofluid sample) or at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a biofluid sample from the healthy subject.

In certain embodiments, the disclosure herein provides a method of diagnosing Friedreich's Ataxia or a frataxin deficiency in a subject, comprising the steps of a) performing an assay on a first sample of a biofluid from the subject to assess the concentration of frataxin protein present in the sample, b) performing an assay on a second sample of a biofluid from the subject to assess the concentration of neurofilament light chain present in the sample, and c) diagnosing the subject with Friedreich's Ataxia or a frataxin deficiency if the concentration of frataxin protein present in the first sample is just above, at, or below the limit of detection (i.e., is not detected in a biofluid sample) and if neurofilament light chain is detected in the second sample at a concentration of at least 40 pg/mL (e.g., at least 40 pg/mL, at least 50 pg/mL, at least 60 pg/mL, at least 70 pg/mL, at least 80 pg/mL, at least 100 pg/mL).

In certain embodiments, the disclosure herein provides a method of diagnosing Friedreich's Ataxia in a subject suspected of having Friedreich's Ataxia, comprising the steps of a) performing an assay on a first sample of a biofluid from the subject suspected of having Friedreich's Ataxia to assess the concentration of frataxin protein present in the sample, b) performing an assay on a second sample of a biofluid from the subject suspected of having Friedreich's Ataxia to assess the concentration of neurofilament light chain present in the sample, c) performing an assay on a first sample of a biofluid from an age-matched healthy subject to assess the concentration of frataxin protein present in the sample, d) performing an assay on a second sample of a biofluid from the age-matched healthy subject to assess the concentration of neurofilament light chain present in the sample, and e) diagnosing the subject with Friedreich's Ataxia if the concentration of frataxin protein present in the first sample from the subject suspected of having Friedreich's Ataxia is lower than the limit of detection (i.e., is not detected in the biofluid sample), approximately equal to the limit of detection, or is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in the first sample from the age-matched healthy subject, and the concentration of neurofilament light chain present in the second sample from the subject suspected of having Friedreich's Ataxia is greater than the concentration of neurofilament light chain present in the second sample from the age-matched healthy subject or the concentration of neurofilament light chain present in the second sample from the subject suspected of having Friedreich's Ataxia is at least 40 pg/mL (e.g., at least 40 pg/mL, at least 50 pg/mL, at least 60 pg/mL, at least 70 pg/mL, at least 80 pg/mL, at least 100 pg/mL).

In some embodiments, the first sample and the second sample of a biofluid from the subject suspected of having Friedreich's Ataxia are the same sample or each is a portion of an initial sample and/or the first sample and the second sample of a biofluid from the healthy subject are the same sample or each is a portion of an initial sample.

In some embodiments, the subject is diagnosed with Friedreich's Ataxia or a frataxin deficiency if the concentration of frataxin protein present in a sample (e.g., biofluid) is just above, at, or below the limit of detection (i.e., is not detected in a biofluid sample) and a neuroimaging assessment demonstrates gray matter volume loss relative to a healthy, age-matched subject. In some embodiments, the relative gray matter volume loss may be at least 5%, e.g., 5%, 10%, 15%, 20%, 25%, 30%, 40%, 50%, 60%, or more than 60% relative to a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates gray matter volume loss relative to a healthy, age-matched subject. In some embodiments, the relative gray matter volume loss may be at least 5%, e.g., 5%, 10%, 15%, 20%, 25%, 30%, 40%, 50%, 60%, or more than 60% relative to a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in a sample (e.g., a biofluid) is just above, at, or below the limit of detection (i.e., is not detected in a biofluid sample) and a neuroimaging assessment demonstrates cerebellar lobular atrophy relative to a healthy, age-matched subject. In some embodiments, the relative cerebellar lobular atrophy (e.g., volume loss) may be at least 5%, e.g., 5%, 10%, 15%, 20%, 25%, 30%, 40%, 50%, 60%, or more than 60% relative to a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates cerebellar lobular atrophy relative to a healthy, age-matched subject. In some embodiments, the relative cerebellar lobular atrophy (e.g., volume loss) may be at least 5%, e.g., 5%, 10%, 15%, 20%, 25%, 30%, 40%, 50%, 60%, or more than 60% relative to a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in a sample (e.g., a biofluid) is just above, at, or below the limit of detection (i.e., is not detected in a biofluid sample) and a neuroimaging assessment demonstrates decreased mean volume of the dentate nucleus relative to a healthy, age-matched subject. In some embodiments, the relative decrease in mean volume is at least 20%, e.g., 20%, 25%, 30%, 35%, 40%, 45%, or more than 45% relative to a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates decreased mean volume of the dentate nucleus relative to a healthy, age-matched subject. In some embodiments, the relative decrease in mean volume is at least 20%, e.g., 20%, 25%, 30%, 35%, 40%, 45%, or more than 45% relative to a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in a sample (e.g., a biofluid) is just above, at, or below the limit of detection (i.e., is not detected in a biofluid sample) and a neuroimaging assessment demonstrates increased mean, radial, and/or axial diffusivity in spinal cord (e.g., cervical spinal cord) relative to a healthy, age-matched subject. In some embodiments, mean diffusivity is increased by at least 15%, e.g., 15%, 20%, 25%, 30%, 35%, 40%, 45%, 50%, 60%, 70%, or more than 70% relative to a healthy, age-matched subject. In some embodiments, radial diffusivity is increased by at least 30%, e g., 30%, 35%, 40%, 45%, 50%, 55%, 60%, 65%, 70%, 75%, or more than 75% relative to a healthy, age-matched subject. In some embodiments, axial diffusivity is increased by at least 5%, e.g., 5%, 10%, 15%, 20%, 25%, or more than 25% relative to a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates increased mean, radial, and/or axial diffusivity in spinal cord (e.g., cervical spinal cord) relative to a healthy, age-matched subject. In some embodiments, mean diffusivity is increased by at least 15%, e.g., 15%, 20%, 25%, 30%, 35%, 40%, 45%, 50%, 60%, 70%, or more than 70% relative to a healthy, age-matched subject. In some embodiments, radial diffusivity is increased by at least 30%, e.g., 30%, 35%, 40%, 45%, 50%, 55%, 60%, 65%, 70%, 75%, or more than 75% relative to a healthy, age-matched subject. In some embodiments, axial diffusivity is increased by at least 5%, e.g., 5%, 10%, 15%, 20%, 25%, or more than 25% relative to a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in the a sample (e.g., a biofluid) is just above, at, or below the limit of detection (i.e., is not detected in a biofluid sample) and a neuroimaging assessment demonstrates increased mean, radial, and/or axial diffusivity in superior, middle, and inferior cerebellar peduncles relative to a healthy, age-matched subject. In some embodiments, mean diffusivity is increased by at least 15%, e.g., 15%, 20%, 25%, 30%, 35%, 40%, 45%, 50%, 60%, 70%, or more than 70% relative to a healthy, age-matched subject. In some embodiments, radial diffusivity is increased by at least 30%, e.g., 30%, 35%, 40%, 45%, 50%, 55%, 60%, 65%, 70%, 75%, or more than 75% relative to a healthy, age-matched subject. In some embodiments, axial diffusivity is increased by at least 5%, e.g., 5%, 10%, 15%, 20%, 25%, or more than 25% relative to a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates increased mean, radial, and/or axial diffusivity in superior, middle, and inferior cerebellar peduncles relative to a healthy, age-matched subject. In some embodiments, mean diffusivity is increased by at least 15%, e.g., 15%, 20%, 25%, 30%, 35%, 40%, 45%, 50%, 60%, 70%, or more than 70% relative to a healthy, age-matched subject. In some embodiments, radial diffusivity is increased by at least 30%, e.g., 30%, 35%, 40%, 45%, 50%, 55%, 60%, 65%, 70%, 75%, or more than 75% relative to a healthy, age-matched subject. In some embodiments, axial diffusivity is increased by at least 5%, e.g., 5%, 10%, 15%, 20%, 25%, or more than 25% relative to a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in the a sample (e.g., a biofluid) is just above, at, or below the limit of detection (i.e., is not detected in a biofluid sample) and a neuroimaging assessment demonstrates reduced fractional anisotropy in white matter relative to a healthy, age-matched subject. In some embodiments, fractional anisotropy is reduced by at least 15%, e.g., 15%, 20%, 25%, 30%, 35%, 40%, 45%, 50%, 60%, 70%, or more than 70% relative to a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates reduced fractional anisotropy in white matter relative to a healthy, age-matched subject. In some embodiments, fractional anisotropy is reduced by at least 15%, e.g., 15%, 20%, 25%, 30%, 35%, 40%, 45%, 50%, 60%, 70%, or more than 70% relative to a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in a sample (e.g., a biofluid) is just above, at, or below the limit of detection (i.e., is not detected in a biofluid sample) and a neuroimaging assessment demonstrates reduced spinal cord cross-sectional area relative to a healthy, age-matched subject. In some embodiments, the thoracic spinal cord cross-sectional area is reduced by at least 5%, e.g., 5%, 10%, 15%, 20%, 25%, 30%, 35%, 40%, 50%, 60%, or more than 60% relative to that of a healthy, age-matched subject. In some embodiments, the cervical spinal cord cross-sectional area is reduced by at least 5%, e.g., 5%, 10%, 15%, 20%, 25%, 30%, 35%, 40%, 50%, 60%, or more than 60% relative to that of a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates reduced spinal cord cross-sectional area relative to a healthy, age-matched subject. In some embodiments, the thoracic spinal cord cross-sectional area is reduced by at least 5%, e.g., 5%, 10%, 15%, 20%, 25%, 30%, 35%, 40%, 50%, 60%, or more than 60% relative to that of a healthy, age-matched subject. In some embodiments, the cervical spinal cord cross-sectional area is reduced by at least 5%, e.g., 5%, 10%, 15%, 20%, 25%, 30%, 35%, 40%, 50%, 60%, or more than 60% relative to that of a healthy, age-matched subject. In some embodiments, diagnosis is further based in part on an assessment of neurofilament light chain concentration in a sample from a subject suspected of having Friedreich's Ataxia or a frataxin deficiency (alone or relative to the neurofilament light chain concentration of a healthy, age-matched subject), as disclosed herein.

In some embodiments, the subject is diagnosed with Friedreich's Ataxia or a frataxin deficiency if the concentration of frataxin protein present in the a sample (e.g., a biofluid) is just above, at, or below the limit of detection (i.e., is not detected in a biofluid sample) and a neuroimaging assessment demonstrates reduced spinal cord volume relative to a healthy, age-matched subject. In some embodiments, the thoracic spinal cord volume is reduced by at least 5%, e.g., 5%, 10%, 15%, 20%, 25%, 30%, 35%, 40%, 50%, 60%, or more than 60% relative to that of a healthy, age-matched subject. In some embodiments, the cervical spinal cord volume is reduced by at least 5%, e.g., 5%, 10%, 15%, 20%, 25%, 30%, 35%, 40%, 50%, 60%, or more than 60% relative to that of a healthy, age-matched subject. In some embodiments, the subject is diagnosed with Friedreich's Ataxia or a frataxin deficiency if the concentration of frataxin protein present in a sample (e.g., biofluid) is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a corresponding sample from a healthy, age-matched subject and a neuroimaging assessment demonstrates reduced spinal cord volume relative to a healthy, age-matched subject. In some embodiments, the thoracic spinal cord volume is reduced by at least 5%, e.g., 5%, 10%, 15%, 20%, 25%, 30%, 35%, 40%, 50%, 60%, or more than 60% relative to that of a healthy, age-matched subject. In some embodiments, the cervical spinal cord volume is reduced by at least 5%, e.g., 5%, 10%, 15%, 20%, 25%, 30%, 35%, 40%, 50%, 60%, or more than 60% relative to that of a healthy, age-matched subject. In some embodiments, diagnosis is further based in part on an assessment of neurofilament light chain concentration of the subject suspected of having Friedreich's Ataxia or a frataxin deficiency (alone or relative to the neurofilament light chain concentration of a healthy, age-matched subject), as disclosed herein.

In some embodiments, the limit of detection of the assay for frataxin is about 2.0 pg/mL or less. In some embodiments, the limit of detection about 1.5 pg/mL or less. In some embodiments, the limit of detection is about 1.0 pg/mL or less. In some embodiments, the limit of detection is about 0.5 pg/mL or less. In certain embodiments, the assay comprises a capture agent that specifically binds to frataxin or a fragment of frataxin, and a detection agent that specifically binds to frataxin or a fragment of frataxin. In some embodiments, the capture agent is Ab-1 and the detection agent is Ab-3.

In some embodiments, the subject diagnosed with Friedreich's Ataxia is administered a therapy capable of increasing frataxin expression or frataxin levels. In some embodiments, the subject diagnosed with Friedreich's Ataxia is administered gene therapy, e.g., comprising a. nucleic acid construct encoding frataxin or a functional fragment thereof. In some embodiments, the subject diagnosed with Friedreich's Ataxia is administered a carrier protein capable of delivering frataxin to mitochondria. In some embodiments, the subject diagnosed with Friedreich's Ataxia is administered a ubiquitin competitor, e.g., a compound that inhibits ubiquitination and degradation of frataxin.

### Methods of Treatment

The assay methods disclosed herein may be used in conjunction with one or more treatments of Friedreich's Ataxia. For instance, the assay may be used before, concurrent with, or after treatment to detect starting levels of frataxin, to design an appropriate therapy that will increase frataxin levels, and/or to monitor and assess the efficacy of a treatment.

In some embodiments, the treatment of Friedreich's Ataxia comprises a gene therapy. In some embodiments, the gene therapy comprises administering a viral vector, e.g., an adeno-associated virus (AAV), comprising a nucleic acid construct that encodes frataxin or a fragment thereof. In some embodiments, the nucleic acid construct comprises a sequence as disclosed in WO2020/069461, the contents of which are incorporated by reference in their entirety.

In certain embodiments, a method of the present disclosure may be used to determine a treatment protocol for treating a subject with barely detectable or undetectable frataxin in a biofluid, e.g., frataxin levels of about or below 3.0 pg/ml or less, 2.0 pg/mL or less, 1.0 pg/mL or less, or 0.5 pg/mL or less. For example, the concentration of frataxin protein in the subject's biofluid sample, e.g., a concentration below a limit of detection (e.g., of about 3.0 pg/ml or less, 2.0 pg/mL or less, about 1.0 pg/mL or less, or about 0.5 pg/mL or less) may be used to determine that a subject should be given a gene therapy treatment regimen suitable to increase frataxin levels. The gene therapy treatment regimen may comprise administration of an AAV comprising a capsid and a viral genome, wherein the viral genome encodes frataxin or a functional fragment of frataxin.

In certain embodiments, a method of the present disclosure may be used to determine a treatment protocol for treating a subject with a concentration of frataxin in the subject's biofluid sample that is at least 2-fold less to at least 2000-fold less (e.g., at least 2 fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 100-fold, at least 150-fold, at least 200-fold, at least 500-fold, at least 750-fold, at least 1000-fold, at least 1500-fold, or at least 2000-fold less) than the concentration of frataxin protein present in a biofluid sample from a healthy individual, optionally an age-matched healthy individual. For example, the subject having 2-fold less to 2000-fold less frataxin in biofluid compared to a healthy individual may be given a gene therapy treatment regimen suitable to increase frataxin levels. In some embodiments, the subject having 2-fold less to 2000-fold less frataxin in biofluid compared to a healthy individual may be given a gene therapy treatment regimen suitable to increase frataxin levels to those of the healthy individual. In some embodiments, the subject having 2-fold less to 2000-fold less frataxin in biofluid compared to a healthy individual may be given a gene therapy treatment regimen suitable to increase frataxin levels to those at least 20%, at least 30%, at least 40%, at least 50%, at least 60%, at least 70%, at least 80%, at least 90%, at least 95%, at least 99%, or 100% of the healthy individual. The gene therapy treatment regimen may comprise administration of an AAV comprising a capsid and a viral genome, wherein the viral genome encodes frataxin or a functional fragment of frataxin.

In some embodiments, a subject is administered gene therapy and a second therapy. For example, the second therapy may comprise means for restoring mitochondrial function, a therapy that reduces mitochondrial dysfunction, a therapy that reduces oxidative stress, or a therapy that reduces pro-inflammatory signaling. For example, the therapy may restore mitochondrial transmembrane potential in fibroblasts. In some embodiments, the therapy activates the Keap1/Nrf2 pathway. In some embodiments, the therapy increases Nrf2 signaling. In some embodiments, the therapy reduces oxidative stress, e.g., by reducing production of reactive oxidation species. In some embodiments, the concentration of frataxin protein in the subject's biofluid sample may be used to determine the selection and/or dose of a therapy, e.g., a small molecule therapy, suitable to at least partially restore motor function, manual dexterity, and/or activities of daily living.

In some embodiments, the second therapy is a small molecule therapy. In some embodiments, the second therapy may be occupational therapy, speech therapy, orthopedic care, bracing, surgery, heart disease medication (e.g., a beta blocker, an angiotensin-converting-enzyme [ACE] inhibitor), an antioxidant (e.g., vitamin E, coenzyme Q₁₀, idebenone, and extract of Ginkgo biloba leaves), treatment to lower blood sugar (e.g., insulin, an oral anti-diabetic drug, modified diet), or physical therapy.

In some embodiments, the second therapy is omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy (e.g., to increase frataxin expression and/or treat cardiac disease associated with Friedreich's Ataxia), acetyl-L-carnitine (ALCAR), rosuvastatin, an incretin analog, indole-3-propionic acid, or erythropoietin or a modified (e.g., carbamylated) form thereof.

In some embodiments, the second therapy comprises omaveloxolone. In some embodiments, a subject is administered omaveloxolone at a dose of about 5 mg/day to about 300 mg/day (e.g., about 5 mg/day, about 10 mg/day, about 15 mg/day, about 20 mg/day, about 30 mg/day, about 40 mg/day, about 50 mg/day, about 60 mg/day, about 70 mg/day, about 80 mg/day, about 90 mg/day, about 100 mg/day, about 120 mg/day, about 135 mg/day, about 150 mg/day, about 60 mg/day, about 175 mg/day, about 200 mg/day, about 250 mg/day, or about 300 mg/day). In some embodiments, a subject is administered omaveloxolone at a high dose, e.g., about 80-300 mg/day, about 80-160 mg/day, or about 200-300 mg/day, for example about 80 mg/day, about 90 mg/day, about 100 mg/day, about 120 mg/day, about 135 mg/day, about 150 mg/day, about 160 mg/day, about 175 mg/day, about 200 mg/day, about 250 mg/day, or about 300 mg/day. In some embodiments, a subject is administered omaveloxolone at a low dose, e.g., about 5-50 mg/day or about 5-15 mg/day, for example, about 5 mg/day, about 10 mg/day, about 15 mg/day, about 20 mg/day, about 30 mg/day, about 40 mg/day, or about 50 mg/day.

In some embodiments, the second therapy comprises alpha-tocotrienol quinone (e.g., EPI-743). In some embodiments, a subject is administered alpha-tocotrienol quinone (e.g., EPI-743) at a high dose, e.g., about 1200 mg/day (e.g., about 400 mg, three times daily). In some embodiments, a subject is administered alpha-tocotrienol quinone (e.g., EPI-743) at a low dose, e.g., less than about 1200 mg/day, e.g., 800 mg/day or 400 mg/day.

In some embodiments, the second therapy comprises a mimetic of a polyunsaturated fatty acid (PUFA). In some embodiments, the mimetic is a deuterated PUFA. In some embodiments, the PUFA mimetic is deuterated ethyl linoleate, e.g., RT001. In some embodiments, a subject is administered the PUFA mimetic (e.g., RT001) at about 1-10 g/day or about 1.8-9 g/day, e.g., about 1 g/day, about 1.5 g/day, about 1.8 g/day, about 2 g/day, about 2.5 g/day, about 3 g/day, about 4 g/day, about 5 g/day, about 6 g/day, about 7 g/day, about 8 g/day, about 8.5 g/day, about 9 g/day, about 9.5 g/day, or about 10 g/day. In some embodiments, a subject is administered a PUFA mimetic (e.g., RT001) at a high dose, e.g., at about 6-10 g/day or about 6-9 g/day (e.g., about 6 g/day, about 7 g/day, about 8 g/day, about 9 g/day, or about 10 g/day). In some embodiments, a subject is administered a PUFA mimetic (e.g., RT001) at a low dose, e.g., less than about 6 g/day, e.g., about 1 g/day, about 1.5 g/day, about 1.8 g/day, about 2 g/day, about 2.5 g/day, about 3 g/day, about 4 g/day, or about 5 g/day.

In some embodiments, the second therapy comprises (+)-epicatechin. In some embodiments, a subject is administered (+)-epicatechin at about 75-150 mg/day, about 75 mg/day, about 100 mg/day, about 125 mg/day, or about 150 mg/day. In some embodiments, a subject is administered (+)-epicatechin at a high dose, e.g., about 75-100 mg/day, e.g., about 75 mg/day or about 100 mg/day. In some embodiments, a subject is administered (+)-epicatechin at a low dose, e.g., about 125-150 mg/day, e.g., about 125 mg/day or about 150 mg/day.

In some embodiments, the second therapy comprises methylprednisolone. In some embodiments, a subject is administered methylprednisolone at a dose of about 8 mg/day to about 48 mg/day (e.g., about 8 mg/day, about 16 mg/day, about 24 mg/day, about 32 mg/day, about 40 mg/day, or about 48 mg/day). In some embodiments, a subject is administered methylprednisolone at a high dose, e.g., about 32-48 mg/day, for example about 32 mg/day, about 40 mg/day, or about 48 mg/day. In some embodiments, a subject is administered methylprednisolone at a low dose, e.g., about 8-24 mg/day, for example about 8 mg/day, about 16 mg/day, or about 24 mg/day.

In some embodiments, the second therapy comprises a D-amino acid oxidase inhibitor, e.g., TAK-831. In some embodiments, a subject is administered a D-amino acid oxidase inhibitor (e.g., TAK-831) at a dose of about 75 mg/day to about 600 mg/day (e.g., about 75 mg/day, about 150 mg/day, about 225 mg/day, about 300 mg/day, about 375 mg/day, about 450 mg/day, about 525 mg/day, or about 600 mg/day). In some embodiments, a subject is administered a D-amino acid oxidase inhibitor (e.g., TAK-831) at a high dose, e.g., about 300 mg/day to about 600 mg/day (e.g., about 300 mg/day, about 375 mg/day, about 450 mg/day, about 525 mg/day, or about 600 mg/day). In some embodiments, a subject is administered a D-amino acid oxidase inhibitor (e.g., TAK-831) at a low dose, e.g., about 75 mg/day to about 225 mg/day (e.g., about 75 mg/day, about 150 mg/day, or about 225 mg/day).

In some embodiments, the second therapy comprises a peroxisome-proliferator activator receptor (PPAR) gamma agonist or ligand. In some embodiments, the PPAR gamma agonist or ligand is MIN-102. In some embodiments, the PPAR gamma agonist or ligand is pioglitazone. In some embodiments, a subject is administered a PPAR gamma agonist or ligand (e.g., MIN-102, pioglitazone) at a dose of about 15 mg/day to about 45 mg/day (e.g., about 15 mg/day, about 30 mg/day, or about 45 mg/day). In some embodiments, a subject is administered a PPAR gamma agonist or ligand (e.g., MIN-102, pioglitazone) at a high dose, e.g., about 30 mg/day to about 45 mg/day (e.g., about 30 mg/day or about 45 mg/day). In some embodiments, a subject is administered PPAR gamma agonist or ligand (e.g., MIN-102, pioglitazone) at a low dose, e.g., about 15 mg/day.

In some embodiments, the second therapy comprises dimethyl fumarate. In some embodiments, a subject is administered dimethyl fumarate at a dose of about 120 mg/day to about 480 mg/day (e.g., about 120 mg/day, about 240 mg/day, about 360 mg/day, or about 480 mg/day). In some embodiments, a subject is administered dimethyl fumarate at a high dose, e.g., about 360 mg/day to about 480 mg/day (e.g., about 360 mg/day or about 480 mg/day). In some embodiments, a subject is administered dimethyl fumarate at a low dose, e.g., about 120 mg/day to about 240 mg/day (e.g., about 120 mg/day or about 240 mg/day).

In some embodiments, the second therapy comprises a carrier protein to deliver frataxin protein to mitochondria (e.g., Trans-Activator of Transcription (TAT), e.g., TAT-frataxin). In some embodiments, TAT-frataxin is CTI-1601. In some embodiments, a subject is administered a carrier protein to deliver frataxin protein to mitochondria (e.g., Trans-Activator of Transcription (TAT), e.g., TAT-frataxin, e.g., CTI-1601), e.g., at a dose of about 0.5-5 mg/kg (e.g., about 0.5 mg/kg, about 1 mg/kg, about 1.5 mg/kg, about 1.6 mg/kg, about 1.7 mg/kg, about 2 mg/kg, about 3 mg/kg, about 4 mg/kg, or about 5 mg/kg). In some embodiments, a subject is administered a carrier protein to deliver frataxin protein to mitochondria (e.g., Trans-Activator of Transcription (TAT), e.g., TAT-frataxin, e.g., CTI-1601) at a high dose, e.g., about 3-5 mg/kg (e.g., about 3 mg/kg, about 4 mg/kg, or about 5 mg/kg). In some embodiments, a subject is administered a carrier protein to deliver frataxin protein to mitochondria (e.g., Trans-Activator of Transcription (TAT), e.g., TAT-frataxin, e.g., CTI-1601) at a low dose, e.g., about 0.5-2 mg/kg, e.g., about 0.5 mg/kg, about 1 mg/kg, about 1.5 mg/kg, about 1.6 mg/kg, about 1.7 mg/kg, or about 2 mg/kg).

In some embodiments, the second therapy comprises a small molecule modulator of a cytokine receptor. In some embodiments, the second therapy comprises a small molecule activator of the tissue-protective erythropoietin receptor. In some embodiments, the second therapy comprises STS-E412 or STS-E424. In some embodiments, a subject is administered a small molecule activator of the tissue-protective erythropoietin receptor, such as STS-E412 or STS-E424, at a high dose. In some embodiments, the second therapy comprises STS-E412 or STS-E424. In some embodiments, a subject is administered a small molecule activator of the tissue-protective erythropoietin receptor, such as STS-E412 or STS-E424, at a low dose.

In some embodiments, the second therapy comprises a ubiquitin competitor. In some embodiments, the second therapy comprises an inhibitor of RNF126. In some embodiments, a subject is administered a ubiquitin competitor, such as an inhibitor of RNF126, at a high dose. In some embodiments, a subject is administered a ubiquitin competitor, such as an inhibitor of RNF126, at a low dose.

In some embodiments, the second therapy comprises etravirine. In some embodiments, a subject is administered etravirine, at a dose of about 100 mg/day to about 400 mg/day (e.g., about 100 mg/day, about 200 mg/day, about 300 mg/day, or about 400 mg/day). In some embodiments, a subject is administered etravirine at a high dose, e.g., about 300 mg/day to about 400 mg/day (e.g., about 300 mg/day or about 400 mg/day). In some embodiments, a subject is administered etravirine at a low dose, e.g., about 100 mg/day to about 200 mg/day (e.g., about 100 mg/day or about 200 mg/day).

In some embodiments, the second therapy comprises resveratrol (e.g., JOT101). In some embodiments, a subject is administered resveratrol (e.g., JOT101) at a dose of about 1 g/day to about 5 g/day (e.g., about 1 g/day, about 1.5 g/day, about 2 g/day, about 2.5 g/day, about 3 g/day, about 3.5 g/day, about 4 g/day, about 4.5 g/day or about 5 g/day). In some embodiments, a subject is administered resveratrol (e.g., JOT101) at a high dose, e.g., about 3 g/day to about 5 g/day (e.g., about 3 g/day, about 3.5 g/day, about 4 g/day, about 4.5 g/day, or about 5 g/day). In some embodiments, a subject is administered resveratrol (e.g., JOT101) at a low dose, e.g., about 1 g/day to about 2.5 g/day (e.g., about 1 g/day, about 1.5 g/day, about 2 g/day, or about 2.5 g/day).

In some embodiments, the second therapy comprises nicotinamide. In some embodiments, a subject is administered nicotinamide at a dose of about 2 g/day to about 8 g/day (e.g., about 2 g/day, about 3 g/day, about 4 g/day, about 5 g/day, about 6 g/day, about 7 g/day, or about 8 g/day). In some embodiments, a subject is administered nicotinamide at a high dose, e.g., about 5 g/day to about 8 g/day (e.g., about 5 g/day, about 6 g/day, about 7 g/day, or about 8 g/day). In some embodiments, a subject is administered nicotinamide at a low dose, e.g., about 2 g/day to about 4 g/day (e.g., about 2 g/day, about 3 g/day, or about 4 g/day).

In some embodiments, the second therapy comprises interferon gamma (e.g., interferon gamma-1b). In some embodiments, the interferon gamma (e.g., interferon gamma-1b) is administered subcutaneously. In some embodiments, a subject is administered interferon gamma (e.g., interferon gamma-1b) at a dose of about 10-100 μg/m² (e.g., about 10 μg/m², about 25 μg/m², about 50 μg/m², or about 100 μg/m²). In some embodiments, a subject is administered interferon gamma (e.g., interferon gamma-1b) at a high dose, e.g., about 50-100 μg/m² (e.g., about 50 μg/m² or about 100 μg/m²). In some embodiments, a subject is administered interferon gamma (e.g., interferon gamma-1b) at a low dose, e.g., about 10-25 μg/m² (e.g., about 10 μg/m² or about 25 μg/m²).

In some embodiments, the second therapy comprises managing one or more symptoms of Friedreich's Ataxia. Such therapies may include, but are not limited to, occupational therapy, speech therapy, orthopedic care, bracing, surgery, treatment for heart disease, an antioxidant, treatment to lower blood sugar, and/or physical therapy. In some embodiments, treatment for heart disease comprises a beta blocker or an angiotensin-converting-enzyme [ACE] inhibitor. In some embodiments, an antioxidant comprises vitamin E, coenzyme Q₁₀, idebenone, or an extract from Ginkgo biloba leaves. In some embodiments, treatment to lower blood sugar comprises insulin, an oral anti-diabetic drug, or a modified diet. See A Cook, P Giunti, Friedreich's ataxia: clinical features, pathogenesis and management, British Medical Bulletin, Volume 124, Issue 1, December 2017, Pages 19-30.

### Methods of Determining Efficacy

In certain embodiments, the assay disclosed herein may be used in a method of determining the efficacy of a treatment for increasing frataxin levels in a subject. In some embodiments, the treatment is a gene therapy, e.g., a frataxin gene therapy. In some embodiments, the treatment is an RNA-based therapy. In some embodiments, the treatment stabilizes frataxin or prevents its degradation (e.g., inhibits ubiquitination of frataxin). In some embodiments, the treatment is a carrier protein for frataxin, e.g., to deliver frataxin to mitochondria. In some embodiments, the stabilization and/or improvement of the subject is determined using an assay disclosed herein, e.g., alone or in conjunction with one or more genetic screens and/or phenotypic assessments of the subject before and/or after treatment.

In some embodiments, a method of determining the efficacy of a treatment for increasing frataxin levels in a subject comprises the steps of assessing a baseline concentration of frataxin and/or neurofilament light chain in a sample of a biofluid of a subject, b) treating the subject with a treatment that results in increased frataxin levels in the subject, c) assessing a subsequent concentration of frataxin and/or neurofilament light chain in a sample of a biofluid of the subject, and d) comparing the baseline concentration of frataxin to the subsequent concentration of frataxin and/or comparing the baseline concentration of neurofilament light chain to the subsequent concentration of neurofilament light chain. In some embodiments, the baseline concentration of frataxin is just above, below, or equal to the limit of detection of the assay (e.g., below 2.0 pg/mL, below 1 pg/mL, or below 0.5 pg/mL), e.g., frataxin is not detectable at baseline, and the subsequent concentration of frataxin is above the limit of detection of the assay (e.g., at least 2.0 pg/mL, at least 1 pg/mL, at least 0.5 pg/mL), e.g., frataxin is detectable in subsequent samples, preferably at least 0.5×-3× higher than baseline (e.g., at least 0.5-1×, at least 1-1.5×, at least 1.5-2×, at least 2-2.5×, at least 2.5-3× higher than baseline). In certain embodiments, the treatment that results in increased frataxin levels in the subject is a frataxin gene therapy.

In some embodiments, a method of determining the efficacy of a treatment for increasing frataxin levels in a subject comprises the steps of assessing a baseline concentration of frataxin in a sample of a biofluid of the subject and performing a baseline neuroimaging assessment of the subject's brain and/or spinal cord, b) treating the subject with a treatment that results in increased frataxin levels in the subject, e.g., a frataxin gene therapy, c) assessing a subsequent concentration of frataxin in a sample of a biofluid of the subject and performing a subsequent neuroimaging assessment of the subject's brain and/or spinal cord, and d) comparing the baseline concentration of frataxin to the subsequent concentration of frataxin and/or comparing the baseline neuroimaging assessment to the subsequent neuroimaging assessment. In some embodiments, the baseline concentration of frataxin is just above, below, or equal to the limit of detection of the assay (e.g., below 2.0 pg/mL, below 1 pg/mL, or below 0.5 pg/mL), e.g., frataxin is not detectable at baseline, and the subsequent concentration of frataxin is above the limit of detection of the assay (e.g., at least 2.0 pg/mL, at least 1 pg/mL, at least 0.5 pg/mL), e.g., frataxin is detectable in subsequent samples, preferably at least 0.5×-3× higher than baseline (e.g., at least 0.5-1×, at least 1-1.5×, at least 1.5-2×, at least 2-2.5×, at least 2.5-3× higher than baseline). In certain embodiments, the treatment that results in increased frataxin levels in the subject is a frataxin gene therapy.

In some embodiments, a neuroimaging assessment is performed before (baseline neuroimaging assessment) and after (subsequent neuroimaging assessment) a subject is administered a treatment for increasing frataxin levels in a subject e.g., a frataxin gene therapy.

In some embodiments, the baseline neuroimaging assessment is compared to a neuroimaging assessment in an age-matched healthy subject. Without being limited by theory, an age-matched healthy subject may be used as a comparator to control for age-related changes in volume, atrophy, diffusivity, anisotropy, etc.

In some embodiment, the baseline neuroimaging assessment shows gray matter volume loss in the subject of at least 5% relative to an age-matched healthy subject, e.g., of 5%, 10%, 15%, 20%, 25%, 30%, 40%, 50%, 60%, or more than 60% relative to an age-matched healthy subject. In some embodiments, the baseline neuroimaging assessment shows lobular atrophy in the subject of at least 5% relative to an age-matched healthy subject, e.g., of 5%, 10%, 15%, 20%, 25%, 30%, 40%, 50%, 60%, or more than 60% relative to an age-matched healthy subject. In some embodiments, the baseline neuroimaging assessment shows decreased dentate nucleus mean volume of at least 20% relative to an age-matched healthy subject, e.g., of 20%, 25%, 30%, 35%, 40%, 45%, or more than 45% relative to an age-matched healthy subject.

In some embodiments, the baseline neuroimaging assessment shows that mean diffusivity (e.g., in spinal cord or in superior, middle, and inferior cerebellar peduncles) is increased by at least 15% relative to an age-matched healthy subject, e.g., by 15%, 20%, 25%, 30%, 35%, 40%, 45%, 50%, 60%, 70%, or more than 70% relative to an age-matched healthy subject. In some embodiments, the baseline neuroimaging assessment shows that radial diffusivity (e.g., in spinal cord or in superior, middle, and inferior cerebellar peduncles) is increased by at least 30% relative to an age-matched healthy subject, e.g., by 30%, 35%, 40%, 45%, 50%, 55%, 60%, 65%, 70%, 75%, or more than 75% relative to an age-matched healthy subject. In some embodiments, the baseline neuroimaging assessment shows that axial diffusivity (e.g., in spinal cord or in superior, middle, and inferior cerebellar peduncles) is increased by at least 5% relative to an age-matched healthy subject, e.g., by 5%, 10%, 15%, 20%, 25%, or more than 25% relative to an age-matched healthy subject.

In some embodiments, the baseline neuroimaging assessment shows reduced fractional anisotropy in white matter of at least 15% relative to an age-matched healthy subject, e.g., of 15%, 20%, 25%, 30%, 35%, 40%, 45%, 50%, 60%, 70%, or more than 70% relative to an age-matched healthy subject.

In some embodiments, the baseline neuroimaging assessment shows that the subject's cervical spinal cord cross-sectional area is reduced by at least 5% relative to that of an age-matched healthy subject, e.g., by 5%, 10%, 15%, 20%, 25%, 30%, 35%, 40%, 50%, 60%, or more than 60% relative to that of an age-matched healthy subject. In some embodiments, the baseline neuroimaging assessment shows that the subject's thoracic spinal cord cross-sectional area is reduced by at least 5% relative to that of an age-matched healthy subject, e.g., by 5%, 10%, 15%, 20%, 25%, 30%, 35%, 40%, 50%, 60%, or more than 60% relative to that of an age-matched healthy subject.

In some embodiments, the baseline neuroimaging assessment shows that the subject's cervical spinal cord volume is reduced by at least 5% relative to that of an age-matched healthy subject, e.g., by 5%, 10%, 15%, 20%, 25%, 30%, 35%, 40%, 50%, 60%, or more than 60% relative to that of an age-matched healthy subject. In some embodiments, the baseline neuroimaging assessment shows that the subject's thoracic spinal cord volume is reduced by at least 5% relative to that of an age-matched healthy subject, e.g., by 5%, 10%, 15%, 20%, 25%, 30%, 35%, 40%, 50%, 60%, or more than 60% relative to that of an age-matched healthy subject.

In some embodiments, there is no significant change in a measured parameter discussed above (i.e., no significant worsening) when comparing a subsequent neuroimaging assessment to a baseline neuroimaging assessment. In some embodiments, there is no significant change (e.g., no significant loss in volume, increase in atrophy, decrease in fractional anisotropy, and/or increase in diffusivity) if the subsequent neuroimaging assessment measure is within 5% of the baseline neuroimaging assessment measure.

In some embodiments, the subsequent neuroimaging assessment shows no significant further loss in gray matter volume compared to the baseline neuroimaging assessment. In some embodiments, the subsequent neuroimaging assessment shows no significant further lobular atrophy compared to the baseline neuroimaging assessment. In some embodiments, the subsequent neuroimaging assessment shows no significant further decrease in dentate nucleus mean volume compared to the baseline neuroimaging assessment.

In some embodiments, the subsequent neuroimaging assessment shows no significant further increase in mean diffusivity (e.g., in spinal cord or in superior, middle, and inferior cerebellar peduncles) compared to the baseline neuroimaging assessment. In some embodiments, the subsequent neuroimaging assessment shows no significant further increase in radial diffusivity (e.g., in spinal cord or in superior, middle, and inferior cerebellar peduncles) compared to the baseline neuroimaging assessment. In some embodiments, the subsequent neuroimaging assessment shows no significant further increase in axial diffusivity (e.g., in spinal cord or in superior, middle, and inferior cerebellar peduncles) compared to the baseline neuroimaging assessment.

In some embodiments, the subsequent neuroimaging assessment shows no significant further decrease in fractional anisotropy in white matter in the subject, compared to the baseline neuroimaging assessment.

In some embodiments, the subsequent neuroimaging assessment shows no significant further decrease in thoracic spinal cord cross-sectional area compared to the baseline neuroimaging assessment. In some embodiments, the subsequent neuroimaging assessment shows no significant further decrease in cervical spinal cord cross-sectional area compared to the baseline neuroimaging assessment. In some embodiments, the subsequent neuroimaging assessment shows no significant further decrease in thoracic spinal cord volume compared to the baseline neuroimaging assessment. In some embodiments, the subsequent neuroimaging assessment shows no significant further decrease in cervical spinal cord volume compared to the baseline neuroimaging assessment. In some embodiments, the concentration of frataxin protein in a subject's biofluid sample is determined before administration of gene therapy. In some embodiments, the concentration of frataxin protein in a subject's biofluid sample is determined after administration of gene therapy. In some embodiments, the concentration of frataxin protein in a subject's biofluid sample is determined before and after administration of gene therapy. In some embodiments, the concentration of neurofilament light chain in a subject's biofluid sample is determined before administration of gene therapy. In some embodiments, the concentration of neurofilament light chain in a subject's biofluid sample is determined after administration of gene therapy. In some embodiments, the concentration of neurofilament light chain in a subject's biofluid sample is determined before and after administration of gene therapy. In some embodiments, neuroimaging assessment is performed before administration of gene therapy. In some embodiments, neuroimaging assessment is performed after administration of gene therapy. In some embodiments, neuroimaging assessment is performed before and after administration of gene therapy.

In some embodiments, efficacy of gene therapy is monitored by determining frataxin concentration in biofluid following gene therapy administration. In some embodiments, efficacy of gene therapy is monitored by determining neurofilament light chain concentration in biofluid following gene therapy administration. For example, in some embodiments, gene therapy is effective if the frataxin concentration in biofluid following gene therapy administration is increased relative to the frataxin concentration in the biofluid before gene therapy. In some embodiments, gene therapy is effective if the frataxin concentration in biofluid following gene therapy is at least 20%, at least 30%, at least 40%, at least 50%, at least 60%, at least 70%, at least 80%, at least 90%, at least 95%, at least 99%, or 100% of the frataxin concentration in biofluid of a healthy subject (e.g., a subject not having Friedreich's Ataxia). In some embodiments, the healthy subject and subject having Friedreich's Ataxia (the subject receiving gene therapy) are age-matched, e.g., to control for age-related changes in neurofilament light chain concentration in biofluid. In some embodiments, gene therapy is effective if the neurofilament light chain concentration in biofluid is not significantly higher (e.g., no more than 1, 2, or 3 pg/mL higher; no more than 3%, 5%, or 7% higher) after gene therapy compared to the neurofilament light chain concentration in biofluid before gene therapy. In some embodiments, gene therapy is effective if the neurofilament light chain concentration in biofluid is unchanged after gene therapy compared to the neurofilament light chain concentration in biofluid before gene therapy. In some embodiments, gene therapy is effective if the neurofilament light chain concentration in biofluid is decreased after gene therapy compared to the neurofilament light chain concentration in biofluid before gene therapy. In some embodiments, gene therapy is effective if the neurofilament light chain concentration in biofluid is not significantly greater than the neurofilament light chain concentration of an age-matched, healthy subject (e.g., a subject not having a neurodegenerative condition or brain injury).

In some embodiments, efficacy of gene therapy is monitored by determining frataxin concentration and neurofilament light chain concentration in biofluid following gene therapy administration, optionally comparing the concentrations after gene therapy to the concentrations before gene therapy. In some embodiments, gene therapy is effective if the frataxin concentration following gene therapy administration is increased relative to frataxin concentration in the biofluid before gene therapy or if the frataxin concentration in biofluid following gene therapy is at least 20%, at least 30%, at least 40%, at least 50%, at least 60%, at least 70%, at least 80%, at least 90%, at least 95%, at least 99%, or 100% of the frataxin concentration in biofluid of a (optionally, age-matched) healthy subject (e.g., a subject not having Friedreich's Ataxia) and if the neurofilament light chain concentration in biofluid after gene therapy is not significantly greater than, is equal to, or is lower than the neurofilament light chain concentration in the biofluid before gene therapy or is not significantly greater than, is equal to, or is lower than the neurofilament light chain concentration of an age-matched, healthy subject (e.g., a subject not having a neurodegenerative condition).

In some embodiments, efficacy of gene therapy is monitored by determining frataxin concentration before and following gene therapy administration and comparing the frataxin concentration before gene therapy administration (baseline frataxin concentration) to the frataxin concentration following gene therapy administration (subsequent frataxin concentration). In some embodiments, efficacy of gene therapy is monitored by determining the neurofilament light chain concentration before and following gene therapy administration and comparing the neurofilament light chain concentration before gene therapy administration (baseline neurofilament light chain concentration) to the neurofilament light chain concentration following gene therapy administration (subsequent neurofilament light chain concentration). In some embodiments, the gene therapy is effective if the subsequent frataxin concentration is higher than the baseline frataxin concentration. In some embodiments, the gene therapy is effective if the subsequent frataxin concentration is at least 0.5×, at least 1×, or at least 1.5× higher than the baseline frataxin concentration. In some embodiments, the gene therapy is effective if the subsequent frataxin concentration is at least 2× higher than the baseline frataxin concentration. In some embodiments, the gene therapy is effective if the subsequent frataxin concentration is at least 2.5× higher than the baseline frataxin concentration. In some embodiments, the gene therapy is effective if the subsequent frataxin concentration is at least 3× higher than the baseline frataxin concentration. In some embodiments, the gene therapy is effective if the subsequent neurofilament light chain concentration is lower than the baseline neurofilament light chain concentration. In some embodiments, the gene therapy is effective if the subsequent neurofilament light chain concentration is at least 0.5×, at least 1×, or at least 1.5× lower than the baseline neurofilament light chain concentration. In some embodiments, the gene therapy is effective if the subsequent neurofilament light chain concentration is at least 2× lower than the baseline neurofilament light chain concentration. In some embodiments, the gene therapy is effective if the subsequent neurofilament light chain concentration is at least 5× lower than the baseline neurofilament light chain concentration. In some embodiments, the gene therapy is effective if the subsequent neurofilament light chain concentration is the same as the baseline neurofilament light chain concentration. In some embodiments, the gene therapy is effective if the subsequent neurofilament light chain concentration is not significantly higher than the baseline neurofilament light chain concentration, e.g., is no more than 1 pg/mL, 2 pg/mL, or 3 pg/mL compared to baseline, or has not increased by more than 3%, 5%, or 7% from baseline. In some embodiments, the gene therapy is effective if the subsequent neurofilament light chain concentration is not significantly higher than the baseline neurofilament light chain concentration, when controlled for expected increases in neurofilament light chain concentration due to aging.

In some embodiments, efficacy of gene therapy is monitored by determining frataxin concentration and neurofilament light chain concentration in biofluid before and following gene therapy administration, comparing the frataxin concentration before gene therapy administration to the frataxin concentration following gene therapy administration, and comparing the neurofilament light chain concentration before gene therapy administration to the neurofilament light chain concentration following gene therapy administration. In some embodiments, the gene therapy is effective if the subsequent frataxin concentration is higher than the baseline frataxin concentration. In some embodiments, the gene therapy is effective if the subsequent frataxin concentration is higher than the baseline frataxin concentration and if the subsequent neurofilament light chain concentration is not significantly higher than, is equal to, or is lower than the baseline neurofilament light chain concentration. For example, in some embodiments, a subsequent frataxin concentration may be at least 0.5×-3× higher than a baseline frataxin concentration (e.g., may be at least 0.5-1×, at least 1-1.5×, at least 1.5-2×, at least 2-2.5×, at least 2.5-3× higher than baseline). In some embodiments, a subsequent neurofilament light chain concentration is not significantly higher than a baseline neurofilament light chain concentration when, e.g., the subsequent concentration is no more than 1 pg/mL, no more than 2 pg/mL, or no more than 3 pg/mL higher than the baseline concentration. In some embodiments, a subsequent neurofilament light chain concentration is not significantly higher than a baseline neurofilament light chain concentration when, the subsequent concentration is no more than 3%, no more than 5%, or no more than 7% higher than the baseline concentration. In some embodiments, the gene therapy is effective if the subsequent frataxin concentration is at least 0.5×-3× higher than the baseline frataxin concentration and if the subsequent neurofilament light chain concentration is not significantly higher than, is equal to, or is lower than the baseline neurofilament light chain concentration. In some embodiments, the gene therapy is effective if the subsequent frataxin concentration is higher (e.g., at least 0.5×-3× higher) than the baseline frataxin concentration.

In some embodiments, the gene therapy is effective if neuroimaging assessment following gene therapy shows no significant volume loss in brain and/or spinal cord relative to baseline. In some embodiments, the gene therapy is effective if neuroimaging assessment following gene therapy shows no significant volume loss in spinal cord cross-sectional area relative to baseline. In some embodiments, the gene therapy is effective if neuroimaging assessment following gene therapy shows no significant increase in diffusivity relative to baseline.

In some embodiments, efficacy of gene therapy is monitored by a combination of frataxin concentration (or change in frataxin concentration) in biofluid and one or more neuroimaging assessments.

In some embodiments, efficacy of gene therapy is monitored by a combination of neurofilament light chain concentration (or change in neurofilament concentration) in biofluid and one or more neuroimaging assessments.

In some embodiments, efficacy of gene therapy is monitored by a combination of frataxin concentration (or change in frataxin concentration) in biofluid, neurofilament light chain concentration (or change in neurofilament concentration) in biofluid, and one or more neuroimaging assessments.

In some embodiments, efficacy of gene therapy is monitored to detect improvement in or reduction of at least one symptom of Friedreich's Ataxia, e.g., improvement in ataxia (balance), improvement or stabilization of gait, improvement in sensory capacity, improvement in an ataxia-associated heart condition, improvement in coordination of movement, improvement in physical strength, improvement in functional capacity, a decrease in feelings of exhaustion, and/or improvement in quality of life. In some embodiments, efficacy is further monitored by one or more neuroimaging assessments.

In some embodiments, efficacy of gene therapy is monitored by a combination of frataxin concentration (or change in frataxin concentration) in biofluid and one or more symptoms of Friedreich's Ataxia. In some embodiments, efficacy is further monitored by one or more neuroimaging assessments.

In some embodiments, efficacy of gene therapy is monitored by a combination of neurofilament light chain concentration (or change in neurofilament concentration) in biofluid and one or more symptoms of Friedreich's Ataxia. In some embodiments, efficacy is further monitored by one or more neuroimaging assessments.

In some embodiments, efficacy of gene therapy is monitored by a combination of frataxin concentration (or change in frataxin concentration) in biofluid, neurofilament light chain concentration (or change in neurofilament concentration) in biofluid, and one or more symptoms of Friedreich's Ataxia. In some embodiments, efficacy is further monitored by one or more neuroimaging assessments.

In some embodiments, frataxin concentration and/or neurofilament light chain concentration is assessed in CSF. In some embodiments, frataxin concentration and/or neurofilament light chain concentration is assessed in blood or a blood component. In some embodiments, the blood component is serum.

In some embodiments, frataxin and neurofilament light chain are detected from the same biofluid sample or sample type (e.g., testing both in CSF or both in serum). In some embodiments, frataxin and neurofilament light chain are detected from different biofluids (e.g., testing frataxin in serum and neurofilament light chain in CSF).

In some embodiments, the gene therapy comprises frataxin gene therapy.

In some embodiments, the frataxin gene therapy comprises a nucleic acid construct as disclosed in International Application Publication No. WO2020/069461, the contents of which are herein incorporated by reference in their entirety.

In some embodiments, a frataxin gene therapy may comprise an AAV, wherein the AAV comprises an AAV capsid and a viral genome. In some embodiments, the viral genome comprises a nucleic acid construct as disclosed herein or as disclosed in WO2020/069461.

In some embodiments, the frataxin gene therapy comprises a nucleic acid construct (e.g., a viral genome) comprising a 5′ ITR sequence region, a promoter region, an intron/exon region, a payload region, an optional tag, up to three miR binding sites, a polyA sequence region, an optional filler sequence, and a 3′ ITR sequence region. In some embodiments, the 5′ ITR of the 5′ ITR sequence region and/or the 3′ ITR of the 3′ ITR sequence region is an AAV2 ITR. In some embodiments, both the 5′ ITR and 3′ ITR are AAV2 ITRs.

In some embodiments the human frataxin expressed by the frataxin gene therapy is encoded by any of SEQ ID NOs: 1-3, or a fragment thereof. In some embodiments, the human frataxin expressed by the frataxin gene therapy is encoded by SEQ ID NO: 1 or a variant having at least 90%, 95%, or 99% identity to SEQ ID NO: 1, or a fragment thereof. In some embodiments, the human frataxin expressed by the frataxin gene therapy is encoded by SEQ ID NO: 1 or a fragment thereof. In some embodiments, the fragment comprises nucleotides 32-664 of SEQ ID NO: 1. In some embodiments, the fragment consists of nucleotides 32-664 of SEQ ID NO: 1. In some embodiments, the human frataxin expressed by the frataxin gene therapy is encoded by SEQ ID NO: 2 or a variant having at least 90%, 95%, or 99% identity to SEQ ID NO: 2, or a fragment thereof. In some embodiments, the human frataxin expressed by the frataxin gene therapy is encoded by SEQ ID NO: 2 or a fragment thereof. In some embodiments, the human frataxin expressed by the frataxin gene therapy is encoded by SEQ ID NO: 3 or a variant having at least 90%, 95%, or 99% identity to SEQ ID NO: 3, or a fragment thereof. In some embodiments, the human frataxin expressed by the frataxin gene therapy is encoded by SEQ ID NO: 3 or a fragment thereof.

In some embodiments, the human frataxin expressed by the frataxin gene therapy comprises any of SEQ ID NOs: 4-6. In some embodiments, the human frataxin expressed by the frataxin gene therapy consists of any of SEQ ID NOs: 4-6. In some embodiments, the human frataxin expressed by the frataxin gene therapy comprises or consists of SEQ ID NO: 4 or a variant having at least 90%, 95%, or 99% identity to SEQ ID NO: 4. In some embodiments, the human frataxin expressed by the frataxin gene therapy comprises or consists of SEQ ID NO: 4. In some embodiments, the human frataxin expressed by the frataxin gene therapy comprises or consists of SEQ ID NO: 5 or a variant having at least 90%, 95%, or 99% identity to SEQ ID NO: 5. In some embodiments, the human frataxin expressed by the frataxin gene therapy comprises or consists of SEQ ID NO: 5. In some embodiments, the human frataxin expressed by the frataxin gene therapy comprises or consists of SEQ ID NO: 6 or a variant having at least 90%, 95%, or 99% identity to SEQ ID NO: 6. In some embodiments, the human frataxin expressed by the frataxin gene therapy comprises or consists of SEQ ID NO: 6.

In some embodiments, the human frataxin expressed by the frataxin gene therapy consists of a cleaved (shorter) variant of SEQ ID NO: 4. In some embodiments, the variant is frataxin intermediate form (42-210), as given by the respective amino acids of SEQ ID NO: 4. In some embodiments, the variant is frataxin (56-210), as given by the respective amino acids of SEQ ID NO: 4. In some embodiments, the variant is frataxin (78-210), as given by the respective amino acids of SEQ ID NO: 4. In some embodiments, the variant is frataxin mature form (81-210), as given by the respective amino acids of SEQ ID NO: 4.

In some embodiments, the frataxin gene therapy construct (e.g., the nucleic acid construct) comprises a truncated CMV promoter driving expression of human frataxin. In some embodiments, the truncated CMV promoter comprises or consists of a CMV-D7 promoter (SEQ ID NO: 7). In some embodiments, the frataxin gene therapy construct comprises a truncated CBA promoter driving expression of human frataxin. In some embodiments, the truncated CBA promoter comprises or consists of a CBA-D8 promoter (SEQ ID NO: 8). In some embodiments, the truncated CBA promoter comprises or consists of a CBA-D4 promoter (SEQ ID NO: 9). In some embodiments, the truncated CBA promoter comprises or consists of a CBA-D6 promoter (SEQ ID NO: 10).

In some embodiments, the frataxin gene therapy construct comprises SEQ ID NO: 11, which comprises a 5′ inverted terminal repeat (ITR) sequence region and a 3′ ITR sequence region, a CMV-D7 promoter region, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence.

In some embodiments, the frataxin gene therapy construct comprises SEQ ID NO: 12, which comprises a 5′ inverted terminal repeat (ITR) sequence region and a 3′ ITR sequence region, a CBA-D8 promoter region, a junction sequence, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence.

In some embodiments, the frataxin gene therapy construct comprises SEQ ID NO: 13, which comprises a 5′ inverted terminal repeat (ITR) sequence region and a 3′ ITR sequence region, a CBA-D4 promoter region, a junction sequence, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence.

In some embodiments, the frataxin gene therapy construct comprises SEQ ID NO: 14, which comprises a 5′ inverted terminal repeat (ITR) sequence region and a 3′ ITR sequence region, a CBA-D6 promoter region, a human beta globin intron/exon region comprising an ie1 exon 1 region, an ie1 intron 1 region, a human beta-globin intron region, a human beta-globin exon region, a human frataxin payload sequence, a miR binding site series comprising three repeats of single miR122 binding site sequences, a human growth hormone polyadenylation sequence, and an albumin filler sequence.

In some embodiments, the frataxin gene therapy comprises an AAV comprising a VOY101 capsid. In some embodiments, the VOY101 capsid comprises or consists of the amino acid sequence SEQ ID NO: 15. In some embodiments, the VOY101 capsid is encoded by the nucleic acid sequence SEQ ID NO: 16. in some embodiments, the frataxin gene therapy comprises an AAV comprising a sequence that is at least 95%, 96%, 97%, 98%, or 99% identical to VOY101. In some embodiments, the capsid comprises or consists of a sequence that is at least 95%, 96%, 97%, 98%, or 99% identical to the amino acid sequence SEQ ID NO: 15. In some embodiments, the capsid is encoded by a nucleic acid sequence that is at least 95%, 96%, 97%, 98%, or 99% identical to SEQ ID NO: 16. In some embodiments, the frataxin gene therapy comprises an AAV comprising a sequence that is at least 99% identical to VOY101. In some embodiments, the capsid comprises or consists of a sequence that is at least 99% identical to the amino acid sequence SEQ ID NO: 15. In some embodiments, the capsid is encoded by a nucleic acid sequence that is at least 99% identical to SEQ ID NO: 16.

In some embodiments, the frataxin gene therapy is formulated to comprise sodium chloride, sodium phosphate, potassium chloride, potassium phosphate and poloxamer 188. In some embodiments, the frataxin gene therapy is formulated to comprise 192 mM sodium chloride, 10 mM sodium phosphate, 2.7 mM potassium chloride, 2 mM potassium phosphate and 0.001% poloxamer 188 (v/v), wherein the pH of the formulation is 7.4.

Second Therapies in Combination with Gene Therapy

In some embodiments, a subject is administered a gene therapy capable of increasing frataxin levels and the choice of the second therapy is determined by the concentration of frataxin in biofluid following gene therapy. For example, if the concentration of frataxin in biofluid before gene therapy treatment is just above, below, or approximately equal to a limit of detection (e.g., a limit of about 3.0 p/mL or less, about 2.0 pg/ml or less, about 1.0 pg/mL or less, 0.5 pg/mL or less) and is not detectable or is barely detectable (e.g., approximately equal to the limit of detection) in biofluid after gene therapy treatment, or if the concentration of frataxin is not significantly increased (e.g., at least 0.5×-3× increased) following gene therapy, the subject may be administered a second therapy wherein the second therapy is capable of reducing oxidative stress, reducing mitochondrial dysfunction, restoring mitochondrial function, reducing inflammation, or resolving inflammation, or wherein the second therapy (e.g., oligonucleotide-based therapy, a stabilizing agent, or a carrier protein) is capable of increasing frataxin levels. In some embodiments, any of these therapies may be administered at a high dose. In some embodiments, the subject also receives one or more therapies to manage one or more symptoms of Friedreich's Ataxia. Such therapies may include, but are not limited to, occupational therapy, speech therapy, orthopedic care, bracing, surgery, heart disease, an antioxidant, treatment to lower blood sugar, and physical therapy.

In some embodiments, if the concentration of frataxin in a subject's biofluid before gene therapy treatment is just above, below, approximately equal to a limit of detection (e.g., a limit of about 3.0 pg/mL or less, about 2.0 pg/ml or less, about 1.0 pg/mL or less, 0.5 pg/mL or less) but is above the limit of detection in biofluid after gene therapy treatment, or if the concentration of frataxin is significantly increased (e.g., at least 0.5×-3× increased) following gene therapy, then the subject may be administered a second therapy, wherein the second therapy is capable of reducing oxidative stress, reducing mitochondrial dysfunction, restoring mitochondrial function, reducing inflammation, or resolving inflammation, or wherein the second therapy (e.g., oligonucleotide-based therapy, a stabilizing agent, or a carrier protein) is capable of increasing frataxin levels, preferably at a low dose. In some embodiments, the subject also receives one or more therapies to manage one or more symptoms of Friedreich's Ataxia. Such therapies may include, but are not limited to, occupational therapy, speech therapy, orthopedic care, bracing, surgery, heart disease, an antioxidant, treatment to lower blood sugar, and physical therapy.

In some embodiments, a significant increase in frataxin from baseline is an increase of at least about 0.5× to about 3× (e.g., at least about 0.5-1×, at least about 1-1.5×, at least about 1.5-2×, at least about 2-2.5×, at least about 2.5-3×) over baseline. In some embodiments, a significant increase in frataxin concentration is at least 1.5×, at least 2× greater, or at least 3× greater than baseline frataxin concentration. In some embodiments, a significant increase in frataxin from baseline is a frataxin concentration that is at least 20%, at least 30%, at least 40%, at least 50%, at least 60%, at least 70%, at least 80%, at least 90%, at least 95%, at least 99%, or 100% of normal levels (i.e., at least 20%, at least 30%, at least 40%, at least 50%, at least 60%, at least 70%, at least 80%, at least 90%, at least 95%, at least 99%, or 100% of the frataxin levels of an individual who does not have Friedreich's Ataxia). In some embodiments, a significant decrease in neurofilament light chain from baseline is about 0.5×-2× or greater, e.g., 0.5×, 1×, 1.5×, 2×, 3×, 5×, 10×, 25×, 50×, or 100×, or more. In some embodiments, a low level of neurofilament light chain is about 20 pg/mL, about 10 pg/mL, about 5 pg/mL, about 2 pg/mL, about 1 pg/mL, or lower. In some embodiments, a high level of neurofilament light chain is about 40 pg/mL, about 45 pg/mL, about 50 pg/mL, about 60 pg/mL, about 70 pg/mL, or higher. In some embodiments, a low or high level of neurofilament light chain is determined based on a comparison to an age-matched healthy subject (e.g., a subject not having a neurodegenerative condition or brain injury).

In some embodiments, if the concentration of frataxin in biofluid is significantly increased (e.g., at least 0.5×-3× higher), at or near normal levels, or at least 20% of normal levels, following gene therapy, the subject may receive one or more therapies to manage one or more remaining symptoms of Friedreich's Ataxia. Such therapies may include, but are not limited to, occupational therapy, speech therapy, orthopedic care, bracing, surgery, heart disease, an antioxidant, treatment to lower blood sugar, and physical therapy. In some embodiments, if the concentration of frataxin in biofluid is significantly increased (e.g., at least 0.5×-3× higher), at or near normal levels, or at least 20% of normal levels, following gene therapy, the subject does not receive any therapy.

In some embodiments, a dose of a second therapy is determined by the change in frataxin concentration (as measured in a subject's biofluid sample using an assay described herein) from baseline, where the change in frataxin concentration is assessed after the subject receives gene therapy comprising a nucleic acid construct driving expression of frataxin or a functional fragment thereof. In some embodiments, a dose of a second therapy is determined by the change in frataxin concentration from baseline and by the concentration or change in neurofilament light chain concentration from baseline.

For example, in some embodiments, i) a subject's baseline frataxin protein levels (and optionally neurofilament light chain levels) are determined in a biofluid using an assay as described herein prior to administration of gene therapy, ii) the subject is administered gene therapy, iii) a significant increase in frataxin concentration is detected in a biofluid using an assay as described herein following gene therapy and/or no significant increase in neurofilament light chain (which may include a significant decrease in or no change in neurofilament light chain concentration) is detected in a biofluid following gene therapy, and iv) the subject is administered a second therapy at a low dose. In some embodiments, i) a subject's baseline frataxin protein levels (and optionally neurofilament light chain levels) are determined in a biofluid using an assay as described herein prior to administration of gene therapy, ii) the subject is administered gene therapy, iii) a significant increase in frataxin concentration is detected in a biofluid using an assay as described herein following gene therapy and/or no detectable neurofilament light chain is found in a biofluid following gene therapy, and iv) the subject is administered a second therapy at a low dose. In some embodiments, i) a subject's baseline frataxin protein levels (and optionally neurofilament light chain levels) are determined in a biofluid using an assay as described herein prior to administration of gene therapy, ii) the subject is administered gene therapy, iii) a significant increase in frataxin concentration is detected in a biofluid using an assay as described herein following gene therapy and/or a low concentration of neurofilament light chain (e.g., less than 40 pg/mL, less than 30 pg/mL, less than 20 pg/mL) is detected in a biofluid following gene therapy, and iv) the subject is administered a second therapy at a low dose.

In certain embodiments, a subject's baseline frataxin protein levels (and optionally neurofilament light chain levels) are determined in a biofluid using an assay as described herein prior to administration of gene therapy, the subject is administered gene therapy, no significant increase in frataxin expression is detected and/or a significant increase in neurofilament light chain (or a high level of neurofilament light chain) is detected in a biofluid using an assay as described herein following gene therapy, and the subject is administered a second therapy at a high dose.

In some embodiments, a subject is tested for frataxin concentration and/or neurofilament light chain concentration before being administered a second therapy. In some embodiments, a subject is administered a second therapy at least once before the subject's biofluid sample is tested for frataxin concentration and/or neurofilament light chain concentration. In some embodiments, a subject is administered a second therapy at least once before and at least once after the subject's biofluid sample is tested for frataxin concentration and/or neurofilament light chain concentration.

In some embodiments, whether to administer a dose of a second therapy is determined by the change in frataxin concentration from baseline and by the change in one or more neuroimaging assessments from baseline. For example, in some embodiments, i) a subject's baseline frataxin protein levels (and optionally neurofilament light chain levels) are determined in a biofluid using an assay as described herein prior to administration of gene therapy and the subject also undergoes one or more neuroimaging assessments (e.g., to assess atrophy or diffusivity), ii) the subject is administered a gene therapy, iii) a significant increase in frataxin concentration is detected in a biofluid using an assay as described herein following gene therapy and/or no significant increase in neurofilament light chain (which may include a significant decrease in or no change in neurofilament light chain concentration) is detected in a biofluid following gene therapy and/or no significant worsening in atrophy or diffusivity is detected by a subsequent neuroimaging assessment, and iv) the subject is administered a second therapy at a low dose. In some embodiments, i) a subject's baseline frataxin protein levels (and optionally neurofilament light chain levels) are determined in a biofluid using an assay as described herein prior to administration of gene therapy and the subject also undergoes one or more neuroimaging assessments (e.g., to assess atrophy or diffusivity), ii) the subject is administered gene therapy, iii) a significant increase in frataxin concentration is detected in a biofluid using an assay as described herein following gene therapy and/or no detectable neurofilament light chain is found in a biofluid following gene therapy and/or no significant worsening in atrophy or diffusivity is detected by a subsequent neuroimaging assessment, and iv) the subject is administered a second therapy at a low dose. In some embodiments, i) a subject's baseline frataxin protein levels (and optionally neurofilament light chain levels) are determined in a biofluid using an assay as described herein prior to administration of gene therapy and the subject also undergoes one or more neuroimaging assessments (e.g., to assess atrophy or diffusivity), ii) the subject is administered gene therapy, iii) a significant increase in frataxin concentration is detected in a biofluid using an assay as described herein following gene therapy and/or a low concentration of neurofilament light chain (e.g., less than 40 pg/mL, less than 30 pg/mL, less than 20 pg/mL) is detected in a biofluid following gene therapy and/or no significant worsening in atrophy or diffusivity is detected by a subsequent neuroimaging assessment, and iv) the subject is administered a second therapy at a low dose.

In some embodiments, no significant worsening of atrophy and/or diffusivity is indicated by no more than 5% worsening in atrophy and/or diffusivity. For example, in some embodiments, no worsening of atrophy means that atrophy has not increased more than 5% compared to atrophy prior to treatment. In some embodiments, no worsening of diffusivity means that diffusivity has not increased more than 5% compared to diffusivity prior to treatment.

In certain embodiments, a subject's baseline frataxin protein levels (and optionally neurofilament light chain levels) are determined in a biofluid using an assay as described herein and the subject undergoes one or more neuroimaging assessments (e.g., to assess atrophy or diffusivity) prior to administering a gene therapy, the subject is administered the gene therapy, no significant increase in frataxin expression is detected and/or a significant increase in neurofilament light chain (or a high level of neurofilament light chain) is detected in a biofluid using an assay as described herein and/or atrophy or diffusivity significantly worsens following gene therapy, and the subject is administered a second therapy at a high dose.

In some embodiments, a subject is tested for frataxin concentration and/or neurofilament light chain concentration before being administered a second therapy. In some embodiments, a subject is administered a second therapy at least once before the subject's biofluid sample is tested for frataxin concentration and/or neurofilament light chain concentration. In some embodiments, a subject is administered a second therapy at least once before and at least once after the subject's biofluid sample is tested for frataxin concentration and/or neurofilament light chain concentration.

In some embodiments, a subject undergoes neuroimaging assessment before being administered a second therapy. In some embodiments, a subject is administered a second therapy at least once before the subject undergoes neuroimaging assessment. In some embodiments, a subject is administered a second therapy at least once before and at least once after the subject undergoes neuroimaging assessment.

### III. DEFINITIONS

At various places in the present disclosure, substituents or properties of compounds of the present disclosure are disclosed in groups or in ranges. It is specifically intended that the present disclosure include each and every individual or sub combination of the members of such groups and ranges.

Unless stated otherwise, the following terms and phrases have the meanings described below. The definitions are not meant to be limiting in nature and serve to provide a clearer understanding of certain aspects of the present disclosure.

About: As used herein, the term “about” means +/−10% of the recited value.

Adeno-associated virus: The term “adeno-associated virus” or “AAV” as used herein refers to members of the dependovirus genus comprising any particle, sequence, gene, protein, or component derived therefrom.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the subject. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals include, but are not limited to, mammals, birds, reptiles, amphibians, fish, and worms. In certain embodiments, the animal is a transgenic animal, genetically-engineered animal, or a clone.

Antibody: As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional,” such as binding an antigen). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include V_{H} and V_{L} domains, scFvs, Fab, Fab′, F(ab′)₂, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Antibody-based composition: As used herein, “antibody-based” or “antibody-derived” compositions are monomeric or multimeric polypeptides which comprise at least one amino-acid region derived from a known or parental antibody sequence and at least one amino acid region derived from a non-antibody sequence, e.g., mammalian protein.

Approximately: As used herein, the term “approximately” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.25%, 0.1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Biofluid: As used herein, the term “biofluid” refers to a liquid from the body of a subject (e.g., human or other animal). Non-limiting examples of biofluids include cerebrospinal fluid (CSF), plasma, serum, blood, saliva, excreta, sweat, tears, vaginal fluid, semen, breast milk, urine, bile, peritoneal fluid, pericardial fluid, pleural fluid, amniotic fluid, synovial fluid, aqueous humor, vitreous humor, gastric fluid, mucus, sputum, nasal discharge, fluid of the throat or lungs, intracellular fluid, extracellular fluid, interstitial fluid, transcellular fluid, and/or lymphatic fluid (e.g., endolymph, perilymph). A biofluid may be a combination of two or more of the aforementioned bodily fluids. A biofluid may be from a human body, or may be from another animal body (e.g., non-human primate, dog, cat, mouse, rat, rabbit, guinea pig, etc.).

Capsid: As used herein, the term “capsid” refers to the protein shell of a virus.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). Codon optimization can be completed using methods and databases known to those in the art.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Delivery: As used herein, “delivery” refers to the act or manner of delivering a therapeutic, an AAV, a compound, substance, entity, moiety, cargo, or payload.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance, and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Dosing regimen: As used herein, a “dosing regimen” is a schedule of administration or physician determined regimen of treatment, prophylaxis, or palliative care.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Epitope: As used herein, an “epitope” refers to a surface or region on a molecule that is capable of interacting with a biomolecule. For example, a protein may contain one or more amino acids, e.g., an epitope, which interacts with an antibody, e.g., a biomolecule. In some embodiments, when referring to a protein or protein module, an epitope may comprise a linear stretch of amino acids or a three-dimensional structure formed by folded amino acid chains.

Formulation: As used herein, a “formulation” includes at least one AAV and a delivery agent or excipient.

Fragment: A “fragment,” as used herein, refers to a portion. For example, fragments of proteins may include polypeptides obtained by digesting full-length protein isolated from cultured cells. A fragment may also refer to a truncation (e.g., an N-terminal and/or C-terminal truncation) of a protein or a truncation (e.g., at the 5′ and/or 3′ end) of a nucleic acid. A protein fragment may be obtained by expression of a truncated nucleic acid, such that the nucleic acid encodes a portion of the full-length protein.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression,” this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% identical for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form, or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in a substance (e.g., frataxin). Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

Modified: As used herein “modified” refers to a changed state or structure of a molecule of the present disclosure. Molecules may be modified in many ways including chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid,” “polynucleotide,” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid,” “polynucleotide,” and “oligonucleotide,” and these terms will be used interchangeably. These terms refer only to the primary structure of the molecule. Thus, these terms include double- and single-stranded DNA, as well as double- and single stranded RNA.

Patient: As used herein, “patient” refers to a subject that may be suffering from, suspected of having, susceptible to, or diagnosed with one or more diseases or who may seek or be in need of treatment, require treatment, be receiving treatment, will receive treatment, or a subject who is under care by a trained professional for a particular disease or condition. In certain embodiments, a patient may have a decreased level of frataxin as compared to a control subject. In certain embodiments, a patient may be diagnosed with Friedreich's ataxia.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure. As used herein, a substance is “pure” if it is substantially free of (substantially isolated from) other components.

Region: As used herein, the term “region” refers to a zone or general area. In certain embodiments, when referring to a protein or protein module, a region may include a linear sequence of amino acids along the protein or protein module or may include a three-dimensional area, an epitope and/or a cluster of epitopes. In certain embodiments, regions include terminal regions. As used herein, the term “terminal region” refers to regions located at the ends or termini of a given agent. When referring to proteins, terminal regions may include N- and/or C-termini. N-termini refer to the end of a protein comprising an amino acid with a free amino group. C-termini refer to the end of a protein comprising an amino acid with a free carboxyl group. N- and/or C-terminal regions may there for include the N- and/or C-termini as well as surrounding amino acids. In certain embodiments, N- and/or C-terminal regions include from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may include any length of amino acids that includes the N-terminus but does not include the C-terminus. In certain embodiments, C-terminal regions may include any length of amino acids, which include the C-terminus, but do not include the N-terminus.

In certain embodiments, when referring to a polynucleotide, a region may include a linear sequence of nucleic acids along the polynucleotide or may include a three-dimensional area, secondary structure, or tertiary structure. In certain embodiments, regions include terminal regions. As used herein, the term “terminal region” refers to regions located at the ends or termini of a given agent. When referring to polynucleotides, terminal regions may include 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for include the 5′ and 3′ termini as well as surrounding nucleic acids. In certain embodiments, 5′ and 3′ terminal regions include from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may include any length of nucleic acids that includes the 5′ terminus but does not include the 3′ terminus. In certain embodiments, 3′ regions may include any length of nucleic acids, which include the 3′ terminus, but does not include the 5′ terminus.

Sample: As used herein, the term “sample” or “biological sample” refers to a subset of its tissues, cells, or component parts (e.g. bodily fluids, including but not limited to blood, mucus, lymphatic fluid, synovial fluid, cerebrospinal fluid, saliva, amniotic fluid, amniotic fluid, urine, vaginal fluid, and semen). The sample may be from a human, or may be from another animal (e.g., non-human primate, dog, cat, mouse, rat, rabbit, guinea pig, etc.). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample may be collected from an organism or may be derived from a manufactured source, such as, but not limited to cultured cells (e.g., CHO, HEK or IPSC). A sample further refers to a medium, such as a nutrient broth or gel, which may contain cellular components, such as proteins or nucleic acid molecules.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. In certain embodiments, a sample or a biofluid may be taken from a subject and analyzed with a method described herein.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” Friedreich's Ataxia may refer to reducing one or more symptoms or hallmarks of Friedreich's Ataxia, such as by reducing or eliminating inflammation, partially or fully restoring motor function and/or dexterity, decreasing mitochondrial dysfunction, increasing frataxin expression and/or protein levels, or other improvements in health or quality of life as disclosed herein. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may include adeno-associated virus (AAV) parent or reference sequence. Such parent or reference AAV sequences may serve as an original, second, third or subsequent sequence for engineering vectors. In non-limiting examples, such parent or reference AAV sequences may include any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” or “viral vector” refers to the nucleic acid sequence(s) encapsulated in an AAV. Viral genomes comprise at least one payload region encoding polypeptides or fragments thereof.

### IV. EXAMPLES

### Example 1. Development of Capture-Reporter Antibody Combinations for a Proof-of-Concept Frataxin Detection Assay

A proof-of-concept frataxin detection assay was developed by first screening and identifying the best performing capture-reporter pair(s) for human frataxin from twenty-five antibody pair combinations. These combinations consisted of five individual anti-frataxin antibodies (Ab-1, Ab-2, Ab-3, Ab-4, and Ab-5, outlined in Table 2 above) that were evaluated as both capture and detector in all possible combinations (outlined in Table 4 below), including conditions in which the same antibody was used as both capture and detector since frataxin can exist as an oligomer. Each capture antibody was conjugated to magnetic beads using a standard 2-step EDC coupling chemistry. Capture beads were conjugated at 4° C. using 0.3 mg/mL EDC and 0.2 mg/mL capture antibody in reaction. Each detector antibody was biotinylated at a molar excess ratio of 40×.

TABLE 4
Antibody orientations screened

| Antibody Pair | Capture | Detector |
|---|---|---|
| Combination | Antibody | Antibody |
| 1 | Ab-4 | Ab-4 |
| 2 | Ab-4 | Ab-3 |
| 3 | Ab-4 | Ab-5 |
| 4 | Ab-4 | Ab-2 |
| 5 | Ab-4 | Ab-1 |
| 6 | Ab-3 | Ab-4 |
| 7 | Ab-3 | Ab-3 |
| 8 | Ab-3 | Ab-5 |
| 9 | Ab-3 | Ab-2 |
| 10 | Ab-3 | Ab-1 |
| 11 | Ab-5 | Ab-4 |
| 12 | Ab-5 | Ab-3 |
| 13 | Ab-5 | Ab-5 |
| 14 | Ab-5 | Ab-2 |
| 15 | Ab-5 | Ab-1 |
| 16 | Ab-2 | Ab-4 |
| 17 | Ab-2 | Ab-3 |
| 18 | Ab-2 | Ab-5 |
| 19 | Ab-2 | Ab-2 |
| 20 | Ab-2 | Ab-1 |
| 21 | Ab-1 | Ab-4 |
| 22 | Ab-1 | Ab-3 |
| 23 | Ab-1 | Ab-5 |
| 24 | Ab-1 | Ab-2 |
| 25 | Ab-1 | Ab-1 |

For initial screening purposes, capture beads were tested at 300,000 beads/well using 150 pM streptavidin-β-galactosidase (SβG) and 0.2 μg/mL detector antibody in reaction with a preliminary calibration curve in a 3-step assay format. An abbreviated four-point calibration curve was used for screening purposes. Initially, standard PBS-based buffers containing BSA were used to dilute the frataxin antigen, capture beads, detector antibody, and SβG concentrate. The antibody pairs were evaluated based on assay background, signal to background ratios, and overall assay functional sensitivity. The Average Enzymes per Bead (AEB) was determined, and an average of the AEB (AVE), standard deviation (SD), and Coefficient of Variation (% CV) were calculated. The signal to background (S/B) was also determined as an indicator of validity or noise. Results of this initial screening are shown in Tables 5-9 below. TMF indicates “too much fluorescence (signal saturation) and NaN indicates “no available number due to lost replicates”.

TABLE 5
Ab-4 capture antibody initial screening results

Assay Format
3-step, 2.0
Beads Ab
Ab-4
Detector Ab

| | Ab-4 | | | Ab-3 | | |
|---|---|---|---|---|---|---|
| [Frataxin] | Ave | | | Ave | | |
| pg/mL | AEB | % CV | S/B | AEB | % CV | S/B |
| 0 | 0.0230 | 25% | 1.00 | 0.0480 | 8% | 1.00 |
| 100 | 0.0213 | 5% | 0.93 | 1.0488 | 0% | 21.85 |
| 1000 | 0.0589 | 3% | 2.55 | 17.2387 | 1% | 359.15 |
| 10000 | 0.8200 | 12% | 35.59 | TMF | — | — |

| | | |
|---|---|---|
| LOD (pg/mL) | 358.01 | 1.30 |

| | Assay Format | | | | | | | | |
|---|---|---|---|---|---|---|---|---|---|
| | 3-step, 2.0 | | | | | | | | |
| | Beads Ab | | | | | | | | |
| | Ab-4 | | | | | | | | |
| | Detector Ab | | | | | | | | |
| | Ab-5 | | | Ab-2 | | | Ab-1 | | |
| [Frataxin] | Ave | | | Ave | | | Ave | | |
| pg/mL | AEB | % CV | S/B | AEB | % CV | S/B | AEB | % CV | S/B |
| 0 | 0.5388 | 1% | 1.00 | 3.6621 | 2% | 1.00 | 3.4311 | 8% | 1.00 |
| 100 | 0.8144 | 1% | 1.51 | 4.7306 | 2% | 1.29 | 4.7153 | 3% | 1.37 |
| 1000 | 3.9459 | 1% | 7.32 | 13.5690 | 1% | 3.71 | 12.4456 | 3% | 3.63 |
| 10000 | 23.7409 | 1% | 44.06 | TMF | — | — | TMF | — | — |

| | | | |
|---|---|---|---|
| LOD (pg/mL) | 52.77 | 89.59 | 66.05 |

TABLE 6
Ab-3 capture antibody initial screening results

Assay Format
3-step, 2.0
Beads Ab
Ab-3
Detector Ab

| | Ab-4 | | | Ab-3 | | |
|---|---|---|---|---|---|---|
| [Frataxin] | Ave | | | Ave | | |
| pg/mL | AEB | % CV | S/B | AEB | % CV | S/B |
| 0 | 0.0580 | 14% | 1.00 | 0.0047 | 23% | 1.00 |
| 100 | 0.6796 | 1% | 11.72 | 0.0152 | 6% | 3.21 |
| 1000 | 8.0243 | 4% | 138.41 | 0.0854 | 1% | 18.06 |
| 10000 | TMF | | | 1.0576 | 10% | 223.59 |

| | | |
|---|---|---|
| LOD (pg/mL) | 2.41 | 11.86 |

| | Assay Format | | | | | | | | |
|---|---|---|---|---|---|---|---|---|---|
| | 3-step, 2.0 | | | | | | | | |
| | Beads Ab | | | | | | | | |
| | Ab-3 | | | | | | | | |
| | Detector Ab | | | | | | | | |
| | Ab-5 | | | Ab-2 | | | Ab-1 | | |
| [Frataxin] | Ave | | | Ave | | | Ave | | |
| pg/mL | AEB | % CV | S/B | AEB | % CV | S/B | AEB | % CV | S/B |
| 0 | 0.2604 | 2% | 1.00 | 1.0263 | 0% | 1.00 | 0.9359 | 2% | 1.00 |
| 100 | 0.4692 | 8% | 1.80 | 2.4240 | 4% | 2.36 | 1.9159 | 4% | 2.05 |
| 1000 | 2.6355 | 1% | 10.12 | 11.5866 | 2% | 11.29 | 10.2921 | 4% | 11.00 |
| 10000 | 20.5163 | 6% | 78.77 | TMF | — | — | TMF | — | — |

| | | | |
|---|---|---|---|
| LOD (pg/mL) | 33.60 | 17.96 | 23.78 |

TABLE 7
Ab-5 capture antibody initial screening results

Assay Format
3-step, 2.0
Beads Ab
Ab-5
Detector Ab

| | Ab-4 | | | Ab-3 | | |
|---|---|---|---|---|---|---|
| [Frataxin] | Ave | | | Ave | | |
| pg/mL | AEB | % CV | S/B | AEB | % CV | S/B |
| 0 | 0.4099 | 2% | 1.00 | 0.1907 | 1% | 1.00 |
| 100 | 2.0228 | 4% | 4.93 | 2.8955 | 1% | 15.18 |
| 1000 | 18.5964 | 4% | 45.36 | 22.9200 | 3% | 120.18 |
| 10000 | TMF | — | — | TMF | — | — |

| | | |
|---|---|---|
| LOD (pg/mL) | 6.44 | 1.73 |

| | Assay Format | | | | | | | | |
|---|---|---|---|---|---|---|---|---|---|
| | 3-step, 2.0 | | | | | | | | |
| | Beads Ab | | | | | | | | |
| | Ab-5 | | | | | | | | |
| | Detector Ab | | | | | | | | |
| | Ab-5 | | | Ab-2 | | | Ab-1 | | |
| [Frataxin] | Ave | | | Ave | | | Ave | | |
| pg/mL | AEB | % CV | S/B | AEB | % CV | S/B | AEB | % CV | S/B |
| 0 | 0.0984 | 3% | 1.00 | 0.0977 | 4% | 1.00 | 0.0892 | 1% | 1.00 |
| 100 | 0.5726 | 0% | 5.82 | 0.9606 | 1% | 9.84 | 0.8342 | 0% | 9.35 |
| 1000 | 5.5268 | 7% | 56.17 | 9.3496 | 2% | 95.74 | 8.5571 | 1% | 95.92 |
| 10000 | 21.6530 | 4% | 220.07 | 29.5234 | 0% | 302.31 | 28.8614 | 3% | 323.53 |

| | | | |
|---|---|---|---|
| LOD (pg/mL) | 7.44 | 4.43 | 4.85 |

TABLE 8
Ab-2 capture antibody initial screening results

Assay Format
3-step, 2.0
Beads Ab
Ab-2
Detector Ab

| | Ab-4 | | | Ab-3 | | |
|---|---|---|---|---|---|---|
| [Frataxin] | Ave | | | Ave | | |
| pg/mL | AEB | % CV | S/B | AEB | % CV | S/B |
| 0 | 0.1797 | 17% | 1.00 | 0.0444 | 21% | 1.00 |
| 100 | 1.0188 | 8% | 5.67 | 2.0316 | 17% | 45.79 |
| 1000 | 7.3180 | 17% | 40.73 | 17.1954 | 13% | 387.56 |
| 10000 | 32.8872 | 5% | 183.03 | 32.1730 | 6% | 725.14 |

| | | |
|---|---|---|
| LOD (pg/mL) | 5.01 | 1.33 |

| | Assay Format | | | | | | | | |
|---|---|---|---|---|---|---|---|---|---|
| | 3-step, 2.0 | | | | | | | | |
| | Beads Ab | | | | | | | | |
| | Ab-2 | | | | | | | | |
| | Detector Ab | | | | | | | | |
| | Ab-5 | | | Ab-2 | | | Ab-1 | | |
| [Frataxin] | Ave | | | Ave | | | Ave | | |
| pg/mL | AEB | % CV | S/B | AEB | % CV | S/B | AEB | % CV | S/B |
| 0 | 0.0158 | 16% | 1.00 | 0.0013 | 11% | 1.00 | 0.0015 | 36% | 1.00 |
| 100 | 0.1153 | 9% | 7.30 | 0.0068 | 2% | 5.41 | 0.0069 | 22% | 4.45 |
| 1000 | 1.0275 | 7% | 65.01 | 0.1012 | 7% | 80.40 | 0.0712 | 25% | 46.25 |
| 10000 | 5.4148 | 18% | 342.61 | 0.8299 | 74% | 659.10 | 1.3629 | 4% | 884.63 |

| | | | |
|---|---|---|---|
| LOD (pg/mL) | 4.62 | 9.76 | 7.78 |

TABLE 9
Ab-1 capture antibody initial screening results

Assay Format
3-step, 2.0
Beads Ab
Ab-1
Detector Ab

| | Ab-4 | | | Ab-3 | | |
|---|---|---|---|---|---|---|
| [Frataxin] | Ave | | | Ave | | |
| pg/mL | AEB | % CV | S/B | AEB | % CV | S/B |
| 0 | 0.2204 | 2% | 1.00 | 0.0495 | 41% | 1.00 |
| 100 | 0.6398 | 46% | 2.90 | 2.4716 | 3% | 49.97 |
| 1000 | 8.7361 | 18% | 39.63 | 17.4185 | 3% | 352.18 |
| 10000 | 30.8743 | 8% | 140.07 | 28.5008 | 5% | 576.25 |

| | | |
|---|---|---|
| LOD (pg/mL) | 23.93 | 1.14 |

| | Assay Format | | | | | | | | |
|---|---|---|---|---|---|---|---|---|---|
| | 3-step, 2.0 | | | | | | | | |
| | Beads Ab | | | | | | | | |
| | Ab-1 | | | | | | | | |
| | Detector Ab | | | | | | | | |
| | Ab-5 | | | Ab-2 | | | Ab-1 | | |
| [Frataxin] | Ave | | | Ave | | | Ave | | |
| pg/mL | AEB | % CV | S/B | AEB | % CV | S/B | AEB | % CV | S/B |
| 0 | 0.0099 | — | 1.00 | 0.0012 | | 1.00 | NaN | — | — |
| 100 | 0.1059 | 24% | 10.75 | 0.0088 | 20% | 7.44 | 0.0070 | 42% | — |
| 1000 | 0.9979 | 8% | 101.31 | 0.1072 | 4% | 90.80 | 0.0953 | 1% | — |
| 10000 | 5.9298 | 8% | 602.00 | 1.0534 | 2% | 891.94 | 1.1903 | 27% | — |

| | | | |
|---|---|---|---|
| LOD (pg/mL) | 2.95 | 5.71 | NaN |

Based on the results from the initial antibody screening of all 25 antibody combinations in the 3-step assay format, six antibody pairs were selected for further testing. Using the same reagent conditions as previously described, six antibody pairs were further evaluated by directly comparing 2-step and 3-step assay formats and by screening a human brain lysate sample with high endogenous concentrations of frataxin protein. Table 10 shows results of three exemplary antibody pairs and assay format identified from this experiment based on assay background, signal to background ratios, and estimated assay LOD. Full results can be found in Tables 11-13 below. The Average Enzymes per Bead (AEB) was determined, and an average of the AEB (AVE), standard deviation (SD), and Coefficient of Variation (% CV) were calculated. The signal to background (S/B) was also determined as an indicator of validity or noise.

As the data in Table 10 show, there is a large discrepancy in brain lysate concentration between the first antibody pair and the latter two antibody pairs. Therefore, dilution linearity of the brain lysate was performed to further evaluate specificity of signal for each antibody pair.

TABLE 10
Three exemplary antibody pairs

| | Assay Format | | | | | |
|---|---|---|---|---|---|---|
| | 2-step, 2.0 (35 min-5 min) | | | | | |
| | Capture Ab | | | | | |
| | Ab-3 | | Ab-2 | | Ab-1 | |
| | Detector Ab | | | | | |
| | Ab-4 | | Ab-3 | | Ab-3 | |
| [Frataxin] | Ave | | Ave | | Ave | |
| pg/mL | AEB | S/B | AEB | S/B | AEB | S/B |
| 0 | 0.0755 | — | 0.0807 | — | 0.0800 | — |
| 100 | 5.0990 | 67.56 | 7.1446 | 88.56 | 7.1054 | 88.85 |
| 1000 | 29.7320 | 393.91 | 37.279 | 462.11 | 30.676 | 383.60 |

| | | | |
|---|---|---|---|
| 10000 | Saturated | Saturated | Saturated |
| LOD (pg/mL) | 0.36 | 0.27 | 0.27 |

| | Ave | Conc. | Ave | Conc. | Ave | Conc. |
|---|---|---|---|---|---|---|
| Sample | AEB | (pg/mL) | AEB | (pg/mL) | AEB | (pg/mL) |
| Brain Lysate | 28.95 | 455791 | 4.396 | 29911 | 4.093 | 27788 |
| (Diluted 500x) | | | | | | |

Average Enzyme per Bead (AEB) values of two replicates and signal-to-background ratios of an abbreviated 4-point frataxin calibration curve run using the top three antibody pairs in a 2-step assay format. Average AEB and concentration values of the 500× diluted human brain lysate sample are also shown. Approximate limit of detection (LOD) is calculated based on 2.5× SD (Standard Deviation) above background signal, assuming a 10% CV (Coefficient of Variation).

TABLE 11
Comparison of capture-detector
capabilities of Ab-4/Ab-3 antibody
pairs using 2-step and 3-step assays

| | Assay Format | | | | | |
|---|---|---|---|---|---|---|
| | 2-step, 2.0 | | | 3-step, 2.0 | | |
| | Beads Ab | | | | | |
| | Ab-4 | | | Ab-4 | | |
| | Detector Ab | | | | | |
| | Ab-3 | | | Ab-3 | | |
| [Frataxin] | Ave | | | Ave | | |
| pg/mL | AEB | % CV | S/B | AEB | % CV | S/B |
| 0 | 0.1226 | 10% | 1.00 | 0.1172 | 3% | 1.00 |
| 100 | 8.4926 | 5% | 69.27 | 2.7935 | 2% | 23.83 |
| 1000 | 23.6515 | — | 192.91 | 25.1537 | 2% | 214.59 |
| 10000 | TMF | — | — | TMF | — | — |

| | | |
|---|---|---|
| LOD (pg/mL) | 0.34 | 1.09 |

| | | | Corrected | | | Corrected |
|---|---|---|---|---|---|---|
| | Dilution | AEB | Conc. | Dilution | AEB | Conc. |
| Sample | Fold | Reps | (pg/mL) | Fold | Reps | (pg/mL) |
| Human | 500 | TMF | TMF | 500 | 23.0496 | 454832 |
| Brain | | | | | | |
| Lysate | | | | | | |

Comparison of capture-detector
capabilities of Ab-3/Ab-4 antibody
pairs using 2-step and 3-step assays

| | Assay Format | | | | | |
|---|---|---|---|---|---|---|
| | 2-step, 2.0 | | | 3-step, 2.0 | | |
| | Beads Ab | | | | | |
| | Ab-3 | | | Ab-3 | | |
| | Detector Ab | | | | | |
| | Ab-4 | | | Ab-4 | | |
| [Frataxin] | Ave | | | Ave | | |
| pg/mL | AEB | % CV | S/B | AEB | % CV | S/B |
| 0 | 0.0755 | 15% | 1.00 | 0.0799 | 2% | 1.00 |
| 100 | 5.0990 | 12% | 67.56 | 1.3418 | 5% | 16.79 |
| 1000 | 29.7320 | 6% | 393.91 | 12.7621 | 0% | 159.70 |
| 10000 | TMF | — | — | TMF | — | — |

| | | |
|---|---|---|
| LOD (pg/mL) | 0.36 | 1.59 |

| | | | Corrected | | | Corrected |
|---|---|---|---|---|---|---|
| | Dilution | AEB | Conc. | Dilution | AEB | Conc. |
| Sample | Fold | Reps | (pg/mL) | Fold | Reps | (pg/mL) |
| Human | 500 | 28.9528 | 455791 | 500 | 26.6980 | >ULOQ |
| Brain | | | | | | |
| Lysate | | | | | | |

TMF: Too much fluorescence (signal saturation)

TABLE 12
Comparison of capture-detector
capabilities of Ab-2/Ab-3 antibody
pairs using 2-step and 3-step assays

| | Assay Format | | | | | |
|---|---|---|---|---|---|---|
| | 2-step, 2.0 | | | 3-step, 2.0 | | |
| | Beads Ab | | | | | |
| | Ab-2 | | | Ab-2 | | |
| | Detector Ab | | | | | |
| | Ab-3 | | | Ab-3 | | |
| [Frataxin] | Ave | | | Ave | | |
| pg/mL | AEB | % CV | S/B | AEB | % CV | S/B |
| 0 | 0.0807 | 5% | 1.00 | 0.0628 | 8% | 1.00 |
| 100 | 7.1446 | 2% | 88.56 | 2.4424 | 2% | 38.90 |
| 1000 | 37.2790 | — | 462.11 | 20.7258 | 1% | 330.14 |
| 10000 | TMF | — | — | TMF | — | — |

| | | |
|---|---|---|
| LOD (pg/mL) | 0.27 | 0.65 |

| | | | | | | Corrected |
|---|---|---|---|---|---|---|
| | Dilution | Dilution | Dilution | Dilution | AEB | Conc. |
| Sample | Fold | Fold | Fold | Fold | Reps | (pg/mL) |
| Human | 500 | 4.3957 | 29911 | 500 | 4.7434 | 99804 |
| Brain | | | | | | |
| Lysate | | | | | | |

Comparison of capture-detector
capabilities of Ab-2/Ab-5 antibody
pairs using 2-step and 3-step assays

| | Assay Format | | | | | |
|---|---|---|---|---|---|---|
| | 2-step, 2.0 | | | 3-step, 2.0 | | |
| | Beads Ab | | | | | |
| | Ab-2 | | | Ab-2 | | |
| | Detector Ab | | | | | |
| | Ab-5 | | | Ab-5 | | |
| [Frataxin] | Ave | | | Ave | | |
| pg/mL | AEB | % CV | S/B | AEB | % CV | S/B |
| 0 | 0.0572 | 12% | 1.00 | 0.0208 | 3% | 1.00 |
| 100 | 1.4934 | 11% | 26.12 | 0.1598 | 3% | 7.69 |
| 1000 | 13.0506 | 7% | 228.25 | 1.5869 | 3% | 76.36 |
| 10000 | 28.4158 | 1% | 496.97 | 9.0259 | 1% | 434.33 |

| | | |
|---|---|---|
| LOD (pg/mL) | 1.88 | 4.90 |

| | | | Corrected | | | Corrected |
|---|---|---|---|---|---|---|
| | Dilution | AEB | Conc. | Dilution | AEB | Conc. |
| Sample | Fold | Reps | (pg/mL) | Fold | Reps | (pg/mL) |
| Human | 500 | 2.5488 | 82532 | 500 | 0.7680 | 241249 |
| Brain | | | | | | |
| Lysate | | | | | | |

TMF: Too much fluorescence (signal saturation)

TABLE 13
Comparison of capture-detector
capabilities of Ab-1/Ab-3 antibody
pairs using 2-step and 3-step assays

| | Assay Format | | | | | |
|---|---|---|---|---|---|---|
| | 2-step, 2.0 | | | 3-step, 2.0 | | |
| | Beads Ab | | | | | |
| | Ab-1 | | | Ab-1 | | |
| | Detector Ab | | | | | |
| | Ab-3 | | | Ab-3 | | |
| [Frataxin] | Ave | | | Ave | | |
| pg/mL | AEB | % CV | S/B | AEB | % CV | S/B |
| 0 | 0.0800 | 7% | 1.00 | 0.0589 | 6% | 1.00 |
| 100 | 7.1054 | 2% | 88.85 | 2.4952 | 0% | 42.34 |
| 1000 | 30.6760 | — | 383.60 | 19.6121 | 0% | 332.81 |
| 10000 | TMF | — | — | TMF | — | — |

| | | |
|---|---|---|
| LOD (pg/mL) | 0.27 | 0.59 |

| | | | Corrected | | | Corrected |
|---|---|---|---|---|---|---|
| | Dilution | AEB | Conc. | Dilution | AEB | Conc. |
| Sample | Fold | Reps | (pg/mL) | Fold | Reps | (pg/mL) |
| Human | 500 | 4.0932 | 27788 | 500 | 4.3029 | 88600 |
| Brain | | | | | | |
| Lysate | | | | | | |

Comparison of capture-detector
capabilities of Ab-1/Ab-5 antibody
pairs using 2-step and 3-step assays

| | Assay Format | | | | | |
|---|---|---|---|---|---|---|
| | 2-step, 2.0 | | | 3-step, 2.0 | | |
| | Beads Ab | | | | | |
| | Ab1 | | | Ab1 | | |
| | Detector Ab | | | | | |
| | Ab-5 | | | Ab-5 | | |
| [Frataxin] | Ave | | | Ave | | |
| pg/mL | AEB | % CV | S/B | AEB | % CV | S/B |
| 0 | 0.0583 | 4% | 1.00 | 0.0242 | 3% | 1.00 |
| 100 | 1.6121 | 4% | 27.63 | 0.1520 | 2% | 6.28 |
| 1000 | 12.3948 | 11% | 212.43 | 1.3056 | 2% | 53.93 |
| 10000 | 29.3039 | 3% | 502.23 | 8.3726 | 8% | 345.84 |

| | | |
|---|---|---|
| LOD (pg/mL) | 1.27 | 5.12 |

| | | | Corrected | | | Corrected |
|---|---|---|---|---|---|---|
| | Dilution | AEB | Conc. | Dilution | AEB | Conc. |
| Sample | Fold | Reps | (pg/mL) | Fold | Reps | (pg/mL) |
| Human | 500 | 2.6260 | 82055 | 500 | 0.7592 | 282871 |
| Brain | | | | | | |
| Lysate | | | | | | |

TMF: Too much fluorescence (signal saturation)

Three exemplary antibody pairs were further evaluated by performing preliminary dilution linearity of the brain lysate. For this experiment, the same reagent conditions were used as previously described: 300,000 capture beads/well, 150 pM SβG, and 0.2 μg/mL detector antibody in reaction. Each of the three antibody pairs were tested using a full 8-point calibration curve. The human brain lysate sample was serially diluted 2-fold from a 100× dilution to a 12800× dilution. Results of this experiment are shown in Table 14 below.

TABLE 14
Assessment of capture-detector capabilities of three exemplary antibody pairs

| | Assay Format | | | | | | | | |
|---|---|---|---|---|---|---|---|---|---|
| | 2-step, 2.0 | | | | | | | | |
| | Beads Ab | | | | | | | | |
| | Ab-3 | | | Ab-2 | | | Ab-1 | | |
| | Detector Ab | | | | | | | | |
| | Ab-4 | | | Ab-3 | | | Ab-3 | | |
| [Frataxin] | Ave | | | Ave | | | Ave | | |
| pg/mL | AEB | % CV | S/B | AEB | % CV | S/B | AEB | % CV | S/B |
| 0 | 0.1149 | 3% | 1.00 | 0.0955 | 4% | 1.00 | 0.1038 | 0% | 1.00 |
| 0.686 | 0.1568 | 0% | 1.36 | 0.1650 | 3% | 1.73 | 0.1651 | 1% | 1.59 |
| 2.06 | 0.2588 | 1% | 2.25 | 0.3183 | 2% | 3.33 | 0.3176 | 1% | 3.06 |
| 6.17 | 0.5599 | 2% | 4.87 | 0.7372 | 0% | 7.72 | 0.7373 | 1% | 7.10 |
| 18.5 | 1.3119 | 3% | 11.42 | 2.1452 | 0% | 22.47 | 1.9811 | 3% | 19.09 |
| 55.6 | 3.7472 | 0% | 32.61 | 5.2136 | 1% | 54.60 | 5.5186 | 0% | 53.17 |
| 167 | 11.1464 | 1% | 96.99 | 14.1147 | 2% | 147.82 | 15.1013 | 2% | 145.49 |

| | | | | | | | |
|---|---|---|---|---|---|---|---|
| 500 | 23.6845 | 1% | 206.08 | 25.1491 | 2% | 263.39 | Saturated |

| | | | |
|---|---|---|---|
| LOD (pg/mL) | 0.44 | 0.23 | 0.28 |

| Sample | | Corrected | % Linearity | | Corrected | % Linearity | | Corrected | % Linearity |
|---|---|---|---|---|---|---|---|---|---|
| Dilution | Ave | Conc. | (from | Ave | Conc. | (from | Ave | Conc. | (from |
| Factor | AEB | (pg/mL) | 400x) | AEB | (pg/mL) | 100x) | AEB | (pg/mL) | 100x) |
| 100x | Saturated | | | 20.8375 | 33418 | | 17.8942 | 20595 | |
| 200x | Saturated | | | 12.0949 | 28893 | 86% | 11.0576 | 23436 | 114% |
| 400x | 24.1386 | 200013 | | 6.6749 | 27564 | 82% | 5.8862 | 23487 | 114% |
| 800x | 13.6025 | 184230 | 92% | 3.2317 | 24408 | 73% | 3.1147 | 23899 | 116% |
| 1600x | 4.8141 | 112989 | 56% | 1.4853 | 21024 | 63% | 1.4656 | 21437 | 104% |
| 3200x | 1.7732 | 77508 | 39% | 0.7665 | 20185 | 60% | 0.7514 | 20422 | 99% |
| 6400x | 0.6686 | 51882 | 26% | 0.4345 | 20448 | 61% | 0.4224 | 20227 | 98% |
| 12800x | 0.3260 | 40018 | 20% | 0.2618 | 20197 | 60% | 0.2644 | 20595 | 100% |

The top half of the table shows average AEB (Average Enzymes per Bead) values of two replicates and signal-to-background ratios of full 8-point frataxin calibration curve run using three exemplary antibody pairs in a 2-step assay format. The bottom half of the table shows the average AEB and concentration values of the serially diluted human brain lysate sample. Approximate limit of detection (LOD) is calculated based on 2.5× SD (Standard Deviation) above background signal, assuming a 10% CV (Coefficient of Variation). Percent linearity was calculated by dividing the observed concentration at a given dilution by the concentration at the lowest dilution factor. Bolded values were above the respective curve range and therefore have concentrations that were extrapolated from the curve.

As seen from the data shown in Table 14, the three antibody pairs have comparable curve performance and estimated sensitivity. However, the third antibody pair (Ab-1 as capture and Ab-3 as detector) is the only pair that gives desirable linearity (80-120%) for the brain lysate sample from 100× to 12800× dilution. The desirable linearity suggests that this antibody pair is specific to the endogenous frataxin protein in the brain lysate sample. Therefore, the third antibody pair was chosen for further development.

The results in Table 14 show that Ab-1 as capture agent and Ab-3 as detector agent has an assay background that is above the desired range (0.005-0.02 AEB). Therefore, various assay buffer and blockers, detector concentrations, and SβG concentrations were evaluated in an effort to decrease the assay background. For each experiment that introduced a new condition, the original assay condition was also included (2-step, 2.0 assay format, 300,000 capture beads/well, 150 pM SβG, and 0.2 μg/mL detector antibody in reaction) within that run for direct comparison. Table 15 below shows that the assay background is within the desired range when the SβG concentration is reduced from 150 pM to 50 pM while keeping all other variables constant. The lower SβG concentration also improves assay sensitivity. Therefore, 50 pM SβG was selected for further testing.

TABLE 15
Assay conditions for improved background
signal-to-background ratio

| | SβG Conc. | | | | | |
|---|---|---|---|---|---|---|
| | 150 pM | | | 50 pM | | |
| [FXN] | Ave | | | Ave | | |
| pg/mL | AEB | % CV | S/B | AEB | % CV | S/B |
| 0 | 0.0449 | 11% | — | 0.0183 | 19% | — |
| 0.686 | 0.0717 | 26% | 1.60 | 0.0421 | 10% | 2.30 |
| 6.17 | 0.5207 | 0% | 11.60 | 0.1942 | 5% | 10.60 |
| 55.6 | 3.6950 | 24% | 82.32 | 1.6483 | 3% | 89.99 |
| 500 | 23.6655 | 2% | 527.27 | 13.6775 | 2% | 746.74 |

| | | |
|---|---|---|
| LOD (pg/mL) | 0.272 | 0.132 |

AEB (Average Enzymes per Bead) values, CVs (Coefficient of Variation), and signal to background ratios of an abbreviated frataxin calibration curve when using 150 pM and 50 pM SβG in reaction. All other conditions previously established (antibody pair, assay format, bead concentration, detector concentration, and assay buffers) were used for both conditions. Approximate limit of detection (LOD) is calculated based on 2.5× SD (Standard Deviation) above background signal, assuming a 10% CV.

In order to assess the ability of the current assay to detect and quantify frataxin in the desired matrices, multiple CSF, plasma, and serum samples were tested. Each sample was serially diluted 2-fold from a 2× to 8× dilution. The previously established conditions were used for this experiment: Ab-1 as capture agent, Ab-3 as detector agent, 2-step, 2.0 (35 min-5 min) assay format, 300,000 capture beads/well, 50 pM SβG, and 0.2 μg/mL detector antibody in reaction. A summary of the results is shown in Table 16.

TABLE 16
Initial testing of frataxin detection in CSF, plasma, and serum

| Matrix | CSF | Plasma | Serum |
|---|---|---|---|
| Total # of Samples Tested | 12 | 5 | 5 |
| # of Samples (%) Quantifiable | 7 | 2 | 2 |
| (>LLOQ) at 2x Dilution | (58%) | (40%) | (40%) |
| # of Samples (%) Between | 5 | 2 | 1 |
| LLOQ and LOD at 2x Dilution | (42%) | (40%) | (20%) |
| # of Samples (%) Below LOD at | 0 | 1 | 2 |
| 2x Dilution | (0%) | (20%) | (40%) |

### Example 2. Development of a Prototype Single Molecule Array Frataxin Detection Assay

Using the antibody pair Ab-1 (capture) and Ab-3 (detector), additional experiments were performed to further improve assay sensitivity. This included efforts toward improving the assay beads to helper beads ratio, sample reaction volume, sample dilution factor, and incubation timing protocol. Based on the results of these experiments, assay conditions were established as outlined in Table 17 below.

TABLE 17
Comparison of previously established assay conditions
and further improved assay conditions

| | | |
|---|---|---|
| Frataxin Assay | Example 1 Assay | Example 2 Assay |
| Condition | Conditions | Conditions |

| | | |
|---|---|---|
| Antibody Pair | Capture: Ab-1 | Capture: Ab-1 |
| | Detector: Ab-3 | Detector: Ab-3 |
| Assay Protocol | 2-step, 2.0 | 2-step, 2.0 |
| | (35 min-5 min) | (75 min-5 min) |
| [Beads] in Bottle | 12 × 10⁶ assay | 8 × 10⁶ assay |
| | beads per mL | beads per mL + |
| | | 12 × 10⁶ helper |
| | | beads per mL |

| | | | | |
|---|---|---|---|---|
| [Detector] in Bottle | 1.45 | μg/mL | 1.45 | μg/mL |
| [SβG] in Bottle | 50 | pM | 50 | pM |
| Calibrator/Sample | 100 | μL | 170 | μL |
| Reaction Volume | | | | |

| | | |
|---|---|---|
| Sample Dilution Factor | 2x | 2x |
| (MRD) | | |

The same CSF, plasma, and serum samples tested in Example 1 were tested again using the further improved assay conditions described on the right in Table 17. A summary of the results compared to the previous results is shown in Table 18 below. An asterisk(*) indicates that one sample had no result as both replicates were lost during testing.

TABLE 18
Comparison of results from initial sample testing using new assay conditions.

| | Example 1 Assay Conditions | | | Example 2 Assay Conditions | | |
|---|---|---|---|---|---|---|
| Matrix | CSF | Plasma | Serum | CSF | Plasma | Serum |
| Total # of Samples Tested | 12 | 5 | 5 | 12 | 5* | 5 |
| # of Samples (%) Quantifiable | 7 | 2 | 2 | 8 | 3 | 2 |
| (>LLOQ) at 2x Dilution | (58%) | (40%) | (40%) | (67%) | (60%) | (40%) |
| # of Samples (%) Between | 5 | 2 | 1 | 4 | 1 | 1 |
| LLOQ and LOD at 2x Dilution | (42%) | (40%) | (20%) | (33%) | (20%) | (20%) |
| # of Samples (%) Below LOD at | 0 | 1 | 2 | 0 | 0 | 2 |
| 2x Dilution | (0%) | (20%) | (40%) | (0%) | (0%) | (40%) |

As Table 18 illustrates, there was some improvement in the percent of CSF and plasma samples that were quantifiable using the further improved assay conditions compared to the initial sample testing. Longer incubation times of the first step of the 2-step assay protocol were also explored. Results from this testing indicated that a 120 min-5 min incubation protocol gave improved sensitivity compared to the previously used 75 min-5 min incubation protocol. Therefore, a 2-step, 2.0 (120 min-5 min) assay protocol was selected for further testing. An example of results obtained using such a protocol is shown in Table 33, below.

Dilutional linearity and spike recovery were preliminarily tested using two individual samples each of CSF, plasma, and serum. Each sample was split in half; one half of the sample was tested for endogenous levels of frataxin while the other half was spiked with 40 pg/mL of recombinant frataxin protein and then serially diluted 2-fold in Simoa® Diluent 1 from a 2× to 8× dilution. Linearity was calculated by multiplying the dilution factor by the observed back-calculated concentration and comparing the result to the observed concentration of the spiked sample at the identified dilution level. Recovery was calculated using the following equation:

$$\% Recovery = \frac{\begin{matrix}Mean Conc . of Spiked Sample - \\ Mean Conc . of Unspiked Sample\end{matrix}}{Nominal Conc . of Spiked Level} \times 100$$

The experiment was run using the Ab-1/Ab-3 antibody pair and the following assay conditions previously established: 2-step (120 min-5 min) assay protocol, 8×10⁶ assay beads with 12×10⁶ helper beads/mL, 1.45 μg/mL detector antibody in bottle, and 50 pM SβG. A full 8-point frataxin calibration curve was used in this experiment. Results of the spiked dilutional linearity testing are presented in Table 19 below.

TABLE 19
Spiked dilutional linearity and recovery in CSF, plasma, and serum samples

| Sample ID | Dilution | Ave | Ave Conc. | Conc. | % Linearity | % Linearity | % Recovery |
|---|---|---|---|---|---|---|---|
| (Matrix) | Factor | AEB | (pg/mL) | % CV | (from 2x) | (from 4x) | (at 2x) |
| Buffer | 2 | 1.5066 | 34.6 | 2% | — | — | 87% |
| | 4 | 0.7372 | 33.3 | 2% | 96% | — | — |
| | 8 | 0.4123 | 36.4 | 2% | 105% | 109% | — |
| HMN17464 | 2 | 0.9304 | 21.1 | 7% | — | — | 50% |
| (CSF) | 4 | 0.6874 | 31.0 | 2% | 147% | — | — |
| | 8 | 0.3946 | 34.8 | 12% | 165% | 112% | — |
| HMN17479 | 2 | 0.8729 | 19.8 | 1% | — | — | 46% |
| (CSF) | 4 | 0.6291 | 28.3 | 10% | 143% | — | — |
| | 8 | 0.3827 | 33.7 | 17% | 170% | 119% | — |
| BRH1530082 | 2 | 0.4342 | 9.62 | 32% | — | — | 21% |
| (Plasma) | 4 | 0.3498 | 15.3 | 26% | 159% | — | — |
| | 8 | 0.2214 | 18.7 | 5% | 194% | 122% | — |
| BRH1530099 | 2 | 0.5258 | 11.7 | 3% | — | — | 28% |
| (Plasma) | 4 | 0.3036 | 13.2 | 15% | 112% | — | — |
| | 8 | 0.2230 | 18.8 | 9% | 160% | 143% | — |
| BRH1577157 | 2 | 0.1324 | 2.57 | N/A | — | — | 6% |
| (Serum) | 4 | 0.1841 | 7.58 | 26% | 294% | — | — |
| | 8 | 0.1282 | 9.89 | 20% | 384% | 131% | — |
| BRH1577160 | 2 | 0.3310 | 7.22 | 3% | — | — | 17% |
| (Serum) | 4 | 0.2915 | 12.6 | 13% | 174% | — | — |
| | 8 | 0.2189 | 18.4 | 4% | 255% | 146% | — |

| | | | |
|---|---|---|---|
| Average | 197% | 129% | 28% |

Spiked dilutional linearity and recovery of 2 CSF, 2 plasma, and 2 serum samples, each spiked with 40 pg/mL recombinant frataxin protein and serially diluted 2-fold in Simoa® Diluent 1 buffer from 2× to 8×. All sample concentrations are corrected for dilutions. The % linearity for each sample was calculated based on the sample concentration at the dilution factor in parentheses. The % recovery was calculated using the endogenous sample readings at a 2× dilution factor.

The preliminary dilutional linearity and spike recovery data shown in Table 19 indicates that the average % linearity and % recovery do not fall within the desired range of 80-120% at a 2× sample dilution factor. The average spiked dilutional linearity from the 4× dilution factor for CSF samples (111%) is within the desired range and that of the plasma samples (121%) is close to the desired range. However, diluting the samples 4× instead of 2× will decrease the assay sensitivity. Therefore, further development of the calibrator and sample diluents was performed in order to achieve desirable linearity and recovery at a 2× sample dilution.

In the first of two experiments, one plasma sample was spiked with 80 pg/mL recombinant frataxin and then serially diluted 2-fold from 2× to 64× using two different sample buffers: Simoa® Diluent 1 with 5% Newborn Calf Serum (NCS) and Simoa® Diluent 1 with 10% NCS. A calibrator curve was created in each of these two buffers. The experiment was run using the antibody pair Ab-1 (capture agent) and Ab-3 (detection agent), the 2-step (120 min-5 min) assay protocol, 8×10⁶ assay beads with 12×10⁶ helper beads/mL, 1.45 μg/mL detector antibody in bottle, and 50 pM SβG. Linearity and recovery were calculated as previously described. The results suggest that the Simoa® Diluent 1 containing 5% or 10% NCS produces desirable linearity (within 80-120%) for the plasma sample tested with an MRD of 2× (Tables 20-23). However, the recovery of the spiked recombinant frataxin protein was lower than desired. Therefore, a similar experiment was performed in which the brain lysate sample with known high endogenous concentrations of frataxin was used as the spiking material instead of the recombinant frataxin protein.

TABLE 20
Frataxin assay diluent calibration with 5% NCS

| | Assay Diluent | | |
|---|---|---|---|
| | Simoa ® Diluent 1 + 5% NCS | | |
| [Frataxin] pg/mL | Ave AEB | % CV | S/B |
| 0 | 0.0181 | 15% | |
| 0.274 | 0.0228 | 3% | 1.26 |
| 0.823 | 0.0324 | 15% | 1.79 |
| 2.47 | 0.0609 | N/A | 3.37 |
| 7.41 | 0.1556 | 3% | 8.62 |
| 22.2 | 0.3949 | 7% | 21.87 |
| 66.7 | 1.2724 | 0% | 70.47 |
| 200 | 4.2428 | N/A | 235.01 |

| | |
|---|---|
| LOD (pg/mL) | 0.268 |

N/A: Not able to be determined due to lost replicate

TABLE 21
Dilutional linearity and spike recovery results using 5% NCS and recombinant frataxin

| | | | | | | Dilution | | | | |
|---|---|---|---|---|---|---|---|---|---|---|
| | Spike | | | | | Corrected | | | | |
| Sample ID | Level | Dilution | Ave | Ave | Conc. | Conc. | % Linearity | % Linearity | % Linearity | |
| (Matrix) | (pg/mL) | Factor | AEB | Conc. | % CV | (pg/mL) | (from 2x) | (from 4x) | (from 8x) | % Recovery |
| BRH1577166 | 0 | 2 | 0.0231 | <LOD | N/A | 0.00 | — | — | — | — |
| (Plasma) | | | | | | | | | | |
| BRH1577166 | 80 | 2 | 0.4375 | 22.82 | 3% | 45.63 | — | — | — | 57% |
| (Plasma) | 80 | 4 | 0.2414 | 12.43 | 6% | 49.72 | 109% | — | — | 62% |
| | 80 | 8 | 0.1431 | 7.10 | 6% | 56.78 | 124% | 114% | — | 71% |
| | 80 | 16 | 0.0718 | 3.13 | 24% | 50.16 | 110% | 101% | 88% | 63% |
| | 80 | 32* | 0.0433* | 1.50* | 18%* | 48.01* | 105%* | 97%* | 85%* | 60%* |
| | 80 | 64* | 0.0354* | 1.04* | 8%* | 66.60* | 146%* | 134%* | 117%* | 83%* |

| | | | | |
|---|---|---|---|---|
| Average | 119% | 111% | 97% | 66% |
| Average (without samples <LLOQ) | 114% | 108% | 88% | 63% |

N/A: Not able to be determined due to sample below LOD
*Below estimated LLOQ

TABLE 22
Frataxin assay diluent calibration with 10% NCS

| | Assay Diluent | | |
|---|---|---|---|
| | Simoa ® Diluent 1 + 10% NCS | | |
| [Frataxin] pg/mL | Ave AEB | % CV | S/B |
| 0 | 0.0182 | 9% | |
| 0.274 | 0.0230 | 12% | 1.27 |
| 0.823 | 0.0296 | 7% | 1.63 |
| 2.47 | 0.0444 | 10% | 2.44 |
| 7.41 | 0.0923 | 0% | 5.08 |
| 22.2 | 0.2000 | 1% | 11.00 |
| 66.7 | 0.6024 | 4% | 33.15 |
| 200 | 2.0312 | 7% | 111.76 |

| | |
|---|---|
| LOD (pg/mL) | 0.323 |

TABLE 23
Dilutional linearity and spike recovery results using 10% NCS and recombinant frataxin

| | | | | | | Dilution | | | | |
|---|---|---|---|---|---|---|---|---|---|---|
| | Spike | | | | | Corrected | | | | |
| Sample ID | Level | Dilution | Ave | Ave | Conc. | Conc. | % Linearity | % Linearity | % Linearity | |
| (Matrix) | (pg/mL) | Factor | AEB | Conc. | % CV | (pg/mL) | (from 2x) | (from 4x) | (from 8x) | % Recovery |
| BRH1577166 | 0 | 2 | 0.0263 | <LOD | N/A | 0.00 | — | — | — | — |
| (Plasma) | | | | | | | | | | |
| BRH1577166 | 80 | 2 | 0.3194 | 34.13 | 16% | 68.25 | — | — | — | 85% |
| (Plasma) | 80 | 4 | 0.1716 | 16.64 | 5% | 66.56 | 98% | — | — | 83% |
| | 80 | 8 | 0.0905 | 7.58 | N/A | 60.64 | 89% | 91% | — | 76% |
| | 80 | 16* | 0.0435* | 2.52* | N/A | 40.28* | 59%* | 61%* | 66%* | 50%* |
| | 80 | 32* | 0.0302* | 1.11* | 56%* | 35.38* | 52%* | 53%* | 58%* | 44%* |
| | 80 | 64* | 0.0241* | 0.47* | N/A | 29.98* | 44%* | 45%* | 49%* | 37%* |
| | | | | | | Average | 68% | 62% | 58% | 63% |

| | | | |
|---|---|---|---|
| Average (without samples <LLOQ) | 93% | 91% | 81% |

N/A: Not able to be determined due to sample below LOD
*Below estimated LLOQ

For the next experiment, a total of 4 plasma samples were spiked with the brain lysate and serially diluted 2-fold from 2× to 8× in either the 5% NCS or 10% NCS buffer. A calibrator curve was created in each of these two buffers as well. The experiment was run using the antibody pair Ab-1 (capture agent) and Ab-3 (detection agent), the 2-step (120 min-5 min) assay protocol, 8×10⁶ assay beads with 12×10⁶ helper beads/mL, 1.45 μg/mL detector antibody in bottle, and 50 pM SβG. Recovery was corrected based on the frataxin concentration of the brain lysate sample when spiked in assay diluent according to the following equation:

$$Corrected \% Recovery = \frac{\begin{matrix}Mean Conc . of Spiked Sample - \\ Mean Conc . of Unspiked Sample\end{matrix}}{Nominal Conc . of Spiked Assay Buffer} \times 100$$

Tables 24 and 25 show the dilutional linearity and spike recovery results using 5% NCS and 10% NCS as both calibrator and sample diluent. Simoa® Diluent 1 with 10% NCS as both calibrator and sample diluent gave desirable average linearity (98%) and recovery (85%) for the 4 plasma samples at 2× MRD. Therefore, Simoa® Diluent 1 with 10% NCS was chosen as the calibrator and sample diluent for this assay.

TABLE 24
Dilutional linearity and spike recovery calibration with 5% and 10% NCS

| | Calibrator Diluent | | | | | |
|---|---|---|---|---|---|---|
| | Simoa ® Diluent 1 + 5% NCS | | | Simoa ® Diluent 1 + 10% NCS | | |
| [Frataxin] | Ave | | | Ave | | |
| pg/mL | AEB | % CV | S/B | AEB | % CV | S/B |
| 0 | 0.0110 | 13% | | 0.0107 | 12% | |
| 1.37 | 0.0200 | 1% | 1.82 | 0.0153 | 8% | 1.42 |
| 4.12 | 0.0434 | 0% | 3.95 | 0.0283 | 1% | 2.63 |
| 12.3 | 0.1232 | 2% | 11.23 | 0.0631 | 2% | 5.87 |
| 37.0 | 0.3413 | 6% | 31.11 | 0.1736 | 3% | 16.17 |
| 111 | 1.0211 | 2% | 93.07 | 0.5154 | 6% | 48.00 |
| 333 | 3.0645 | 3% | 279.32 | 1.5565 | 6% | 144.94 |
| 1000 | 8.9467 | 1% | 815.46 | 5.1798 | 9% | 482.33 |

| | | |
|---|---|---|
| LOD (pg/mL) | 0.419 | 0.759 |

TABLE 25
Dilutional linearity and spike recovery results with brain lysate frataxin

| | | Calibrator Curve Diluent | | | |
|---|---|---|---|---|---|
| | | Simoa ® Diluent 1 + 5% NCS | | | |
| | | Sample Diluent | | | |
| | | Simoa ® Diluent 1 + 5% NCS | | | |
| Sample ID | Dilution | Ave | Ave Conc | % Linearity | Corrected |
| (Matrix) | Factor | AEB | (pg/mL) | (2x) | % Recovery |
| Buffer | 2 | 0.2524 | 55.2 | — | 100% |
| BRH1577166 | 2 | 0.1868 | 40.8 | — | 74% |
| (Plasma) | 4 | 0.1065 | 45.6 | 112% | — |
| | 8 | 0.0607 | 49.0 | 120% | — |
| BRH1577171 | 2 | 0.1940 | 42.3 | — | 77% |
| (Plasma) | 4 | 0.1110 | 47.6 | 112% | — |
| | 8 | 0.0620 | 50.3 | 119% | — |
| | 2 | 0.1922 | 41.9 | — | 76% |
| BRH1577167 | 4 | 0.1178 | 50.7 | 121% | — |
| (Plasma) | 8 | 0.0547 | 43.3 | 103% | — |
| BRH1577168 | 2 | 0.1873 | 40.9 | — | 74% |
| (Plasma) | 4 | 0.1061 | 45.4 | 111% | — |
| | 8 | 0.0593 | 47.7 | 117% | — |
| | | | Average | 114% | 75% |
| | | Calibrator Curve Diluent | | | |
| | | Simoa ® Diluent 1 + 10% NCS | | | |
| | | Sample Diluent | | | |
| | | Simoa ® Diluent 1 + 10% NCS | | | |
| Sample ID | Dilution | Ave | Ave Conc | % Linearity | Corrected |
| (Matrix) | Factor | AEB | (pg/mL) | (2x) | % Recovery |
| Buffer | 2 | 0.2154 | 92.5 | | 100% |
| BRH1577166 | 2 | 0.1691 | 72.3 | | 78% |
| (Plasma) | 4 | 0.1006 | 83.8 | 116% | |
| | 8 | 0.0520 | 79.6 | 110% | |
| BRH1577171 | 2 | 0.1759 | 75.2 | | 81% |
| (Plasma) | 4 | 0.0861 | 70.8 | 94% | |
| | 8 | 0.0499 | 75.5 | 100% | |
| BRH1577167 | 2 | 0.2071 | 88.9 | | 96% |
| (Plasma) | 4 | 0.0790 | 64.3 | 72% | |
| | 8 | 0.0510 | 77.5 | 87% | |
| | 2 | 0.1783 | 76.3 | | 82% |
| BRH1577168 | 4 | 0.0904 | 74.7 | 98% | |
| (Plasma) | 8 | 0.0511 | 77.8 | 102% | |
| | | | Average | 98% | 85% |

TABLE 26
Calibration of frataxin detection for LLOQ assessment in plasma

| [Frataxin] | | AEB | AEB | AEB | Ave | | | |
|---|---|---|---|---|---|---|---|---|
| pg/mL | Weight | rep1 | rep2 | rep3 | AEB | SD | % CV | S/B |
| 0 | 1 | 0.0095 | 0.0075 | 0.0077 | 0.0083 | 0.0011 | 14% | 1.00 |
| 2.74 | 1 | 0.0165 | 0.0248 | 0.0202 | 0.0205 | 0.0041 | 20% | 2.48 |
| 8.23 | 1 | 0.0466 | 0.0480 | 0.0517 | 0.0487 | 0.0026 | 5% | 5.91 |
| 24.7 | 1 | 0.1293 | 0.1175 | 0.1416 | 0.1294 | 0.0121 | 9% | 15.69 |
| 74.1 | 1 | 0.3575 | 0.3563 | 0.3364 | 0.3501 | 0.0119 | 3% | 42.42 |
| 222 | 1 | 1.0148 | 1.0773 | 1.0430 | 1.0450 | 0.0313 | 3% | 126.63 |
| 667 | 1 | 3.5741 | 3.9861 | 3.6149 | 3.7250 | 0.2270 | 6% | 451.39 |
| 2000 | 1 | 9.7966 | 10.1129 | 9.7970 | 9.9021 | 0.1825 | 2% | 1199.91 |

| | |
|---|---|
| LOD (pg/mL) | 0.465 |

TABLE 27
Preliminary assessment of frataxin LLOQ in plasma

| | | Spike | | | | | | |
|---|---|---|---|---|---|---|---|---|
| Sample ID | Dilution | Level | AEB | Ave | AEB | Conc. | Ave | Conc. |
| (Matrix) | Factor | (pg/mL) | Reps | AEB | % CV | Reps | Conc. | % CV |
| BRH1577168 | 2 | 10 | 0.0508 | 0.0452 | 12% | 9.07 | 7.88 | 14.9% |
| (Plasma) | | | 0.0451 | | | 7.87 | | |
| | | | 0.0396 | | | 6.71 | | |
| | 4 | 10 | 0.0286 | 0.0262 | 11% | 4.39 | 3.89 | 15.5% |
| | | | 0.0231 | | | 3.22 | | |
| | | | 0.0270 | | | 4.05 | | |
| | 8 | 10 | 0.0229 | 0.0211 | 16% | 3.18 | 2.78 | 25.6%* |
| | | | 0.0172 | | | 1.96 | | |
| | | | 0.0230 | | | 3.20 | | |
| | 16 | 10 | 0.0173 | 0.0173 | 2% | 1.98 | 1.97 | 3.3% |
| | | | 0.0169 | | | 1.89 | | |
| | | | 0.0175 | | | 2.02 | | |
| | 32 | 10 | 0.0150 | 0.0159 | 5% | 1.48 | 1.67 | 11.0% |
| | | | 0.0159 | | | 1.68 | | |
| | | | 0.0167 | | | 1.85 | | |
| BRH1530099 | 2 | 10 | 0.0383 | 0.0358 | 6% | 6.44 | 5.91 | 8.0% |
| (Plasma) | | | 0.0340 | | | 5.53 | | |
| | | | 0.0351 | | | 5.76 | | |
| | 4 | 10 | 0.0262 | 0.0248 | 9% | 3.88 | 3.57 | 13.3% |
| | | | 0.0259 | | | 3.82 | | |
| | | | 0.0222 | | | 3.03 | | |
| | 8 | 10 | 0.0211 | 0.0213 | 4% | 2.79 | 2.84 | 5.8% |
| | | | 0.0222 | | | 3.03 | | |
| | | | 0.0207 | | | 2.71 | | |
| | 16 | 10 | 0.0120 | 0.0150 | 24% | 0.83 | 1.48 | 52.5%* |
| | | | 0.0190 | | | 2.34 | | |
| | | | 0.0140 | | | 1.27 | | |
| | 32 | 10 | 0.0131 | 0.0144 | 14% | 1.07 | 1.35 | 31.1%* |
| | | | 0.0134 | | | 1.14 | | |
| | | | 0.0166 | | | 1.83 | | |
| BRH1530100 | 2 | 10 | 0.0374 | 0.0388 | 4% | 6.25 | 6.54 | 4.9% |
| (Plasma) | | | 0.0404 | | | 6.88 | | |
| | | | 0.0385 | | | 6.48 | | |
| | 4 | 10 | 0.0311 | 0.0278 | 10% | 4.92 | 4.23 | 14.3% |
| | | | 0.0259 | | | 3.82 | | |
| | | | 0.0265 | | | 3.94 | | |
| | 8 | 10 | 0.0183 | 0.0216 | 17% | 2.19 | 2.91 | 27.1%* |
| | | | 0.0210 | | | 2.77 | | |
| | | | 0.0256 | | | 3.75 | | |
| | 16 | 10 | 0.0190 | 0.0141 | 32% | 2.34 | 1.29 | 74.3%* |
| | | | 0.0103 | | | 0.46 | | |
| | | | 0.0131 | | | 1.07 | | |
| | 32 | 10 | 0.0155 | 0.0165 | 9% | 1.59 | 1.80 | 18.0% |
| | | | 0.0182 | | | 2.17 | | |
| | | | 0.0157 | | | 1.64 | | |
| BRH1530071 | 2 | 10 | 0.0450 | 0.0424 | 13% | 7.85 | 7.30 | 15.8% |
| (Plasma) | | | 0.0361 | | | 5.98 | | |
| | | | 0.0461 | | | 8.08 | | |
| | 4 | 10 | 0.0268 | 0.0257 | 6% | 4.01 | 3.77 | 9.3% |
| | | | 0.0265 | | | 3.94 | | |
| | | | 0.0238 | | | 3.37 | | |
| | 8 | 10 | 0.0194 | 0.0217 | 10% | 2.43 | 2.91 | 15.0% |
| | | | 0.0234 | | | 3.28 | | |
| | | | 0.0222 | | | 3.03 | | |
| | 16 | 10 | 0.0157 | 0.0157 | 31% | 1.64 | 1.63 | 64.8%* |
| | | | 0.0108 | | | 0.57 | | |
| | | | 0.0206 | | | 2.69 | | |
| | 32 | 10 | 0.0147 | 0.0161 | 10% | 1.42 | 1.72 | 18.9% |
| | | | 0.0159 | | | 1.68 | | |
| | | | 0.0177 | | | 2.07 | | |
| BRH1530074 | 2 | 10 | 0.0501 | 0.0423 | 16% | 8.92 | 7.27 | 19.8% |
| (Plasma) | | | 0.0375 | | | 6.27 | | |
| | | | 0.0392 | | | 6.63 | | |
| | 4 | 10 | 0.0258 | 0.0273 | 6% | 3.80 | 4.12 | 8.6% |
| | | | 0.0271 | | | 4.07 | | |
| | | | 0.0291 | | | 4.50 | | |
| | 8 | 10 | 0.0202 | 0.0211 | 4% | 2.60 | 2.79 | 7.4% |
| | | | 0.0221 | | | 3.01 | | |
| | | | 0.0209 | | | 2.75 | | |
| | 16 | 10 | 0.0227 | 0.0186 | 20% | 3.14 | 2.26 | 36.1%* |
| | | | 0.0179 | | | 2.11 | | |
| | | | 0.0152 | | | 1.53 | | |
| | 32 | 10 | 0.0142 | 0.0123 | 26% | 1.31 | 0.90 | 78.5%* |
| | | | 0.0086 | | | 0.08 | | |
| | | | 0.0142 | | | 1.31 | | |

*Coefficient of Variation >20%

Using the antibody pair Ab-1 (capture) and Ab-3 (detector), the 2-step (120 min-5 min) assay protocol, 8×10⁶ assay beads with 12×10⁶ helper beads/mL, 1.45 μg/mL detector antibody in bottle, and 50 pM SβG, the preliminary Lower Limit of Quantitation (LLOQ) of the assay was determined. Five (5) individual plasma samples were each spiked with brain lysate and then serially diluted 2-fold from 2× to 32× dilution. For each sample, the lowest concentration at which the CV was less than 20% (and did not have a previous dilution where the CV was greater than 20%) was selected as the LLOQ for that sample. The assay LLOQ was determined as the average of LLOQ values of the 5 individual plasma samples. The results of this testing (shown in Tables 26 and 27 above) indicate that the preliminary LLOQ of the frataxin assay for plasma is 3.33 pg/mL. The average lower limit of quantitation (LLOQ) calculated from bolded average concentration values in Table 27 was 3.33.

### Example 3. Frataxin Protein Assay Procedure

The single molecule array technology employed two primary steps: an initial analyte capture step conducted with paramagnetic beads, followed by isolation of individual beads in arrays of femtoliter-sized reaction wells for digital imaging. Isolation of the individual beads in microwells permits the buildup of fluorescent product from the enzyme label such that signal from a single immunocomplex is readily detected using a CCD camera, such as the Simoa® HD-1 Analyzer. This approach permits counting of single molecules when frataxin protein concentrations are low enough that the ratio of bound labeled peptide per bead is much less than one. In this concentration realm, Poisson statistics predict that bead-containing microwells in the array will contain either a single labeled frataxin protein molecule or no labeled frataxin protein molecules, resulting in a binary signal. Due to the amplified sensitivity for detecting label molecules afforded by confining fluorescent product buildup to the microwells, concentrations of label (detector anti-frataxin antibody and enzyme label) can be reduced relative to standard ELISAs. Lowered concentrations of labeling reagents reduce their interaction with capture beads, resulting in reduced nonspecific binding enabling high signal to background ratios, even at extremely low concentrations of biomarker.

Prior to beginning the experiment, all calibrators and controls were allowed to come to room temperature before loading onto the Simoa® HD-1 Analyzer. All reagents were also allowed to come to room temperature before loading on the Simoa® HD-1 Analyzer. Alternatively, if the Analyzer were equipped with a cooled reagent bay, then the reagents would not be required to be at room temperature. Prior to use, all Homebrew Bead Stock, Detector Stock, SBG Concentrate, and Calibrator Concentrate was briefly centrifuged to remove liquid from the vial caps. Utilizing the Quanterix Simoa® platform requires use of several proprietary reagents, such as the Homebrew Diluent, SBG Concentrate, and Calibrator Concentrate. Further, the Quanterix HD-1 Analyzer actively runs the assay steps, after the user inserts the reagents and samples into the Analyzer. Samples, reagents, and accessories were prepared in accordance with the HD-1 Analyzer guide, provided by the manufacturer.

The reagents were prepared in anticipation for use in the Analyzer. The Homebrew Beads were washed prior to the procedure. One vial of the Homebrew Beads was mixed with 1 mL of Homebrew Bead Diluent and mixed by gentle inversion. The vial was placed against a magnet, associated with the Analyzer for one minute, then the diluent was carefully removed. The Homebrew Beads were then resuspended in 1 mL of Homebrew Bead Diluent and the Beads were vortexed to mix. Next, 4 mL of Homebrew Bead Diluent was added to an amber reagent bottle, supplied by the manufacturer, and vortexed. Once mixed, the Homebrew Bead Reagent was placed into the Analyzer. The capture agent bound to the beads was Ab-1, FXN/Frataxin antibody (sold by LSBio, Catalogue no. LS-C760752-100, see Table 2 above.)

The detector agent used was Ab-3, anti-Frataxin antibody (18A5DB1; sold by Abcam; Catalogue no. ab110328, see Table 2 above). The stock concentration of the Detector antibody was diluted to 1.45 μg/mL into a 15 mL conical polypropylene centrifuge tube, 11.86 mL of Simoa® Diluent 1 was added along with 144 μL of the diluted Detector. The combination was vortexed to mix. The Detector Reagent was transferred to a clear reagent bottle supplied by the Analyzer manufacturer, carefully to avoid transferring bubbles.

The SBG Reagent was made with SBG Concentrate and SBG Dilution Buffer, both supplied by the manufacturer. The calculation is as follows: 12 mL (reagent volume)×0.05 nM (final concentration)÷SBG concentrate nM (from vial)=Vol. of SBG Conc mL. 12 mL of the SBG Dilution Buffer was added to a 15 mL conical polypropylene centrifuge tube. The calculated volume of SBG concentrate was pipetted into the tube and vortexed. The SBG Reagent was then transferred into a clear reagent bottled supplied by the manufacturer.

The Sample Diluent Reagent was prepared by transferring 14 mL of Simoa® Diluent 1 to a clear reagent bottle supplied by the manufacturer. Care was taken not to transfer any bubbles.

All of the Reagents were loaded into the Analyzer as per manufacturer's specifications and instructions and in the quantities as shown in Table 28.

TABLE 28
Preliminary CSF, Serum, Plasma sample testing

| | | |
|---|---|---|
| Reagent (96 samples) | Stock Volume | Diluent Volume |

| | | | | |
|---|---|---|---|---|
| Homebrew Beads | 1 | mL | 4.0 | mL |
| (1 mL post wash) | | | | |
| Detector | 144 | μL | 11.86 | mL |

| | | | |
|---|---|---|---|
| SBG | Calculated volume | 12 | mL |
| Sample Diluent | NA | 14 | mL |

The assay was set up to run on the instrument as per manufacturer's specifications and instructions. Calibration of the instrument, including loading of calibration reagents, was also done to manufacturer's specifications.

The frataxin standard (Recombinant Human Frataxin protein; see Table 2 above) curve was prepared by transferring 0.6 mL of Simoa® Diluent 1 to eight (8) 1.5 mL Eppendorf tubes, being careful not to transfer any bubbles. The standard was diluted to 500 pg/mL in the first tube. From the first tube, 0.3 mL of the diluted standard was added to the second tube and diluted to 167 pg/mL. From the second tube, 0.3 mL of the diluted standard was added to the third tube and diluted to 55.5 pg/mL. From the third tube, 0.3 mL of the diluted standard was added to the fourth tube and diluted to 18.5 pg/mL. From the fourth tube, 0.3 mL of the diluted standard was added to the fifth tube and diluted to 6.17 pg/mL. From the fifth tube, 0.3 mL of the diluted standard was added to the sixth tube and diluted to 2.06 pg/mL. From the sixth tube, 0.3 mL of the diluted standard was added to the seventh tube and diluted to 0.685 pg/mL. From the seventh tube, 0.3 mL of the diluted standard was added to the eighth tube and diluted to 0.229 pg/mL.

The standards were run on the Analyzer with detection limits shown in Table 29. Each dilution of the standard was run twice. The Average Enzymes per Bead (AEB) was determined for each repetition, and an average of the AEB (AVE), standard deviation (SD), and Coefficient of Variation (% CV) were calculated. The signal to background (S/B) was also determined as an indicator of validity or noise. A Limit of Detection (LOD) was calculated to be 0.551 pg/mL of the frataxin standard on the Simoa® platform. The estimated Lower Limit of Quantitation (LLOQ) was determined to be 1.16 pg/mL.

TABLE 29
Preliminary Frataxin Standard Preparation

| [Frataxin] | | AEB | AEB | Ave | | | |
|---|---|---|---|---|---|---|---|
| pg/mL | weight | Rep1 | Rep2 | AEB | SD | % CV | S/B |
| 0 | 1 | 0.0238 | 0.0214 | 0.0226 | 0.0017 | 7% | 1.00 |
| 0.686 | 1 | 0.0328 | 0.0280 | 0.0304 | 0.0034 | 11% | 1.34 |
| 2.06 | 1 | 0.0405 | 0.0430 | 0.0418 | 0.0018 | 4% | 1.85 |
| 6.17 | 1 | 0.1183 | 0.1415 | 0.1299 | 0.0164 | 13% | 5.74 |
| 18.5 | 1 | 0.3631 | 0.3622 | 0.3626 | 0.0006 | 0% | 16.03 |
| 55.6 | 1 | 1.1629 | 0.6080 | 0.8854 | 0.3924 | 44% | 39.14 |
| 167 | 1 | 2.4243 | 2.1015 | 2.2629 | 0.2282 | 10% | 100.03 |
| 500 | 1 | 7.9398 | 5.0833 | 6.5115 | 2.0198 | 31% | 287.84 |

| | |
|---|---|
| LOD (pg/mL) | 0.551 |
| Estimated LLOQ | 1.16 |
| (pg/mL) | |

After the frataxin standard was prepared, various samples from human circulating biofluids were tested with the data shown in Table 30. The selected fluids included cerebrospinal fluid (CSF), blood plasma (plasma), and blood serum (serum). The first samples were prepared in three dilutions: 2:1, 4:1, and 8:1. Each dilution factor was tested in two repetitions, each time measuring the average enzyme per bead (AEB) and calculating the average AEB. Like with the frataxin standard, the Coefficient of Variation (% CV) and concentration of frataxin (Conc) were calculated. The concentration of frataxin (pg/mL) was calculated based on the dilution factor.

TABLE 30
Preliminary CSF, Serum, Plasma sample testing

| | | | | | | | | Dilution | | |
|---|---|---|---|---|---|---|---|---|---|---|
| | | | | | | | | Corrected | | |
| Sample ID | Dilution | AEB | Ave | AEB | Conc. | Ave | Conc. | Conc. | % Linearity | |
| (Matrix) | Factor | Reps | AEB | % CV | Reps | Conc. | % CV | (pg/mL) | (from 2x) | Notes |
| HMN42511 | 2 | 0.0373 | 0.0385 | 4% | 1.20 | 1.29 | 9% | 2.57 | | |
| (CSF) | | 0.0397 | | | 1.37 | | | | | |
| | 4 | 0.0336 | 0.0307 | 13% | 0.94 | 0.94 | | <LLOQ | | Sample |
| | | 0.0278 | | | <LOD | | | | | below |
| | | | | | | | | | | LLOQ; One |
| | | | | | | | | | | replicate |
| | | | | | | | | | | below LOD |
| | 8 | 0.0319 | 0.0292 | 13% | 0.82 | 0.82 | | <LLOQ | | Sample |
| | | 0.0266 | | | <LOD | | | | | below |
| | | | | | | | | | | LLOQ; One |
| | | | | | | | | | | replicate |
| | | | | | | | | | | below LOD |
| HMN42512 | 2 | 0.0367 | 0.0364 | 1% | 1.16 | 1.14 | 3% | <LLOQ | | Sample |
| (CSF) | | 0.0361 | | | 1.12 | | | | | below LLOQ |
| | 4 | 0.0294 | 0.0305 | 5% | 0.63 | 0.71 | 16% | <LLOQ | | Sample |
| | | 0.0316 | | | 0.79 | | | | | below LLOQ |
| | 8 | 0.0232 | 0.0247 | 8% | <LOD | <LOD | | <LOD | | Sample |
| | | 0.0262 | | | <LOD | | | | | below LOD |
| HMN42532 | 2 | 0.0357 | 0.0361 | 2% | 1.09 | 1.12 | 4% | <LLOQ | | Sample |
| (CSF) | | 0.0366 | | | 1.15 | | | | | below LLOQ |
| | 4 | 0.0215 | 0.0265 | 26% | <LOD | 0.78 | | <LLOQ | | Sample |
| | | 0.0314 | | | 0.78 | | | | | below |
| | | | | | | | | | | LLOQ; One |
| | | | | | | | | | | replicate |
| | | | | | | | | | | below LOD |
| | 8 | 0.0198 | 0.0242 | 26% | <LOD | 0.57 | | <LLOQ | | Sample |
| | | 0.0286 | | | 0.57 | | | | | below |
| | | | | | | | | | | LLOQ; One |
| | | | | | | | | | | replicate |
| | | | | | | | | | | below LOD |
| HMN42533 | 2 | 0.0449 | 0.0427 | 7% | 1.74 | 1.58 | 14% | 3.16 | | |
| (CSF) | | 0.0405 | | | 1.43 | | | | | |
| | 4 | 0.0331 | 0.0355 | 9% | 0.90 | 1.07 | 22% | <LLOQ | | Sample |
| | | 0.0378 | | | 1.24 | | | | | below |
| | | | | | | | | | | LLOQ; One |
| | | | | | | | | | | replicate |
| | | | | | | | | | | within cal |
| | | | | | | | | | | range |
| | 8 | 0.0271 | 0.0274 | 2% | <LOD | <LOD | | <LOD | | Sample |
| | | 0.0278 | | | <LOD | | | | | below LOD |
| HMN42530 | 2 | 0.0541 | 0.0503 | 11% | 2.37 | 2.11 | 18% | 4.21 | | |
| (CSF) | | 0.0465 | | | 1.85 | | | | | |
| | 4 | 0.0306 | 0.0316 | 4% | 0.72 | 0.79 | 12% | <LLOQ | | Sample |
| | | 0.0325 | | | 0.86 | | | | | below LLOQ |
| | 8 | 0.0270 | 0.0290 | 10% | <LOD | 0.75 | | <LLOQ | | Sample |
| | | 0.0310 | | | 0.75 | | | | | below |
| | | | | | | | | | | LLOQ; One |
| | | | | | | | | | | replicate |
| | | | | | | | | | | below LOD |
| HMN42531 | 2 | 0.0368 | 0.0384 | 6% | 1.17 | 1.28 | 12% | 2.56 | | |
| (CSF) | | 0.0400 | | | 1.39 | | | | | |
| | 4 | 0.0257 | 0.0261 | 2% | <LOD | <LOD | | <LOD | | Sample |
| | | 0.0265 | | | <LOD | | | | | below LOD |
| | 8 | 0.0233 | 0.0252 | 11% | <LOD | <LOD | | <LOD | | Sample |
| | | 0.0271 | | | <LOD | | | | | below LOD |
| HMN42505 | 2 | 0.0459 | 0.0463 | 1% | 1.80 | 1.83 | 2% | 3.66 | | |
| (CSF) | | 0.0467 | | | 1.86 | | | | | |
| | 4 | 0.0399 | 0.0360 | 15% | 1.39 | 1.11 | 35% | <LLOQ | | Sample |
| | | 0.0322 | | | 0.84 | | | | | below |
| | | | | | | | | | | LLOQ; One |
| | | | | | | | | | | replicate |
| | | | | | | | | | | within cal |
| | | | | | | | | | | range; |
| | | | | | | | | | | CV >25% |
| | 8 | 0.0196 | 0.0194 | 1% | <LOD | <LOD | | <LOD | | Sample |
| | | 0.0192 | | | <LOD | | | | | below LOD |
| HMN42506 | 2 | 0.0361 | 0.0337 | 10% | 1.12 | 0.94 | 26% | <LLOQ | | Sample |
| (CSF) | | 0.0313 | | | 0.77 | | | | | below |
| | | | | | | | | | | LLOQ; |
| | | | | | | | | | | CV >25% |
| | 4 | 0.0258 | 0.0291 | 16% | <LOD | 0.85 | | <LLOQ | | Sample |
| | | 0.0324 | | | 0.85 | | | | | below |
| | | | | | | | | | | LLOQ; One |
| | | | | | | | | | | replicate |
| | | | | | | | | | | below LOD |
| | 8 | 0.0284 | 0.0267 | 9% | 0.56 | 0.56 | | <LLOQ | | Sample |
| | | 0.0249 | | | <LOD | | | | | below |
| | | | | | | | | | | LLOQ; One |
| | | | | | | | | | | replicate |
| | | | | | | | | | | below LOD |
| HMN42507 | 2 | 0.0360 | 0.0353 | 3% | 1.11 | 1.06 | 7% | <LLOQ | | Sample |
| (CSF) | | 0.0345 | | | 1.00 | | | | | below LLOQ |
| | 4 | 0.0328 | 0.0325 | 1% | 0.88 | 0.86 | 3% | <LLOQ | | Sample |
| | | 0.0323 | | | 0.84 | | | | | below LLOQ |
| | 8 | 0.0284 | 0.0292 | 4% | 0.56 | 0.62 | 14% | <LLOQ | | Sample |
| | | | | | | | | | | below LLOQ |
| | | 0.0301 | | | 0.68 | | | | | |
| HMN42508 | 2 | 0.0541 | 0.0541 | | 2.37 | 2.37 | | 4.74 | | One replicate |
| (CSF) | | NA | | | NA | | | | | lost due to |
| | | | | | | | | | | imaging error |
| | 4 | 0.0359 | 0.0392 | 12% | 1.10 | 1.33 | 24% | 5.33 | 112% | One replicate |
| | | 0.0424 | | | 1.56 | | | | | below LLOQ |
| | 8 | 0.0350 | 0.0347 | 1% | 1.04 | 1.02 | 3% | <LLOQ | | Sample |
| | | 0.0344 | | | 1.00 | | | | | below LLOQ |
| HMN42509 | 2 | 0.0502 | 0.0475 | 8% | 2.10 | 1.91 | 14% | 3.82 | | |
| (CSF) | | 0.0447 | | | 1.72 | | | | | |
| | 4 | 0.0419 | 0.0421 | 1% | 1.53 | 1.54 | 1% | 6.15 | 161% | |
| | | 0.0422 | | | 1.55 | | | | | |
| | 8 | 0.0354 | 0.0345 | 4% | 1.07 | 1.00 | 9% | <LLOQ | | Sample |
| | | 0.0336 | | | 0.94 | | | | | below LLOQ |
| HMN42510 | 2 | 0.0322 | 0.0343 | 9% | 0.84 | 0.99 | 22% | <LLOQ | | Sample |
| (CSF) | | 0.0364 | | | 1.14 | | | | | below LLOQ |
| | 4 | 0.0358 | 0.0352 | 2% | 1.10 | 1.05 | 6% | <LLOQ | | Sample |
| | | 0.0346 | | | 1.01 | | | | | below LLOQ |
| | 8 | 0.0314 | 0.0307 | 3% | 0.78 | 0.73 | 9% | <LLOQ | | Sample |
| | | | | | | | | | | below LLOQ |
| | | 0.0301 | | | 0.68 | | | | | |
| HMN42547 | 2 | 0.0269 | 0.0282 | 7% | <LOD | 0.64 | | <LLOQ | | Sample |
| (Plasma) | | 0.0295 | | | 0.64 | | | | | below |
| | | | | | | | | | | LLOQ; One |
| | | | | | | | | | | replicate |
| | | | | | | | | | | below LOD |
| | 4 | 0.0284 | 0.0289 | 3% | 0.56 | 0.60 | 10% | <LLOQ | | Sample |
| | | 0.0295 | | | 0.64 | | | | | below LLOQ |
| | 8 | 0.0244 | 0.0222 | 14% | <LOD | <LOD | | <LOD | | Sample |
| | | 0.0201 | | | <LOD | | | | | below LOD |
| HMN42526 | 2 | 0.0476 | 0.0442 | 11% | 1.92 | 1.69 | 20% | 3.38 | | |
| (Plasma) | | 0.0409 | | | 1.46 | | | | | |
| | 4 | 0.0457 | 0.0413 | 15% | 1.79 | 1.48 | 29% | 5.93 | 176% | CV >25% |
| | | 0.0369 | | | 1.17 | | | | | |
| | 8 | 0.0293 | 0.0290 | 2% | 0.63 | 0.60 | 6% | <LLOQ | | Sample |
| | | 0.0286 | | | 0.57 | | | | | below LLOQ |
| HMN42527 | 2 | 0.0207 | 0.0196 | 8% | <LOD | <LOD | | <LOD | | Sample |
| (Plasma) | | 0.0185 | | | <LOD | | | | | below LOD |
| | 4 | 0.0189 | 0.0194 | 4% | <LOD | <LOD | | <LOD | | Sample |
| | | 0.0199 | | | <LOD | | | | | below LOD |
| | 8 | 0.0214 | 0.0210 | 2% | <LOD | <LOD | | <LOD | | Sample |
| | | 0.0207 | | | <LOD | | | | | below LOD |
| HMN42548 | 2 | 0.0543 | 0.0569 | 6% | 2.38 | 2.56 | 10% | 5.11 | | |
| (Plasma) | | 0.0594 | | | 2.73 | | | | | |
| | 4 | 0.0414 | 0.0422 | | 1.49 | 1.55 | 5% | 6.20 | 121% | |
| | | 0.0431 | | | 1.61 | | | | | |
| | 8 | 0.0385 | 0.0376 | 3% | 1.29 | 1.23 | 7% | 9.81 | 192% | |
| | | 0.0368 | | | 1.17 | | | | | |
| HMN42546 | 2 | 0.0334 | 0.0316 | 8% | 0.92 | 0.79 | 23% | <LLOQ | | Sample |
| (Plasma) | | 0.0298 | | | 0.66 | | | | | below LLOQ |
| | 4 | 0.0253 | 0.0276 | 12% | <LOD | 0.67 | | <LLOQ | | Sample |
| | | 0.0299 | | | 0.67 | | | | | below |
| | | | | | | | | | | LLOQ; One |
| | | | | | | | | | | replicate |
| | | | | | | | | | | below LOD |
| | 8 | NA | 0.0262 | | NA | | | <LOD | | Sample |
| | | 0.0262 | | | <LOD | | | | | below LOD; |
| | | | | | | | | | | One replicate |
| | | | | | | | | | | lost due to |
| | | | | | | | | | | imaging error |
| HMN42515 | 2 | 2.1536 | 2.1831 | 2% | 138.99 | 141 | 2% | 282 | | |
| (Serum) | | 2.2127 | | | 143.15 | | | | | |
| | 4 | 1.1044 | 1.1240 | 2% | 68.78 | 70.0 | 2% | 280 | 99% | |
| | | 1.1436 | | | 71.29 | | | | | |
| | 8 | 0.5277 | 0.5293 | 0% | 32.41 | 32.5 | 0% | 260 | 92% | |
| | | 0.5309 | | | 32.61 | | | | | |
| HMN42516 | 2 | 0.0237 | 0.0242 | 3% | <LOD | <LOD | | <LOD | | Sample |
| (Serum) | | 0.0247 | | | <LOD | | | | | below LOD |
| | 4 | NA | 0.0263 | | NA | <LOD | | <LOD | | Sample |
| | | 0.0263 | | | <LOD | | | | | below LOD; |
| | | | | | | | | | | One replicate |
| | | | | | | | | | | lost due to |
| | | | | | | | | | | imaging error |
| | 8 | 0.0252 | 0.0279 | 14% | <LOD | 0.72 | | <LLOQ | | Sample |
| | | 0.0306 | | | 0.72 | | | | | below |
| | | | | | | | | | | LLOQ; One |
| | | | | | | | | | | replicate |
| | | | | | | | | | | below LOD |
| | 2 | 0.0676 | 0.0724 | 9% | 3.28 | 3.60 | 12% | 7.20 | | |
| HMN42517 | | 0.0771 | | | 3.92 | | | | | |
| (Serum) | 4 | 0.0583 | 0.0555 | 7% | 2.66 | 2.46 | 11% | 9.84 | 137% | |
| | | 0.0526 | | | 2.27 | | | | | |
| | 8 | 0.0436 | 0.0449 | 4% | 1.64 | 1.73 | 7% | 13.88 | 193% | |
| | | 0.0462 | | | 1.83 | | | | | |
| HMN42522 | 2 | 0.0402 | 0.0365 | 14% | 1.41 | 1.14 | 33% | <LLOQ | | Sample |
| (Serum) | | 0.0328 | | | 0.88 | | | | | below |
| | | | | | | | | | | LLOQ; One |
| | | | | | | | | | | replicate |
| | | | | | | | | | | within cal |
| | | | | | | | | | | range; |
| | | | | | | | | | | CV >25% |
| | 4 | 0.0373 | 0.0360 | 5% | 1.20 | 1.11 | 11% | <LLOQ | | Sample |
| | | 0.0348 | | | 1.02 | | | | | below |
| | | | | | | | | | | LLOQ; One |
| | | | | | | | | | | replicate |
| | | | | | | | | | | within cal |
| | | | | | | | | | | range |
| | 8 | 0.0309 | 0.0299 | 5% | 0.74 | 0.67 | 15% | <LLOQ | | Sample |
| | | 0.0289 | | | 0.60 | | | | | below LLOQ |
| HMN42543 | 2 | 0.0268 | 0.02.65 | 2% | <LOD | <LOD | | <LOD | | Sample |
| (Serum) | | 0.0261 | | | <LOD | | | | | below LOD |
| | 4 | 0.0145 | 0.0207 | 42% | <LOD | <LOD | | <LOD | | Sample |
| | | 0.0269 | | | <LOD | | | | | below LOD |
| | 8 | 0.0297 | 0.0290 | 3% | 0.66 | 0.61 | 11% | <LLOQ | | Sample |
| | | 0.0284 | | | 0.56 | | | | | below LLOQ |

An improved frataxin standard was created using helper beads. Helper beads are dye-encoded and are associated with the Simoa® platform, and specifically with Homebrew assays, to increase the sensitivity of the assay. The helper beads are mixed with the assay beads (bound with the capture antibody) when exposed to the sample. The Simoa® software, run on the Analyzer, distinguishes between the helper beads and the fluorescing assay beads, bound to frataxin.

When the frataxin standard was improved with helper beads, the effective limit of detection (LOD) was further reduced, increasing the sensitivity of the assay to low concentrations. Similarly, the lower limit of quantitation (LLOQ) was also reduced, increasing sensitivity, see Table 31.

TABLE 31
Frataxin Standard

| [Frataxin] | | AEB | AEB | Ave | | | |
|---|---|---|---|---|---|---|---|
| pg/mL | weight | Rep1 | Rep2 | AEB | SD | % CV | S/B |
| 0 | 1 | 0.0268 | 0.0219 | 0.0244 | 0.0035 | 14% | 1.00 |
| 0.686 | 1 | 0.0586 | 0.0638 | 0.0612 | 0.0037 | 6% | 2.51 |
| 2.06 | 1 | 0.1458 | 0.1430 | 0.1444 | 0.0020 | 1% | 5.93 |
| 6.17 | 1 | 0.3865 | 0.3990 | 0.3928 | 0.0089 | 2% | 16.13 |
| 18.5 | 1 | 1.1372 | 1.1881 | 1.1627 | 0.0360 | 3% | 47.75 |
| 55.6 | 1 | 3.4713 | 3.2790 | 3.3752 | 0.1360 | 4% | 138.61 |
| 167 | 1 | 9.1915 | 9.1927 | 9.1921 | 0.0008 | 0% | 377.49 |
| 500 | 1 | 22.7900 | 22.7385 | 22.7643 | 0.0365 | 0% | 934.85 |

| | |
|---|---|
| LOD (pg/mL) | 0.117 |
| Estimated | 0.321 |
| LLOQ (pg/mL) | |

The assay conditions followed the Simoa® 2-step protocol with a 35-minute incubation of the beads with the sample and detection antibody, followed by a wash step, then a 5-minute incubation of the beads with the soluble β-galactosidase (SBG) reagent. The assay (see Table 32) uses 8×10⁶ assay beads per mL and 12×10⁶ helper beads per mL.

TABLE 32
Frataxin Assay Conditions
Frataxin Assay Conditions

| Assay Protocol | 2-step, 2.0 (35 min-5 min) | |
|---|---|---|
| [Beads] in bottle | 8 × 10⁶ assay beads/mL + | |
| | 12 × 10⁶ helper beads/mL | |
| Bead Diluent | Homebrew Bead Diluent | |
| Bead Reaction Volume | 25 | μL |
| [Detector] in bottle | 1.45 | μg/mL |
| Detector Diluent | Simoa ® Diluent 1 | |
| Detector Reaction Volume | 20 | μL |
| [SBG] in bottle | 50 | pM |
| SBG Diluent | SBG Dilution Buffer | |
| SBG Reaction Volume | 100 | μL |
| Calibrator Levels | 0, 0.686, 2.06, 6.17, 18.5, 55.6, 167, 500 pg/mL | |
| Calibrator Diluent | Simoa ® Diluent 1 | |
| Sample Diluent | Simoa ® Diluent 1 | |
| Calibrator/Sample Reaction | 170 | μL |
| Volume | | |

| | |
|---|---|
| Sample Dilution Factor (MRD) | 2x |

Having improved the frataxin standard, the samples of human circulating biofluids were again assayed. Based on the results of the preliminary sample testing (Table 29), the samples were diluted to a final minimum required dilution (MRD) of 2, at a final volume of 200 μL in Homebrew Diluent 1 (100 μL sample with 100 μL Diluent). The samples were transferred to a 96-well plate and run in the Simoa® HD-1 Analyzer.

With the protocol comprising helper beads, the CSF, plasma, and serum samples were run again, with an improved LOD and LLOQ, shown in Table 33.

TABLE 33
CSF, Serum, Plasma sample testing

| | | | | | | | | Dilution | |
|---|---|---|---|---|---|---|---|---|---|
| | | | | | | | | Corrected | |
| Sample ID | Dilution | AEB | Ave | AEB | Conc. | Ave | Conc. | Conc. | |
| (Matrix) | Factor | Reps | AEB | % CV | Reps | Conc. | % CV | (pg/mL) | Notes |
| HMN42511 | 2 | 0.0455 | 0.0469 | 4% | 0.39 | 0.41 | 9% | 0.823 | |
| (CSF) | | 0.0484 | | | 0.44 | | | | |
| HMN42512 | 2 | 0.0397 | 0.0382 | 6% | 0.28 | 0.26 | 15% | <LLOQ | Sample |
| (CSF) | | 0.0367 | | | 0.23 | | | | below LLOQ |
| HMN42532 | 2 | 0.0453 | 0.0465 | 4% | 0.38 | 0.40 | 8% | 0.809 | |
| (CSF) | | 0.0478 | | | 0.43 | | | | |
| HMN42533 | 2 | 0.0520 | 0.0531 | 3% | 0.50 | 0.52 | 5% | 1.04 | |
| (CSF) | | 0.0543 | | | 0.54 | | | | |
| | 2 | 0.1147 | 0.1102 | 6% | 1.58 | 1.50 | 7% | 3.00 | |
| HMN42530 | | 0.1057 | | | 1.43 | | | | |
| (CSF) | | | | | | | | | |
| HMN42531 | 2 | 0.0870 | 0.0914 | 7% | 1.11 | 1.18 | 9% | 2.36 | |
| (CSF) | | 0.0959 | | | 1.26 | | | | |
| HMN42505 | 2 | 0.0587 | 0.0582 | 1% | 0.62 | 0.61 | 2% | 1.22 | |
| (CSF) | | 0.0576 | | | 0.60 | | | | |
| HMN42506 | 2 | 0.0356 | 0.0319 | 17% | 0.21 | 0.21 | | <LLOQ | Sample |
| (CSF) | | 0.0281 | | | <LOD | | | | below |
| | | | | | | | | | LLOQ; One |
| | | | | | | | | | replicate |
| | | | | | | | | | below LOD |
| HMN42507 | 2 | 0.0353 | 0.0351 | 1% | 0.20 | 0.20 | 3% | <LLOQ | Sample |
| (CSF) | | 0.0349 | | | 0.20 | | | | below LLOQ |
| HMN42508 | 2 | NA | 0.0562 | | NA | 0.57 | | 1.15 | One replicate |
| (CSF) | | 0.0562 | | | 0.57 | | | | lost due to |
| | | | | | | | | | imaging |
| | | | | | | | | | error |
| HMN42509 | 2 | 0.0522 | 0.0502 | 5% | 0.50 | 0.47 | 10% | 0.939 | |
| (CSF) | | 0.0483 | | | 0.44 | | | | |
| HMN42510 | 2 | NA | 0.0370 | | NA | 0.24 | | <LLOQ | Sample |
| (CSF) | | 0.0370 | | | 0.24 | | | | below |
| | | | | | | | | | LLOQ; One |
| | | | | | | | | | replicate lost |
| | | | | | | | | | due to |
| | | | | | | | | | imaging |
| | | | | | | | | | error |
| HMN42547 | 2 | 0.0489 | 0.0475 | 4% | 0.45 | 0.42 | 8% | 0.844 | |
| (Plasma) | | 0.0462 | | | 0.40 | | | | |
| HMN42526 | 2 | NA | 0.0415 | | NA | 0.32 | | <LLOQ | Sample |
| (Plasma) | | 0.0415 | | | 0.32 | | | | below |
| | | | | | | | | | LLOQ; One |
| | | | | | | | | | replicate lost |
| | | | | | | | | | due to |
| | | | | | | | | | imaging |
| | | | | | | | | | error |
| HMN42527 | 2 | NA | | | NA | | | | Both |
| (Plasma) | | NA | | | NA | | | | replicates |
| | | | | | | | | | lost due to |
| | | | | | | | | | imaging |
| | | | | | | | | | error |
| HMN42548 | 2 | NA | 0.0874 | | NA | 1.11 | | 2.23 | One replicate |
| (Plasma) | | 0.0874 | | | 1.11 | | | | lost due to |
| | | | | | | | | | imaging |
| | | | | | | | | | error |
| HMN42546 | 2 | 0.0477 | 0.0477 | | 0.42 | 0.42 | | 0.850 | One replicate |
| (Plasma) | | NA | | | NA | | | | lost due to |
| | | | | | | | | | imaging |
| | | | | | | | | | error |
| HMN42515 | 2 | 4.8392 | 4.9825 | 4% | 80.95 | 83.49 | 4% | 167 | |
| (Serum) | | 5.1258 | | | 86.02 | | | | |
| HMN42516 | 2 | 0.0212 | 0.0229 | 11% | <LOD | <LOD | | <LOD | Below LOD |
| (Serum) | | 0.0247 | | | <LOD | | | | |
| HMN42517 | 2 | 0.0554 | 0.0667 | 2.4% | 0.56 | 0.76 | 37% | 1.51 | CV >25% |
| (Serum) | | 0.0780 | | | 0.95 | | | | |
| HMN42522 | 2 | 0.0420 | 0.0381 | 14% | 0.32 | 0.25 | 39% | <LLOQ | Sample |
| (Serum) | | 0.0342 | | | 0.19 | | | | below |
| | | | | | | | | | LLOQ: One |
| | | | | | | | | | replicate |
| | | | | | | | | | within curve |
| | | | | | | | | | range; |
| | | | | | | | | | CV >25% |
| HMN42543 | 2 | NA | 0.0303 | | NA | 0.11 | | <LOD | Below LOD; |
| (Serum) | | 0.0303 | | | 0.11 | | | | One replicate |
| | | | | | | | | | lost due to |
| | | | | | | | | | imaging |
| | | | | | | | | | error |

### Example 4. Parallelism Studies of Frataxin Detection in CSF, Serum, and Plasma

The detection of frataxin in human CSF, serum, and plasma samples was assessed using the Quanterix Simoa® Platform and the assay conditions as described in Table 34 below.

TABLE 34
Frataxin parallelism studies assay conditions.
Frataxin Parallelism Study Assay Conditions

| Assay Protocol | 2-step, 2.0 (120 min-5 min) | |
|---|---|---|
| [Beads] in bottle | 4 × 10⁶ assay beads/mL + | |
| | 6 × 10⁶ helper beads/mL | |
| Bead Diluent | Homebrew Bead Diluent | |
| Bead Reaction Volume | 25 | μL |
| [Detector] in bottle | 2.0 | μg/mL |
| Detector Diluent | SuperBlock ™ PBS | |
| Detector Reaction Volume | 20 | μL |
| [SBG] in bottle | 50 | pM |
| SBG Diluent | SBG Dilution Buffer | |
| SBG Reaction Volume | 100 | μL |
| Calibrator Levels | 0, 0.14, 0.41, 1.23, 3.70, 11.1, 33.3, 100 pg/mL | |
| Calibrator Diluent | SuperBlock ™ PBS | |
| Sample Diluent | SuperBlock ™ PBS | |
| Calibrator/Sample Reaction | 170 | μL |
| Volume | | |

| | |
|---|---|
| Sample Dilution Factor (MRD) | 4x |

SBG = streptavidin-β-galactosidase

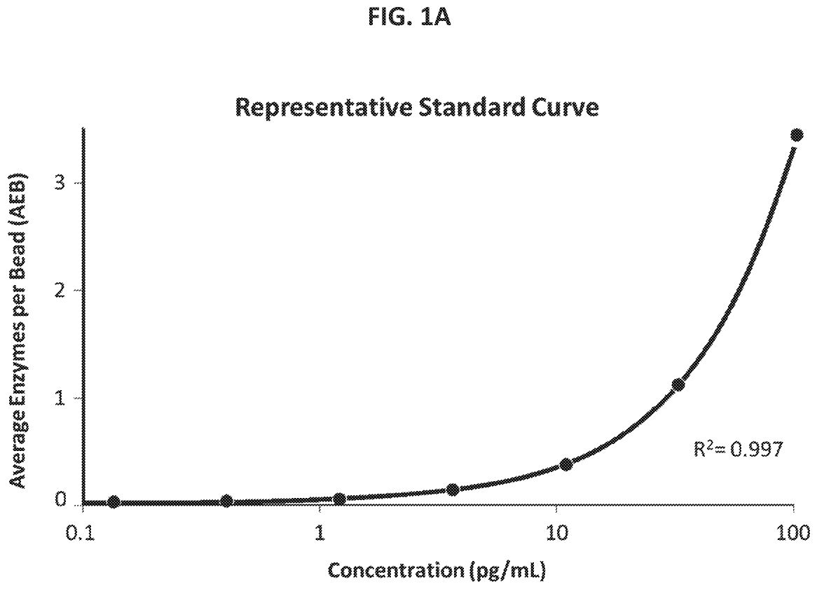

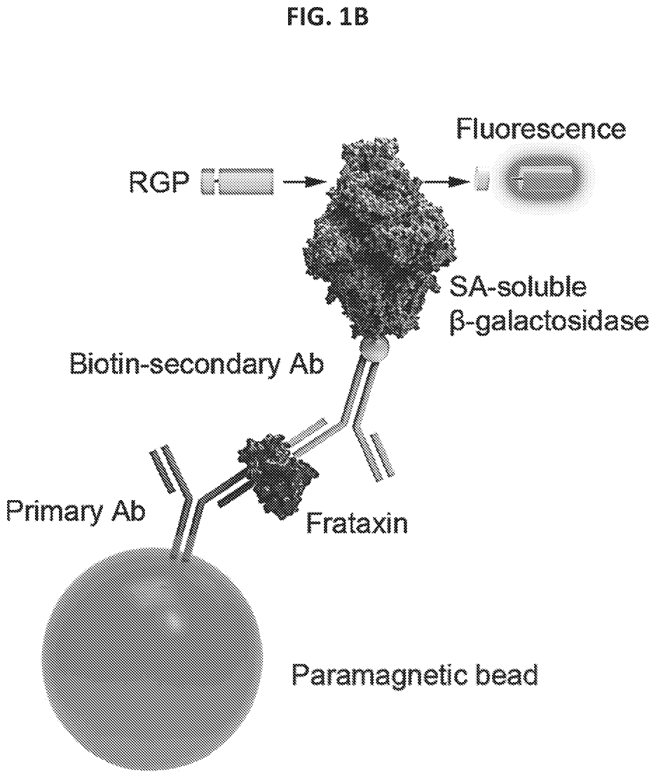

Assay range and QC reproducibility were tested using a frataxin recombinant protein standard (Table 2). Five samples were run as plate controls (QC samples) with four concentrations distinct from calibration dilutions. The results demonstrated a maximum % CV (Coefficient of Variation) of 12.038 at the upper limit of quantitation (ULoQ) and less than 10% relative error in duplicate runs. Two representative calibration curves aligned with an R² value of 0.997 over seven frataxin values ranging from 100-0.14 pg/mL in 3× dilution (FIG. 1A and Table 35).

TABLE 35
Assay Qualification using recombinant human Frataxin

| QC Sample | | Replicate | | | |
|---|---|---|---|---|---|
| ID | pg/mL | Mean | SE | % CV | % RE |
| ULoQ | 100 | 97.645 | 4.156 | 12.038 | 2.356 |
| High QC | 80 | 76.574 | 2.776 | 10.252 | 4.283 |
| Mid QC | 10 | 9.412 | 0.161 | 4.831 | 5.885 |
| Low QC | 2 | 1.824 | 0.008 | 1.202 | 8.825 |
| LLoQ | 0.5 | 0.465 | 0.001 | 0.608 | 7.000 |

Parallelism, defined as a difference of no greater than 25% between a reference sample and two of the three remaining dilution samples, was first tested in human CSF and serum. Results from the study (Table 36 below) showed that a single CSF sample failed the parallelism test while all serum samples passed. Based on these results, further frataxin analyses proceeded using CSF and serum samples.

TABLE 36
Parallelism study results in CSF and serum

| | | | | |
|---|---|---|---|---|
| CSF01 | CSF02 | CSF03 | CSF04 | CSF05 |

| | Conc. | | Conc. | | Conc. | | Conc. | | Conc. | |
|---|---|---|---|---|---|---|---|---|---|---|
| MRD | (pg/mL) | % RE | (pg/mL) | % RE | (pg/mL) | % RE | (pg/mL) | % RE | (pg/mL) | % RE |
| 1:2 | 2.38 | 39.8 | 3.00 | N/A | 3.06 | N/A | 3.46 | N/A | 6.79 | N/A |
| 1:4 | 1.70 | N/A | 3.53 | 17.6 | 3.79 | 23.7 | 3.04 | −12.2 | 8.21 | 20.8 |
| 1:8 | 1.41 | −17.2 | 3.61 | 20.2 | 1.79 | −41.6 | 2.91 | −16 | 8.58 | 26.3 |
| 1:16 | 0.88 | −48.5 | 3.25 | 8.4 | 3.29 | 7.4 | 3.64 | 5.1 | 7.21 | 6.2 |

| | Sera01 | | Sera02 | | Sera03 | | Sera04 | | Sera05 | |
|---|---|---|---|---|---|---|---|---|---|---|
| | Conc. | | Conc. | | Conc. | | Conc. | | Conc. | |
| MRD | (pg/mL) | % RE | (pg/mL) | % RE | (pg/mL) | % RE | (pg/mL) | % RE | (pg/mL) | % RE |
| 1:2 | 53.26 | −24.2 | 3.61 | N/A | 39.85 | N/A | 5.16 | N/A | 3.61 | N/A |
| 1:4 | 70.22 | N/A | 3.67 | 1.6 | 48.36 | 21.4 | 4.85 | −6.0 | 3.43 | −4.9 |
| 1:8 | 71.07 | 1.2 | 3.58 | −0.7 | 52.26 | 8.1 | NR | — | 3.35 | −7.1 |
| 1:16 | 75.89 | 8.1 | 2.05 | −43.3 | 54.69 | 13.1 | 5.3 | 9.2 | 4.37 | 21.0 |

All concentrations shown have been corrected for dilution. MRD=sample dilution factor, NR=no reading; N/A=comparator sample for % RE, calculation not applicable; RE=relative error; bolded text indicates samples with no % RE or % RE>±25%.

Parallelism tests using plasma samples failed in four out of five plasma samples. An initial experiment yielded only one successful sample and many samples with no reading. To mitigate a potential matrix or contaminant effect, further dilutions were included in a subsequence test. However, while two samples passed at higher dilutions, the original successful sample failed. Results are shown in Table 37 below.

TABLE 37
Parallelism study results in plasma

| | Plasma01 | | Plasma02 | | Plasma03 | | Plasma04 | | Plasma05 | |
|---|---|---|---|---|---|---|---|---|---|---|
| | Conc. | | Conc. | | Conc. | | Conc. | | Conc. | |
| MRD | (pg/mL) | % RE | (pg/mL) | % RE | (pg/mL) | % RE | (pg/mL) | % RE | (pg/mL) | % RE |
| 1:2 | 70.55 | N/A | 4.33 | N/A | 22.79 | N/A | NR | — | NR | — |
| 1:4 | 65.10 | −7.7 | NR | — | 85.23 | 274.0 | NR | — | NR | — |
| 1:8 | 59.88 | −15.1 | NR | — | 43.97 | 93.0 | NR | — | NR | — |
| 1:16 | 62.17 | −11.9 | NR | — | NR | — | 41.96 | N/A | 39.39 | N/A |
| 1:2 | 52.37 | −27.9 | 26.83 | −29.2 | 45.36 | −22.6 | 34.26 | −6.4 | 30.77 | −10.9 |
| 1:4 | NR | — | 27.74 | −26.8 | 58.62 | N/A | 36.60 | N/A | 34.53 | N/A |
| 1:8 | 72.64 | N/A | 37.91 | N/A | 74.79 | 27.6 | 40.89 | 11.7 | 42.63 | 23.5 |
| 1:16 | 94.08 | 29.5 | 40.20 | 6.0 | 113.30 | 93.3 | 53.26 | 45.5 | 46.29 | 34.1 |

Values for samples indicate frataxin (pg/mL);
NR = no reading

To determine whether K₂EDTA may be a potential cause of interference in the plasma parallelism test, samples of serum and plasma obtained from single donors with three common anticoagulants, including K₂EDTA, were assayed for frataxin. Results showed that serum frataxin failed to correlate with measured plasma frataxin from any of the three coagulant data sets (Table 38 below).

TABLE 38
Examination of multiple anticoagulants in plasma.

| | | | | | | Spearman r |
|---|---|---|---|---|---|---|
| Sample | Donor #1 | Donor #2 | Donor #3 | Donor #4 | Donor #5 | vs Serum |
| Serum | 4.05 | 3.44 | 3.40 | 3.12 | 20.31 | — |
| Plasma-K₂EDTA | 1.83 | 1.02 | 1.97 | 2.43 | 11.63 | 0.1 |
| Plasma-Heparin | 1.72 | NR | 3.14 | 3.78 | 13.93 | 0.2 |
| Plasma-Citrate | 0.57 | 1.16 | 2.13 | 2.29 | 9.01 | 0.0 |

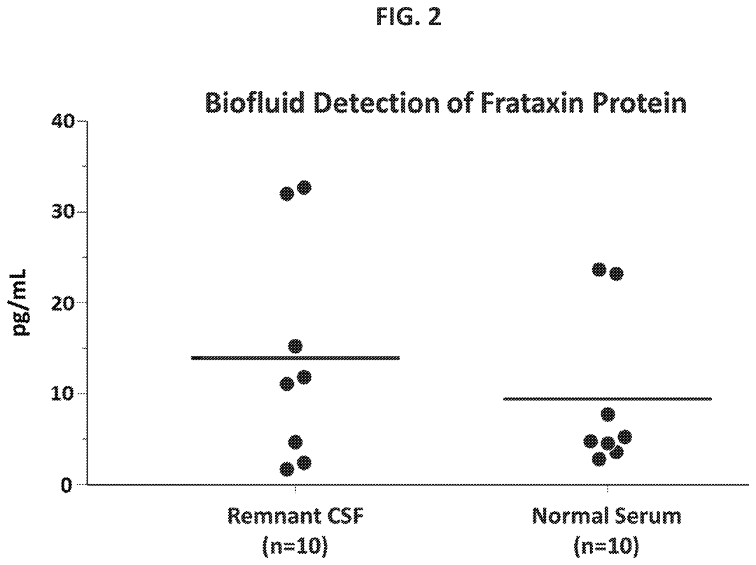

Based on the success of frataxin parallelism studies in CSF and serum samples, frataxin was then assayed in 10 remnant CSF samples and 10 normal serum samples from human donors. Assay results demonstrated that frataxin was detected within both CSF (8/10 samples) and serum samples at previously undetectable levels (FIG. 2 and Table 39).

TABLE 39
Biofluid Detection of Frataxin Protein

| | Mean | Range |
|---|---|---|
| Sample | (pg/mL) | (pg/mL) |
| Remnant CSF | 13.95 | 1.69-32.68 |
| Normal Serum | 9.44 | 2.81-23.64 |

Notably, the serum frataxin mean of the current study (9.44 pg/mL) is a roughly three-log reduction in level compared to reported non-erythrocyte blood cell levels of 7.1±1.0 ng/mL² (See Blair et al. Future Sci OA, 2019 July; 5(6) and Guo et al. Sci Rep. 2018 Nov. 19; 8(1):17043, the contents of which are herein incorporated by reference in their entirety).

### Example 5. Dual Assessment of Frataxin and Neurofilament Light Chain

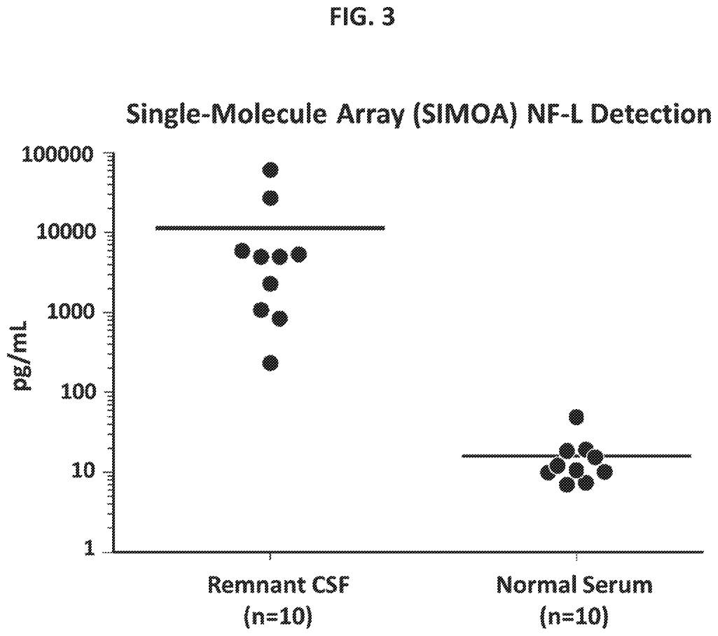

Possible combination assessment of frataxin with neurofilament light chain (NF-L) was assessed using the Quanterix Simoa® NF-Light® platform and protocol. NF-L protein was successfully measured in both CSF and serum, with detection values being notably higher in CSF samples compared to serum samples. This higher detection level of NF-L in CSF was expected given its association with neurons. Frataxin was still detectable albeit at a roughly three-log lower level. Results of this initial dual assessment study is shown in FIG. 3 and Table 40.

TABLE 40
Biofluid Detection of NFL Protein

| | Mean | Range |
|---|---|---|
| Sample | (pg/mL) | (pg/mL) |
| Remnant CSF | 11351.06 | 232.00-60886.30 |
| Normal Serum | 15.97 | 7.02-49.18 |

Given that NF-L is detectable in both CSF and serum, a potential correlation of NF-L and frataxin was measured. Results of the correlation studies showed that correlation of frataxin and NF-L was stronger in CSF than in serum where NF-L levels are significantly lower. Variability in NF-L levels within the remnant CSF samples and small sample size are potential factors that may have impacted this assessment. Table 41 below summarizes the results of this correlation study.

TABLE 41
Correlation of measured frataxin and NF-L

| | | |
|---|---|---|
| Frataxin | NF-L | Spearman |

| | | |
|---|---|---|
| Sample | pg/mL | Coefficient |

| | | | |
|---|---|---|---|
| Remnant | 11.83 | 60886.3 | 0.64 |
| CSF | 2.43 | 838.9 | |
| | 15.22 | 2293.6 | |
| | 4.69 | 1074.0 | |
| | 11.08 | 27032.3 | |
| | 1.69 | 232.0 | |
| | 31.99 | 4973.9 | |
| | 32.68 | 4975.4 | |
| Normal | 5.26 | 19.1 | 0.24 |
| Serum | 3.60 | 10.2 | |
| | 4.77 | 7.4 | |
| | 2.81 | 49.2 | |
| | 4.54 | 7.0 | |
| | 23.19 | 12.1 | |
| | 23.64 | 10.7 | |
| | 7.74 | 15.6 | |

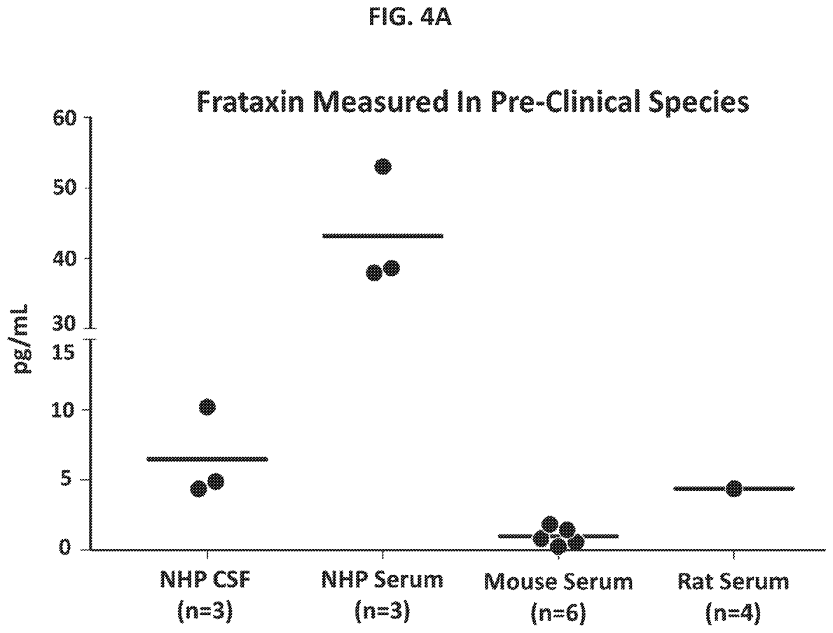

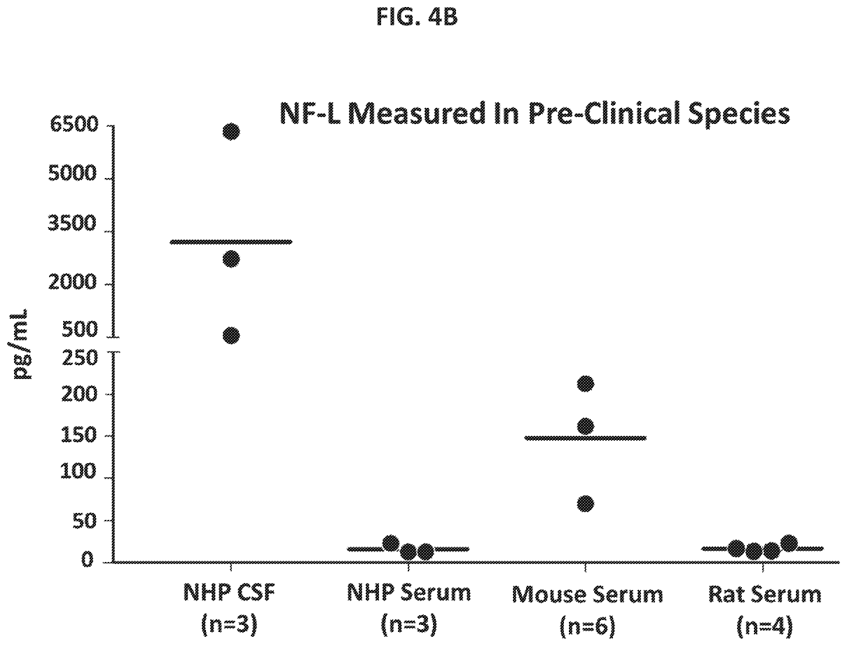

Preliminary assessment of frataxin and NF-L in pre-clinical in vivo studies showed that both frataxin and NF-L were readily detected in CSF samples but not all serum samples (FIG. 4A and FIG. 4B). Detection of frataxin and NF-L was assessed in 3 non-human primate CSF and serum samples, 6 mouse serum samples, and 4 rat serum samples. Frataxin was detected in NHP CSF at 4.37-10.19 pg/mL and in serum at 37.92-53.05 pg/mL. Frataxin was also detected in mouse serum at 0.25-1.85 pg/mL (5/6 samples) and in a single sample of rat serum at 4.39 pg/mL (1/4 samples). NF-L detection failed in three mouse serum samples but was again notably higher in CSF relative to serum.

### Example 6. Biovalidation Studies

### Animal Models

The assay described herein for the detection of frataxin protein in a biofluid such as CSF is used in biovalidation studies. Bio samples (tissues and biofluids) are collected from an animal model (healthy or disease model; non-human primate, cat, dog, rat, mouse, ferret, pig, sheep, etc.) and tested for the detection and quantification of frataxin protein levels. The animal is then administered a therapeutic agent designed to increase levels of frataxin in the animal model. After a pre-determined incubation period, samples are collected and tested for the detection and quantification of frataxin protein levels. Pre-treatment and post-treatment frataxin levels are compared to determine efficacy of treatment.

One might anticipate a substantially increased quantification of frataxin protein levels in an animal treated with a therapeutically effective amount of a therapeutic agent for increasing frataxin levels.

### Human Samples

Bio samples are collected from Friedreich's Ataxia patients or other individuals with a frataxin deficiency and age-matched controls. The assay described herein for the detection and quantification of frataxin in biofluids is used to establish the magnitude of difference in frataxin between a patient and the age-matched control.

The assay may be then used to design an appropriate treatment regimen to raise frataxin levels in the patient, e.g., via gene therapy.

### Example 7. Patient Validation Studies

A. Frataxin Quantification in Samples Collected from Patients Diagnosed with Friedreich's Ataxia

Samples of CSF and serum were collected from five Caucasian patients (Aged 23-52; 1 M, 4 F) diagnosed with Friedreich's Ataxia having GAA repeat numbers in the range of 445-780. Age of onset in this patient group ranged from 7 years of age to 23 years of age. Disease state was characterized using SARA scores, which ranged from 11-33. Frataxin was measured using the Quanterix Simoa® platform and protocol described in Example 4. Mean, standard deviation (SD), and coefficient of variance (% CV) average enzyme per bead (AEB) and mean, SD, and % CV frataxin concentration were measured in five CSF and five serum samples obtained from patients with Friedreich's Ataxia. Frataxin levels detected in patient samples ranged from 0.65-1.2 pg/mL in the CSF and 0.42-1.45 in serum, respectively. Mean FXN levels were 0.70 pg/mL in patient serum samples and 0.86 pg/mL in patient CSF samples. Results are summarized in Table 42 below.

TABLE 42
Frataxin assay in patient CSF and serum samples.

| | | | | | | | Dilution |
|---|---|---|---|---|---|---|---|
| | | | | Mean | SD | % CV | corr. |
| | Mean | SD | % CV | [FXN] | [FXN] | [FXN] | [FXN] |
| Sample | AEB | AEB | AEB | (pg/ml)* | (pg/mL) | (pg/mL) | (pg/mL) |
| CSF-1 | 0.013477 | 0.000618 | 0.045818 | 0.222996 | 0.015992 | 0.071713 | 0.89 |
| CSF-2 | 0.016475 | 0.001417 | 0.08599 | 0.300955 | 0.036966 | 0.12283 | 1.20 |
| CSF-3 | 0.012585 | 0.000371 | 0.029514 | 0.199926 | 0.009593 | 0.047984 | 0.80 |
| CSF-4 | 0.012227 | 0.000556 | 0.045439 | 0.190685 | 0.014332 | 0.075162 | 0.76 |
| CSF-5 | 0.011177 | 0.002178 | 0.194899 | 0.163742 | 0.055965 | 0.341785 | 0.65 |
| Serum-1 | 0.018832 | 0.003211 | 0.170515 | 0.362714 | 0.084183 | 0.232092 | 1.45 |
| Serum-2 | 0.010755 | 0.00123 | 0.114319 | 0.152842 | 0.031539 | 0.206353 | 0.61 |
| Serum-3 | 0.009144 | 0.003785 | 0.413968 | 0.112169 | 0.096197 | 0.85761 | 0.45 |
| Seram-4 | 0.010428 | 0.002581 | 0.247455 | 0.144616 | 0.066081 | 0.456943 | 0.58 |
| Serum-5 | 0.008854 | 0.001395 | 0.157549 | 0.104329 | 0.035441 | 0.339704 | 0.42 |

B. Frataxin Quantification in Samples Collected from Patients Diagnosed with Neurodegenerative Conditions

As a comparison to the FXN quantification obtained using serum and CSF samples collected from FA patients, samples were additionally collected from healthy controls and individuals diagnosed with other neurodegenerative conditions, e.g., Alzheimer Disease (AD), Multiple Sclerosis (MS), and Parkinson's Disease (PD). Patient demographics and disease severity are as noted in Table 43 below, with FA demographics of the group noted above included as reference. In Table 43, AD=Alzheimer's Disease, MS=Multiple Sclerosis, PD=Parkinson's Disease, FA=Friedreich's Ataxia, F=female, M=male, MMSE=Mini Mental State Exam, and SARA=Scale for Assessment and Rating of Ataxia. Values with asterisks were calculated without dilution factor. Values after accounting for the dilution factor appear in the far-right column.

TABLE 43
Patient Demographics and Disease Severity.

| | Control | AD | MS | PD | FA |
|---|---|---|---|---|---|
| N | 10 | 5 | 5 | 5 | 5 |
| Age | 20-54 | 57-84 | 44-67 | 57-66 | 23-52 |
| Sex | 6M, 4F | 2M, 3F | 2M, 3F | 3M, 2F | 1M, 4F |
| | | | Chronic | | GAA1: 445-780; |
| Disease | N/A | MMSE 20-22 | Disease-2; | MMSE 25-30 | Onset 7-23; |
| Severity | | | Unknown-3 | | SARA: 11-33 |
| Race | | | | | |
| Black | 10 | 0 | 0 | 0 | 0 |
| Caucasian | 0 | 2 | 5 | 4 | 5 |
| Hispanic | 0 | 3 | 0 | 1 | 0 |

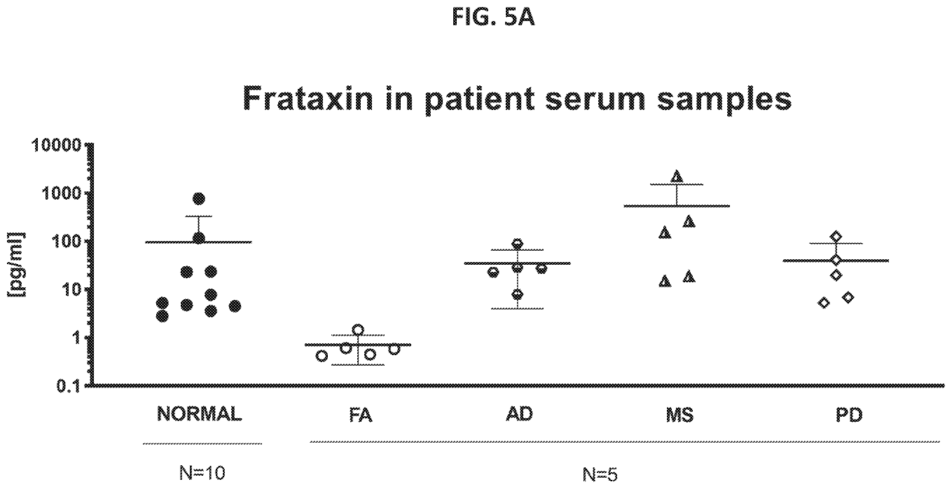

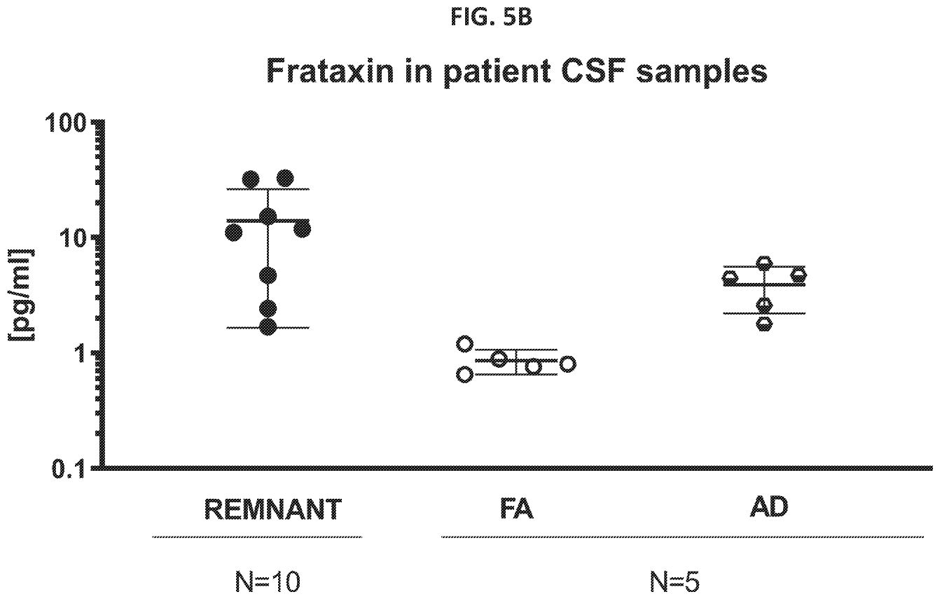

Frataxin was measured in each of the serum and CSF samples collected from the healthy controls and diseased individuals using the Quanterix Simoa® platform and protocol described in Example 4. The results are shown in Tables 44 and 45, respectively and given as concentration of Frataxin (pg/mL). Comparable data for FXN (pg/mL) in serum and CSF samples collected from patients diagnosed with FA are shown in Table 42 above. FXN quantification in serum and CSF collected from healthy controls and all patient groups (FA, AD, MS, PD) is also shown in FIG. 5A and FIG. 5B.

TABLE 44
Frataxin [pg/mL] in control and patient serum samples.

| | Control | | FA | AD | MS | PD |
|---|---|---|---|---|---|---|
| Serum FXN | 5.26 | 4.54 | 1.45 | 28.65 | 261.52 | 5.30 |
| [pg/mL] | 3.6 | 23.19 | 0.61 | 27.31 | 2254.01 | 41.02 |
| | 4.77 | 23.64 | 0.45 | 7.91 | 18.71 | 19.80 |
| | 2.81 | 7.74 | 0.58 | 22.90 | 15.12 | 6.83 |
| | 764.74* | 115.59* | 0.42 | 88.80 | 153.92 | 125.57 |

| | | | | | |
|---|---|---|---|---|---|
| Mean | 9.44 | 0.70 | 35.11 | 540.7 | 39.71 |
| Median | 5.02 | 0.58 | 27.31 | 153.92 | 19.8 |

*Outliers excluded in determining mean and median values.

TABLE 45
Frataxin [pg/mL] in control and patient CSF samples.

| | | |
|---|---|---|
| Control | FA | AD |

| | | | | |
|---|---|---|---|---|
| CSF FXN | 11.83 | 4.69 | 0.89 | 4.46 |
| [pg/mL] | — | 11.08 | 1.20 | 4.71 |
| | 2.43 | 1.69 | 0.80 | 5.96 |
| | 15.22 | 31.99 | 0.76 | 1.78 |
| | — | 32.68 | 0.65 | 2.59 |

| | | | |
|---|---|---|---|
| Mean | 13.95 | 0.86 | 3.90 |
| Median | 11.46 | 0.80 | 4.46 |

Frataxin protein was successfully detected in serum and CSF samples of healthy controls, FA patients and patients diagnosed with other neurodegenerative disease. Frataxin protein levels in serum and CSF samples collected from FA patients were lower than those evident in samples collected from healthy controls and patients diagnosed with other neurodegenerative disease. Mean frataxin protein levels were 0.70 pg/mL in FA patient serum samples and 0.86 pg/mL in FA patient CSF samples, as compared to 9.44 pg/mL and 13.95 pg/mL in control serum and CSF samples, respectively. Similarly, median frataxin protein levels were 0.58 pg/mL (range 0.42-145 pg/mL) in FA patient serum samples and 0.80 pg/mL (range 0.65-1.20 pg/mL) in FA patient CSF, as compared to 5.02 pg/mL (range 2.81-23.64 pg/mL) in control serum samples and 11.46 pg/mL (range 1.69-32.68 pg/mL) in control CSF samples. No frataxin was detected in two of the non-FA control CSF samples (noted as “-” in Table 45, above). A ROUT analysis (Q=0.1%) flagged two high non-FA control serum data points (115.6 and 764.74 pg/mL, indicated by asterisks) as outliers.

C. Neurofilament Light Quantification in Samples Collected from Patients and Healthy Controls

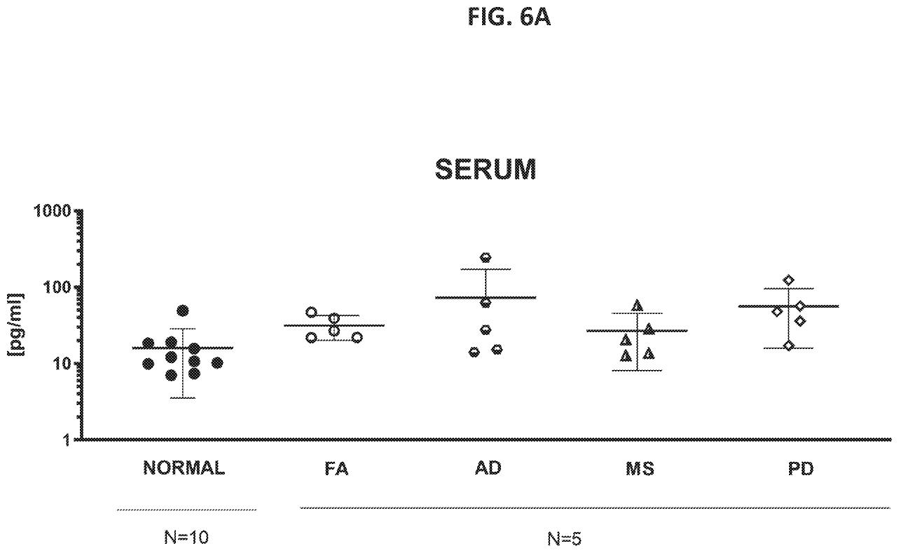

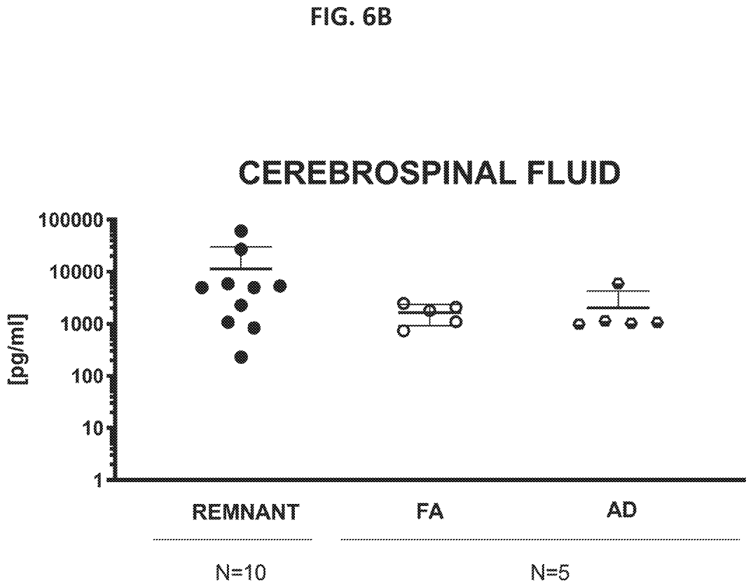

For each of the individuals noted in Table 43, neurofilament light chain was also measured in the collected serum and CSF samples using the Quanterix Simoa® platform. The results are shown in Tables 46 and 47, respectively and given as concentration of NFL (pg/mL). NFL quantification in serum and CSF collected from healthy controls and all patient groups (FA, AD, MS, PD) is also shown in FIG. 6A and FIG. 6B.

TABLE 46
Neurofilament Light [pg/mL] in control and patient serum samples.

| | Control | | AD | MS | PD | FA |
|---|---|---|---|---|---|---|
| Serum NFL | 19.10 | 7.00 | 244.82 | 28.60 | 17.20 | 22.00 |
| [pg/mL] | 10.20 | 12.10 | 15.31 | 20.60 | 35.90 | 39.30 |
| | 7.40 | 10.70 | 14.07 | 13.70 | 48.00 | 26.90 |
| | 49.20 | 15.60 | 27.67 | 12.80 | 123.30 | 21.80 |
| | 9.90 | 18.50 | 62.44 | 58.20 | 56.60 | 46.80 |

| | | | | | |
|---|---|---|---|---|---|
| Avg. | 15.97 | 72.86 | 26.78 | 56.20 | 31.36 |

TABLE 47
Neurofllament Light [pg/mL] in control and patient CSF samples.

| | | |
|---|---|---|
| Control | AD | FA |

| | | | | |
|---|---|---|---|---|
| CSF NFL | 60886.30 | 1074.00 | 1060.52 | 1099.10 |
| [pg/mL] | 5881.10 | 27032.30 | 1135.99 | 2111.00 |
| | 838.90 | 232.00 | 6001.17 | 2487.50 |
| | 2293.60 | 4973.90 | 1024.98 | 735.90 |
| | 5323.10 | 4975.40 | 988.79 | 1812.30 |

| | | | |
|---|---|---|---|
| Avg. | 11351.06 | 2042.29 | 1649.16 |

### Example 8. Dual Assessment of Frataxin and Neuroimaging Studies

A frataxin assay as described herein (e.g., protocol described in Example 4), will be used as a component in a 5-year longitudinal natural history neuroimaging study of brain and spinal cord changes in individuals with Friedreich's Ataxia (TRACK-FA). Biofluid samples, such as CSF, blood, serum and/or plasma will be collected for quantification of frataxin and/or neurofilament light chain and the readout (i.e., FXN quantification) may be correlated to the neuroimaging, clinical, and genetic findings.

An assay for quantification of platelet FXN levels in whole blood samples will be conducted in parallel and used for direct comparison to biofluid FXN protein measurements determined using the assay described herein. The platelet FXN assay is described in Guo, et al., Anal. Chem. 2018, 90, 2216-2223, the contents of which are herein incorporated by reference in their entirety. In brief, the method uses an immuno-purification two dimensional-nano-ultra high-performance liquid chromatography-parallel reaction monitoring/mass spectrometry (IP 2Dnano-HPLC-PRM/MS) method to quantify FXN protein present in platelets of whole blood samples.

Approximately 200 individuals diagnosed with FA aged 10 or greater and approximately 100 control participants will be recruited for magnetic resonance imaging (baseline, 12 months, and 24 months) at one of four (or more) international sites. Patients with early stage FA and patients with late stage FA will be included in the study (preferably 50/50 split). A 1.5-hour multimodal magnetic resonance imaging protocol will be used to assess brain morphometry of the cerebellum and brainstem, spinal cord morphometry, brain and spinal cord diffusion, dentate iron content (brain QSM), and spinal cord spectroscopy. Additional measures including clinical scales and assessments of upper limb function, speech, cognition, mood, and vision will be also be used.

A smaller cohort of individuals diagnosed with FA aged 5-10, each with an age-matched control, will be assessed using a shorter imaging protocol.

For inclusion in the study, individuals diagnosed with FA will be at least 5 years of age with genetically confirmed homozygosity for GAA repeat expansion in intron 1 of the FXN gene. Individuals with an ataxia staging score of equal to or less than 5 and total mFARS score of equal to or less than 65 may be included in the study. FA patients aged less than 5 years, with disease onset later than age 25, or with disease duration greater than or equal to 25 years will not be included in the study. Compound heterozygosity for an expanded GAA repeat and/or point mutations of the FXN gene, additional neurological conditions apart from FA, MR contraindications and/or inability to provide written informed consent will each be considered as exclusion criteria.

Three-tesla magnetic resonance imaging (MRI) scanners (e.g., Siemens Skyra 3T, Siemens Prisma 3T, Philips Achieva 3T, etc.) will be used for data acquisition according to the protocol and sequences outlined in Table 48 below, wherein QSM is Quantitative Susceptibility Maps (biometal imaging), GRE is gradient echo, MP-RAGE is a magnetization-prepared rapid acquisition GRE sequence, SPACE is sampling perfection with application optimized contrasts using different flip angle evolution, SE-EPI is spin echo-echo planar imaging, DTI is diffusion tensor imaging, MRS is magnetic resonance spectroscopy, and GRAPPA is generalized auto-calibrating partial parallel acquisition.

TABLE 48
MRI Protocol

| | | | Main | Time |
|---|---|---|---|---|
| Region | Modality | Sequence | parameters | (min) |
| Dentate Nuclei | Morphometry | QSM (GRE | 0.8 mm isotropic | 8 |
| | | multiecho) | | |
| Whole cerebellum and | Morphometry | T1 MP-RAGE (3D) | 0.8 mm isotropic | 11 |
| brainstem | | T2 SPACE | | |
| Superior, middle, and | DTI | SE-EPI | Multi-shell, | 11 |
| inferior cerebellar | | | MB = 3 | |
| peduncles | | | 197 Directions | |
| | | | 1.5 mm isotropic | |
| Cervical and thoracic | Morphometry | T2 SPACE | 0.8 mm isotropic | 8 |
| spinal cord | | | GRAPPA = 3 | |
| Cervical spinal cord | DTI | SE-EPI with | 0.9 × 0.9 × | 13 |
| | | ZoomIt | 3 mm³ | |
| Cervical spinal cord | MRS | Semilaser with | TE = 28 ms, | 20 |
| | | metabolite cycling | TR 3 s, nt 256 | |

Demographic information such as current age, gender, body mass index (BMI), height, weight, occupation, and years of education will be collected. Clinical assessments will include age of disease onset, disease duration, GAA repeat length and clinical scales FARS/SARA, ADL (assessment of daily living) and ataxia staging. Balance and gait will be assessed with the FARS upright stability test and upper limb function with the 9-hole peg test. Measure of dysarthria will be used to assess speech and the Sloan Low Contrast Acuity Chart for vision. Cognitive and mood assessments will be measured using the Cerebellar Cognitive Affective/Schmahmann syndrome scale and the Hospital Anxiety and Depression Scale.

Based on the assessments of this study, it is anticipated that the relationships between frataxin levels as measured by the assay and metrics such as clinical onset, progression, and severity, the FA genetic mutation, neuroimaging, and other markers of progression and severity may be validated.

### V. EQUIVALENTS AND SCOPE

Those skilled in the art will recognize, or be able to ascertain, using no more than routine experimentation, many equivalents to the certain embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes some embodiments in which exactly one member of the group is present in, employed in, or otherwise relevant to a given product or process. The disclosure includes some embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### VI. SEQUENCES

TABLE 49
Representative Promoters

| Promoter Name | Length of Promoter | SEQ ID NO |
|---|---|---|
| CMV-D7 | 109 | 7 |
| CBA-D8 | 100 | 8 |
| CBA-D4 | 332 | 9 |
| CBA-D6 | 260 | 10 |

TABLE 50
Representative ITR to ITR sequences

| | | | | | miR-122 | | | | SEQ |
|---|---|---|---|---|---|---|---|---|---|
| 5′ITR | Promoter | Intron | Payload | Tag | BS (3x) | Poly(A) | Filler | 3′ITR | ID NO |
| + | CMV-D7 | hβglobin | hFXN | − | + | hGH | Alb2264 | + | 11 |
| + | CBA-D8 | hβglobin | hFXN | − | + | hGH | Alb2266 | + | 12 |
| + | CBA-D4 | hβglobin | hFXN | − | + | hGH | Alb2034 | + | 13 |
| + | CBA-D6 | hβglobin | hFXN | − | + | hGH | Alb2106 | + | 14 |

TABLE 51
Representative sequences

| SEQ ID NO | Name/Descriptor | Sequence |
|---|---|---|
| 1 | Nucleic acid sequence | [symbol] |
| | encoding human | |
| | frataxin | |
| 2 | Nucleic acid sequence | [symbol] |
| | encoding human | |
| | frataxin | |
| 3 | Nucleic acid sequence | [symbol] |
| | encoding human | |
| | frataxin | |
| 4 | Amino acid sequence | MWTLGRRAVAGLLASPSPAQAQTLTRVPRPAELAPLCGRRGLRTDIDATC |
| | encoding human | TPRRASSNQRGLNQIWNVKKQSVYLMNLRKSGTLGHPGSLDETTYERLAE |
| | frataxin | ETLDSLAEFFEDLADKPYTFEDYDVSFGSGVLTVKLGGDLGTYVINKQTP |
| | | NKQIWLSSPSSGPKRYDWTGKNWVYSHDGVSLHELLAAELTKALKTKLDL |
| | | SSLAYSGKDA |
| 5 | Amino acid sequence | MWTLGRRAVAGLLASPSPAQAQTLTRVPRPAELAPLCGRRGLRTDIDATC |
| | encoding human | TPRRASSNQRGLNQIWNVKKQSVYLMNLRKSGTLGHPGSLDETTYERLAE |
| | frataxin | ETLDSLAEFFEDLADKPYTFEDYDVSFGSGVLTVKLGGDLGTYVINKQTP |
| | | NKQIWLSSPSRYVVDLSVMTGLGKTGCTPTTACPSMSCWPQSSLKP |
| 6 | Amino acid sequence | MWTLGRRAVAGLLASPSPAQAQTLTRVPRPAELAPLCGRRGLRTDIDATC |
| | encoding human | TPRRASSNQRGLNQIWNVKKQSVYLMNLRKSGTLGHPGSLDETTYERLAE |
| | frataxin | ETLDSLAEFFEDLADKPYTFEDYDVSFGSGVLTVKLGGDLGTYVINKQTP |
| | | NKQIWLSSPSRLTWLLWLFHP |
| 7 | CMV-D7 promoter | ttttggcacc aaaatcaacg ggactttcca aaatgtcgta |
| | | acaactccgc cccattgacg caaatgggcg gtaggcgtgt |
| | | acggtgggag gtctatataa gcagagctc |
| 8 | CBA-D8 promoter | ggtgcggcggcagccaatcagagcggcgcgctccgaaagtttccttttat |
| | | ggcgaggcggcggcggcggcggccctataaaaagcgaagcgcgcggcggg |
| 9 | CBA-D4 promoter | tacatgacct tatgggactt tcctacttgg cagtacatct |
| | | acgtattagt catcgctatt accatgtcga ggccacgttc |
| | | tgcttcactc tccccatctc ccccccctcc ccacccccaa |
| | | ttttgtattt atttattttt taattatttt gtgcagcgat |
| | | gggggcgggg ggggggggcg cgcgccaggc ggggcggggc |
| | | ggggcgaggg gcggggcggg gcgaggcgga gaggtgcggc |
| | | ggcagccaat cagagcggcg cgctccgaaa gtttcctttt |
| | | atggcgaggc ggcggcggcg gcggccctat aaaaagcgaa |
| | | gcgcgcggcg gg |
| 10 | CBA-D6 promoter | ccacgttctgcttcactctccccatctcccccccctccccacccccaatt |
| | | ttgtatttatttattttttaattattttgtgcagcgatgggggcgggggg |
| | | ggggggcgcgcgccaggcggggcggggcggggcgaggggcggggcggggc |
| | | gaggcggagaggtgcggcggcagccaatcagagcggcgcgctccgaaagt |
| | | ttccttttatggcgaggcggcggcggcggcggccctataaaaagcgaagc |
| | | gcgcggcggg |
| 11 | ITR to ITR sequence | [symbol] |
| | encoding human | |
| | frataxin payload | |
| 12 | ITR to ITR sequence | [symbol] |
| | encoding human | |
| | frataxin payload | |
| 13 | ITR to ITR sequence | [symbol] |
| | encoding human | |
| | frataxin payload | |
| 14 | ITR to ITR sequence | [symbol] |
| | encoding human | |
| | frataxin payload | |
| 15 | Amino acid sequence | [symbol] |
| | of VOY101 capsid | |
| 16 | Nucleic acid squence | [symbol] |
| | encoding VOY101 | |
| | capsid | |

[symbol]

indicates data missing or illegible when filed

1. A method of determining the amount of frataxin protein in a biofluid comprising:
performing an assay on a sample of a biofluid to determine the concentration of frataxin protein present in the sample; wherein a limit of detection of frataxin of the assay is about 2.0 pg/mL or less;
wherein the assay comprises a capture agent that specifically binds to frataxin or a fragment of frataxin, and a detection agent that specifically binds to frataxin or a fragment of frataxin.

2. The method of claim 1, wherein the capture agent and/or the detection agent comprises an antibody or fragment thereof.

3. The method of claim 1 or claim 2, wherein the capture agent and/or the detection agent comprises an anti-frataxin antibody or a frataxin-binding fragment thereof.

4. The method of any one of claims 1-3, wherein the capture agent comprises an antibody selected from the group consisting of Ab-1, Ab-2, Ab-3, Ab-4, Ab-5, and Ab-6.

5. The method of any one of claims 1-4, wherein the detection agent comprises an antibody selected from the group consisting of Ab-1, Ab-2, Ab-3, Ab-4, Ab-5, and Ab-6.

6. The method of any one of claims 1-5, wherein the capture agent comprises an antibody selected from the group consisting of Ab-1, Ab-2, Ab-3, Ab-4, Ab-5, and Ab-6, and the detection agent comprises an antibody selected from the group consisting of Ab-1, Ab-2, Ab-3, Ab-4, Ab-5, and Ab-6.

7. The method of any one of claims 1-6, wherein the capture agent is Ab-1 and the detection agent is Ab-3; wherein the capture agent is Ab-1 and the detection agent is Ab-1; wherein the capture agent is Ab-1 and the detection agent is Ab-2; wherein the capture agent is Ab-1 and the detection agent is Ab-4; wherein the capture agent is Ab-1 and the detection agent is Ab-5; wherein the capture agent is Ab-1 and the detection agent is Ab-6; wherein the capture agent is Ab-2 and the detection agent is Ab-1; wherein the capture agent is Ab-2 and the detection agent is Ab-2; wherein the capture agent is Ab-2 and the detection agent is Ab-3; wherein the capture agent is Ab-2 and the detection agent is Ab-4; wherein the capture agent is Ab-2 and the detection agent is Ab-5; wherein the capture agent is Ab-2 and the detection agent is Ab-6; wherein the capture agent is Ab-3 and the detection agent is Ab-1; wherein the capture agent is Ab-3 and the detection agent is Ab-2; wherein the capture agent is Ab-3 and the detection agent is Ab-3; wherein the capture agent is Ab-3 and the detection agent is Ab-4; wherein the capture agent is Ab-3 and the detection agent is Ab-5; wherein the capture agent is Ab-3 and the detection agent is Ab-6; wherein the capture agent is Ab-4 and the detection agent is Ab-1; wherein the capture agent is Ab-4 and the detection agent is Ab-2; wherein the capture agent is Ab-4 and the detection agent is Ab-3; wherein the capture agent is Ab-4 and the detection agent is Ab-4; wherein the capture agent is Ab-4 and the detection agent is Ab-5; wherein the capture agent is Ab-4 and the detection agent is Ab-6; wherein the capture agent is Ab-5 and the detection agent is Ab-1; wherein the capture agent is Ab-5 and the detection agent is Ab-2; wherein the capture agent is Ab-5 and the detection agent is Ab-3; wherein the capture agent is Ab-5 and the detection agent is Ab-4; wherein the capture agent is Ab-5 and the detection agent is Ab-5; wherein the capture agent is Ab-5 and the detection agent is Ab-6; wherein the capture agent is Ab-6 and the detection agent is Ab-1; wherein the capture agent is Ab-6 and the detection agent is Ab-1; wherein the capture agent is Ab-6 and the detection agent is Ab-2; wherein the capture agent is Ab-6 and the detection agent is Ab-3; wherein the capture agent is Ab-6 and the detection agent is Ab-4; wherein the capture agent is Ab-6 and the detection agent is Ab-5; or wherein the capture agent is Ab-6 and the detection agent is Ab-6.

8. The method of any one of claims 1-7, wherein the capture agent is Ab-1 and the detection agent is Ab-3.

9. The method of any one of claims 1-8, wherein the biofluid is cerebrospinal fluid (CSF).

10. The method as in any of claims 1-9, wherein the biofluid is blood or a blood component.

11. The method of claim 10, wherein the blood component is plasma.

12. The method of claim 10, wherein the blood component is serum.

13. The method of any one of claims 1-12, wherein the assay comprises the steps of
a) incubating a substrate with the capture agent;
b) incubating the substrate with the biofluid sample and the detection agent;
c) incubating the substrate with a substance capable of engaging with the detection agent and creating a signal, wherein the signal is detectable and quantifiable.

14. The method of claim 13, wherein the signal is a fluorescence, which is visible, detectable, and quantifiable.

15. The method of claim 13 or claim 14, wherein the assay comprises assay beads and helper beads.

16. The method of claim 15, wherein the assay comprises a greater number of helper beads than assay beads.

17. The method of claim 15, wherein a ratio of helper beads to assay beads is about 1:5 to about 5:1, optionally about 1:1 to about 2:1, e.g., about 1:1, about 1.5:1, or about 2:1.

18. The method of any one of claims 13-17, wherein the incubating step of b) occurs for about 10-150 minutes and the incubating step of c) occurs for about 1-30 minutes.

19. The method of any one of claims 13-18, wherein the incubating step of b) occurs for about 35 minutes and the incubating step of c) occurs for about 5 minutes, wherein the incubating step of b) occurs for about 75 minutes and the incubating step of c) occurs for about 5 minutes, wherein the incubating step of b) occurs for about 120 minutes and the incubating step of c) occurs for about 30 minutes, or wherein the incubating step of b) occurs for about 120 minutes and the incubating step of c) occurs for about 5 minutes.

20. The method of claim 19, wherein the incubating step of b) occurs for about 120 minutes and the incubating step of c) occurs for about 5 minutes.

21. The method of any one of the preceding claims, wherein the limit of detection of frataxin is about 1.5 pg/mL or less.

22. The method of any one of the preceding claims, wherein the limit of detection of frataxin is about 1.0 pg/mL or less.

23. The method of any one of the preceding claims, wherein the limit of detection of frataxin is about 0.5 pg/mL or less.

24. The method of any one of the preceding claims, wherein the method further comprises determining the amount of neurofilament light chain in a sample of a biofluid, optionally wherein the biofluid is cerebrospinal fluid (CSF) or serum.

25. The method of claim 24, wherein the amount of frataxin protein and the amount of neurofilament light chain are determined from a single sample of a biofluid.

26. A method of diagnosing Friedreich's Ataxia in a subject, comprising the steps of:
performing an assay on a sample of a biofluid from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
diagnosing the subject with Friedreich's Ataxia if the concentration of frataxin protein present in the sample is just above, below, or equal to the limit of detection.

27. A method of diagnosing Friedreich's Ataxia in a subject suspected of having Friedreich's Ataxia, comprising the steps of:
performing an assay on a sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
performing an assay on a sample of a biofluid from a healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
diagnosing the subject with Friedreich's Ataxia if the concentration of frataxin protein present in the sample from the subject suspected of having Friedreich's Ataxia is at least 2-fold less to at least 2000-fold less than the concentration of frataxin protein present in the sample from the healthy subject.

28. A method of diagnosing Friedreich's Ataxia in a subject, comprising the steps of:
performing an assay on a first sample of a biofluid from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
performing an assay on a second sample of a biofluid from the subject to assess the concentration of neurofilament light chain present in the sample; and
diagnosing the subject with Friedreich's Ataxia if the concentration of frataxin protein present in the first sample is just above, below, or equal to the limit of detection and if neurofilament light chain is detected in the second sample at a concentration of at least 20 pg/mL.

29. A method of diagnosing Friedreich's Ataxia in a subject suspected of having Friedreich's Ataxia, comprising the steps of:
performing an assay on a first sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
performing an assay on a second sample of a biofluid from the subject suspected of having Friedreich's Ataxia to assess the concentration of neurofilament light chain present in the sample; and
performing an assay on a first sample of a biofluid from an age-matched healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
performing an assay on a second sample of a biofluid from the age-matched healthy subject to assess the concentration of neurofilament light chain present in the sample; and
diagnosing the subject with Friedreich's Ataxia if the concentration of frataxin protein present in the first sample from the subject suspected of having Friedreich's Ataxia is at least 2-fold less to at least 2000-fold less than the concentration of frataxin protein present in the first sample from the age-matched healthy subject, and the concentration of neurofilament light chain present in the second sample from the subject suspected of having Friedreich's Ataxia is greater than the concentration of neurofilament light chain present in the second sample from the age-matched healthy subject.

30. The method of claim 28 or claim 29, wherein the first sample and the second sample of a biofluid from the subject suspected of having Friedreich's Ataxia are the same sample or each is a portion of an initial sample, and/or the first sample and the second sample of a biofluid from the healthy subject are the same sample or each is a portion of an initial sample.

31. The method of any one of claims 26-30, wherein the capture agent and/or the detection agent comprises an anti-frataxin antibody or a frataxin-binding fragment thereof.

32. The method of claim 31, wherein the capture agent comprises an antibody selected from the group consisting of Ab-1, Ab-2, Ab-3, Ab-4, Ab-5, and Ab-6, and the detection agent comprises an antibody selected from the group consisting of Ab-1, Ab-2, Ab-3, Ab-4, Ab-5, and Ab-6.

33. The method of claim 32, wherein the capture agent is Ab-1 and the detection agent is Ab-3.

34. The method of any one of claims 26-33, wherein the biofluid is CSF.

35. The method of any one of claims 26-33, wherein the biofluid is blood or a blood component.

36. The method of claim 35, wherein the blood component is plasma.

37. The method of claim 35, wherein the blood component is serum.

38. The method of any one of claims 26-37, wherein the assay comprises the steps of:
a) incubating a substrate with the capture agent;
b) incubating the substrate with the biofluid sample and the detection agent;
c) incubating the substrate with a substance capable of engaging with the detection agent and creating a signal, wherein the signal is detectable and quantifiable.

39. The method of claim 38, wherein the signal is a fluorescence, which is visible, detectable, and quantifiable.

40. The method of claim 38 or claim 39, wherein the substrate comprises assay beads and helper beads.

41. The method of claim 40, wherein the assay comprises a greater number of helper beads than assay beads.

42. The method of claim 40, wherein a ratio of helper beads to assay beads is about 1:5 to about 5:1, optionally about 1:1 to about 2:1, e.g., about 1:1, about 1.5:1, or about 2:1.

43. The method of any one of claims 38-42, wherein the incubating step of b) occurs for about 10-150 minutes and the incubating step of c) occurs for about 1-30 minutes.

44. The method of any one of claims 38-43, wherein the incubating step of b) occurs for about 35 minutes and the incubating step of c) occurs for about 5 minutes, wherein the incubating step of b) occurs for about 75 minutes and the incubating step of c) occurs for about 5 minutes, wherein the incubating step of b) occurs for about 120 minutes and the incubating step of c) occurs for about 30 minutes, or wherein the incubating step of b) occurs for about 120 minutes and the incubating step of c) occurs for about 5 minutes.

45. The method of claim 44, wherein the incubating step of b) occurs for about 120 minutes and the incubating step of c) occurs for about 5 minutes.

46. The method of any one of claims 26-45, wherein the limit of detection of frataxin is about 1.5 pg/mL or less, about 1.0 pg/mL or less, or about 0.5 pg/mL or less.

47. A method of treating Friedreich's Ataxia in a subject, comprising administering a frataxin gene therapy to a subject lacking detectable frataxin in a biofluid sample at a limit of detection of frataxin of about 2.0 pg/mL or less, wherein the amount of frataxin is determined using an assay.

48. A method of treating Friedreich's Ataxia in a subject, comprising the steps of:
a) determining whether a subject has frataxin in a biofluid sample using an assay having a limit of detection of frataxin of about 2.0 pg/mL or less; and
b) administering a suitable treatment to a subject determined as having frataxin in a biofluid sample at a concentration at just above, below, or equal to the limit of detection of the assay.

49. The method of claim 47 or claim 48, wherein the assay comprises a capture agent and a detection agent, wherein the capture agent and/or the detection agent is an antibody or fragment thereof, optionally wherein the capture agent and/or the detection agent is an anti-frataxin antibody or a frataxin-binding fragment thereof.

50. The method of claim 49, wherein the capture agent comprises an antibody selected from the group consisting of Ab-1, Ab-2, Ab-3, Ab-4, Ab-5, and Ab-6.

51. The method of claim 49, wherein the detection agent comprises an antibody selected from the group consisting of Ab-1, Ab-2, Ab-3, Ab-4, Ab-5, and Ab-6.

52. The method of claim 49, wherein the capture agent comprises an antibody selected from the group consisting of Ab-1, Ab-2, Ab-3, Ab-4, Ab-5, and Ab-6, and the detection agent comprises an antibody selected from the group consisting of Ab-1, Ab-2, Ab-3, Ab-4, Ab-5, and Ab-6.

53. The method of claim 52, wherein the capture agent is Ab-1 and the detection agent is Ab-3.

54. The method of any one of claims 47-53, wherein the biofluid is CSF.

55. The method as in any of claims 47-53, wherein the biofluid is blood or a blood component.

56. The method of claim 55, wherein the blood component is plasma.

57. The method of claim 55, wherein the blood component is serum.

58. The method as in any of claims 47-57, wherein the assay comprises the steps of
a) incubating a substrate with the capture agent;
b) incubating the substrate with the biofluid sample and the detection agent;
c) incubating the substrate with a substance capable of engaging with the detection agent and creating a signal, wherein the signal is detectable and quantifiable.

59. The method of claim 58, wherein the signal is a fluorescence, which is visible, detectable, and quantifiable.

60. The method of claim 58 or claim 59, wherein the substrate comprises assay beads and helper beads.

61. The method of claim 60, wherein the assay comprises a greater number of helper beads than assay beads.

62. The method of claim 60, wherein a ratio of helper beads to assay beads is about 1:5 to about 5:1, optionally about 1:1 to about 2:1, e.g., about 1:1, about 1.5:1, or about 2:1.

63. The method of any one of claims 58-62, wherein the incubating step of b) occurs for about 10-150 minutes and the incubating step of c) occurs for about 1-30 minutes.

64. The method of any one of claims 58-63, wherein the incubating step of b) occurs for about 35 minutes and the incubating step of c) occurs for about 5 minutes, wherein the incubating step of b) occurs for about 75 minutes and the incubating step of c) occurs for about 5 minutes, wherein the incubating step of b) occurs for about 120 minutes and the incubating step of c) occurs for about 30 minutes, or wherein the incubating step of b) occurs for about 120 minutes and the incubating step of c) occurs for about 5 minutes.

65. The method of any one of claims 58-64, wherein the incubating step of b) occurs for about 120 minutes and the incubating step of c) occurs for about 5 minutes.

66. The method of any one of claims 47-65, wherein the limit of detection of frataxin is about 1.5 pg/mL or less, about 1.0 pg/mL or less, or about 0.5 pg/mL or less.

67. The method of any one of claims 47-66, wherein the method further comprises determining the amount of neurofilament light chain in a sample of a biofluid, optionally wherein the biofluid is cerebrospinal fluid (CSF) or serum.

68. The method of claim 67, wherein the amount of frataxin protein and the amount of neurofilament light chain are determined from a single sample of a biofluid.

69. The method of claim 67 or claim 68, wherein the frataxin gene therapy or suitable treatment is administered to the subject if neurofilament light chain is detected in the sample at a concentration of at least 20 pg/mL.

70. The method of any one of claims 48-69, wherein the suitable treatment comprises frataxin gene therapy.

71. The method of any one of claims 48-69, wherein the suitable treatment comprises omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia, acetyl-L-carnitine (ALCAR), rosuvastatin, an incretin analog, indole-3-propionic acid, and/or erythropoietin or a modified (e.g., carbamylated) form thereof.

72. The method of any one of claims 48-69, wherein the suitable treatment comprises frataxin gene therapy and omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia, acetyl-L-carnitine (ALCAR), rosuvastatin, an incretin analog, indole-3-propionic acid, and/or erythropoietin or a modified (e.g., carbamylated) form thereof.

73. A method of determining the efficacy of a treatment to increase frataxin levels in a subject having Friedreich's Ataxia, comprising the steps of:
a) determining a baseline concentration of frataxin in a first sample of a biofluid of a subject, wherein the biofluid is optionally cerebrospinal fluid (CSF) or serum;
b) treating the subject with a treatment which results in increased frataxin levels in the subject;
c) determining a subsequent concentration of frataxin in a second sample of the biofluid of the subject;
d) comparing the baseline concentration of frataxin to the subsequent concentration of frataxin; and
e) determining treatment efficacy, wherein the treatment is effective if the subsequent concentration of frataxin is greater than the baseline concentration.

74. The method of claim 73, wherein the treatment is effective if the subsequent concentration of frataxin is at least 0.5×-3× greater than the baseline concentration.

75. The method of claim 73, wherein the treatment is effective if the subsequent concentration of frataxin is at least 3× greater than the baseline concentration.

76. A method of determining the efficacy of a treatment to increase frataxin levels in a subject having Friedreich's Ataxia, comprising the steps of:
a) determining a baseline concentration of frataxin in a first sample of a biofluid of a subject, wherein the biofluid is optionally cerebrospinal fluid (CSF) or serum;
b) determining a baseline concentration of neurofilament light chain in a second sample of a biofluid of the subject, wherein the biofluid is optionally CSF or serum;
c) treating the subject with a treatment which results in increased frataxin levels in the subject;
d) determining a subsequent concentration of frataxin in a third sample of a biofluid of the subject, wherein the biofluid of d) is CSF if the biofluid of a) is CSF, and wherein the biofluid of d) is serum if the biofluid of a) is serum;
e) determining a subsequent concentration of neurofilament light chain in a fourth sample of a biofluid of the subject, wherein the biofluid of e) is CSF if the biofluid of b) is CSF, and wherein the biofluid of e) is serum if the biofluid of b) is serum;
f) comparing the baseline concentration of frataxin to the subsequent concentration of frataxin and comparing the baseline concentration of neurofilament light chain to the subsequent concentration of neurofilament light chain; and
g) determining treatment efficacy, wherein the treatment is effective if the subsequent concentration of frataxin is greater than the baseline concentration of frataxin and the subsequent concentration of neurofilament light chain is not significantly higher than the baseline concentration of neurofilament light chain.

77. The method of claim 76, wherein the treatment is effective if the subsequent concentration of frataxin is at least 0.5× greater than the baseline concentration of frataxin and the subsequent concentration of neurofilament light chain is equal to or lower than the baseline concentration of neurofilament light chain.

78. The method of any one of claims 73-77, wherein the treatment comprises a frataxin gene therapy.

79. A method of treating Friedreich's Ataxia in a subject, comprising the steps of:
a) determining a baseline concentration of frataxin in a first sample of a biofluid of a subject, wherein the biofluid is optionally cerebrospinal fluid (CSF) or serum;
b) treating the subject with a frataxin gene therapy;
c) determining a subsequent concentration of frataxin in a second sample of the biofluid of the subject; and
d) comparing the baseline concentration of frataxin to the subsequent concentration of frataxin;
wherein the frataxin gene therapy is effective if the subsequent concentration of frataxin is greater than the baseline concentration of frataxin.

80. A method of treating Friedreich's Ataxia in a subject, comprising the steps of:
a) determining a baseline concentration of frataxin in a first sample of a biofluid of a subject and determining a baseline concentration of neurofilament light chain in a second sample of a biofluid of the subject, wherein the biofluid is optionally cerebrospinal fluid (CSF) or serum and wherein the first sample and the second sample are optionally the same sample;
b) treating the subject with a frataxin gene therapy;
c) determining a subsequent concentration of frataxin in a third sample of a biofluid of the subject and determining a subsequent concentration of neurofilament light chain in a fourth sample of a biofluid in the subject, wherein the biofluid of c) is CSF if the biofluid of a) is CSF, and wherein the biofluid of c) is serum if the biofluid of a) is serum, and wherein the third sample and the fourth sample are optionally the same sample; and
d) comparing the baseline concentration of frataxin to the subsequent concentration of frataxin and comparing the baseline concentration of neurofilament light chain to the subsequent concentration of neurofilament light chain;
wherein the frataxin gene therapy is effective if the subsequent concentration of frataxin is greater than the baseline concentration and the subsequent concentration of neurofilament light chain is not significantly higher than, is lower than, or is equal to the baseline concentration of neurofilament light chain.

81. The method of claim 79 or claim 80, wherein if the frataxin gene therapy is not effective, the subject is administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

82. The method of claim 79 or claim 80, wherein if the frataxin gene therapy is not effective, the subject is administered a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

83. The method of claim 79 or claim 80, wherein if the frataxin gene therapy is effective, the subject is administered a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

84. The method of claim 79 or claim 80, wherein if the frataxin gene therapy is effective, the subject is administered a treatment comprising managing one or more symptoms of Friedreich's Ataxia comprising occupational therapy, speech therapy, orthopedic care, bracing, surgery, treatment for heart disease, an antioxidant, treatment to lower blood sugar, and/or physical therapy.

85. The method of any one of claims 81-83, wherein the second therapy comprises omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia), acetyl-L-carnitine (ALCAR), rosuvastatin, an incretin analog, indole-3-propionic acid, and/or erythropoietin or a modified (e.g., carbamylated) form thereof.

86. The method of any one of claims 81-83, wherein the second therapy comprises omaveloxolone, wherein the low dose of omaveloxolone is about 5 mg/day, and wherein the high dose of omaveloxolone is about 300 mg/day.

87. The method of any one of claims 79-86, wherein assessing a baseline or subsequent concentration of frataxin comprises
i) incubating a substrate with a capture agent;
ii) incubating the substrate with a sample and a detection agent;
iii) incubating the substrate with a substance capable of engaging with the detection agent and creating a signal, wherein the signal is detectable and quantifiable.

88. The method of claim 87, wherein the capture agent comprises an antibody selected from the group consisting of Ab-1, Ab-2, Ab-3, Ab-4, Ab-5, and Ab-6; and the detection agent comprises an antibody selected from the group consisting of Ab-1, Ab-2, Ab-3, Ab-4, Ab-5, and Ab-6.

89. The method of claim 88, wherein the capture agent is Ab-1 and the detection agent is Ab-3.

90. The method of any one of claims 87-89, wherein the substrate comprises assay beads and helper beads.

91. The method of claim 90, wherein the substrate comprises a greater number of helper beads than assay beads.

92. The method of claim 90, wherein a ratio of helper beads to assay beads is about 1:5 to about 5:1, optionally about 1:1 to about 2:1, e.g., about 1:1, about 1.5:1, or about 2:1.

93. The method of any one of claims 87-92, wherein the incubating step of ii) occurs for about 10-150 minutes and the incubating step of iii) occurs for about 1-30 minutes.

94. The method of any one of claims 87-93, wherein the incubating step of ii) occurs for about 35 minutes and the incubating step of iii) occurs for about 5 minutes, wherein the incubating step of ii) occurs for about 75 minutes and the incubating step of iii) occurs for about 5 minutes, wherein the incubating step of ii) occurs for about 120 minutes and the incubating step of iii) occurs for about 30 minutes, or wherein the incubating step of ii) occurs for about 120 minutes and the incubating step of iii) occurs for about 5 minutes.

95. The method of claim 94, wherein the incubating step of ii) occurs for about 120 minutes and the incubating step of iii) occurs for about 5 minutes.

96. Use of an assay in determining the concentration of frataxin in a biofluid from a subject, wherein the assay comprises a capture agent and a detection agent, wherein optionally the capture agent and the detection agent are each selected from Ab-1, Ab-2, Ab-3, Ab-4, Ab-5, and Ab-6; wherein the limit of detection of frataxin of the assay is about 2.0 pg/mL or less.

97. The use of claim 96, further comprising selecting a therapy capable of increasing frataxin concentration in the subject, wherein the therapy optionally comprises a frataxin gene therapy.

98. The use of claim 97, further comprising determining the efficacy of the therapy, wherein the therapy is effective if the frataxin concentration in a biofluid of the subject is increased following administration of the therapy.

99. The method of any one of claims 1-25, wherein the method further comprises a neuroimaging assessment of one or more regions of a subject's central nervous system.

100. The method of claim 99, wherein the neuroimaging assessment comprises magnetic resonance imaging (MRI) or spectroscopy (MRS), position emission tomography (PET), computed tomography (CT), ultrasound, or diffusion tensor imaging (DTI).

101. The method of claim 99, wherein the neuroimaging assessment comprises brain morphometry of the cerebellum and brainstem, spinal cord morphometry, brain and spinal cord diffusion, dentate iron content (brain QSM), or spinal cord spectroscopy.

102. The method of any one of claims 99-101, wherein the one or more regions of the subject's central nervous system is selected from the group consisting of lobule V, lobule VI, lobule VII, lobule VIII, crus of cerebellum, posterior lobe of vermis, flocculi bilaterally, cerebellar left tonsil, cervical spinal cord, thoracic spinal cord, cerebellar peduncles, dentate nucleus, and a combination thereof.

103. A method of diagnosing Friedreich's Ataxia in a subject, comprising the steps of:
a) performing an assay on a sample of a biofluid from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin;
b) performing a neuroimaging assessment on the subject; and
c) diagnosing the subject with Friedreich's Ataxia if the concentration of frataxin protein present in the sample is just above, below, or equal to the limit of detection of frataxin and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei.

104. A method of diagnosing Friedreich's Ataxia in a subject suspected of having Friedreich's Ataxia, comprising the steps of:
a) performing an assay on a sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) performing a neuroimaging assessment on the subject suspected of having Friedreich's Ataxia; and
c) performing an assay on a sample of a biofluid from a healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
d) performing a neuroimaging assessment on the healthy subject; and
e) diagnosing the subject suspected of having Friedreich's Ataxia with Friedreich's Ataxia if the concentration of frataxin protein present in the sample from the subject suspected of having Friedreich's Ataxia is at least 2-fold less to at least 2000-fold less than the concentration of frataxin protein present in the sample from the healthy subject and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei in the subject suspected of having Friedreich's Ataxia compared to the healthy subject.

105. A method of diagnosing Friedreich's Ataxia in a subject, comprising the steps of:
a) performing an assay on a first sample of a biofluid from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) performing an assay on a second sample of a biofluid from the subject to assess the concentration of neurofilament light chain present in the sample; and
c) performing a neuroimaging assessment on the subject; and
d) diagnosing the subject with Friedreich's Ataxia if the concentration of frataxin protein present in the first sample is just above, below, or equal to the limit of detection of frataxin, if neurofilament light chain is detected in the second sample at a concentration of at least 20 pg/mL, and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei.

106. A method of diagnosing Friedreich's Ataxia in a subject suspected of having Friedreich's Ataxia, comprising the steps of:
a) performing an assay on a first sample of a biofluid from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) performing an assay on a second sample of a biofluid from the subject suspected of having Friedreich's Ataxia to assess the concentration of neurofilament light chain present in the sample; and
c) performing a neuroimaging assessment on the subject suspected of having Friedreich's Ataxia; and
d) performing an assay on a first sample of a biofluid from an age-matched healthy subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
e) performing an assay on a second sample of a biofluid from the age-matched healthy subject to assess the concentration of neurofilament light chain present in the sample; and
f) performing a neuroimaging assessment on the age-matched healthy subject; and
g) diagnosing the subject suspected of having Friedreich's Ataxia with Friedreich's Ataxia if the concentration of frataxin protein present in the first sample from the subject suspected of having Friedreich's Ataxia is at least 2-fold less to at least 2000-fold less than the concentration of frataxin protein present in the first sample from the age-matched healthy subject; the concentration of neurofilament light chain present in the second sample from the subject suspected of having Friedreich's Ataxia is greater than the concentration of neurofilament light chain present in the second sample from the age-matched healthy subject; and if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei in the subject suspected of having Friedreich's Ataxia compared to the age-matched healthy subject.

107. The method of claim 105 or claim 106, wherein the first sample and the second sample of a biofluid from the subject suspected of having Friedreich's Ataxia are the same sample or each is a portion of an initial sample, and/or the first sample and the second sample of a biofluid from the healthy subject are the same sample or each is a portion of an initial sample.

108. The method of any one of claims 103-107, wherein the capture agent and/or the detection agent is an anti-frataxin antibody or a frataxin-binding fragment thereof.

109. The method of claim 108, wherein the capture agent comprises an antibody selected from the group consisting of Ab-1, Ab-2, Ab-3, Ab-4, Ab-5, and Ab-6, and the detection agent comprises an antibody selected from the group consisting of Ab-1, Ab-2, Ab-3, Ab-4, Ab-5, and Ab-6.

110. The method of claim 109, wherein the capture agent is Ab-1 and the detection agent is Ab-3.

111. The method of any one of claims 103-110, wherein the biofluid is CSF.

112. The method as in any of claims 103-110, wherein the biofluid is blood or a blood component.

113. The method of claim 112, wherein the blood component is plasma.

114. The method of claim 112, wherein the blood component is serum.

115. The method as in any of claims 103-114, wherein the assay comprises the steps of
i) incubating a substrate with the capture agent;
ii) incubating the substrate with the biofluid sample and the detection agent;
iii) incubating the substrate with a substance capable of engaging with the detection agent and creating a signal, wherein the signal is detectable and quantifiable.

116. The method of claim 115, wherein the signal is a fluorescence, which is visible, detectable, and quantifiable.

117. The method of claim 115 or claim 116, wherein the substrate comprises assay beads and helper beads.

118. The method of claim 117, wherein the assay comprises a greater number of helper beads than assay beads.

119. The method of claim 117, wherein a ratio of helper beads to assay beads is about 1:5 to about 5:1, optionally about 1:1 to about 2:1, e.g., about 1:1, about 1.5:1, or about 2:1.

120. The method of any one of claims 115-119, wherein the incubating step of b) occurs for about 10-150 minutes and the incubating step of c) occurs for about 1-30 minutes.

121. The method of any one of claims 115-120, wherein the incubating step of b) occurs for about 35 minutes and the incubating step of c) occurs for about 5 minutes, wherein the incubating step of b) occurs for about 75 minutes and the incubating step of c) occurs for about 5 minutes, wherein the incubating step of b) occurs for about 120 minutes and the incubating step of c) occurs for about 30 minutes, or wherein the incubating step of b) occurs for about 120 minutes and the incubating step of c) occurs for about 5 minutes.

122. The method of claim 121, wherein the incubating step of b) occurs for about 120 minutes and the incubating step of c) occurs for about 5 minutes.

123. The method of any one of claims 103-122, wherein the limit of detection of frataxin is about 1.5 pg/mL or less, about 1.0 pg/mL or less, or about 0.5 pg/mL or less.

124. The method of claim 104 or claim 106, wherein the subject suspected of having Friedreich's Ataxia has gray matter volume loss of at least 5%, e.g., 5% 10%, 15%, 20%, 25%, 30%, 40%, 50%, 60%, or more than 60%, relative to the healthy subject.

125. The method of claim 104 or claim 106, wherein the subject suspected of having Friedreich's Ataxia has lobular atrophy of at least 5%, e.g., 5%, 10%, 15%, 20%, 25%, 30%, 40%, 50%, 60%, or more than 60%, relative to the healthy subject.

126. The method of claim 104 or claim 106, wherein the subject suspected of having Friedreich's Ataxia has a decreased mean volume in the dentate nucleus of at least 20%, e.g., 20%, 25%, 30%, 35%, 40%, 45%, or more than 45%, relative to the healthy subject.

127. The method of claim 104 or claim 106, wherein the subject suspected of having Friedreich's Ataxia has increased mean diffusivity in cervical spinal cord of at least 15%, e.g., 15%, 20%, 25%, 30%, 35%, 40%, 45%, 50%, 60%, 70%, or more than 70%, relative to the healthy subject.

128. The method of claim 104 or claim 106, wherein the subject suspected of having Friedreich's Ataxia has increased mean diffusivity in superior, middle, and inferior cerebellar peduncles of at least 15%, e.g., 15%, 20%, 25%, 30%, 35%, 40%, 45%, 50%, 60%, 70%, or more than 70%, relative to the healthy subject.

129. The method of claim 104 or claim 106, wherein the subject suspected of having Friedreich's Ataxia has increased radial diffusivity in cervical spinal cord of at least 30%, e.g., 30%, 35%, 40%, 45%, 50%, 55%, 60%, 65%, 70%, 75%, or more than 75%, relative to the healthy subject.

130. The method of claim 104 or claim 106, wherein the subject suspected of having Friedreich's Ataxia has increased radial diffusivity in superior, middle, and inferior cerebellar peduncles of at least 30%, e.g., 30%, 35%, 40%, 45%, 50%, 55%, 60%, 65%, 70%, 75%, or more than 75%, relative to the healthy subject.

131. The method of claim 104 or claim 106, wherein the subject suspected of having Friedreich's Ataxia has increased axial diffusivity in cervical spinal cord of at least 5%, e.g., 5%, 10%, 15%, 20%, 25%, or more than 25%, relative to the healthy subject.

132. The method of claim 104 or claim 106, wherein the subject suspected of having Friedreich's Ataxia has increased axial diffusivity in superior, middle, and inferior cerebellar peduncles of at least 5%, e.g., 5%, 10%, 15%, 20%, 25%, or more than 25%, relative to the healthy subject.

133. The method of claim 104 or claim 106, wherein the subject suspected of having Friedreich's Ataxia has reduced fractional anisotropy of white matter of at least 15%, e.g., 15%, 20%, 25%, 30%, 35%, 40%, 45%, 50%, 60%, 70%, or more than 70%, relative to the healthy subject.

134. The method of claim 104 or claim 106, wherein the thoracic spinal cord cross-sectional area of the subject suspected of having Friedreich's Ataxia is reduced by at least 5%, e.g., 5%, 10%, 15%, 20%, 25%, 30%, 35%, 40%, 50%, 60%, or more than 60%, relative to that of the healthy subject.

135. The method of claim 104 or claim 106, wherein the cervical spinal cord cross-sectional area of the subject suspected of having Friedreich's Ataxia is reduced by at least 5%, e.g., 5%, 10%, 15%, 20%, 25%, 30%, 35%, 40%, 50%, 60%, or more than 60%, relative to that of the healthy subject.

136. The method of claim 104 or claim 106, wherein the thoracic spinal cord volume of the subject suspected of having Friedreich's Ataxia is reduced by 5%, e.g., 5%, 10%, 15%, 20%, 25%, 30%, 35%, 40%, 50%, 60%, or more than 60%, relative to that of the healthy subject.

137. The method of claim 104 or claim 106, wherein the cervical spinal cord volume of the subject suspected of having Friedreich's Ataxia is reduced by at least 5%, e.g., 5%, 10%, 15%, 20%, 25%, 30%, 35%, 40%, 50%, 60%, or more than 60%, relative to that of the healthy subject.

138. The method of any one of claims 47-72, further comprising a neuroimaging assessment of one or more regions of the central nervous system of a subject.

139. The method of claim 138, wherein the neuroimaging assessment comprises magnetic resonance imaging (MRI) or spectroscopy (MRS), position emission tomography (PET), computed tomography (CT), ultrasound, or diffusion tensor imaging (DTI).

140. The method of claim 138, wherein the neuroimaging assessment comprises brain morphometry of the cerebellum and brainstem, spinal cord morphometry, brain and spinal cord diffusion, dentate iron content (brain QSM), or spinal cord spectroscopy.

141. The method of any one of claims 138-140, wherein the one or more regions of the subject's central nervous system is selected from the group consisting of lobule V, lobule VI, lobule VII, lobule VIII, crus of cerebellum, posterior lobe of vermis, flocculi bilaterally, cerebellar left tonsil, cervical spinal cord, thoracic spinal cord, cerebellar peduncles, dentate nucleus, and a combination thereof.

142. A method of determining the efficacy of a treatment to increase frataxin levels in a subject having Friedreich's Ataxia, comprising the steps of:
a) determining a baseline concentration of frataxin in a first sample of a biofluid of a subject, wherein the biofluid is optionally cerebrospinal fluid (CSF) or serum;
b) performing a baseline neuroimaging assessment on the subject;
c) treating the subject with a treatment which results in increased frataxin levels in the subject;
d) determining a subsequent concentration of frataxin in a second sample of the biofluid of the subject;
e) performing a subsequent neuroimaging assessment on the subject;
f) comparing the baseline concentration of frataxin to the subsequent concentration of frataxin;
g) comparing the baseline neuroimaging assessment to the subsequent neuroimaging assessment; and
h) determining treatment efficacy, wherein the treatment is effective if the subsequent concentration of frataxin is greater than the baseline concentration and the subsequent neuroimaging assessment shows no significant worsening (e.g., no more than 5% worsening) of atrophy and/or diffusivity compared to the baseline neuroimaging assessment.

143. The method of claim 142, wherein the treatment is effective if the subsequent concentration of frataxin is at least 0.5×-3× greater than the baseline concentration and the subsequent neuroimaging assessment shows no significant worsening (e.g., no more than 5% worsening) of atrophy and/or diffusivity compared to the baseline neuroimaging assessment.

144. The method of claim 142, wherein the treatment is effective if the subsequent concentration of frataxin is at least 3× greater than the baseline concentration and the subsequent neuroimaging assessment shows no significant worsening (e.g., no more than 5% worsening) of atrophy and/or diffusivity compared to the baseline neuroimaging assessment.

145. A method of determining the efficacy of a treatment to increase frataxin levels in a subject having Friedreich's Ataxia, comprising the steps of:
a) determining a baseline concentration of frataxin in a first sample of a biofluid of a subject, wherein the biofluid is optionally cerebrospinal fluid (CSF) or serum;
b) determining a baseline concentration of neurofilament light chain in a second sample of a biofluid of the subject, wherein the biofluid is optionally CSF or serum;
c) performing a baseline neuroimaging assessment on the subject;
d) treating the subject with a treatment which results in increased frataxin levels in the subject;
e) determining a subsequent concentration of frataxin in a third sample of a biofluid of the subject, wherein the biofluid of d) is CSF if the biofluid of a) is CSF, and wherein the biofluid of d) is serum if the biofluid of a) is serum;
f) determining a subsequent concentration of neurofilament light chain in a fourth sample of a biofluid of the subject, wherein the biofluid of e) is CSF if the biofluid of b) is CSF, and wherein the biofluid of e) is serum if the biofluid of b) is serum;
g) performing a subsequent neuroimaging assessment on the subject;
h) comparing the baseline concentration of frataxin to the subsequent concentration of frataxin, comparing the baseline concentration of neurofilament light chain to the subsequent concentration of neurofilament light chain, and comparing the baseline neuroimaging assessment to the subsequent neuroimaging assessment; and
i) determining treatment efficacy, wherein the treatment is effective if the subsequent concentration of frataxin is greater than the baseline concentration of frataxin, the subsequent concentration of neurofilament light chain is not significantly higher than the baseline concentration of neurofilament light chain, and the subsequent neuroimaging assessment shows no significant worsening (e.g., no more than 5% worsening) of atrophy and/or diffusivity compared to the baseline neuroimaging assessment.

146. The method of claim 145, wherein the treatment is effective if the subsequent concentration of frataxin is at least 0.5× greater than the baseline concentration of frataxin, the subsequent concentration of neurofilament light chain is equal to or lower than the baseline concentration of neurofilament light chain, and the subsequent neuroimaging assessment shows no significant worsening (e.g., no more than 5% worsening) of atrophy and/or diffusivity compared to the baseline neuroimaging assessment.

147. The method of any one of claims 142-146, wherein the treatment comprises a frataxin gene therapy.

148. A method of treating Friedreich's Ataxia in a subject, comprising the steps of:
a) determining a baseline concentration of frataxin in a first sample of a biofluid of a subject, wherein the biofluid is optionally cerebrospinal fluid (CSF) or serum;
b) performing a baseline neuroimaging assessment on the subject;
c) treating the subject with a frataxin gene therapy;
d) determining a subsequent concentration of frataxin in a second sample of the biofluid of the subject;
e) performing a subsequent neuroimaging assessment on the subject; and
f) comparing the baseline concentration of frataxin to the subsequent concentration of frataxin and comparing the baseline neuroimaging assessment to the subsequent neuroimaging assessment;
wherein the frataxin gene therapy is effective if the subsequent concentration of frataxin is greater than the baseline concentration of frataxin and the subsequent neuroimaging assessment shows no significant worsening (e.g., no more than 5% worsening) of atrophy and/or diffusivity compared to the baseline neuroimaging assessment.

149. A method of treating Friedreich's Ataxia in a subject, comprising the steps of:
a) determining a baseline concentration of frataxin in a first sample of a biofluid of a subject and determining a baseline concentration of neurofilament light chain in a second sample of a biofluid of the subject, wherein the biofluid is optionally cerebrospinal fluid (CSF) or serum and wherein the first sample and the second sample are optionally the same sample;
b) performing a baseline neuroimaging assessment on the subject;
c) treating the subject with a frataxin gene therapy;
d) determining a subsequent concentration of frataxin in a third sample of a biofluid of the subject and determining a subsequent concentration of neurofilament light chain in a fourth sample of a biofluid in the subject, wherein the biofluid of d) is CSF if the biofluid of a) is CSF, and wherein the biofluid of d) is serum if the biofluid of a) is serum, and wherein the third sample and the fourth sample are optionally the same sample;
e) performing a subsequent neuroimaging assessment on the subject; and
comparing the baseline concentration of frataxin to the subsequent concentration of frataxin, comparing the baseline concentration of neurofilament light chain to the subsequent concentration of neurofilament light chain, and comparing the baseline neuroimaging assessment to the subsequent neuroimaging assessment;
wherein the frataxin gene therapy is effective if the subsequent concentration of frataxin is greater than the baseline concentration, the subsequent concentration of neurofilament light chain is not significantly higher than, is lower than, or is equal to the baseline concentration of neurofilament light chain, and the subsequent neuroimaging assessment shows no significant worsening (e.g., no more than 5% worsening) of atrophy and/or diffusivity compared to the baseline neuroimaging assessment.

150. The method of claim 148 or claim 149, wherein if the frataxin gene therapy is not effective, the subject is administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

151. The method of claim 148 or claim 149, wherein if the frataxin gene therapy is not effective, the subject is administered a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

152. The method of claim 148 or claim 149, wherein if the frataxin gene therapy is effective, the subject is administered a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

153. The method of claim 148 or claim 149, wherein if the frataxin gene therapy is effective, the subject is administered a treatment comprising managing one or more symptoms of Friedreich's Ataxia comprising occupational therapy, speech therapy, orthopedic care, bracing, surgery, treatment for heart disease, an antioxidant, treatment to lower blood sugar, and/or physical therapy.

154. The method of any one of claims 150-152, wherein the second therapy comprises omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia), acetyl-L-carnitine (ALCAR), rosuvastatin, an incretin analog, indole-3-propionic acid, and/or erythropoietin or a modified (e.g., carbamylated) form thereof.

155. The method of claim 150-152, wherein the second therapy comprises omaveloxolone, wherein the low dose of omaveloxolone is about 5 mg/day, and wherein the high dose of omaveloxolone is about 300 mg/day.

156. The method of any one of claims 142-155, wherein determining a baseline or subsequent concentration of frataxin comprises
i) incubating a substrate with a capture agent;
ii) incubating the substrate with a sample and a detection agent;
iii) incubating the substrate with a substance capable of engaging with the detection agent and creating a signal, wherein the signal is detectable and quantifiable.

157. The method of claim 156, wherein the capture agent comprises an antibody selected from the group consisting of Ab-1, Ab-2, Ab-3, Ab-4, Ab-5, and Ab-6; and the detection agent comprises an antibody selected from the group consisting of Ab-1, Ab-2, Ab-3, Ab-4, Ab-5, and Ab-6.

158. The method of claim 157, wherein the capture agent is Ab-1 and the detection agent is Ab-3.

159. The method of any one of claims 156-158, wherein the substrate comprises assay beads and helper beads.

160. The method of claim 159, wherein the assay comprises a greater number of helper beads than assay beads.

161. The method of claim 159, wherein a ratio of helper beads to assay beads is about 1:5 to about 5:1, optionally about 1:1 to about 2:1, e.g., about 1:1, about 1.5:1, or about 2:1.

162. The method of any one of claims 156-161, wherein the incubating step of ii) occurs for about 10-150 minutes and the incubating step of iii) occurs for about 1-30 minutes.

163. The method of any one of claims 156-162, wherein the incubating step of ii) occurs for about 35 minutes and the incubating step of iii) occurs for about 5 minutes, wherein the incubating step of ii) occurs for about 75 minutes and the incubating step of iii) occurs for about 5 minutes, wherein the incubating step of ii) occurs for about 120 minutes and the incubating step of iii) occurs for about 30 minutes, or wherein the incubating step of ii) occurs for about 120 minutes and the incubating step of iii) occurs for about 5 minutes.

164. The method of claim 163, wherein the incubating step of ii) occurs for about 120 minutes and the incubating step of iii) occurs for about 5 minutes.

165. The method of any one of claims 47-94 or 142-164, wherein the therapy or treatment comprises a nucleic acid construct comprising a fragment of any of SEQ ID NOs: 1-3, optionally nucleotides 32-664 of SEQ ID NO: 1.

166. The method of any one of claims 47-94 or 142-165, wherein the therapy or treatment comprises a nucleic acid construct encoding any of SEQ ID NOs: 4-6, optionally SEQ ID NO: 4.

167. The method of any one of claims 47-94 or 142-166, wherein the therapy or treatment comprises a nucleic acid construct comprising any of SEQ ID NOs: 7-10.

168. The method of any one of claims 47-94 or 142-167, wherein the therapy or treatment comprises a nucleic acid construct comprising any of SEQ ID NOs: 11-14.

169. The method of any one of claims 47-94 or 142-168, wherein the therapy or treatment comprises an AAV comprising an AAV capsid and a viral genome, wherein the viral genome comprises a nucleic acid construct encoding any of SEQ ID NOs 4-6, optionally SEQ ID NO: 4.

170. The method of claim 169, wherein the nucleic acid construct comprises any of SEQ ID NOs: 7-10.

171. The method of claim 169 or claim 170, wherein the nucleic acid construct comprises any of SEQ ID NOs: 11-14.

172. The method of any one of claims 47-94 or 142-171, wherein the therapy or treatment comprises an AAV capsid comprising a sequence that is at least 99% identical to SEQ ID NO: 15 and/or encoded by SEQ ID NO: 16.

173. The method of any one of claims 47-94 or 142-172, wherein the therapy or treatment is formulated to comprise sodium chloride, sodium phosphate, potassium chloride, potassium phosphate and poloxamer 188; wherein optionally the therapy or treatment is formulated to comprise 192 mM sodium chloride, 10 mM sodium phosphate, 2.7 mM potassium chloride, 2 mM potassium phosphate and 0.001% poloxamer 188 (v/v), wherein the pH of the formulation is 7.4.

174. The use of any one of claims 96-98, wherein the therapy comprises a nucleic acid construct comprising a fragment of any of SEQ ID NOs: 1-3.

175. The use of claim 174, wherein the therapy comprises a nucleic acid construct encoding any of SEQ ID NOs: 4-6, optionally SEQ ID NO: 4.

176. The use of claim 174 or claim 175, wherein the therapy comprises a nucleic acid construct comprising any of SEQ ID NOs: 7-10.

177. The use of any one of claims 174-176, wherein the therapy comprises a nucleic acid construct comprising any of SEQ ID NOs: 11-14.

178. The use of claim any one of claims 174-177, wherein the therapy comprises an AAV comprising an AAV capsid and a viral genome, wherein the viral genome comprises a nucleic acid construct encoding any of SEQ ID NOs 4-6, optionally SEQ ID NO: 4.

179. The use of claim 178, wherein the nucleic acid construct comprises any of SEQ ID NOs: 7-10.

180. The use of claim 178 or claim 179, wherein the nucleic acid construct comprises any of SEQ ID NOs: 11-14.

181. The use of any of claims 174-180, wherein the therapy comprises an AAV capsid comprising a sequence that is at least 99% identical to SEQ ID NO: 15 and/or encoded by SEQ ID NO: 16.

182. The use of any of claims 174-181, wherein the therapy is formulated to comprise sodium chloride, sodium phosphate, potassium chloride, potassium phosphate and poloxamer 188; wherein optionally the therapy is formulated to comprise 192 mM sodium chloride, 10 mM sodium phosphate, 2.7 mM potassium chloride, 2 mM potassium phosphate and 0.001% poloxamer 188 (v/v), wherein the pH of the formulation is 7.4.

183. A method of diagnosing Friedreich's Ataxia in a subject, comprising the steps of:
a) performing an assay on a sample, e.g., a serum sample, from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) diagnosing the subject with Friedreich's Ataxia if the concentration of frataxin protein present in the sample is below 10 pg/mL.

184. The method of claim 183, wherein the concentration of frataxin protein present in the sample is below 5 pg/mL.

185. The method of claim 183 or claim 184, wherein the concentration of frataxin protein present in the sample is below 2 pg/mL.

186. The method of any one of claims 183-185, wherein the concentration of frataxin protein present in the sample is below 1 pg/mL.

187. The method of any one of claims 183-186, wherein the subject is diagnosed with Friedreich's Ataxia if the frataxin protein cannot be detected in the sample from the subject (e.g., below level of detection).

188. A method of diagnosing Friedreich's Ataxia in a subject, comprising the steps of:
a) performing an assay on a sample, e.g. CSF sample, from the subject to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) diagnosing the subject with Friedreich's Ataxia if the concentration of frataxin protein present in the sample is below 5 pg/mL.

189. The method of claim 188, wherein the concentration of frataxin protein present in the sample is below 2 pg/mL.

190. The method of claim 188 or claim 189, wherein the concentration of frataxin protein present in the sample is below 1 pg/mL.

191. The method of any one of claims 188-190, wherein the subject is diagnosed with Friedreich's Ataxia if the frataxin protein cannot be detected in the sample from the subject (e.g., below level of frataxin detection).

192. A method of diagnosing Friedreich's Ataxia in a subject suspected of having Friedreich's Ataxia, comprising the steps of:
a) performing an assay on a first sample of serum from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) performing an assay on a second sample of serum from the subject suspected of having Friedreich's Ataxia to assess the concentration of neurofilament light chain present in the sample; and
c) diagnosing the subject suspected of having Friedreich's Ataxia with Friedreich's Ataxia if the concentration of frataxin protein present in the first sample of serum is below 10 pg/mL and the concentration of neurofilament light chain present in the second sample of serum is at least 20 pg/mL.

193. The method of claim 192, concentration of frataxin protein present in the first sample of serum is below 5 pg/mL.

194. The method of claim 192 or claim 193, wherein the concentration of frataxin protein present in the first sample of serum is below 2 pg/mL.

195. The method of any one of claims 192-194, wherein the concentration of frataxin protein present in the first sample of serum is below 1 pg/mL.

196. The method of any one of claims 192-195, wherein frataxin protein cannot be detected from the first sample of serum (e.g., below level of frataxin detection).

197. A method of diagnosing Friedreich's Ataxia in a subject suspected of having Friedreich's Ataxia, comprising the steps of:
a) performing an assay on a first sample of CSF from the subject suspected of having Friedreich's Ataxia to assess the concentration of frataxin protein present in the sample; wherein a limit of detection of frataxin of the assay is about 2.0 pg/mL or less; wherein the assay comprises a capture agent that specifically binds to frataxin or a fragment of frataxin, and a detection agent that specifically binds to frataxin or a fragment of frataxin; and
b) performing an assay on a second sample of CSF from the subject suspected of having Friedreich's Ataxia to assess the concentration of neurofilament light chain present in the sample; and
c) diagnosing the subject suspected of having Friedreich's Ataxia with Friedreich's Ataxia if the concentration of frataxin protein present in the first sample of CSF is below 5 pg/mL and the concentration of neurofilament light chain present in the second sample is at least 1200 pg/mL.

198. The method of claim 197, wherein the concentration of frataxin protein present in the first sample of CSF is below 2 pg/mL.

199. The method of claim 197 or claim 198, wherein the concentration of frataxin protein present in the first sample of serum is below 1 pg/mL.

200. The method of any one of claims 197-199, wherein frataxin protein cannot be detected from the first sample of CSF (e.g., below level of frataxin detection).

201. The method of any one of claims 192-196, wherein the first sample and the second sample from the subject suspected of having Friedreich's Ataxia are the same sample or each is a portion of an initial sample.

202. The method of any one of claims 183-201, further comprising the steps of:
a) performing a neuroimaging assessment on the subject suspected of having Friedreich's Ataxia; and
b) performing a neuroimaging assessment on a healthy subject; and
c) diagnosing the subject suspected of having Friedreich's Ataxia with Friedreich's Ataxia if the neuroimaging assessment shows one or more of: gray matter volume loss, lobular atrophy, spinal cord atrophy, mean volume loss of dentate nucleus, reduced fractional anisotropy in white matter, increased mean diffusivity in white matter, increased axial diffusivity in white matter, increased radial diffusivity in white matter, increased mean diffusivity in cervical spinal cord, increased axial diffusivity in cervical spinal cord, increased radial diffusivity in cervical spinal cord, increased mean diffusivity in cerebellar peduncles, increased axial diffusivity in cerebellar peduncles, increased radial diffusivity in cerebellar peduncles, accumulation of iron in dentate nuclei, and increased admixture of iron, copper, and zinc in dentate nuclei in the subject suspected of having Friedreich's Ataxia compared to the healthy subject.

203. The method of any one of claims 183-202, wherein the capture agent and/or the detection agent is an anti-frataxin antibody or a frataxin-binding fragment thereof.

204. The method of claim 203, wherein the capture agent comprises an antibody selected from the group consisting of Ab-1, Ab-2, Ab-3, Ab-4, Ab-5, and Ab-6, and the detection agent comprises an antibody selected from the group consisting of Ab-1, Ab-2, Ab-3, Ab-4, Ab-5, and Ab-6.

205. The method of claim 204, wherein the capture agent is Ab-1 and the detection agent is Ab-3.

206. The method of any of claims 183-205, wherein the assay comprises the steps of
a) incubating a substrate with the capture agent;
b) incubating the substrate with the biofluid sample and the detection agent;
c) incubating the substrate with a substance capable of engaging with the detection agent and creating a signal, wherein the signal is detectable and quantifiable.

207. The method of claim 206, wherein the signal is a fluorescence, which is visible, detectable, and quantifiable.

208. The method of claim 206 or claim 207, wherein the substrate comprises assay beads and helper beads.

209. The method of claim 208, wherein the assay comprises a greater number of helper beads than assay beads.

210. The method of claim 208, wherein a ratio of helper beads to assay beads is about 1:5 to about 5:1, optionally about 1:1 to about 2:1, e.g., about 1:1, about 1.5:1, or about 2:1.

211. The method of any one of claims 206-210, wherein the incubating step of b) occurs for about 10-150 minutes and the incubating step of c) occurs for about 1-30 minutes.

212. The method of any one of claims 206-211, wherein the incubating step of b) occurs for about 35 minutes and the incubating step of c) occurs for about 5 minutes, wherein the incubating step of b) occurs for about 75 minutes and the incubating step of c) occurs for about 5 minutes, wherein the incubating step of b) occurs for about 120 minutes and the incubating step of c) occurs for about 30 minutes, or wherein the incubating step of b) occurs for about 120 minutes and the incubating step of c) occurs for about 5 minutes.

213. The method of claim 212, wherein the incubating step of b) occurs for about 120 minutes and the incubating step of c) occurs for about 5 minutes.

214. The method of any one of claims 183-213, wherein the limit of detection of frataxin is about 1.5 pg/mL or less, about 1.0 pg/mL or less, or about 0.5 pg/mL or less.

215. The method of claim 202, wherein the subject suspected of having Friedreich's Ataxia has gray matter volume loss of at least 5%, e.g., 5%, 10%, 15%, 20%, 25%, 30%, 40%, 50%, 60%, or more than 60%, relative to the healthy subject.

216. The method of claim 202, wherein the subject suspected of having Friedreich's Ataxia has lobular atrophy of at least 5%, e.g., 5%, 10%, 15%, 20%, 25%, 30%, 40%, 50%, 60%, or more than 60%, relative to the healthy subject.

217. The method of claim 202, wherein the subject suspected of having Friedreich's Ataxia has a decreased mean volume in the dentate nucleus of at least 20%, e.g., 20%, 25%, 30%, 35%, 40%, 45%, or more than 45%, relative to the healthy subject.

218. The method of claim 202, wherein the subject suspected of having Friedreich's Ataxia has increased mean diffusivity in cervical spinal cord of at least 15%, e.g., 15%, 20%, 25%, 30%, 35%, 40%, 45%, 50%, 60%, 70%, or more than 70%, relative to the healthy subject.

219. The method of claim 202, wherein the subject suspected of having Friedreich's Ataxia has increased mean diffusivity in superior, middle, and inferior cerebellar peduncles of at least 15%, e.g., 15%, 20%, 25%, 30%, 35%, 40%, 45%, 50%, 60%, 70%, or more than 70%, relative to the healthy subject.

220. The method of claim 202, wherein the subject suspected of having Friedreich's Ataxia has increased radial diffusivity in cervical spinal cord of at least 30%, e.g., 30%, 35%, 40%, 45%, 50%, 55%, 60%, 65%, 70%, 75%, or more than 75%, relative to the healthy subject.

221. The method of claim 202, wherein the subject suspected of having Friedreich's Ataxia has increased radial diffusivity in superior, middle, and inferior cerebellar peduncles of at least 30%, e.g., 30%, 35%, 40%, 45%, 50%, 55%, 60%, 65%, 70%, 75%, or more than 75%, relative to the healthy subject.

222. The method of claim 202, wherein the subject suspected of having Friedreich's Ataxia has increased axial diffusivity in cervical spinal cord of at least 5%, e.g., 5%, 10%, 15%, 20%, 25%, or more than 25%, relative to the healthy subject.

223. The method of claim 202, wherein the subject suspected of having Friedreich's Ataxia has increased axial diffusivity in superior, middle, and inferior cerebellar peduncles of at least 5%, e.g., 5%, 10%, 15%, 20%, 25%, or more than 25%, relative to the healthy subject.

224. The method of claim 202, wherein the subject suspected of having Friedreich's Ataxia has reduced fractional anisotropy of white matter of at least 15%, e.g., 15%, 20%, 25%, 30%, 35%, 40%, 45%, 50%, 60%, 70%, or more than 70%, relative to the healthy subject.

225. The method of claim 202, wherein a thoracic spinal cord cross-sectional area of the subject suspected of having Friedreich's Ataxia is reduced by at least 5%, e.g., 5%, 10%, 15%, 20%, 25%, 30%, 40%, 50%, 60%, or more than 60%, relative to that of the healthy subject.

226. The method of claim 202, wherein a cervical spinal cord cross-sectional area of the subject suspected of having Friedreich's Ataxia is reduced by at least 5%, e.g., 5%, 10%, 15%, 20%, 25%, 30%, 35%, 40%, 50%, 60%, or more than 60%, relative to that of the healthy subject.

227. The method of claim 202, wherein a thoracic spinal cord volume of the subject suspected of having Friedreich's Ataxia is reduced by 5%, e.g., 5%, 10%, 15%, 20%, 25%, 30%, 35%, 40%, 50%, 60%, or more than 60%, relative to that of the healthy subject.

228. The method of claim 202, wherein a cervical spinal cord volume of the subject suspected of having Friedreich's Ataxia is reduced by at least 5%, e.g., 5%, 10%, 15%, 20%, 25%, 30%, 35%, 40%, 50%, 60%, or more than 60%, relative to that of the healthy subject.

229. The method of any one of claims 183-201, further comprising a neuroimaging assessment of one or more regions of the central nervous system of a subject.

230. The method of claim 229, wherein the neuroimaging assessment comprises magnetic resonance imaging (MRI) or spectroscopy (MRS), position emission tomography (PET), computed tomography (CT), ultrasound, and/or diffusion tensor imaging (DTI).

231. The method of claim 229, wherein the neuroimaging assessment comprises brain morphometry of the cerebellum and brainstem, spinal cord morphometry, brain and spinal cord diffusion, dentate iron content (brain QSM), and/or spinal cord spectroscopy.

232. The method of any one of claims 229-231, wherein the one or more regions of the subject's central nervous system is selected from the group consisting of lobule V, lobule VI, lobule VII, lobule VIII, crus of cerebellum, posterior lobe of vermis, flocculi bilaterally, cerebellar left tonsil, cervical spinal cord, thoracic spinal cord, cerebellar peduncles, dentate nucleus, and a combination thereof.

233. A method of treating Friedreich's Ataxia in a subject, comprising the steps of:
a) determining the concentration of frataxin in a serum sample from the subject using an assay having a limit of detection of frataxin of about 2.0 pg/mL or less; wherein the assay comprises a capture agent that specifically binds to frataxin or a fragment of frataxin, and a detection agent that specifically binds to frataxin or a fragment of frataxin; and
b) administering a suitable treatment to the subject if the concentration of frataxin protein present in the serum sample is below 10 pg/mL.

234. The method of claim 233, wherein the concentration of frataxin protein present in the serum sample is below 5 pg/mL.

235. The method of claim 233 or claim 234, wherein the concentration of frataxin protein present in the serum sample is below 2 pg/mL.

236. The method of any one of claims 233-235, wherein the concentration of frataxin protein present in the serum sample is below 1 pg/mL.

237. The method of any one of claims 233-236, wherein frataxin protein cannot be detected in the serum sample (e.g., below level of detection).

238. The method of any one of claims 233-237, further comprising determining the concentration of neurofilament light chain in a serum sample from the subject, wherein the sample is optionally the same serum sample or part of the same initial serum sample as the sample from which frataxin concentration is determined, and wherein a suitable treatment is administered to the subject if the concentration of frataxin in the serum sample is below 10 pg/mL, below 5 pg/mL, below 2 pg/mL, below 1 pg/mL, or less (e.g., below level of detection), and if the concentration of neurofilament light chain in the serum sample is at least 20 pg/mL.

239. A method of treating Friedreich's Ataxia in a subject, comprising the steps of:
a) determining the concentration of frataxin in a CSF sample from the subject using an assay having a limit of detection of frataxin of about 2.0 pg/mL or less; wherein the assay comprises a capture agent that specifically binds to frataxin or a fragment of frataxin, and a detection agent that specifically binds to frataxin or a fragment of frataxin; and
b) administering a suitable treatment to the subject if the concentration of frataxin protein present in the CSF sample is below 5 pg/mL.

240. The method of claim 239, wherein the concentration of frataxin protein present in the CSF sample is below 2 pg/mL.

241. The method of claim 239 or claim 240, wherein the concentration of frataxin protein present in the CSF sample is below 1 pg/mL.

242. The method of any one of claims 239-241, wherein frataxin protein cannot be detected in the CSF sample (e.g., below level of detection).

243. The method of any one of claims 239-242, further comprising determining the concentration of neurofilament light chain in a CSF sample from the subject, wherein the sample is optionally the same CSF sample or part of the same initial CSF sample as the sample from which frataxin concentration is determined, and wherein a suitable treatment is administered to the subject if the concentration of frataxin in the CSF sample is below 5 pg/mL, below 2 pg/mL, below 1 pg/mL, or less (e.g., below level of detection), and if the concentration of neurofilament light chain in the CSF sample is at least 1200 pg/mL.

244. The method of any one of claims 233-243, wherein the suitable treatment comprises omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia, acetyl-L-carnitine (ALCAR), rosuvastatin, an incretin analog, indole-3-propionic acid, and/or erythropoietin or a modified (e.g., carbamylated) form thereof.

245. The method of any one of claims 233-243, wherein the suitable treatment comprises frataxin gene therapy and omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia, acetyl-L-carnitine (ALCAR), rosuvastatin, an incretin analog, indole-3-propionic acid, and/or erythropoietin or a modified (e.g., carbamylated) form thereof.

246. A method of determining the efficacy of a treatment to increase frataxin levels in a subject having Friedreich's Ataxia using an assay having a limit of detection of frataxin of about 2.0 pg/mL or less, wherein the assay comprises a capture agent that specifically binds to frataxin or a fragment of frataxin, and a detection agent that specifically binds to frataxin or a fragment of frataxin, comprising the steps of:
a) determining a baseline concentration of frataxin in a first serum sample from the subject;
b) treating the subject with a treatment which results in increased frataxin levels in the subject;
c) determining a subsequent concentration of frataxin in a second serum sample from the subject;
d) comparing the baseline concentration of frataxin in the first serum sample to the subsequent concentration of frataxin in the second serum sample;
e) determining treatment efficacy, wherein the treatment is effective if the subsequent concentration of frataxin in the second serum sample is greater than the baseline concentration in the first serum sample.

247. The method of claim 246, wherein the baseline concentration of frataxin in a first serum sample from the subject is below 10 pg/mL.

248. The method of claim 246 or claim 247, wherein the baseline concentration of frataxin in a first serum sample from the subject is below 5 pg/mL.

249. The method of any one of claims 246-248, wherein the baseline concentration of frataxin in a first serum sample from the subject is below 2 pg/mL.

250. The method of any one of claims 246-249, wherein the baseline concentration of frataxin in a first serum sample from the subject is below 1 pg/mL.

251. The method of any one of claims 246-250, wherein the baseline concentration of frataxin in a first serum sample from the subject is about 0.5-1.5 pg/mL.

252. A method of determining the efficacy of a treatment to increase frataxin levels in a subject having Friedreich's Ataxia using an assay having a limit of detection of frataxin of about 2.0 pg/mL or less, wherein the assay comprises a capture agent that specifically binds to frataxin or a fragment of frataxin, and a detection agent that specifically binds to frataxin or a fragment of frataxin, comprising the steps of:
a) determining a baseline concentration of frataxin in a first CSF sample from the subject;
b) treating the subject with a treatment which results in increased frataxin levels in the subject;
c) determining a subsequent concentration of frataxin in a second CSF sample from the subject;
d) comparing the baseline concentration of frataxin in the first CSF sample to the subsequent concentration of frataxin in the second CSF sample;
e) determining treatment efficacy, wherein the treatment is effective if the subsequent concentration of frataxin in the second CSF sample is greater than the baseline concentration in the first CSF sample.

253. The method of claim 252, wherein the baseline concentration of frataxin in a first CSF sample from the subject is below 5 pg/mL.

254. The method of claim 252 or claim 253, wherein the baseline concentration of frataxin in a first CSF sample from the subject is below 2 pg/mL.

255. The method of any one of claims 252-254, wherein the baseline concentration of frataxin in a first CSF sample from the subject is below 1 pg/mL.

256. The method of any one of claims 252-255, wherein the baseline concentration of frataxin in a first CSF sample from the subject is about 0.5-1.5 pg/mL.

257. The method of any one of claims 246-256, wherein the treatment is effective if the subsequent concentration of frataxin is at least 0.5×-3× greater than the baseline concentration.

258. The method of any one of claims 246-257, wherein the treatment is effective if the subsequent concentration of frataxin is at least 3× greater than the baseline concentration.

259. The method of any one of claims 246-258, further comprising the steps of:
a) performing a baseline neuroimaging assessment on the subject prior to treating the subject with a treatment which results in increased frataxin levels in the subject;
b) performing a subsequent neuroimaging assessment on the subject after treating the subject with a treatment which results in increased frataxin levels in the subject;
c) comparing the baseline neuroimaging assessment to the subsequent neuroimaging assessment; and
d) determining treatment efficacy, wherein the treatment is effective if the subsequent neuroimaging assessment shows no significant worsening (e.g., no more than 5% worsening) of atrophy and/or diffusivity compared to the baseline neuroimaging assessment.

260. A method of determining the efficacy of a treatment to increase frataxin levels in a subject having Friedreich's Ataxia using an assay having a limit of detection of frataxin of about 2.0 pg/mL or less, wherein the assay comprises a capture agent that specifically binds to frataxin or a fragment of frataxin, and a detection agent that specifically binds to frataxin or a fragment of frataxin, comprising the steps of:
a) determining a baseline concentration of frataxin in a first serum sample of the subject;
b) determining a baseline concentration of neurofilament light chain in a second serum sample of the subject; wherein the second serum sample is optionally the same sample as the first serum sample;
c) treating the subject with a treatment which results in increased frataxin levels in the subject;
d) determining a subsequent concentration of frataxin in a third serum sample of the subject;
e) determining a subsequent concentration of neurofilament light chain in a fourth serum sample of the subject; wherein the fourth serum sample is optionally the same sample as the third serum sample;
f) comparing the baseline concentration of frataxin to the subsequent concentration of frataxin, comparing the baseline concentration of neurofilament light chain to the subsequent concentration of neurofilament light chain; and
g) determining treatment efficacy, wherein the treatment is effective if the subsequent concentration of frataxin is greater than the baseline concentration of frataxin and the subsequent concentration of neurofilament light chain is lower than or equal to the baseline concentration of neurofilament light chain.

261. The method of claim 260, wherein the baseline concentration of frataxin in a first serum sample from the subject is below 10 pg/mL.

262. The method of claim 260 or claim 261, wherein the baseline concentration of frataxin in a first serum sample from the subject is below 5 pg/mL.

263. The method of any one of claims 260-262, wherein the baseline concentration of frataxin in a first serum sample from the subject is below 2 pg/mL.

264. The method of any one of claims 260-263, wherein the baseline concentration of frataxin in a first serum sample from the subject is below 1 pg/mL.

265. The method of any one of claims 260-264, wherein the baseline concentration of frataxin in a first serum sample from the subject is about 0.5-1.5 pg/mL.

266. The method of any one of claims 260-265, wherein the baseline concentration of neurofilament light chain is at least 20 pg/mL.

267. A method of determining the efficacy of a treatment to increase frataxin levels in a subject having Friedreich's Ataxia using an assay having a limit of detection of frataxin of about 2.0 pg/mL or less, wherein the assay comprises a capture agent that specifically binds to frataxin or a fragment of frataxin, and a detection agent that specifically binds to frataxin or a fragment of frataxin, comprising the steps of:
a) determining a baseline concentration of frataxin in a first CSF sample of the subject;
b) determining a baseline concentration of neurofilament light chain in a second CSF sample of the subject; wherein the second CSF sample is optionally the same sample as the first CSF sample;
c) treating the subject with a treatment which results in increased frataxin levels in the subject;
d) determining a subsequent concentration of frataxin in a third CSF sample of the subject;
e) determining a subsequent concentration of neurofilament light chain in a fourth CSF sample of the subject; wherein the four CSF sample is optionally the same sample as the third CSF sample;
f) comparing the baseline concentration of frataxin to the subsequent concentration of frataxin, comparing the baseline concentration of neurofilament light chain to the subsequent concentration of neurofilament light chain; and
g) determining treatment efficacy, wherein the treatment is effective if the subsequent concentration of frataxin is greater than the baseline concentration of frataxin and the subsequent concentration of neurofilament light chain is lower than or equal to the baseline concentration of neurofilament light chain.

268. The method of claim 267, wherein the baseline concentration of frataxin in a first CSF sample from the subject is below 5 pg/mL.

269. The method of claim 267 or claim 268, wherein the baseline concentration of frataxin in a first CSF sample from the subject is below 2 pg/mL.

270. The method of any one of claims 267-269, wherein the baseline concentration of frataxin in a first CSF sample from the subject is below 1 pg/mL.

271. The method of any one of claims 267-270, wherein the baseline concentration of frataxin in a first CSF sample from the subject is about 0.5-1.5 pg/mL.

272. The method of any one of claims 267-271, wherein the baseline concentration of neurofilament light chain is at least 1200 pg/mL.

273. The method of any one of claims 260-272, further comprising the steps of:
a) performing a baseline neuroimaging assessment on the subject prior to treating the subject with a treatment which results in increased frataxin levels in the subject;
b) performing a subsequent neuroimaging assessment on the subject after treating the subject with a treatment which results in increased frataxin levels in the subject;
c) comparing the baseline neuroimaging assessment to the subsequent neuroimaging assessment; and
d) determining treatment efficacy, wherein the treatment is effective if the subsequent neuroimaging assessment shows no significant worsening (e.g., no more than 5% worsening) of atrophy and/or diffusivity compared to the baseline neuroimaging assessment.

274. The method of any one of claims 260-273, wherein the treatment is effective if the subsequent concentration of frataxin is at least 0.5×-3× greater than the baseline concentration of frataxin, the subsequent concentration of neurofilament light chain is lower than or equal to the baseline concentration of neurofilament light chain, and the subsequent neuroimaging assessment shows no significant worsening (e.g., no more than 5% worsening) of atrophy and/or diffusivity compared to the baseline neuroimaging assessment.

275. The method of any one of claims 260-274, wherein the treatment is effective if the subsequent concentration of frataxin is at least 3× greater than the baseline concentration of frataxin, the subsequent concentration of neurofilament light chain is lower than or equal to the baseline concentration of neurofilament light chain, and the subsequent neuroimaging assessment shows no significant worsening (e.g., no more than 5% worsening) of atrophy and/or diffusivity compared to the baseline neuroimaging assessment.

276. The method of any one of claims 246-275, wherein the treatment comprises a frataxin gene therapy.

277. A method of treating Friedreich's Ataxia in a subject, comprising the steps of:
a) determining a baseline concentration of frataxin in a first sample of serum from the subject;
b) treating the subject with a frataxin gene therapy;
c) determining a subsequent concentration of frataxin in a second sample of serum from the subject; and
d) comparing the baseline concentration of frataxin to the subsequent concentration of frataxin;
wherein the frataxin gene therapy is effective if the subsequent concentration of frataxin is greater than the baseline concentration of frataxin.

278. The method of claim 277, wherein the baseline concentration of frataxin is below 10 pg/mL, below 5 pg/mL, below 2 pg/mL, or less.

279. The method of claim 277 or claim 278, wherein the baseline concentration of frataxin is below 1 pg/mL.

280. A method of treating Friedreich's Ataxia in a subject, comprising the steps of:
a) determining a baseline concentration of frataxin in a first sample of CSF from the subject;
treating the subject with a frataxin gene therapy;
c) determining a subsequent concentration of frataxin in a second sample of CSF from the subject; and
d) comparing the baseline concentration of frataxin to the subsequent concentration of frataxin;
wherein the frataxin gene therapy is effective if the subsequent concentration of frataxin is greater than the baseline concentration of frataxin.

281. The method of claim 280, wherein the baseline concentration of frataxin is below 5 pg/mL, below 2 pg/mL, or less.

282. The method of claim 270 or claim 281, wherein the baseline concentration of frataxin is below 1 pg/mL.

283. A method of treating Friedreich's Ataxia in a subject, comprising the steps of:
a) determining a baseline concentration of frataxin in a first sample of serum from the subject and determining a baseline concentration of neurofilament light chain in a second sample of serum from the subject, wherein the first sample and the second sample are optionally the same sample;
b) treating the subject with a frataxin gene therapy;
c) determining a subsequent concentration of frataxin in a third sample of serum from the subject and determining a subsequent concentration of neurofilament light chain in a fourth sample of serum from the subject, wherein the third sample and the fourth sample are optionally the same sample; and
d) comparing the baseline concentration of frataxin to the subsequent concentration of frataxin and comparing the baseline concentration of neurofilament light chain to the subsequent concentration of neurofilament light chain;
wherein the frataxin gene therapy is effective if the subsequent concentration of frataxin is greater than the baseline concentration and the subsequent concentration of neurofilament light chain is lower than or equal to the baseline concentration of neurofilament light chain.

284. The method of claim 283, wherein the baseline concentration of frataxin is below 10 pg/mL, below 5 pg/mL, below 2 pg/mL, or less, and wherein baseline concentration of neurofilament light chain is at least 20 pg/mL.

285. The method of claim 284, wherein the baseline concentration of frataxin is below 1 pg/mL.

286. A method of treating Friedreich's Ataxia in a subject, comprising the steps of:
a) determining a baseline concentration of frataxin in a first sample of CSF from the subject and determining a baseline concentration of neurofilament light chain in a second sample of CSF from the subject, wherein the first sample and the second sample are optionally the same sample;
b) treating the subject with a frataxin gene therapy;
c) determining a subsequent concentration of frataxin in a third sample of CSF from the subject and determining a subsequent concentration of neurofilament light chain in a fourth sample of CSF from the subject, wherein the third sample and the fourth sample are optionally the same sample; and
d) comparing the baseline concentration of frataxin to the subsequent concentration of frataxin and comparing the baseline concentration of neurofilament light chain to the subsequent concentration of neurofilament light chain;
wherein the frataxin gene therapy is effective if the subsequent concentration of frataxin is greater than the baseline concentration and the subsequent concentration of neurofilament light chain is lower than or equal to the baseline concentration of neurofilament light chain.

287. The method of claim 286, wherein the baseline concentration of frataxin is below 5 pg/mL, below 2 pg/mL, or less, and wherein the baseline concentration of neurofilament light chain is at least 1200 pg/mL.

288. The method of claim 287, wherein the baseline concentration of frataxin is below 1 pg/mL.

289. The method of any one of claims 277-288, further comprising the steps of:
a) performing a baseline neuroimaging assessment on the subject prior to treating the subject with the frataxin gene therapy;
b) performing a subsequent neuroimaging assessment on the subject after treating the subject with the frataxin gene therapy;
c) comparing the baseline neuroimaging assessment to the subsequent neuroimaging assessment;
wherein the frataxin gene therapy is effective if the subsequent neuroimaging assessment shows no significant worsening (e.g., no more than 5% worsening) of atrophy and/or diffusivity compared to the baseline neuroimaging assessment.

290. The method of any one of claims 277-288, wherein if the frataxin gene therapy is not effective, the subject is administered a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

291. The method of any one of claims 277-288, wherein if the frataxin gene therapy is not effective, the subject is administered a high dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

292. The method of any one of claims 277-288, wherein if the frataxin gene therapy is effective, the subject is administered a low dose of a second therapy capable of reducing oxidative stress, reducing mitochondrial dysfunction, restoring mitochondrial function, reducing inflammation, and/or resolving inflammation, and optionally managing one or more symptoms of Friedreich's Ataxia using occupational therapy, speech therapy, orthopedic care, bracing, surgery, treatment for heart disease, an antioxidant, treatment to lower blood sugar, and/or physical therapy.

293. The method of any one of claims 277-288, wherein if the frataxin gene therapy is effective, the subject is administered a treatment comprising managing one or more symptoms of Friedreich's Ataxia comprising occupational therapy, speech therapy, orthopedic care, bracing, surgery, treatment for heart disease, an antioxidant, treatment to lower blood sugar, and/or physical therapy.

294. The method of any one of claims 290-292, wherein the second therapy comprises omaveloxolone, alpha-tocotrienol quinone, a polyunsaturated fatty acid mimetic (e.g., deuterated linoleic acid ethyl ester), (+)-epicatechin, methylprednisone, a D-amino acid oxidase inhibitor, a peroxisome-proliferator activator receptor (PPAR) gamma agonist or ligand, dimethyl fumarate, a carrier protein to deliver frataxin protein to mitochondria (e.g., Trans-Activator of Transcription (TAT)), a small molecule modulator of a cytokine receptor (e.g., an activator of the tissue-protective erythropoietin receptor), a ubiquitin competitor (e.g., an inhibitor of RNF126), etravirine, resveratrol, nicotinamide, interferon gamma, a histone deacetylase (HDAC) inhibitor, a chromatin modulation therapy, a therapy that degrades non-coding RNA responsible for directing localized epigenetic silencing of the frataxin gene, granulocyte colony stimulating factor, gene therapy to treat cardiac disease associated with Friedreich's Ataxia), acetyl-L-carnitine (ALCAR), rosuvastatin, an incretin analog, indole-3-propionic acid, and/or erythropoietin or a modified (e.g., carbamylated) form thereof.

295. The method of any one of claims 290-292, wherein the second therapy comprises omaveloxolone, wherein the low dose of omaveloxolone is about 5 mg/day, and wherein the high dose of omaveloxolone is about 300 mg/day.

296. The method of any one of claims 277-295, wherein determining a baseline or subsequent concentration of frataxin comprises
i) incubating a substrate with a capture agent;
ii) incubating the substrate with a sample and a detection agent;
iii) incubating the substrate with a substance capable of engaging with the detection agent and creating a signal, wherein the signal is detectable and quantifiable.

297. The method of claim 296, wherein the capture agent comprises an antibody selected from the group consisting of Ab-1, Ab-2, Ab-3, Ab-4, Ab-5, and Ab-6; and the detection agent comprises an antibody selected from the group consisting of Ab-1, Ab-2, Ab-3, Ab-4, Ab-5, and Ab-6.

298. The method of claim 297, wherein the capture agent is Ab-1 and the detection agent is Ab-3.

299. The method of any one of claims 296-298, wherein the substrate comprises assay beads and helper beads.

300. The method of claim 299, wherein the assay comprises a greater number of helper beads than assay beads.

301. The method of claim 300, wherein a ratio of helper beads to assay beads is about 1:5 to about 5:1, optionally about 1:1 to about 2:1, e.g., about 1:1, about 1.5:1, or about 2:1.

302. The method of any one of claims 297-301, wherein the incubating step of ii) occurs for about 10-150 minutes and the incubating step of iii) occurs for about 1-30 minutes.

303. The method of any one of claims 297-302, wherein the incubating step of ii) occurs for about 35 minutes and the incubating step of iii) occurs for about 5 minutes, wherein the incubating step of ii) occurs for about 75 minutes and the incubating step of iii) occurs for about 5 minutes, wherein the incubating step of ii) occurs for about 120 minutes and the incubating step of iii) occurs for about 30 minutes, or wherein the incubating step of ii) occurs for about 120 minutes and the incubating step of iii) occurs for about 5 minutes.

304. The method of claim 303, wherein the incubating step of ii) occurs for about 120 minutes and the incubating step of iii) occurs for about 5 minutes.

305. The method of any one of claims 233-288, wherein the treatment comprises a nucleic acid construct comprising a fragment of any of SEQ ID NOs: 1-3, optionally nucleotides 32-664 of SEQ ID NO: 1.

306. The method of any one of claims 233-288, wherein the treatment comprises a nucleic acid construct encoding any of SEQ ID NOs: 4-6, optionally SEQ ID NO: 4.

307. The method of any one of claims 233-288, wherein the treatment comprises a nucleic acid construct comprising any of SEQ ID NOs: 7-10.

308. The method of any one of claims 233-288, wherein the treatment comprises a nucleic acid construct comprising any of SEQ ID NOs: 11-14.

309. The method of any one of claims 233-288, wherein the treatment comprises an AAV comprising an AAV capsid and a viral genome, wherein the viral genome comprises a nucleic acid construct encoding any of SEQ ID NOs 4-6, optionally SEQ ID NO: 4.

310. The method of claim 309, wherein the nucleic acid construct comprises any of SEQ ID NOs: 7-10.

311. The method of claim 309 or claim 310, wherein the nucleic acid construct comprises any of SEQ ID NOs: 11-14.

312. The method of any one of claims 233-288, wherein the treatment comprises an AAV capsid comprising a sequence that is at least 99% identical to SEQ ID NO: 15 and/or encoded by SEQ ID NO: 16.

313. The method of any one of claims 233-288, wherein the treatment is administered in a formulation comprising sodium chloride, sodium phosphate, potassium chloride, potassium phosphate and poloxamer 188; wherein optionally the therapy or treatment is formulated to comprise 192 mM sodium chloride, 10 mM sodium phosphate, 2.7 mM potassium chloride, 2 mM potassium phosphate and 0.001% poloxamer 188 (v/v), wherein the pH of the formulation is 7.4.

314. A frataxin gene therapy for use in the treatment of Friedreich's Ataxia in a subject, wherein the treatment comprises administering the frataxin gene therapy to a subject lacking detectable frataxin in a biofluid sample at a limit of detection of frataxin of about 2.0 pg/mL or less, wherein the amount of frataxin is determined using an assay.

315. A frataxin gene therapy for use in the treatment of Friedreich's Ataxia in a subject, wherein the treatment comprises the steps of:
a) determining whether a subject has frataxin in a biofluid sample using an assay having a limit of detection of frataxin of about 2.0 pg/mL or less; and
b) administering the frataxin gene therapy to a subject determined as having frataxin in the biofluid sample at a concentration at just above, below, or equal to the limit of detection of frataxin of the assay.

316. A frataxin gene therapy for use in the treatment of Friedreich's Ataxia in a subject, wherein the treatment comprises the steps of:
a) determining a baseline concentration of frataxin in a first sample of a biofluid of a subject using an assay having a limit of detection of frataxin of about 2.0 pg/mL or less, wherein the biofluid is optionally cerebrospinal fluid (CSF) or serum;
b) treating the subject with the frataxin gene therapy;
c) determining a subsequent concentration of frataxin in a second sample of the biofluid of the subject using an assay having a limit of detection of frataxin of about 2.0 pg/mL or less, wherein the biofluid of c) is CSF if the biofluid of a) is CSF, and wherein the biofluid of c) is serum if the biofluid of a) is serum; and
d) comparing the baseline concentration of frataxin to the subsequent concentration of frataxin;
wherein the frataxin gene therapy is effective if the subsequent concentration of frataxin is greater than the baseline concentration of frataxin.

317. A frataxin gene therapy for use in the treatment of Friedreich's Ataxia in a subject, wherein the treatment comprises the steps of:
a) determining a baseline concentration of frataxin in a first sample of a biofluid of a subject and determining a baseline concentration of neurofilament light chain in a second sample of a biofluid of the subject, wherein the biofluid is optionally cerebrospinal fluid (CSF) or serum and wherein the first sample and the second sample are optionally the same sample;
b) treating the subject with the frataxin gene therapy;
c) determining a subsequent concentration of frataxin in a third sample of a biofluid of the subject and determining a subsequent concentration of neurofilament light chain in a fourth sample of a biofluid in the subject, wherein the biofluid of c) is CSF if the biofluid of a) is CSF, and wherein the biofluid of c) is serum if the biofluid of a) is serum, and wherein the third sample and the fourth sample are optionally the same sample; and
d) comparing the baseline concentration of frataxin to the subsequent concentration of frataxin and comparing the baseline concentration of neurofilament light chain to the subsequent concentration of neurofilament light chain;
wherein the frataxin gene therapy is effective if the subsequent concentration of frataxin is greater than the baseline concentration and the subsequent concentration of neurofilament light chain is not significantly higher than, is lower than, or is equal to the baseline concentration of neurofilament light chain.

318. A frataxin gene therapy for use in the treatment of Friedreich's Ataxia in a subject, wherein the treatment comprises the steps of:
a) determining a baseline concentration of frataxin in a first sample of a biofluid of a subject and determining a baseline concentration of neurofilament light chain in a second sample of a biofluid of the subject, wherein the biofluid is optionally cerebrospinal fluid (CSF) or serum and wherein the first sample and the second sample are optionally the same sample;
b) performing a baseline neuroimaging assessment on the subject;
c) treating the subject with the frataxin gene therapy;
d) determining a subsequent concentration of frataxin in a third sample of a biofluid of the subject and determining a subsequent concentration of neurofilament light chain in a fourth sample of a biofluid in the subject, wherein the biofluid of d) is CSF if the biofluid of a) is CSF, and wherein the biofluid of d) is serum if the biofluid of a) is serum, and wherein the third sample and the fourth sample are optionally the same sample;
e) performing a subsequent neuroimaging assessment on the subject; and
f) comparing the baseline concentration of frataxin to the subsequent concentration of frataxin, comparing the baseline concentration of neurofilament light chain to the subsequent concentration of neurofilament light chain, and comparing the baseline neuroimaging assessment to the subsequent neuroimaging assessment;
wherein the frataxin gene therapy is effective if the subsequent concentration of frataxin is greater than the baseline concentration, the subsequent concentration of neurofilament light chain is not significantly higher than, is lower than, or is equal to the baseline concentration of neurofilament light chain, and the subsequent neuroimaging assessment shows no significant worsening (e.g., no more than 5% worsening) of atrophy and/or diffusivity compared to the baseline neuroimaging assessment.

319. A frataxin gene therapy for use in the treatment of Friedreich's Ataxia in a subject, wherein the treatment comprises the steps of:
a) determining the concentration of frataxin in a serum sample from the subject using an assay having a limit of detection of frataxin of about 2.0 pg/mL or less; wherein the assay comprises a capture agent that specifically binds to frataxin or a fragment of frataxin, and a detection agent that specifically binds to frataxin or a fragment of frataxin; and
b) administering the frataxin gene therapy to the subject if the concentration of frataxin protein present in the serum sample is below 10 pg/mL.

320. A frataxin gene therapy for use in the treatment of Friedreich's Ataxia in a subject, wherein the treatment comprises the steps of:
a) determining the concentration of frataxin in a CSF sample from the subject using an assay having a limit of detection of frataxin of about 2.0 pg/mL or less; wherein the assay comprises a capture agent that specifically binds to frataxin or a fragment of frataxin, and a detection agent that specifically binds to frataxin or a fragment of frataxin; and
b) administering the frataxin gene therapy to the subject if the concentration of frataxin protein present in the CSF sample is below 5 pg/mL.
